SEQUENCE LISTING (189 sequences)

SEQ ID NO: 1 - protein, 201 aa, synthetic construct
  CARBOHYD 33: note = N is linked to the N-linked glycan structure Hex(6)HexNAc(5)Fuc(0)NeuAc(1)
  CARBOHYD 33: note = N is linked to the N-linked glycan structure Hex(6)HexNAc(5)Fuc(0)NeuAc(2)
  CARBOHYD 93: note = N is linked to the N-linked glycan structure Hex(7)HexNAc(6)Fuc(0)NeuAc(4)
MALSWVLTVLSLLPLLEAQIPLCANLVPVPITNATLDQITGKWFYIASAFRNEEYNKSVQEIQATFFYFTPNKTEDTIFLREYQTRQDQCIYNTTYLNVQRENGTISRYVGGQEHFAHLLILRDTKTYMLAFDVNDEKNWGLSVYADKPETTKEQLGEFYEALDCLRIPKSDVVYTDWKKDKCEPLEKQHEKERKQEEGES

SEQ ID NO: 2 - protein, 99 aa, synthetic construct
  CARBOHYD 74: note = W is linked to the O-linked glycan structure Hex(1)HexNAc(1)Fuc(0)NeuAc(2)
  CARBOHYD 74: note = W is linked to the O-linked glycan structure Hex(1)HexNAc(1)Fuc(1)NeuAc(1)
MQPRVLLVVALLALLASARASEAEDASLLSFMQGYMKHATKTAKDALSSVQESQVAQQARGWVTDGFSSLKDYWSTVKDKFSEFWDLDPEVRPTSAVAA

SEQ ID NO: 3 - protein, 119 aa, synthetic construct
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 4 - protein, 330 aa, synthetic construct [sequence omitted]

SEQ ID NO: 5 - protein, 42 aa, synthetic construct
  CARBOHYD 33: note = N is linked to the N-linked glycan structure Hex(6)HexNAc(5)Fuc(0)NeuAc(1)
MALSWVLTVLSLLPLLEAQIPLCANLVPVPITNATLDQITGK

SEQ ID NO: 6 - protein, 42 aa, synthetic construct; identical to SEQ ID NO: 5
  CARBOHYD 33: note = N is linked to the N-linked glycan structure Hex(6)HexNAc(5)Fuc(0)NeuAc(2)

SEQ ID NO: 7 - protein, 15 aa, synthetic construct
  CARBOHYD 7: note = N is linked to the N-linked glycan structure Hex(7)HexNAc(6)Fuc(0)NeuAc(4)
QDQCIYNTTYLNVQR

SEQ ID NO: 8 - protein, 19 aa, synthetic construct
  CARBOHYD 14: note = T is linked to the O-linked glycan structure Hex(1)HexNAc(1)Fuc(0)NeuAc(2)
FSEFWDLDPEVRPTSAVAA

SEQ ID NO: 9 - protein, 10 aa, synthetic construct
VNHVTLSQPK

SEQ ID NO: 10 - protein, 7 aa, synthetic construct
DYWSTVK

SEQ ID NO: 11 - protein, 7 aa, synthetic construct
DTLMISR

SEQ ID NO: 12 - protein, 11 aa, synthetic construct
GWVTDGFSSLK

SEQ ID NO: 13 - protein, 99 aa, synthetic construct; identical to SEQ ID NO: 2
  CARBOHYD 74: note = W is linked to the O-linked glycan structure Hex(1)HexNAc(1)Fuc(1)NeuAc(1)
  CARBOHYD 74: note = W is linked to the O-linked glycan structure Hex(1)HexNAc(1)Fuc(0)NeuAc(2)

SEQ ID NO: 14 - protein, 367 aa, synthetic construct
  CARBOHYD 156: note = N is linked to the N-linked glycan structure Hex(5)HexNAc(4)Fuc(0)NeuAc(1)
  CARBOHYD 156: note = N is linked to the N-linked glycan structure Hex(6)HexNAc(5)Fuc(0)NeuAc(2)
  CARBOHYD 156: note = N is linked to the N-linked glycan structure Hex(6)HexNAc(4)Fuc(0)NeuAc(1)
MKSLVLLLCLAQLWGCHSAPHGPGLIYRQPNCDDPETEEAALVAIDYINQNLPWGYKHTLNQIDEVKVWPQQPSGELFEIEIDTLETTCHVLDPTPVARCSVRQLKEHAVEGDCDFQLLKLDGKFSVVYAKCDSSPDSAEDVRKVCQDCPLLAPLNDTRVVHAAKAALAAFNAQNNGSNFQLEEISRAQLVPLPPSTYVEFTVSGTDCVAKEATEAAKCNLLAEKQYGFCKATLSEKLGGAEVAVTCMVFQTQPVSSQPQPEGANEAVPTPVVDPDAPPSPPLGAPGLPPAGSPPDSHVLLAAPPGHQLHRAHYDLRHTFMGVVSLGSPSGEVSHPRKTRTVVQPSVGAAAGPVVPPCPGRIRHFKV

SEQ ID NO: 15 - protein, 402 aa, synthetic construct
  CARBOHYD 396: note = N is linked to the N-linked glycan structure Hex(5)HexNAc(4)Fuc(0)NeuAc(2)
MESRGPLATSRLLLLLLLLLLRHTRQGWALRPVLPTQSAHDPPAVHLSNGPGQEPIAVMTFDLTKITKTSSSFEVRTWDPEGVIFYGDTNPKDDWFMLGLRDGRPEIQLHNHWAQLTVGAGPRLDDGRWHQVEVKMEGDSVLLEVDGEEVLRLRQVSGPLTSKRHPIMRIALGGLLFPASNLRLPLVPALDGCLRRDSWLDKQAEISASAPTSLRSCDVESNPGIFLPPGTQAEFNLRDIPQPHAEPWAFSLDLGLKQAAGSGHLLALGTPENPSWLSLHLQDQKVVLSSGSGPGLDLPLVLGLPLQLKLSMSRVVLSQGSKMKALALPPLGLAPLLNLWAKPQGRLFLGALPGEDSSTSFCLNGLWAQGQRLDVDQALNRSHEIWTHSCPQSPGNGTDASH

SEQ ID NO: 16 - protein, 19 aa, synthetic construct
  CARBOHYD 14: note = T is linked to the O-linked glycan structure Hex(1)HexNAc(1)Fuc(1)NeuAc(1)
FSEFWDLDPEVRPTSAVAA

SEQ ID NO: 17 - protein, 22 aa, synthetic construct
  CARBOHYD 11: note = N is linked to the N-linked glycan structure Hex(5)HexNAc(4)Fuc(0)NeuAc(1)
AALAAFNAQNNGSNFQLEEISR

SEQ ID NO: 18 - protein, 22 aa, synthetic construct
  CARBOHYD 11: note = N is linked to the N-linked glycan structure Hex(6)HexNAc(5)Fuc(0)NeuAc(2)
AALAAFNAQNNGSNFQLEEISR

SEQ ID NO: 19 - protein, 22 aa, synthetic construct
  CARBOHYD 11: note = N is linked to the N-linked glycan structure Hex(6)HexNAc(4)Fuc(0)NeuAc(1)
AALAAFNAQNNGSNFQLEEISR

SEQ ID NO: 20 - protein, 14 aa, synthetic construct
IALGGLLFPASNLR

SEQ ID NO: 21 - protein, 9 aa, synthetic construct
DDWFMLGLR

SEQ ID NO: 22 - protein, 201 aa, synthetic construct
  CARBOHYD 103: note = N is linked to the N-linked glycan structure Hex(5)HexNAc(4)Fuc(0)NeuAc(2)
  CARBOHYD 56: note = N is linked to the N-linked glycan structure Hex(5)HexNAc(4)Fuc(1)NeuAc(2
  CARBOHYD 72: note = N is linked to the N-linked glycan structure Hex(5)HexNAc(4)Fuc(0)NeuAc(2)
  CARBOHYD 72: note = N is linked to the N-linked glycan structure Hex(6)HexNAc(5)Fuc(0)NeuAc(1)
  CARBOHYD 72: note = N is linked to the N-linked glycan structure Hex(6)HexNAc(5)Fuc(0)NeuAc(2)
MALSWVLTVLSLLPLLEAQIPLCANLVPVPITNATLDRITGKWFYIASAFRNEEYNKSVQEIQATFFYFTPNKTEDTIFLREYQTRQDQCIYNTTYLNVQRENGTISRYVGGQEHFAHLLILRDTKTYMLAFDVNDEKNWGLSVYADKPETTKEQLGEFYEALDCLRIPKSDVVYTDWKKDKCEPLEKQHEKERKQEEGES

SEQ ID NO: 23 - protein, 418 aa, synthetic construct
  CARBOHYD 271: note = N is linked to the N-linked glycan structure Hex(4)HexNAc(3)Fuc(0)NeuAc(0)
  CARBOHYD 271: note = N is linked to the N-linked glycan structure Hex(4)HexNAc(3)Fuc(0)NeuAc(1)
  CARBOHYD 271: note = N is linked to the N-linked glycan structure Hex(6)HexNAc(5)Fuc(0)NeuAc(2)
  CARBOHYD 70: note = N is linked to the N-linked glycan structure Hex(4)HexNAc(3)Fuc(0)NeuAc(1)
  CARBOHYD 70: note = N is linked to the N-linked glycan structure Hex(6)HexNAc(5)Fuc(0)NeuAc(2)
  CARBOHYD 271: note = N is linked to the N-linked glycan structure Hex(5)HexNAc(4)Fuc(0)NeuAc(1)
  CARBOHYD 271: note = N is linked to the N-linked glycan structure Hex(5)HexNAc(4)Fuc(0)NeuAc(2)
  CARBOHYD 271: note = N is linked to the N-linked glycan structure Hex(5)HexNAc(4)Fuc(1)NeuAc(2)
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 24 - protein, 1474 aa, synthetic construct
  CARBOHYD 1424: note = N is linked to the N-linked glycan structure Hex(5)HexNAc(4)Fuc(0)NeuAc(0)
  CARBOHYD 869: note = N is linked to the N-linked glycan structure Hex(4)HexNAc(4)Fuc(0)NeuAc(1)
  CARBOHYD 869: note = N is linked to the N-linked glycan structure Hex(5)HexNAc(4)Fuc(0)NeuAc(0)
  CARBOHYD 1424: note = N is linked to the N-linked glycan structure Hex(5)HexNAc(4)Fuc(1)NeuAc(2)
  CARBOHYD 247: note = N is linked to the N-linked glycan structure Hex(5)HexNAc(4)Fuc(0)NeuAc(1)
  CARBOHYD 247: note = N is linked to the N-linked glycan structure Hex(5)HexNAc(4)Fuc(0)NeuAc(2)
  CARBOHYD 869: note = N is linked to the N-linked glycan structure Hex(5)HexNAc(4)Fuc(0)NeuAc(1)
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDNEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEIKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 25 - protein, 423 aa, synthetic construct
  CARBOHYD 106: note = N is linked to the N-linked glycan structure Hex(6)HexNAc(5)Fuc(0)NeuAc(2)
  CARBOHYD 93: note = N is linked to the N-linked glycan structure Hex(5)HexNAc(4)Fuc(0)NeuAc(2)
  CARBOHYD 271: note = N is linked to the N-linked glycan structure Hex(6)HexNAc(5)Fuc(0)NeuAc(2)
MERMLPLLALGLLAAGFCPAVLCHPNSPLDEENLTQENQDRGTHVDLGLASANVDFAFSLYKQLVLKAPDKNVIFSPLSISTALAFLSLGAHNTTLTEILKGLKFNLTETSEAEIHQSFQHLLRTLNQSSDELQLSMGNAMFVKEQLSLLDRFTEDAKRLYGSEAFATDFQDSAAAKKLINDYVKNGTRGKITDLIKDLDSQTMMVLVNYIFFKAKWEMPFDPQDTHQSRFYLSKKKWVMVPMMSLHHLTIPYFRDEELSCTVVELKYTGNASALFILPDQDKMEEVEAMLLPETLKRWRDSLEFREIGELYLPKFSISRDYNLNDILLQLGIEEAFTSKADLSGITGARNLAVSQVVHKAVLDVFEEGTEASAATAVKITLLSALVETRTIVRFNRPFLMIIVPTDTQNIFFMSKVTNPKQA

SEQ ID NO: 26 - protein, 352 aa, synthetic construct
  CARBOHYD 250: note = N is linked to the N-linked glycan structure Hex(5)HexNAc(4)Fuc(0)NeuAc(2)
MRSLGALLLLLSACLAVSAGPVPTPPDNIQVQENFNISRIYGKWYNLAIGSTCPWLKKIMDRMTVSTLVLGEGATEAEISMTSTRWRKGVCEETSGAYEKTDTDGKFLYHKSKWNITMESYVVHTNYDEYAIFLTKKFSRHHGPTITAKLYGRAPQLRETLLQDFRVVAQGVGIPEDSIFTMADRGECVPGEQEPEPILIPRVRRAVLPQEEEGSGGGQLVTEVTKKEDSCQLGYSAGPCMGMTSRYFYNGTSMACETFQYGGCMGNGNNFVTEKECLQTCRTVAACNLPIVRGPCRAFIQLWAFDAVKGKCVLFPYGGCQGNGNKFYSEKECREYCGVPGDGDEELLRFSN

SEQ ID NO: 27 - protein, 476 aa, synthetic construct
  CARBOHYD 161: note = N is linked to the N-linked glycan structure Hex(5)HexNAc(4)Fuc(0)NeuAc(2)
  CARBOHYD 38: note = N is linked to the N-linked glycan structure Hex(6)HexNAc(5)Fuc(0)NeuAc(2)
MAPAGVSLRATILCLLAWAGLAAGDRVYIHPFHLVIHNESTCEQLAKANAGKPKDPTFIPAPIQAKTSPVDEKALQDQLVLVAAKLDTEDKLRAAMVGMLANFLGFRIYGMHSELWGVVHGATVLSPTAVFGTLASLYLGALDHTADRLQAILGVPWKDKNCTSRLDAHKVLSALQAVQGLLVAQGRADSQAQLLLSTVVGVFTAPGLHLKQPFVQGLALYTPVVLPRSLDFTELDVAAEKIDRFMQAVTGWKTGCSLMGASVDSTLAFNTYVHFQGKMKGFSLLAEPQEFWVDNSTSVSVPMLSGMGTFQHWSDIQDNFSVTQVPFTESACLLLIQPHYASDLDKVEGLTFQQNSLNWMKKLSPRTIHLTMPQLVLQGSYDLQDLLAQAELPAILHTELNLQKLSNDRIRVGEVLNSIFFELEADEREPTESTQQLNKPEVLEVTLNRPFLFAVYDQSATALHFLGRVANPLSTA

SEQ ID NO: 28 - protein, 464 aa, synthetic construct
  CARBOHYD 128: note = N is linked to the N-linked glycan structure Hex(5)HexNAc(4)Fuc(0)NeuAc(1)
  CARBOHYD 128: note = N is linked to the N-linked glycan structure Hex(5)HexNAc(4)Fuc(0)NeuAc(2)
MYSNVIGTVTSGKRKVYLLSLLLIGFWDCVTCHGSPVDICTAKPRDIPMNPMCIYRSPEKKATEDEGSEQKIPEATNRRVWELSKANSRFATTFYQHLADSKNDNDNIFLSPLSISTAFAMTKLGACNDTLQQLMEVFKFDTISEKTSDQIHFFFAKLNCRLYRKANKSSKLVSANRLFGDKSLTFNETYQDISELVYGAKLQPLDFKENAEQSRAAINKWVSNKTEGRITDVIPSEAINELTVLVLVNTIYFKGLWKSKFSPENTRKELFYKADGESCSASMMYQEGKFRYRRVAEGTQVLELPFKGDDITMVLILPKPEKSLAKVEKELTPEVLQEWLDELEEMMLVVHMPRFRIEDGFSLKEQLQDMGLVDLFSPEKSKLPGIVAEGRDDLYVSDAFHKAFLEVNEEGSEAAASTAVVIAGRSLNPNRVTFKANRPFLVFIREVPLNTIIFMGRVANPCVK

SEQ ID NO: 29 - protein, 423 aa, synthetic construct
  CARBOHYD 76: note = N is linked to the N-linked glycan structure Hex(5)HexNAc(4)Fuc(0)NeuAc(1)
MSASAVFILDVKGKPLISRNYKGDVAMSKIEHFMPLLVQREEEGALAPLLSHGQVHFLWIKHSNLYLVATTSKNANASLVYSFLYKTIEVFCEYFKELEEESIRDNFVIVYELLDELMDFGFPQTTDSKILQEYITQQSNKLETGKSRVPPTVTNAVSWRSEGIKYKKNEVFIDVIESVNLLVNANGSVLLSEIVGTIKLKVFLSGMPELRLGLNDRVLFELTGRSKNKSVELEDVKFHQCVRLSRFDNDRTISFIPPDGDFELMSYRLSTQVKPLIWIESVIEKFSHSRVEIMVKAKGQFKKQSVANGVEISVPVPSDADSPRFKTSVGSAKYVPERNVVIWSIKSFPGGKEYLMRAHFGLPSVEKEEVEGRPPIGVKFEIPYFTVSGIQVRYMKIIEKSGYQALPWVRYITQSGDYQLRTS

SEQ ID NO: 30 - protein, 4563 aa, synthetic construct
  CARBOHYD 3224: note = N is linked to the N-linked glycan structure Hex(5)HexNAc(4)Fuc(0)NeuAc(1)
  CARBOHYD 983: note = N is linked to the N-linked glycan structure Hex(5)HexNAc(4)Fuc(1)NeuAc(0)
MDPPRPALLALLALPALLLLLLAGARAEEEMLENVSLVCPKDATRFKHLRKYTYNYEAESSSGVPGTADSRSATRINCKVELEVPQLCSFILKTSQCTLKEVYGFNPEGKALLKKTKNSEEFAAAMSRYELKLAIPEGKQVFLYPEKDEPTYILNIKRGIISALLVPPETEEAKQVLFLDTVYGNCSTHFTVKTRKGNVATEISTERDLGQCDRFKPIRTGISPLALIKGMTRPLSTLISSSQSCQYTLDAKRKHVAEAICKEQHLFLPFSYKNKYGMVAQVTQTLKLEDTPKINSRFFGEGTKKMGLAFESTKSTSPPKQAEAVLKTLQELKKLTISEQNIQRANLFNKLVTELRGLSDEAVTSLLPQLIEVSSPITLQALVQCGQPQCSTHILQWLKRVHANPLLIDVVTYLVALIPEPSAQQLREIFNMARDQRSRATLYALSHAVNNYHKTNPTGTQELLDIANYLMEQIQDDCTGDEDYTYLILRVIGNMGQTMEQLTPELKSSILKCVQSTKPSLMIQKAAIQALRKMEPKDKDQEVLLQTFLDDASPGDKRLAAYLMLMRSPSQADINKIVQILPWEQNEQVKNFVASHIANILNSEELDIQDLKKLVKEALKESQLPTVMDFRKFSRNYQLYKSVSLPSLDPASAKIEGNLIFDPNNYLPKESMLKTTLTAFGFASADLIEIGLEGKGFEPTLEALFGKQGFFPDSVNKALYWVNGQVPDGVSKVLVDHFGYTKDDKHEQDMVNGIMLSVEKLIKDLKSKEVPEARAYLRILGEELGFASLHDLQLLGKLLLMGARTLQGIPQMIGEVIRKGSKNDFFLHYIFMENAFELPTGAGLQLQISSSGVIAPGAKAGVKLEVANMQAELVAKPSVSVEFVTNMGIIIPDFARSGVQMNTNFFHESGLEAHVALKAGKLKFIIPSPKRPVKLLSGGNTLHLVSTTKTEVIPPLIENRQSWSVCKQVFPGLNYCTSGAYSNASSTDSASYYPLTGDTRLELELRPTGEIEQYSVSATYELQREDRALVDTLKFVTQAEGAKQTEATMTFKYNRQSMTLSSEVQIPDFDVDLGTILRVNDESTEGKTSYRLTLDIQNKKITEVALMGHLSCDTKEERKIKGVISIPRLQAEARSEILAHWSPAKLLLQMDSSATAYGSTVSKRVAWHYDEEKIEFEWNTGTNVDTKKMTSNFPVDLSDYPKSLHMYANRLLDHRVPQTDMTFRHVGSKLIVAMSSWLQKASGSLPYTQTLQDHLNSLKEFNLQNMGLPDFHIPENLFLKSDGRVKYTLNKNSLKIEIPLPFGGKSSRDLKMLETVRTPALHFKSVGFHLPSREFQVPTFTIPKLYQLQVPLLGVLDLSTNVYSNLYNWSASYSGGNTSTDHFSLRARYHMKADSVVDLLSYNVQGSGETTYDHKNTFTLSYDGSLRHKFLDSNIKFSHVEKLGNNPVSKGLLIFDASSSWGPQMSASVHLDSKKKQHLFVKEVKIDGQFRVSSFYAKGTYGLSCQRDPNTGRLNGESNLRFNSSYLQGTNQITGRYEDGTLSLTSTSDLQSGIIKNTASLKYENYELTLKSDTNGKYKNFATSNKMDMTFSKQNALLRSEYQADYESLRFFSLLSGSLNSHGLELNADILGTDKINSGAHKATLRIGQDGISTSATTNLKCSLLVLENELNAELGLSGASMKLTTNGRFREHNAKFSLDGKAALTELSLGSAYQAMILGVDSKNIFNFKVSQEGLKLSNDMMGSYAEMKFDHTNSLNIAGLSLDFSSKLDNIYSSDKFYKQTVNLQLQPYSLVTTLNSDLKYNALDLTNNGKLRLEPLKLHVAGNLKGAYQNNEIKHIYAISSAALSASYKADTVAKVQGVEFSHRLNTDIAGLASAIDMSTNYNSDSLHFSNVFRSVMAPFTMTIDAHTNGNGKLALWGEHTGQLYSKFLLKAEPLAFTFSHDYKGSTSHHLVSRKSISAALEHKVSALLTPAEQTGTWKLKTQFNNNEYSQDLDAYNTKDKIGVELTGRTLADLTLLDSPIKVPLLLSEPINIIDALEMRDAVEKPQEFTIVAFVKYDKNQDVHSINLPFFETLQEYFERNRQTIIVVLENVQRNLKHINIDQFVRKYRAALGKLPQQANDYLNSFNWERQVSHAKEKLTALTKKYRITENDIQIALDDAKINFNEKLSQLQTYMIQFDQYIKDSYDLHDLKIAIANIIDEIIEKLKSLDEHYHIRVNLVKTIHDLHLFIENIDFNKSGSSTASWIQNVDTKYQIRIQIQEKLQQLKRHIQNIDIQHLAGKLKQHIEAIDVRVLLDQLGTTISFERINDILEHVKHFVINLIGDFEVAEKINAFRAKVHELIERYEVDQQIQVLMDKLVELAHQYKLKETIQKLSNVLQQVKIKDYFEKLVGFIDDAVKKLNELSFKTFIEDVNKFLDMLIKKLKSFDYHQFVDETNDKIREVTQRLNGEIQALELPQKAEALKLFLEETKATVAVYLESLQDTKITLIINWLQEALSSASLAHMKAKFRETLEDTRDRMYQMDIQQELQRYLSLVGQVYSTLVTYISDWWTLAAKNLTDFAEQYSIQDWAKRMKALVEQGFTVPEIKTILGTMPAFEVSLQALQKATFQTPDFIVPLTDLRIPSVQINFKDLKNIKIPSRFSTPEFTILNTFHIPSFTIDFVEMKVKIIRTIDQMLNSELQWPVPDIYLRDLKVEDIPLARITLPDFRLPEIAIPEFIIPTLNLNDFQVPDLHIPEFQLPHISHTIEVPTFGKLYSILKIQSPLFTLDANADIGNGTTSANEAGIAASITAKGESKLEVLNFDFQANAQLSNPKINPLALKESVKFSSKYLRTEHGSEMLFFGNAIEGKSNTVASLHTEKNTLELSNGVIVKINNQLTLDSNTKYFHKLNIPKLDFSSQADLRNEIKTLLKAGHIAWTSSGKGSWKWACPRFSDEGTHESQISFTIEGPLTSFGLSNKINSKHLRVNQNLVYESGSLNFSKLEIQSQVDSQHVGHSVLTAKGMALFGEGKAEFTGRHDAHLNGKVIGTLKNSLFFSAQPFEITASTNNEGNLKVRFPLRLTGKIDFLNNYALFLSPSAQQASWQVSARFNQYKYNQNFSAGNNENIMEAHVGINGEANLDFLNIPLTIPEMRLPYTIITTPPLKDFSLWEKTGLKEFLKTTKQSFDLSVKAQYKKNKHRHSITNPLAVLCEFISQSIKSFDRHFEKNRNNALDFVTKSYNETKIKFDKYKAEKSHDELPRTFQIPGYTVPVVNVEVSPFTIEMSAFGYVFPKAVSMPSFSILGSDVRVPSYTLILPSLELPVLHVPRNLKLSLPDFKELCTISHIFIPAMGNITYDFSFKSSVITLNTNAELFNQSDIVAHLLSSSSSVIDALQYKLEGTTRLTRKRGLKLATALSLSNKFVEGSHNSTVSLTTKNMEVSVATTTKAQIPILRMNFKQELNGNTKSKPTVSSSMEFKYDFNSSMLYSTAKGAVDHKLSLESLTSYFSIESSTKGDVKGSVLSREYSGTIASEANTYLNSKSTRSSVKLQGTSKIDDIWNLEVKENFAGEATLQRIYSLWEHSTKNHLQLEGLFFTNGEHTSKATLELSPWQMSALVQVHASQPSSFHDFPDLGQEVALNANTKNQKIRWKNEVRIHSGSFQSQVELSNDQEKAHLDIAGSLEGHLRFLKNIILPVYDKSLWDFLKLDVTTSIGRRQHLRVSTAFVYTKNPNGYSFSIPVKVLADKFIIPGLKLNDLNSVLVMPTFHVPFTDLQVPSCKLDFREIQIYKKLRTSSFALNLPTLPEVKFPEVDVLTKYSQPEDSLIPFFEITVPESQLTVSQFTLPKSVSDGIAALDLNAVANKIADFELPTIIVPEQTIEIPSIKFSVPAGIVIPSFQALTARFEVDSPVYNATWSASLKNKADYVETVLDSTCSSTVQFLEYELNVLGTHKIEDGTLASKTKGTFAHRDFSAEYEEDGKYEGLQEWEGKAHLNIKSPAFTDLHLRYQKDKKGISTSAASPAVGTVGMDMDEDDDFSKWNFYYSPQSSPDKKLTIFKTELRVRESDEETQIKVNWEEEAASGLLTSLKDNVPKATGVLYDYVNKYHWEHTGLTLREVSSKLRRNLQNNAEWVYQGAIRQIDDIDVRFQKAASGTTGTYQEWKDKAQNLYQELLTQEGQASFQGLKDNVFDGLVRVTQEFHMKVKHLIDSLIDFLNFPRFQFPGKPGIYTREELCTMFIREVGTVLSQVYSKVHNGSEILFSYFQDLVITLPFELRKHKLIDVISMYRELLKDLSKEAQEVFKAIQSLKTTEVLRNLQDLLQFIFQLIEDNIKQLKEMKFTYLINYIQDEINTIFSDYIPYVFKLLKENLCLNLHKFNEFIQNELQEASQELQQIHQYIMALREEYFDPSIVGWTVKYYELEEKIVSLIKNLLVALKDFHSEYIVSASNFTSQLSSQVEQFLHRNIQEYLSILTDPDGKGKEKIAELSATAQEIIKSQAIATKKIISDYHQQFRYKLQDFSDQLSDYYEKFIAESKRLIDLSIQNYHTFLIYITELLKKLQSTTVMNPYMKLAPGELTIIL

SEQ ID NO: 31 - protein, 1065 aa, synthetic construct
  CARBOHYD 762: note = N is linked to the N-linked glycan structure Hex(5)HexNAc(4)Fuc(0)NeuAc(1)
  CARBOHYD 397: note = N is linked to the N-linked glycan structure Hex(5)HexNAc(4)Fuc(1)NeuAc(2)
  CARBOHYD 762: note = N is linked to the N-linked glycan structure Hex(5)HexNAc(4)Fuc(0)NeuAc(2)
  CARBOHYD 762: note = N is linked to the N-linked glycan structure Hex(5)HexNAc(4)Fuc(1)NeuAc(2)
  CARBOHYD 762: note = N is linked to the N-linked glycan structure Hex(6)HexNAc(5)Fuc(0)NeuAc(2)
MKILILGIFLFLCSTPAWAKEKHYYIGIIETTWDYASDHGEKKLISVDTEHSNIYLQNGPDRIGRLYKKALYLQYTDETFRTTIEKPVWLGFLGPIIKAETGDKVYVHLKNLASRPYTFHSHGITYYKEHEGAIYPDNTTDFQRADDKVYPGEQYTYMLLATEEQSPGEGDGNCVTRIYHSHIDAPKDIASGLIGPLIICKKDSLDKEKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKHRGVYSSDVFDIFPGTYQTLEMFPRTPGIWLLHCHVTDHIHAGMETTYTVLQNEDTKSG

SEQ ID NO: 32 - protein, 764 aa, synthetic construct
  CARBOHYD 378: note = N is linked to the N-linked glycan structure Hex(5)HexNAc(4)Fuc(0)NeuAc(2)
MGSNLSPQLCLMPFILGLLSGGVTTTPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFL

SEQ ID NO: 33 - protein, 1744 aa, synthetic construct
  CARBOHYD 1391: note = N is linked to the N-linked glycan structure Hex(5)HexNAc(4)Fuc(0)NeuAc(2)
  CARBOHYD 226: note = N is linked to the N-linked glycan structure Hex(5)HexNAc(2)Fuc(0)NeuAc(0)
  CARBOHYD 226: note = N is linked to the N-linked glycan structure Hex(9)HexNAc(2)Fuc(0)NeuAc(0)
MRLLWGLIWASSFFTLSLQKPRLLLFSPSVVHLGVPLSVGVQLQDVPRGQVVKGSVFLRNPSRNNVPCSPKVDFTLSSERDFALLSLQVPLKDAKSCGLHQLLRGPEVQLVAHSPWLKDSLSRTTNIQGINLLFSSRRGHLFLQTDQPIYNPGQRVRYRVFALDQKMRPSTDTITVMVENSHGLRVRKKEVYMPSSIFQDDFVIPDISEPGTWKISARFSDGLESNSSTQFEVKKYVLPNFEVKITPGKPYILTVPGHLDEMQLDIQARYIYGKPVQGVAYVRFGLLDEDGKKTFFRGLESQTKLVNGQSHISLSKAEFQDALEKLNMGITDLQGLRLYVAAAIIESPGGEMEEAELTSWYFVSSPFSLDLSKTKRHLVPGAPFLLQALVREMSGSPASGIPVKVSATVSSPGSVPEVQDIQQNTDGSGQVSIPIIIPQTISELQLSVSAGSPHPAIARLTVAAPPSGGPGFLSIERPDSRPPRVGDTLNLNLRAVGSGATFSHYYYMILSRGQIVFMNREPKRTLTSVSVFVDHHLAPSFYFVAFYYHGDHPVANSLRVDVQAGACEGKLELSVDGAKQYRNGESVKLHLETDSLALVALGALDTALYAAGSKSHKPLNMGKVFEAMNSYDLGCGPGGGDSALQVFQAAGLAFSDGDQWTLSRKRLSCPKEKTTRKKRNVNFQKAINEKLGQYASPTAKRCCQDGVTRLPMMRSCEQRAARVQQPDCREPFLSCCQFAESLRKKSRDKGQAGLQRALEILQEEDLIDEDDIPVRSFFPENWLWRVETVDRFQILTLWLPDSLTTWEIHGLSLSKTKGLCVATPVQLRVFREFHLHLRLPMSVRRFEQLELRPVLYNYLDKNLTVSVHVSPVEGLCLAGGGGLAQQVLVPAGSARPVAFSVVPTAAAAVSLKVVARGSFEFPVGDAVSKVLQIEKEGAIHREELVYELNPLDHRGRTLEIPGNSDPNMIPDGDFNSYVRVTASDPLDTLGSEGALSPGGVASLLRLPRGCGEQTMIYLAPTLAASRYLDKTEQWSTLPPETKDHAVDLIQKGYMRIQQFRKADGSYAAWLSRDSSTWLTAFVLKVLSLAQEQVGGSPEKLQETSNWLLSQQQADGSFQDPCPVLDRSMQGGLVGNDETVALTAFVTIALHHGLAVFQDEGAEPLKQRVEASISKANSFLGEKASAGLLGAHAAAITAYALTLTKAPVDLLGVAHNNLMAMAQETGDNLYWGSVTGSQSNAVSPTPAPRNPSDPMPQAPALWIETTAYALLHLLLHEGKAEMADQASAWLTRQGSFQGGFRSTQDTVIALDALSAYWIASHTTEERGLNVTLSSTGRNGFKSHALQLNNRQIRGLEEELQFSLGSKINVKVGGNSKGTLKVLRTYNVLDMKNTTCQDLQIEVTVKGHVEYTMEANEDYEDYEYDELPAKDDPDAPLQPVTPLQLFEGRRNRRRREAPKVVEEQESRVHYTVCIWRNGKVGLSGMAIADVTLLSGFHALRADLEKLTSLSDRYVSHFETEGPHVLLYFDSVPTSRECVGFEAVQEVPVGLVQPASATLYDYYNPERRCSVFYGAPSKSRLLATLCSAEVCQCAEGKCPRQRRALERGLQDEDGYRMKFACYYPRVEYGFQVKVLREDSRAAFRLFETKITQVLHFTKDVKAAANQMRNFLVRASCRLRLEPGKEYLIMGLDGATYDLEGHPQYLLDSNSWIEEMPSERLCRSTRQRAACAQLNDFLQEYGTQGCQV

SEQ ID NO: 34 - protein, 491 aa, synthetic construct
  CARBOHYD 394: note = N is linked to the N-linked glycan structure Hex(2)HexNAc(2)Fuc(0)NeuAc(0)
  CARBOHYD 394: note = N is linked to the N-linked glycan structure Hex(3)HexNAc(2)Fuc(0)NeuAc(0)
  CARBOHYD 394: note = N is linked to the N-linked glycan structure Hex(4)HexNAc(3)Fuc(0)NeuAc(1)
  CARBOHYD 395: note = R is linked to the N-linked glycan structure Hex(4)HexNAc(3)Fuc(0)NeuAc(0)
  CARBOHYD 395: note = R is linked to the N-linked glycan structure Hex(5)HexNAc(4)Fuc(0)NeuAc(1)
MKRMVSWSFHKLKTMKHLLLLLLCVFLVKSQGVNDNEEGFFSARGHRPLDKKREEAPSLRPAPPPISGGGYRARPAKAAATQKKVERKAPDAGGCLHADPDLGVLCPTGCQLQEALLQQERPIRNSVDELNNNVEAVSQTSSSSFQYMYLLKDLWQKRQKQVKDNENVVNEYSSELEKHQLYIDETVNSNIPTNLRVLRSILENLRSKIQKLESDVSAQMEYCRTPCTVSCNIPVVSGKECEEIIRKGGETSEMYLIQPDSSVKPYRVYCDMNTENGGWTVIQNRQDGSVDFGRKWDPYKQGFGNVATNTDGKNYCGLPGEYWLGNDKISQLTRMGPTELLIEMEDWKGDKVKAHYGGFTVQNEANKYQISVNKYRGTAGNALMDGASQLMGENRTMTIHNGMFFSTYDRDNDGWLTSDPRKQCSKEDGGGWWYNRCHAANPNGRYYWGGQYTWDMAKHGTDDGVVWMNWKGSWYSMRKMSMKIRPFFPQQ

SEQ ID NO: 35 - protein, 2477 aa, synthetic construct
  CARBOHYD 430: note = N is linked to the N-linked glycan structure Hex(5)HexNAc(4)Fuc(0)NeuAc(1)
  CARBOHYD 542: note = N is linked to the N-linked glycan structure Hex(5)HexNAc(4)Fuc(0)NeuAc(1)
  CARBOHYD 542: note = N is linked to the N-linked glycan structure Hex(5)HexNAc(4)Fuc(0)NeuAc(2)
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 36 - protein, 462 aa, synthetic construct
  CARBOHYD 240: note = N is linked to the N-linked glycan structure Hex(5)HexNAc(4)Fuc(0)NeuAc(2)
  CARBOHYD 453: note = W is linked to the N-linked glycan structure Hex(6)HexNAc(5)Fuc(0)NeuAc(2)
MARVLGAPVALGLWSLCWSLAIATPLPPTSAHGNVAEGETKPDPDVTERCSDGWSFDATTLDDNGTMLFFKGEFVWKSHKWDRELISERWKNFPSPVDAAFRQGHNSVFLIKGDKVWVYPPEKKEKGYPKLLQDEFPGIPSPLDAAVECHRGECQAEGVLFFQGDREWFWDLATGTMKERSWPAVGNCSSALRWLGRYYCFQGNQFLRFDPVRGEVPPRYPRDVRDYFMPCPGRGHGHRNGTGHGNSTHHGPEYMRCSPHLVLSALTSDNHGATYAFSGTHYWRLDTSRDGWHSWPIAHQWPQGPSAVDAAFSWEEKLYLVQGTQVYVFLTKGGYTLVSGYPKRLEKEVGTPHGIILDSVDAAFICPGSSRLHIMAGRRLWWLDLKSGAQATWTELPWPHEKVDGALCMEKSLGPNSCSANGPGLYLIHGPNLYCYSDVEKLNAAKALPQPQNVTSLLGCTH

SEQ ID NO: 37 - protein, 500 aa, synthetic construct
  CARBOHYD 238: note = N is linked to the N-linked glycan structure Hex(5)HexNAc(4)Fuc(0)NeuAc(1)
  CARBOHYD 253: note = N is linked to the N-linked glycan structure Hex(5)HexNAc(4)Fuc(0)NeuAc(2)
  CARBOHYD 352: note = N is linked to the N-linked glycan structure Hex(5)HexNAc(4)Fuc(0)NeuAc(2)
MASRLTLLTLLLLLLAGDRASSNPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGENTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA

SEQ ID NO: 38 - protein, 453 aa, synthetic construct
  CARBOHYD 440: note = S is linked to the N-linked glycan structure Hex(6)HexNAc(2)Fuc(0)NeuAc(0)
  CARBOHYD 209: note = N is linked to the N-linked glycan structure Hex(5)HexNAc(4)Fuc(0)NeuAc(2)
  CARBOHYD 209: note = N is linked to the N-linked glycan structure Hex(5)HexNAc(4)Fuc(1)NeuAc(0)
  CARBOHYD 209: note = N is linked to the N-linked glycan structure Hex(6)HexNAc(4)Fuc(0)NeuAc(1)
  CARBOHYD 209: note = N is linked to the N-linked glycan structure Hex(6)HexNAc(5)Fuc(0)NeuAc(1)
  CARBOHYD 209: note = N is linked to the N-linked glycan structure Hex(6)HexNAc(5)Fuc(0)NeuAc(2)
  CARBOHYD 209: note = N is linked to the N-linked glycan structure Hex(5)HexNAc(4)Fuc(0)NeuAc(1)
  CARBOHYD 209: note = N is linked to the N-linked glycan structure Hex(5)HexNAc(4)Fuc(1)NeuAc(2)
  CARBOHYD 209: note = N is linked to the N-linked glycan structure Hex(5)HexNAc(5)Fuc(0)NeuAc(1)
  CARBOHYD 209: note = N is linked to the N-linked glycan structure Hex(5)HexNAc(5)Fuc(1)NeuAc(0)
  CARBOHYD 46: note = N is linked to the N-linked glycan structure Hex(5)HexNAc(4)Fuc(0)NeuAc(1)
  CARBOHYD 46: note = N is linked to the N-linked glycan structure Hex(5)HexNAc(4)Fuc(1)NeuAc(1)
  CARBOHYD 46: note = N is linked to the N-linked glycan structure Hex(5)HexNAc(5)Fuc(0)NeuAc(1)
  CARBOHYD 46: note = N is linked to the N-linked glycan structure Hex(6)HexNAc(3)Fuc(1)NeuAc(1)
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 39 - protein, 911 aa, synthetic construct
  CARBOHYD 588: note = N is linked to the N-linked glycan structure Hex(5)HexNAc(4)Fuc(0)NeuAc(1)
  CARBOHYD 588: note = N is linked to the N-linked glycan structure Hex(5)HexNAc(4)Fuc(1)NeuAc(2)
MDGAMGPRGLLLCMYLVSLLILQAMPALGSATGRSKSSEKRQAVDTAVDGVFIRSLKVNCKVTSRFAHYVVTSQVVNTANEAREVAFDLEIPKTAFISDFAVTADGNAFIGDIKDKVTAWKQYRKAAISGENAGLVRASGRTMEQFTIHLTVNPQSKVTFQLTYEEVLKRNHMQYEIVIKVKPKQLVHHFEIDVDIFEPQGISKLDAQASFLPKELAAQTIKKSFSGKKGHVLFRPTVSQQQSCPTCSTSLLNGHFKVTYDVSRDKICDLLVANNHFAHFFAPQNLTNMNKNVVFVIDISGSMRGQKVKQTKEALLKILGDMQPGDYFDLVLFGTRVQSWKGSLVQASEANLQAAQDFVRGFSLDEATNLNGGLLRGIEILNQVQESLPELSNHASILIMLTDGDPTEGVTDRSQILKNVRNAIRGRFPLYNLGFGHNVDFNFLEVMSMENNGRAQRIYEDHDATQQLQGFYSQVAKPLLVDVDLQYPQDAVLALTQNHHKQYYEGSEIVVAGRIADNKQSSFKADVQAHGEGQEFSITCLVDEEEMKKLLRERGHMLENHVERLWAYLTIQELLAKRMKVDREERANLSSQALQMSLDYGFVTPLTSMSIRGMADQDGLKPTIDKPSEDSPPLEMLGPRRTFVLSALQPSPTHSSSNTQRLPDRVTGVDTDPHFIIHVPQKEDTLCFNINEEPGVILSLVQDPNTGFSVNGQLIGNKARSPGQHDGTYFGRLGIANPATDFQLEVTPQNITLNPGFGGPVFSWRDQAVLRQDGVVVTINKKRNLVVSVDDGGTFEVVLHRVWKGSSVHQDFLGFYVLDSHRMSARTHGLLGQFFHPIGFEVSDIHPGSDPTKPDATMVVRNRRLTVTRGLQKDYSKDPWHGAEVSCWFIHNNGAGLIDGAYTDYIVPDIF

SEQ ID NO: 40 - protein, 648 aa, synthetic construct
  CARBOHYD 458: note = N is linked to the N-linked glycan structure Hex(4)HexNAc(3)Fuc(0)NeuAc(1)
  CARBOHYD 458: note = Hex(5)HexNAc(4)Fuc(0)NeuAc(1)
MSRKASENVEYTLRSLSSLMGERRRKQPEPDAASAAGECSLLAAAESSTSLQSAGAGGGGVGDLERAARRQFQQDETPAFVYVVAVFSALGGFLFGYDTGVVSGAMLLLKRQLSLDALWQELLVSSTVGAAAVSALAGGALNGVFGRRAAILLASALFTAGSAVLAAANNKETLLAGRLVVGLGIGIASMTVPVYIAEVSPPNLRGRLVTINTLFITGGQFFASVVDGAFSYLQKDGWRYMLGLAAVPAVIQFFGFLFLPESPRWLIQKGQTQKARRILSQMRGNQTIDEEYDSIKNNIEEEEKEVGSAGPVICRMLSYPPTRRALIVGCGLQMFQQLSGINTIMYYSATILQMSGVEDDRLAIWLASVTAFTNFIFTLVGVWLVEKVGRRKLTFGSLAGTTVALIILALGFVLSAQVSPRITFKPIAPSGQNATCTRYSYCNECMLDPDCGFCYKMNKSTVIDSSCVPVNKASTNEAAWGRCENETKFKTEDIFWAYNFCPTPYSWTALLGLILYLVFFAPGMGPMPWTVNSEIYPLWARSTGNACSSGINWIFNVLVSLTFLHTAEYLTYYGAFFLYAGFAAVGLLFIYGCLPETKGKKLEEIESLFDNRLCTCGTSDSDEGRYIEYIRVKGSNYHLSDNDASDVE

SEQ ID NO: 41 - protein, 840 aa, synthetic construct
  CARBOHYD 604: note = N is linked to the N-linked glycan structure Hex(6)HexNAc(5)Fuc(0)NeuAc(2)
MSAFRLWPGLLIMLGSLCHRGSPCGLSTHVEIGHRALEFLQLHNGRVNYRELLLEHQDAYQAGIVFPDCFYPSICKGGKFHDVSESTHWTPFLNASVHYIRENYPLPWEKDTEKLVAFLFGITSHMAADVSWHSLGLEQGFLRTMGAIDFHGSYSEAHSAGDFGGDVLSQFEFNFNYLARRWYVPVKDLLGIYEKLYGRKVITENVIVDCSHIQFLEMYGEMLAVSKLYPTYSTKSPFLVEQFQEYFLGGLDDMAFWSTNIYHLTSFMLENGTSDCNLPENPLFIACGGQQNHTQGSKMQKNDFHRNLTTSLTESVDRNINYTERGVFFSVNSWTPDSMSFIYKALERNIRTMFIGGSQLSQKHVSSPLASYFLSFPYARLGWAMTSADLNQDGHGDLVVGAPGYSRPGHIHIGRVYLIYGNDLGLPPVDLDLDKEAHRILEGFQPSGRFGSALAVLDFNVDGVPDLAVGAPSVGSEQLTYKGAVYVYFGSKQGGMSSSPNITISCQDIYCNLGWTLLAADVNGDSEPDLVIGSPFAPGGGKQKGIVAAFYSGPSLSDKEKLNVEAANWTVRGEEDFSWFGYSLHGVTVDNRTLLLVGSPTWKNASRLGHLLHIRDEKKSLGRVYGYFPPNGQSWFTISGDKAMGKLGTSLSSGHVLMNGTLKQVLLVGAPTYDDVSKVAFLTVTLHQGGATRMYALTSDAQPLLLSTFSGDRRFSRFGGVLHLSDLDDDGLDEIIMAAPLRIADVTSGLIGGEDGRVYVYNGKETTLGDMTGKCKSWITPCPEEKAQYVLISPEASSRFGSSLITVRSKAKNQVVIAAGRSSLGARLSGALHVYSLGSD

SEQ ID NO: 42 - protein, 419 aa, synthetic construct
  CARBOHYD 259: note = N is linked to the N-linked glycan structure Hex(5)HexNAc(4)Fuc(1)NeuAc(1)
  CARBOHYD 268: note = N is linked to the N-linked glycan structure Hex(5)HexNAc(4)Fuc(1)NeuAc(1)
  CARBOHYD 303: note = N is linked to the N-linked glycan structure Hex(5)HexNAc(4)Fuc(1)NeuAc(1)
  CARBOHYD 303: note = N is linked to the N-linked glycan structure Hex(5)HexNAc(4)Fuc(2)NeuAc(2)
MGTLSAPPCTQRIKWKGLLLTASLLNFWNLPTTAQVTIEAEPTKVSEGKDVLLLVHNLPQNLTGYIWYKGQMRDLYHYITSYVVDGEIIIYGPAYSGRETAYSNASLLIQNVTREDAGSYTLHIIKGDDGTRGVTGRFTFTLHLETPKPSISSSNLNPRETMEAVSLTCDPETPDASYLWWMNGQSLPMTHSLKLSETNRTLFLLGVTKYTAGPYECEIRNPVSASRSDPVTLNLLPKLPKPYITINNLNPRENKDVLNFTCEPKSENYTYIWWLNGQSLPVSPRVKRPIENRILILPSVTRNETGPYQCEIRDRYGGIRSDPVTLNVLYGPDLPRIYPSFTYYRSGEVLYLSCSADSNPPAQYSWTINEKFQLPGQKLFIRHITTKHSGLYVCSVRNSATGKESSKSMTVEVSDWTVP

SEQ ID NO: 43 - protein, 419 aa, synthetic construct
  CARBOHYD 268: note = N is linked to the N-linked glycan structure Hex(5)HexNAc(4)Fuc(1)NeuAc(2)
  CARBOHYD 303: note = N is linked to the N-linked glycan structure Hex(5)HexNAc(4)Fuc(1)NeuAc(2)
  CARBOHYD 303: note = N is linked to the N-linked glycan structure Hex(5)HexNAc(4)Fuc(2)NeuAc(2)
MGPLSAPPCTQHITWKGLLLTASLLNFWNPPTTAQVTIEAQPPKVSEGKDVLLLVHNLPQNLTGYIWYKGQIRDLYHYVTSYIVDGQIIKYGPAYSGRETVYSNASLLIQNVTQEDTGSYTLHIIKRGDGTGGVTGRFTFTLYLETPKPSISSSNFNPREATEAVILTCDPETPDASYLWWMNGQSLPMTHSLQLSETNRTLYLFGVTNYTAGPYECEIRNPVSASRSDPVTLNLLPKLPKPYITINNLNPRENKDVSTFTCEPKSENYTYIWWLNGQSLPVSPRVKRRIENRILILPSVTRNETGPYQCEIRDRYGGIRSDPVTLNVLYGPDLPRIYPSFTYYHSGQNLYLSCFADSNPPAQYSWTINGKFQLSGQKLSIPQITTKHSGLYACSVRNSATGKESSKSVTVRVSDWTLP

SEQ ID NO: 44 - protein, 1482 aa, synthetic construct
  CARBOHYD 54: note = N is linked to the N-linked glycan structure Hex(5)HexNAc(4)Fuc(0)NeuAc(1)
  CARBOHYD 54: note = N is linked to the N-linked glycan structure Hex(5)HexNAc(4)Fuc(0)NeuAc(2)
MRKDRLLHLCLVLLLILLSASDSNSTEPQYMVLVPSLLHTEAPKKGCVLLSHLNETVTVSASLESGRENRSLFTDLVAEKDLFHCVSFTLPRISASSEVAFLSIQIKGPTQDFRKRNTVLVLNTQSLVFVQTDKPMYKPGQTVRFRVVSVDENFRPRNELIPLIYLENPRRNRIAQWQSLKLEAGINQLSFPLSSEPIQGSYRVVVQTESGGRIQHPFTVEEFVLPKFEVKVQVPKIISIMDEKVNITVCGEYTYGKPVPGLATVSLCRKLSRVLNCDKQEVCEEFSQQLNSNGCITQQVHTKMLQITNTGFEMKLRVEARIREEGTDLEVTANRISEITNIVSKLKFVKVDSHFRQGIPFFAQVLLVDGKGVPIPNKLFFISVNDANYYSNATTNEQGLAQFSINTTSISVNKLFVRVFTVHPNLCFHYSWVAEDHQGAQHTANRVFSLSGSYIHLEPVAGTLPCGHTETITAHYTLNRQAMGELSELSFHYLIMAKGVIVRSGTHTLPVESGDMKGSFALSFPVESDVAPIARMFIFAILPDGEVVGDSEKFEIENCLANKVDLSFSPAQSPPASHAHLQVAAAPQSLCALRAVDQSVLLMKPEAELSVSSVYNLLTVKDLTNFPDNVDQQEEEQGHCPRPFFIHNGAIYVPLSSNEADIYSFLKGMGLKVFTNSKIRKPKSCSVIPSVSAGAVGQGYYGAGLGVVERPYVPQLGTYNVIPLNNEQSSGPVPETVRSYFPETWIWELVAVNSSGVAEVGVTVPDTITEWKAGAFCLSEDAGLGISSTASLRAFQPFFVELTMPYSVIRGEVFTLKATVLNYLPKCIRVSVQLKASPAFLASQNTKGEESYCICGNERQTLSWTVTPKTLGNVNFSVSAEAMQSLELCGNEVVEVPEIKRKDTVIKTLLVEAEGIEQEKTFSSMTCASGANVSEQLSLKLPSNVVKESARASFSVLGDILGSAMQNIQNLLQMPYGCGEQNMVLFAPNIYVLNYLNETQQLTQEIKAKAVGYLITGYQRQLNYKHQDGSYSTFGERYGRNQGNTWLTAFVLKTFAQARSYIFIDEAHITQSLTWLSQMQKDNGCFRSSGSLLNNAIKGGVEDEATLSAYVTIALLEIPLPVTNPIVRNALFCLESAWNVAKEGTHGSHVYTKALLAYAFSLLGKQNQNREILNSLDKEAVKEDNLVHWERPQRPKAPVGHLYQTQAPSAEVEMTSYVLLAYLTAQPAPTSGDLTSATNIVKWIMKQQNAQGGFSSTQDTVVALHALSRYGAATFTRTEKTAQVTVQDSQTFSTNFQVDNNNLLLLQQISLPELPGEYVITVTGERCVYLQTSMKYNILPEKEDSPFALKVQTVPQTCDGHKAHTSFQISLTISYTGNRPASNMVIVDVKMVSGFIPLKPTVKMLERSSSVSRTEVSNNHVLIYVEQVTNQTLSFSFMVLQDIPVGDLKPAIVKVYDYYETDESVVAEYIAPCSTDTEHGNV

SEQ ID NO: 45 - protein, 402 aa, synthetic construct; identical to SEQ ID NO: 15
  CARBOHYD 396: note = N is linked to the N-linked glycan structure Hex(5)HexNAc(4)Fuc(0)NeuAc(2)

SEQ ID NO: 46 - protein, 698 aa, synthetic construct
  CARBOHYD 432: note = N is linked to the N-linked glycan structure Hex(4)HexNAc(3)Fuc(0)NeuAc(0)
  CARBOHYD 432: note = N is linked to the N-linked glycan structure Hex(6)HexNAc(5)Fuc(0)NeuAc(2)
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAIAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 47 - protein, 478 aa, synthetic construct
  CARBOHYD 242: note = N is linked to the N-linked glycan structure Hex(5)HexNAc(4)Fuc(0)NeuAc(1)
  CARBOHYD 169: note = N is linked to the N-linked glycan structure Hex(5)HexNAc(4)Fuc(0)NeuAc(1)
  CARBOHYD 169: note = N is linked to the N-linked glycan structure Hex(5)HexNAc(4)Fuc(0)NeuAc(2)
  CARBOHYD 242: note = N is linked to the N-linked glycan structure Hex(5)HexNAc(4)Fuc(1)NeuAc(2)
MAPLRPLLILALLAWVALADQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 48 - protein, 298 aa, synthetic construct
  CARBOHYD 109: note = N is linked to the N-linked glycan structure Hex(5)HexNAc(4)Fuc(0)NeuAc(2)
  CARBOHYD 109: note = N is linked to the N-linked glycan structure Hex(5)HexNAc(4)Fuc(0)NeuAc(2)
MVRMVPVLLSLLLLLGPAVPQENQDGRYSLTYIYTGLSKHVEDVPAFQALGSLNDLQFFRYNSKDRKSQPMGLWRQVEGMEDWKQDSQLQKAREDIFMETLKDIVEYYNDSNGSHVLQGRFGCEIENNRSSGAFWKYYYDGKDYIEFNKEIPAWVPFDPAAQITKQKWEAEPVYVQRAKAYLEEECPATLRKYLKYSKNILDRQDPPSVVVTSHQAPGEKKKLKCLAYDFYPGKIDVHWTRAGEVQEPELRGDVLHNGNGTYQSWVVVAVPPQDTAPYSCHVQHSSLAQPLVVPWEAS

SEQ ID NO: 49 - protein, 599 aa, synthetic construct
  CARBOHYD 109: note = N is linked to the N-linked glycan structure Hex(5)HexNAc(4)Fuc(0)NeuAc(2)
MKLLKLTGFIFFLFFLTESLTLPTQPRDIENFNSTQKFIEDNIEYITIIAFAQYVQEATFEEMEKLVKDMVEYKDRCMADKTLPECSKLPNNVLQEKICAMEGLPQKHNFSHCCSKVDAQRRLCFFYNKKSDVGFLPPFPTLDPEEKCQAYESNRESLLNHFLYEVARRNPFVFAPTLLTVAVHFEEVAKSCCEEQNKVNCLQTRAIPVTQYLKAFSSYQKHVCGALLKFGTKVVHFIYIAILSQKFPKIEFKELISLVEDVSSNYDGCCEGDVVQCIRDTSKVMNHICSKQDSISSKIKECCEKKIPERGQCIINSNKDDRPKDLSLREGKFTDSENVCQERDADPDTFFAKFTFEYSRRHPDLSIPELLRIVQIYKDLLRNCCNTENPPGCYRYAEDKFNETTEKSLKMVQQECKHFQNLGKDGLKYHYLIRLTKIAPQLSTEELVSLGEKMVTAFTTCCTLSEEFACVDNLADLVFGELCGVNENRTINPAVDHCCKTNFAFRRPCFESLKADKTYVPPPFSQDLFTFHADMCQSQNEELQRKTDRFLVNLVKLKHELTDEELQSLFTNFANVVDKCCKAESPEVCFNEESPKIGN

SEQ ID NO: 50 - protein, 583 aa, synthetic construct
  CARBOHYD 70: note = N is linked to the N-linked glycan structure Hex(5)HexNAc(4)Fuc(0)NeuAc(1)
MKLLHVFLLFLCFHLRFCKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 51 - protein, 1663 aa, synthetic construct
  CARBOHYD 85: note = N is linked to the N-linked glycan structure Hex(5)HexNAc(2)Fuc(0)NeuAc(0)
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 52 - protein, 367 aa, synthetic construct; identical to SEQ ID NO: 14
  CARBOHYD 156: note = N is linked to the N-linked glycan structure Hex(5)HexNAc(4)Fuc(0)NeuAc(1)
  CARBOHYD 156: note = N is linked to the N-linked glycan structure Hex(6)HexNAc(5)Fuc(0)NeuAc(2)
  CARBOHYD 156: note = N is linked to the N-linked glycan structure Hex(6)HexNAc(4)Fuc(0)NeuAc(1)

SEQ ID NO: 53 - protein, 499 aa, synthetic construct
  CARBOHYD 49: note = N is linked to the N-linked glycan structure Hex(5)HexNAc(4)Fuc(0)NeuAc(2)
  CARBOHYD 49: note = N is linked to the N-linked glycan structure Hex(5)HexNAc(4)Fuc(1)NeuAc(2)
MKHSLNALLIFLIITSAWGGSKGPLDQLEKGGETAQSADPQWEQLNNKNLSMPLLPADFHKENTVTNDWIPEGEEDDDYLDLEKIFSEDDDYIDIVDSLSVSPTDSDVSAGNILQLFHGKSRIQRLNILNAKFAFNLYRVLKDQVNTFDNIFIAPVGISTAMGMISLGLKGETHEQVHSILHFKDFVNASSKYEITTIHNLFRKLTHRLFRRNFGYTLRSVNDLYIQKQFPILLDFKTKVREYYFAEAQIADFSDPAFISKTNNHIMKLTKGLIKDALENIDPATQMMILNCIYFKGSWVNKFPVEMTHNHNFRLNEREVVKVSMMQTKGNFLAANDQELDCDILQLEYVGGISMLIVVPHKMSGMKTLEAQLTPRVVERWQKSMTNRTREVLLPKFKLEKNYNLVESLKLMGIRMLFDKNGNMAGISDQRIAIDLFKHQGTITVNEEGTQATTVTTVGFMPLSTQVRFTVDRPFLFLIYEHRTSCLLFMGRVANPSRS

SEQ ID NO: 54 - protein, 406 aa, synthetic construct
  CARBOHYD 184: note = N is linked to the N-linked glycan structure Hex(6)HexNAc(5)Fuc(0)NeuAc(2)
MSALGAVIALLLWGQLFAVDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 55 - protein, 353 aa, synthetic construct
  CARBOHYD 144: note = N is linked to the N-linked glycan structure Hex(5)HexNAc(4)Fuc(0)NeuAc(1)
  CARBOHYD 144: note = N is linked to the N-linked glycan structure Hex(5)HexNAc(4)Fuc(0)NeuAc(2)
  CARBOHYD 144: note = N is linked to the N-linked glycan structure Hex(5)HexNAc(5)Fuc(0)NeuAc(1)
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 56 - protein, 326 aa, synthetic construct
  CARBOHYD 176: note = N is linked to the N-linked glycan structure Hex(3)HexNAc(4)Fuc(1)NeuAc(0)
  CARBOHYD 176: note = N is linked to the N-linked glycan structure Hex(4)HexNAc(4)Fuc(1)NeuAc(0)
  CARBOHYD 176: note = N is linked to the N-linked glycan structure Hex(5)HexNAc(4)Fuc(1)NeuAc(0)
  CARBOHYD 176: note = N is linked to the N-linked glycan structure Hex(5)HexNAc(4)Fuc(1)NeuAc(1)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 57 - protein, 159 aa, synthetic construct
  CARBOHYD 71: note = N is linked to the N-linked glycan structure Hex(5)HexNAc(4)Fuc(0)NeuAc(1)
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 58 - protein, 638 aa, synthetic construct
  CARBOHYD 127: note = N is linked to the N-linked glycan structure Hex(5)HexNAc(4)Fuc(0)NeuAc(2)
MILFKQATYFISLFATVSCGCLTQLYENAFFRGGDVASMYTPNAQYCQMRCTFHPRCLLFSFLPASSINDMEKRFGCFLKDSVTGTLPKVHRTGAVSGHSLKQCGHQISACHRDIYKGVDMRGVNFNVSKVSSVEECQKRCTNNIRCQFFSYATQTFHKAEYRNNCLLKYSPGGTPTAIKVLSNVESGFSLKPCALSEIGCHMNIFQHLAFSDVDVARVLTPDAFVCRTICTYHPNCLFFTFYTNVWKIESQRNVCLLKTSESGTPSSSTPQENTISGYSLLTCKRTLPEPCHSKIYPGVDFGGEELNVTFVKGVNVCQETCTKMIRCQFFTYSLLPEDCKEEKCKCFLRLSMDGSPTRIAYGTQGSSGYSLRLCNTGDNSVCTTKTSTRIVGGTNSSWGEWPWQVSLQVKLTAQRHLCGGSLIGHQWVLTAAHCFDGLPLQDVWRIYSGILNLSDITKDTPFSQIKEIIIHQNYKVSEGNHDIALIKLQAPLNYTEFQKPICLPSKGDTSTIYTNCWVTGWGFSKEKGEIQNILQKVNIPLVTNEECQKRYQDYKITQRMVCAGYKEGGKDACKGDSGGPLVCKHNGMWRLVGITSWGEGCARREQPGVYTKVAEYMDWILEKTQSSDGKAQMQSPA

SEQ ID NO: 59 - protein, 415 aa, synthetic construct
  CARBOHYD 36: note = N is linked to the N-linked glycan structure Hex(5)HexNAc(4)Fuc(0)NeuAc(2)
MSPFLYLVLLVLGLHATIHCASPEGKVTACHSSQPNATLYKMSSINADFAFNLYRRFTVETPDKNIFFSPVSISAALVMLSFGACCSTQTEIVETLGFNLTDTPMVEIQHGFQHLICSLNFPKKELELQIGNALFIGKHLKPLAKFLNDVKTLYETEVFSTDFSNISAAKQEINSHVEMQTKGKVVGLIQDLKPNTIMVLVNYIHFKAQWANPFDPSKTEDSSSFLIDKTTTVQVPMMHQMEQYYHLVDMELNCTVLQMDYSKNALALFVLPKEGQMESVEAAMSSKTLKKWNRLLQKGWVDLFVPKFSISATYDLGATLLKMGIQHAYSENADFSGLTEDNGLKLSNAAHKAVLHIGEKGTEAAAVPEVELSDQPENTFLHPIIQIDRSFMLLILERSTRSILFLGKVVNPTEA

SEQ ID NO: 60 - protein, 345 aa, synthetic construct
  CARBOHYD 162: note = N is linked to the N-linked glycan structure Hex(5)HexNAc(4)Fuc(0)NeuAc(1)
MISPVLILFSSFLCHVAIAGRTCPKPDDLPFSTVVPLKTFYEPGEEITYSCKPGYVSRGGMRKFICPLTGLWPINTLKCTPRVCPFAGILENGAVRYTTFEYPNTISFSCNTGFYLNGADSAKCTEEGKWSPELPVCAPIICPPPSIPTFATLRVYKPSAGNNSLYRDTAVFECLPQHAMFGNDTITCTTHGNWTKLPECREVKCPFPSRPDNGFVNYPAKPTLYYKDKATFGCHDGYSLDGPEEIECTKLGNWSAMPSCKASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 61 - protein, 1691 aa, synthetic construct
  CARBOHYD 1330: note = N is linked to the N-linked glycan structure Hex(6)HexNAc(5)Fuc(0)NeuAc(1)
MASWTSPWWVLIGMVFMHSPLPQTTAEKSPGAYFLPEFALSPQGSFLEDTTGEQFLTYRYDDQTSRNTRSDEDKDGNWDAWGDWSDCSRTCGGGASYSLRRCLTGRNCEGQNIRYKTCSNHDCPPDAEDFRAQQCSAYNDVQYQGHYYEWLPRYNDPAAPCALKCHAQGQNLVVELAPKVLDGTRCNTDSLDMCISGICQAVGCDRQLGSNAKEDNCGVCAGDGSTCRLVRGQSKSHVSPEKREENVIAVPLGSRSVRITVKGPAHLFIESKTLQGSKGEHSFNSPGVFLVENTTVEFQRGSERQTFKIPGPLMADFIFKTRYTAAKDSVVQFFFYQPISHQWRQTDFFPCTVTCGGGYQLNSAECVDIRLKRVVPDHYCHYYPENVKPKPKLKECSMDPCPSSDGFKEIMPYDHFQPLPRWEHNPWTACSVSCGGGIQRRSFVCVEESMHGEILQVEEWKCMYAPKPKVMQTCNLFDCPKWIAMEWSQCTVTCGRGLRYRVVLCINHRGEHVGGCNPQLKLHIKEECVIPIPCYKPKEKSPVEAKLPWLKQAQELEETRIATEEPTFIPEPWSACSTTCGPGVQVREVKCRVLLTFTQTETELPEEECEGPKLPTERPCLLEACDESPASRELDIPLPEDSETTYDWEYAGFTPCTATCVGGHQEAIAVCLHIQTQQTVNDSLCDMVHRPPAMSQACNTEPCPPRWHVGSWGPCSATCGVGIQTRDVYCLHPGETPAPPEECRDEKPHALQACNQFDCPPGWHIEEWQQCSRTCGGGTQNRRVTCRQLLTDGSFLNLSDELCQGPKASSHKSCARTDCPPHLAVGDWSKCSVSCGVGIQRRKQVCQRLAAKGRRIPLSEMMCRDLPGLPLVRSCQMPECSKIKSEMKTKLGEQGPQILSVQRVYIQTREEKRINLTIGSRAYLLPNTSVIIKCPVRRFQKSLIQWEKDGRCLQNSKRLGITKSGSLKIHGLAAPDIGVYRCIAGSAQETVVLKLIGTDNRLIARPALREPMREYPGMDHSEANSLGVTWHKMRQMWNNKNDLYLDDDHISNQPFLRALLGHCSNSAGSTNSWELKNKQFEAAVKQGAYSMDTAQFDELIRNMSQLMETGEVSDDLASQLIYQLVAELAKAQPTHMQWRGIQEETPPAAQLRGETGSVSQSSHAKNSGKLTFKPKGPVLMRQSQPPSISFNKTINSRIGNTVYITKRTEVINILCDLITPSEATYTWTKDGTLLQPSVKIILDGTGKIQIQNPTRKEQGIYECSVANHLGSDVESSSVLYAEAPVILSVERNITKPEHNHLSVVVGGIVEAALGANVTIRCPVKGVPQPNITWLKRGGSLSGNVSLLFNGSLLLQNVSLENEGTYVCIATNALGKAVATSVLHLLERRWPESRIVFLQGHKKYILQATNTRTNSNDPTGEPPPQEPFWEPGNWSHCSATCGHLGARIQRPQCVMANGQEVSEALCDHLQKPLAGFEPCNIRDCPARWFTSVWSQCSVSCGEGYHSRQVTCKRTKANGTVQVVSPRACAPKDRPLGRKPCFGHPCVQWEPGNRCPGRCMGRAVRMQQRHTACQHNSSDSNCDDRKRPTLRRNCTSGACDVCWHTGPWKPCTAACGRGFQSRKVDCIHTRSCKPVAKRHCVQKKKPISWRHCLGPSCDRDCTDTTHYCMFVKHLNLCSLDRYKQRCCQSCQEG

SEQ ID NO: 62 - protein, 449 aa, synthetic construct
  CARBOHYD 299: note = N is linked to the N-linked glycan structure Hex(3)HexNAc(4)Fuc(1)NeuAc(0)
  CARBOHYD 299: note = N is linked to the N-linked glycan structure Hex(4)HexNAc(3)Fuc(0)NeuAc(0)
  CARBOHYD 299: note = N is linked to the N-linked glycan structure Hex(5)HexNAc(4)Fuc(0)NeuAc(0)
  CARBOHYD 299: note = N is linked to the N-linked glycan structure Hex(5)HexNAc(4)Fuc(1)NeuAc(0)
  CARBOHYD 299: note = N is linked to the N-linked glycan structure Hex(5)HexNAc(4)Fuc(1)NeuAc(1)
QVQLVQSGGGVVQPGRSLRLSCAASGFTFSRYTIHWVRQAPGKGLEWVAVMSYNGNNKHYADSVNGRFTISRNDSKNTLYLNMNSLRPEDTAVYYCARIRDTAMFFAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 340 aa, synthetic construct
  CARBOHYD 92: note = N is linked to the N-linked glycan structure Hex(5)HexNAc(4)Fuc(1)NeuAc(1)
  CARBOHYD 92: note = N is linked to the N-linked glycan structure Hex(5)HexNAc(4)Fuc(1)NeuAc(2)
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 64 - protein, 644 aa, synthetic construct
  CARBOHYD 169: note = N is linked to the N-linked glycan structure Hex(6)HexNAc(5)Fuc(0)NeuAc(2)
  CARBOHYD 205: note = N is linked to the N-linked glycan structure Hex(6)HexNAc(5)Fuc(0)NeuAc(2)
MKLITILFLCSRLLLSLTQESQSEEIDCNDKDLFKAVDAALKKYNSQNQSNNQFVLYRITEATKTVGSDTFYSFKYEIKEGDCPVQSGKTWQDCEYKDAAKAATGECTATVGKRSSTKFSVATQTCQITPAEGPVVTAQYDCLGCVHPISTQSPDLEPILRHGIQYFNNNTQHSSLFMLNEVKRAQRQVVAGLNFRITYSIVQTNCSKENFLFLTPDCKSLWNGDTGECTDNAYIDIQLRIASFSQNCDIYPGKDFVQPPTKICVGCPRDIPTNSPELEETLTHTITKLNAENNATFYFKIDNVKKARVQVVAGKKYFIDFVARETTCSKESNEELTESCETKKLGQSLDCNAEVYVVPWEKKIYPTVNCQPLGMISLMKRPPGFSPFRSSRIGEIKEETTVSPPHTSMAPAQDEERDSGKEQGHTRRHDWGHEKQRKHNLGHGHKHERDQGHGHQRGHGLGHGHEQQHGLGHGHKFKLDDDLEHQGGHVLDHGHKHKHGHGHGKHKNKGKKNGKHNGWKTEHLASSSEDSTTPSAQTQEKTEGPTPIPSLAKPGVTVTFSDFQDSDLIATMMPPISPAPIQSDDDWIPDIQIDPNGLSFNPISDFPDTTSPKCPGRPWKSVSEINPTTQMKESYYFDLTDGLS

SEQ ID NO: 65 - protein, 7 aa, synthetic construct
  CARBOHYD 2: note = N is linked to the N-linked glycan structure Hex(5)HexNAc(4)Fuc(0)NeuAc(2)
ENGTISR

SEQ ID NO: 66 - protein, 6 aa, synthetic construct
  CARBOHYD 5: note = N is linked to the N-linked glycan structure Hex(5)HexNAc(4)Fuc(1)NeuAc(2)
NEEYNK

SEQ ID NO: 67 - protein, 16 aa, synthetic construct
  CARBOHYD 15: note = N is linked to the N-linked glycan structure Hex(5)HexNAc(4)Fuc(0)NeuAc(2)
SVQEIQATFFYFTPNK

SEQ ID NO: 68 - protein, 16 aa, synthetic construct
  CARBOHYD 15: note = N is linked to the N-linked glycan structure Hex(6)HexNAc(5)Fuc(0)NeuAc(1)
SVQEIQATFFYFTPNK

SEQ ID NO: 69 - protein, 16 aa, synthetic construct
  CARBOHYD 15: note = N is linked to the N-linked glycan structure Hex(6)HexNAc(5)Fuc(0)NeuAc(2)
SVQEIQATFFYFTPNK

SEQ ID NO: 70 - protein, 16 aa, synthetic construct
  CARBOHYD 4: note = N is linked to the N-linked glycan structure Hex(4)HexNAc(3)Fuc(0)NeuAc(0)
YLGNATAIFFLPDEGK

SEQ ID NO: 71 - protein, 16 aa, synthetic construct
  CARBOHYD 4: note = N is linked to the N-linked glycan structure Hex(4)HexNAc(3)Fuc(0)NeuAc(1)
YLGNATAIFFLPDEGK

SEQ ID NO: 72 - protein, 16 aa, synthetic construct
  CARBOHYD 4: note = N is linked to the N-linked glycan structure Hex(6)HexNAc(5)Fuc(0)NeuAc(2)
YLGNATAIFFLPDEGK

SEQ ID NO: 73 - protein, 30 aa, synthetic construct
  CARBOHYD 7: note = N is linked to the N-linked glycan structure Hex(4)HexNAc(3)Fuc(0)NeuAc(1)
QLAHQSNSTNIFFSPVSIATAFAMLSLGTK

SEQ ID NO: 74 - protein, 30 aa, synthetic construct; identical to SEQ ID NO: 73
  CARBOHYD 7: note = N is linked to the N-linked glycan structure Hex(6)HexNAc(5)Fuc(0)NeuAc(2)

SEQ ID NO: 75 - protein, 19 aa, synthetic construct
  CARBOHYD 3: note = N is linked to the N-linked glycan structure Hex(5)HexNAc(4)Fuc(0)NeuAc(0)
VSNQTLSLFFTVLQDVPVR

SEQ ID NO: 76 - protein, 33 aa, synthetic construct
  CARBOHYD 6: note = N is linked to the N-linked glycan structure Hex(4)HexNAc(4)Fuc(0)NeuAc(1)
SLGNVNFTVSAEALESQELCGTEVPSVPEHGRK

SEQ ID NO: 77 - protein, 33 aa, synthetic construct; identical to SEQ ID NO: 76
  CARBOHYD 6: note = N is linked to the N-linked glycan structure Hex(5)HexNAc(4)Fuc(0)NeuAc(0)

SEQ ID NO: 78 - protein, 20 aa, synthetic construct
  CARBOHYD 2: note = N is linked to the N-linked glycan structure Hex(6)HexNAc(5)Fuc(0)NeuAc(2)
FNLTETSEAEIHQSFQHLLR

SEQ ID NO: 79 - protein, 34 aa, synthetic construct
  CARBOHYD 26: note = N is linked to the N-linked glycan structure Hex(5)HexNAc(4)Fuc(0)NeuAc(2)
APDKNVIFSPLSISTALAFLSLGAHNTTLTEILK

SEQ ID NO: 80 - protein, 36 aa, synthetic construct
  CARBOHYD 4: note = N is linked to the N-linked glycan structure Hex(5)HexNAc(4)Fuc(0)NeuAc(2)
YFYNGTSMACETFQYGGCMGNGNNFVTEKECLQTCR

SEQ ID NO: 81 - protein, 7 aa, synthetic construct
  CARBOHYD 3: note = N is linked to the N-linked glycan structure Hex(5)HexNAc(4)Fuc(0)NeuAc(2)
DKNCTSR

SEQ ID NO: 82 - protein, 16 aa, synthetic construct
  CARBOHYD 5: note = N is linked to the N-linked glycan structure Hex(5)HexNAc(4)Fuc(0)NeuAc(1)
LGACNDTLQQLMEVFK

SEQ ID NO: 83 - protein, 23 aa, synthetic construct
  CARBOHYD 3: note = N is linked to the N-linked glycan structure Hex(5)HexNAc(4)Fuc(0)NeuAc(1)
NANASLVYSFLYKTIEVFCEYFK

SEQ ID NO: 84 - protein, 6 aa, synthetic construct
  CARBOHYD 3: note = N is linked to the N-linked glycan structure Hex(5)HexNAc(4)Fuc(0)NeuAc(1)
SYNETK

SEQ ID NO: 85 - protein, 25 aa, synthetic construct
  CARBOHYD 9: note = N is linked to the N-linked glycan structure Hex(5)HexNAc(4)Fuc(0)NeuAc(1)
ELHHLQEQNVSNAFLDKGEFYIGSK

SEQ ID NO: 86 - protein, 23 aa, synthetic construct
  CARBOHYD 22: note = N is linked to the N-linked glycan structure Hex(5)HexNAc(4)Fuc(0)NeuAc(2)
KALQAVYSMMSWPDDVPPEGWNR

SEQ ID NO: 87 - protein, 22 aa, synthetic construct
  CARBOHYD 9: note = N is linked to the N-linked glycan structure Hex(5)HexNAc(4)Fuc(0)NeuAc(2)
TYNVLDMKNTTCQDLQIEVTVK

SEQ ID NO: 88 - protein, 16 aa, synthetic construct
  CARBOHYD 8: note = N is linked to the N-linked glycan structure Hex(5)HexNAc(2)Fuc(0)NeuAc(0)
FSDGLESNSSTQFEVK

SEQ ID NO: 89 - protein, 17 aa, synthetic construct
  CARBOHYD 8: note = N is linked to the N-linked glycan structure Hex(9)HexNAc(2)Fuc(0)NeuAc(0)
FSDGLESNSSTQFEVKK

SEQ ID NO: 90 - protein, 19 aa, synthetic construct
  CARBOHYD 18: note = N is linked to the N-linked glycan structure Hex(2)HexNAc(2)Fuc(0)NeuAc(0)
GTAGNALMDGASQLMGENR

SEQ ID NO: 91 - protein, 19 aa, synthetic construct
  CARBOHYD 18: note = N is linked to the N-linked glycan structure Hex(3)HexNAc(2)Fuc(0)NeuAc(0)
GTAGNALMDGASQLMGENR

SEQ ID NO: 92 - protein, 19 aa, synthetic construct
  CARBOHYD 18: note = N is linked to the N-linked glycan structure Hex(4)HexNAc(3)Fuc(0)NeuAc(1)
GTAGNALMDGASQLMGENR

SEQ ID NO: 93 - protein, 21 aa, synthetic construct
  CARBOHYD 21: note = R is linked to the N-linked glycan structure Hex(4)HexNAc(3)Fuc(0)NeuAc(0)
YRGTAGNALMDGASQLMGENR

SEQ ID NO: 94 - protein, 21 aa, synthetic construct
  CARBOHYD 21: note = R is linked to the N-linked glycan structure Hex(5)HexNAc(4)Fuc(0)NeuAc(1)
YRGTAGNALMDGASQLMGENR

SEQ ID NO: 95 - protein, 29 aa, synthetic construct
  CARBOHYD 19: note = N is linked to the N-linked glycan structure Hex(5)HexNAc(4)Fuc(0)NeuAc(1)
GGNSNGALCHFPFLYNNHNYTDCTSEGRR

SEQ ID NO: 96 - protein, 16 aa, synthetic construct
  CARBOHYD 8: note = N is linked to the N-linked glycan structure Hex(5)HexNAc(4)Fuc(0)NeuAc(1)
HEEGHMLNCTCFGQGR

SEQ ID NO: 97 - protein, 17 aa, synthetic construct
  CARBOHYD 9: note = N is linked to the N-linked glycan structure Hex(5)HexNAc(4)Fuc(0)NeuAc(1)
RHEEGHMLNCTCFGQGR

SEQ ID NO: 98 - protein, 22 aa, synthetic construct
  CARBOHYD 6: note = N is linked to the N-linked glycan structure Hex(5)HexNAc(4)Fuc(0)NeuAc(2)
GHGHRNGTGHGNSTHHGPEYMR

SEQ ID NO: 99 - protein, 8 aa, synthetic construct
  CARBOHYD 5: note = N is linked to the N-linked glycan structure Hex(5)HexNAc(4)Fuc(0)NeuAc(1)
DTFVNASR

SEQ ID NO: 100 - protein, 21 aa, synthetic construct
  CARBOHYD 7: note = N is linked to the N-linked glycan structure Hex(5)HexNAc(4)Fuc(0)NeuAc(2)
GLTFQQNASSMCVPDQDTAIR

SEQ ID NO: 101 - protein, 21 aa, synthetic construct
  CARBOHYD 7: note = N is linked to the N-linked glycan structure Hex(5)HexNAc(4)Fuc(1)NeuAc(0)
GLTFQQNASSMCVPDQDTAIR

SEQ ID NO: 102 - protein, 21 aa, synthetic construct
  CARBOHYD 7: note = N is linked to the N-linked glycan structure Hex(6)HexNAc(4)Fuc(0)NeuAc(1)
GLTFQQNASSMCVPDQDTAIR

SEQ ID NO: 103 - protein, 21 aa, synthetic construct
  CARBOHYD 7: note = N is linked to the N-linked glycan structure Hex(6)HexNAc(5)Fuc(0)NeuAc(1)
GLTFQQNASSMCVPDQDTAIR

SEQ ID NO: 104 - protein, 21 aa, synthetic construct
  CARBOHYD 7: note = N is linked to the N-linked glycan structure Hex(6)HexNAc(5)Fuc(0)NeuAc(2)
GLTFQQNASSMCVPDQDTAIR

SEQ ID NO: 105 - protein, 26 aa, synthetic construct
  CARBOHYD 2: note = N is linked to the N-linked glycan structure Hex(5)HexNAc(4)Fuc(0)NeuAc(1)
ANLSSQALQMSLDYGFVTPLTSMSIR

SEQ ID NO: 106 - protein, 26 aa, synthetic construct
  CARBOHYD 2: note = N is linked to the N-linked glycan structure Hex(5)HexNAc(4)Fuc(1)NeuAc(2)
ANLSSQALQMSLDYGFVTPLTSMSIR

SEQ ID NO: 107 - protein, 16 aa, synthetic construct
  CARBOHYD 2: note = N is linked to the N-linked glycan structure Hex(4)HexNAc(3)Fuc(0)NeuAc(1)
MNKSTVIDSSCVPVNK

SEQ ID NO: 108 - protein, 16 aa, synthetic construct
  CARBOHYD 2: note = N is linked to the N-linked glycan structure Hex(5)HexNAc(4)Fuc(0)NeuAc(1)
MNKSTVIDSSCVPVNK

SEQ ID NO: 109 - protein, 4 aa, synthetic construct
  CARBOHYD 1: note = N is linked to the N-linked glycan structure Hex(6)HexNAc(5)Fuc(0)NeuAc(2)
NASR

SEQ ID NO: 110 - protein, 13 aa, synthetic construct
  CARBOHYD 7: note = N is linked to the N-linked glycan structure Hex(5)HexNAc(4)Fuc(1)NeuAc(1)
ENKDVLNFTCEPK

SEQ ID NO: 111 - protein, 20 aa, synthetic construct
  CARBOHYD 3: note = N is linked to the N-linked glycan structure Hex(5)HexNAc(4)Fuc(1)NeuAc(1)
SENYTYIWWLNGQSLPVSPR

SEQ ID NO: 112 - protein, 20 aa, synthetic construct
  CARBOHYD 10: note = N is linked to the N-linked glycan structure Hex(5)HexNAc(4)Fuc(1)NeuAc(1)
ILILPSVTRNETGPYQCEIR

SEQ ID NO: 113 - protein, 20 aa, synthetic construct
  CARBOHYD 10: note = N is linked to the N-linked glycan structure Hex(5)HexNAc(4)Fuc(2)NeuAc(2)
ILILPSVTRNETGPYQCEIR

SEQ ID NO: 114 - protein, 20 aa, synthetic construct
  CARBOHYD 3: note = N is linked to the N-linked glycan structure Hex(5)HexNAc(4)Fuc(1)NeuAc(2)
SENYTYIWWLNGQSLPVSPR

SEQ ID NO: 115 - protein, 20 aa, synthetic construct
  CARBOHYD 10: note = N is linked to the N-linked glycan structure Hex(5)HexNAc(4)Fuc(1)NeuAc(2)
ILILPSVTRNETGPYQCEIR

SEQ ID NO: 116 - protein, 20 aa, synthetic construct
  CARBOHYD 10: note = N is linked to the N-linked glycan structure Hex(5)HexNAc(4)Fuc(2)NeuAc(2)
ILILPSVTRNETGPYQCEIR

SEQ ID NO: 117 - protein, 23 aa, synthetic construct
  CARBOHYD 10: note = N is linked to the N-linked glycan structure Hex(5)HexNAc(4)Fuc(0)NeuAc(1)
KGCVLLSHLNETVTVSASLESGR

SEQ ID NO: 118 - protein, 23 aa, synthetic construct
  CARBOHYD 10: note = N is linked to the N-linked glycan structure Hex(5)HexNAc(4)Fuc(0)NeuAc(2)
KGCVLLSHLNETVTVSASLESGR

SEQ ID NO: 119 - protein, 20 aa, synthetic construct
  CARBOHYD 15: note = N is linked to the N-linked glycan structure Hex(5)HexNAc(4)Fuc(0)NeuAc(2)
SHEIWTHSCPQSPGNGTDAS

SEQ ID NO: 120 - protein, 13 aa, synthetic construct
  CARBOHYD 12: note = N is linked to the N-linked glycan structureHex(4)HexNAc(3)Fuc(0)NeuAc(0)
CGLVPVLAENYNK

SEQ ID NO: 121 - protein, 27 aa, synthetic construct
  CARBOHYD 1: note = N is linked to the N-linked glycan structure Hex(5)HexNAc(4)Fuc(0)NeuAc(1)
NISDGFDGIPDNVDAALALPAHSYSGR

SEQ ID NO: 122 - protein, 29 aa, synthetic construct
  CARBOHYD 18: note = N is linked to the N-linked glycan structure Hex(5)HexNAc(4)Fuc(0)NeuAc(2)
AREDIFMETLKDIVEYYNDSNGSHVLQGR

SEQ ID NO: 123 - protein, 18 aa, synthetic construct
  CARBOHYD 7: note = N is linked to the N-linked glycan structure Hex(5)HexNAc(4)Fuc(0)NeuAc(2)
DIVEYYNDSNGSHVLQGR

SEQ ID NO: 124 - protein, 16 aa, synthetic construct
  CARBOHYD 4: note = N is linked to the N-linked glycan structure Hex(5)HexNAc(4)Fuc(0)NeuAc(1)
YLGNATAIFFLPDEGK

SEQ ID NO: 125 - protein, 16 aa, synthetic construct
  CARBOHYD 4: note = N is linked to the N-linked glycan structure Hex(5)HexNAc(4)Fuc(0)NeuAc(2)
YLGNATAIFFLPDEGK

SEQ ID NO: 126 - protein, 16 aa, synthetic construct
  CARBOHYD 4: note = N is linked to the N-linked glycan structure Hex(5)HexNAc(4)Fuc(1)NeuAc(2)
YLGNATAIFFLPDEGK

SEQ ID NO: 127 - protein, 19 aa, synthetic construct
  CARBOHYD 3: note = N is linked to the N-linked glycan structure Hex(5)HexNAc(4)Fuc(1)NeuAc(2)
VSNQTLSLFFTVLQDVPVR

SEQ ID NO: 128 - protein, 33 aa, synthetic construct
  CARBOHYD 10: note = N is linked to the N-linked glycan structure Hex(5)HexNAc(4)Fuc(0)NeuAc(1)
IITILEEEMNVSVCGLYTYGKPVPGHVTVSICR

SEQ ID NO: 129 - protein, 21 aa, synthetic construct
  CARBOHYD 10: note = N is linked to the N-linked glycan structure Hex(5)HexNAc(4)Fuc(0)NeuAc(2)
IITILEEEMNVSVCGLYTYGK

SEQ ID NO: 130 - protein, 33 aa, synthetic construct; identical to SEQ ID NO: 128
  CARBOHYD 10: note = N is linked to the N-linked glycan structure Hex(5)HexNAc(4)Fuc(0)NeuAc(2)

SEQ ID NO: 131 - protein, 33 aa, synthetic construct; identical to SEQ ID NO: 76
  CARBOHYD 6: note = N is linked to the N-linked glycan structure Hex(5)HexNAc(4)Fuc(0)NeuAc(1)

SEQ ID NO: 132 - protein, 31 aa, synthetic construct
  CARBOHYD 4: note = N is linked to the N-linked glycan structure Hex(6)HexNAc(5)Fuc(0)NeuAc(2)
YTGNASALFILPDQDKMEEVEAMLLPETLKR

SEQ ID NO: 133 - protein, 9 aa, synthetic construct
  CARBOHYD 2: note = N is linked to the N-linked glycan structure Hex(5)HexNAc(4)Fuc(0)NeuAc(2)
HNFSHCCSK

SEQ ID NO: 134 - protein, 19 aa, synthetic construct
  CARBOHYD 12: note = N is linked to the N-linked glycan structure Hex(5)HexNAc(4)Fuc(0)NeuAc(2)
ICAMEGLPQKHNFSHCCSK

SEQ ID NO: 135 - protein, 23 aa, synthetic construct
  CARBOHYD 5: note = N is linked to the N-linked glycan structure Hex(5)HexNAc(4)Fuc(0)NeuAc(2)
LGACNDTLQQLMEVFKFDTISEK

SEQ ID NO: 136 - protein, 33 aa, synthetic construct
  CARBOHYD 16: note = N is linked to the N-linked glycan structure Hex(5)HexNAc(4)Fuc(0)NeuAc(1)
QVFPGLNYCTSGAYSNASSTDSASYYPLTGDTR

SEQ ID NO: 137 - protein, 20 aa, synthetic construct
  CARBOHYD 2: note = N is linked to the N-linked glycan structure Hex(5)HexNAc(4)Fuc(1)NeuAc(2)
ENLTAPGSDSAVFFEQGTTR

SEQ ID NO: 138 - protein, 17 aa, synthetic construct
  CARBOHYD 9: note = N is linked to the N-linked glycan structure Hex(5)HexNAc(4)Fuc(0)NeuAc(2)
ELHHLQEQNVSNAFLDK

SEQ ID NO: 139 - protein, 17 aa, synthetic construct
  CARBOHYD 9: note = N is linked to the N-linked glycan structure Hex(5)HexNAc(4)Fuc(1)NeuAc(2)
ELHHLQEQNVSNAFLDK

SEQ ID NO: 140 - protein, 17 aa, synthetic construct
  CARBOHYD 9: note = N is linked to the N-linked glycan structure Hex(6)HexNAc(5)Fuc(0)NeuAc(2)
ELHHLQEQNVSNAFLDK

SEQ ID NO: 141 - protein, 10 aa, synthetic construct
  CARBOHYD 1: note = N is linked to the N-linked glycan structure Hex(5)HexNAc(4)Fuc(0)NeuAc(1)
NGTAVCATNR

SEQ ID NO: 142 - protein, 21 aa, synthetic construct
  CARBOHYD 9: note = H is linked to the N-linked glycan structure Hex(5)HexNAc(2)Fuc(0)NeuAc(0)
TVLTPATNHMGNVTFTIPANR

SEQ ID NO: 143 - protein, 15 aa, synthetic construct
  CARBOHYD 12: note = N is linked to the N-linked glycan structure Hex(6)HexNAc(5)Fuc(0)NeuAc(2)
VCQDCPLLAPLNDTR

SEQ ID NO: 144 - protein, 16 aa, synthetic construct
  CARBOHYD 8: note = N is linked to the N-linked glycan structure Hex(5)HexNAc(4)Fuc(0)NeuAc(2)
HEEGHMLNCTCFGQGR

SEQ ID NO: 145 - protein, 17 aa, synthetic construct
  CARBOHYD 9: note = N is linked to the N-linked glycan structure Hex(5)HexNAc(4)Fuc(0)NeuAc(2)
RHEEGHMLNCTCFGQGR

SEQ ID NO: 146 - protein, 16 aa, synthetic construct
  CARBOHYD 7: note = N is linked to the N-linked glycan structure Hex(6)HexNAc(5)Fuc(0)NeuAc(2)
ALPQPQNVTSLLGCTH

SEQ ID NO: 147 - protein, 31 aa, synthetic construct
  CARBOHYD 19: note = N is linked to the N-linked glycan structure Hex(5)HexNAc(4)Fuc(0)NeuAc(2)
GGETAQSADPQWEQLNNKNLSMPLLPADFHK

SEQ ID NO: 148 - protein, 31 aa, synthetic construct; identical to SEQ ID NO: 147
  CARBOHYD 19: note = N is linked to the N-linked glycan structure Hex(5)HexNAc(4)Fuc(1)NeuAc(2)

SEQ ID NO: 149 - protein, 24 aa, synthetic construct
  CARBOHYD 6: note = N is linked to the N-linked glycan structure Hex(6)HexNAc(5)Fuc(0)NeuAc(2)
MVSHHNLTTGATLINEQWLLTTAK

SEQ ID NO: 150 - protein, 19 aa, synthetic construct
  CARBOHYD 4: note = N is linked to the N-linked glycan structure Hex(5)HexNAc(4)Fuc(0)NeuAc(2)
VLSNNSDANLELINTWVAK

SEQ ID NO: 151 - protein, 21 aa, synthetic construct
  CARBOHYD 9: note = N is linked to the N-linked glycan structure Hex(5)HexNAc(4)Fuc(0)NeuAc(2)
VGQLQLSHNLSLVILVPQNLK

SEQ ID NO: 152 - protein, 27 aa, synthetic construct
  CARBOHYD 18: note = N is linked to the N-linked glycan structure Hex(5)HexNAc(4)Fuc(0)NeuAc(1)
LSLHRPALEDLLLGSEANLTCTLTGLR

SEQ ID NO: 153 - protein, 27 aa, synthetic construct
  CARBOHYD 18: note = N is linked to the N-linked glycan structure Hex(5)HexNAc(4)Fuc(0)NeuAc(2)
LSLHRPALEDLLLGSEANLTCTLTGLR

SEQ ID NO: 154 - protein, 27 aa, synthetic construct
  CARBOHYD 18: note = N is linked to the N-linked glycan structure Hex(5)HexNAc(5)Fuc(0)NeuAc(1)
LSLHRPALEDLLLGSEANLTCTLTGLR

SEQ ID NO: 155 - protein, 9 aa, synthetic construct
  CARBOHYD 5: note = N is linked to the N-linked glycan structure Hex(3)HexNAc(4)Fuc(1)NeuAc(0)
EEQFNSTFR

SEQ ID NO: 156 - protein, 9 aa, synthetic construct
  CARBOHYD 5: note = N is linked to the N-linked glycan structure Hex(4)HexNAc(4)Fuc(1)NeuAc(0)
EEQFNSTFR

SEQ ID NO: 157 - protein, 13 aa, synthetic construct
  CARBOHYD 9: note = N is linked to the N-linked glycan structure Hex(4)HexNAc(4)Fuc(1)NeuAc(0)
TKPREEQFNSTFR

SEQ ID NO: 158 - protein, 13 aa, synthetic construct
  CARBOHYD 9: note = N is linked to the N-linked glycan structure Hex(5)HexNAc(4)Fuc(1)NeuAc(0)
TKPREEQFNSTFR

SEQ ID NO: 159 - protein, 13 aa, synthetic construct
  CARBOHYD 9: note = N is linked to the N-linked glycan structure Hex(5)HexNAc(4)Fuc(1)NeuAc(1)
TKPREEQFNSTFR

SEQ ID NO: 160 - protein, 11 aa, synthetic construct
  CARBOHYD 2: note = N is linked to the N-linked glycan structure Hex(5)HexNAc(4)Fuc(0)NeuAc(1)
ENISDPTSPLR

SEQ ID NO: 161 - protein, 21 aa, synthetic construct
  CARBOHYD 7: note = N is linked to the N-linked glycan structure Hex(5)HexNAc(4)Fuc(0)NeuAc(1)
GLTFQQNASSMCVPDQDTAIR

SEQ ID NO: 162 - protein, 21 aa, synthetic construct
  CARBOHYD 7: note = N is linked to the N-linked glycan structure Hex(5)HexNAc(4)Fuc(1)NeuAc(2)
GLTFQQNASSMCVPDQDTAIR

SEQ ID NO: 163 - protein, 21 aa, synthetic construct
  CARBOHYD 7: note = N is linked to the N-linked glycan structure Hex(5)HexNAc(5)Fuc(0)NeuAc(1) Hex(5)HexNAc(5)Fuc(0)NeuAc(1)
GLTFQQNASSMCVPDQDTAIR

SEQ ID NO: 164 - protein, 21 aa, synthetic construct
  CARBOHYD 7: note = N is linked to the N-linked glycan structure Hex(5)HexNAc(5)Fuc(1)NeuAc(0)
GLTFQQNASSMCVPDQDTAIR

SEQ ID NO: 165 - protein, 11 aa, synthetic construct
  CARBOHYD 3: note = N is linked to the N-linked glycan structure Hex(5)HexNAc(4)Fuc(0)NeuAc(1)
YKNNSDISSTR

SEQ ID NO: 166 - protein, 11 aa, synthetic construct
  CARBOHYD 3: note = N is linked to the N-linked glycan structure Hex(5)HexNAc(4)Fuc(0)NeuAc(1)
YKNNSDISSTR

SEQ ID NO: 167 - protein, 9 aa, synthetic construct
  CARBOHYD 1: note = N is linked to the N-linked glycan structure Hex(5)HexNAc(4)Fuc(1)NeuAc(1)
NNSDISSTR

SEQ ID NO: 168 - protein, 11 aa, synthetic construct
  CARBOHYD 3: note = N is linked to the N-linked glycan structure Hex(5)HexNAc(5)Fuc(0)NeuAc(1)
YKNNSDISSTR

SEQ ID NO: 169 - protein, 11 aa, synthetic construct
  CARBOHYD 3: note = N is linked to the N-linked glycan structure Hex(6)HexNAc(3)Fuc(1)NeuAc(1)
YKNNSDISSTR

SEQ ID NO: 170 - protein, 8 aa, synthetic construct
  CARBOHYD 5: note = N is linked to the N-linked glycan structure Hex(5)HexNAc(4)Fuc(0)NeuAc(2)
GVNFNVSK

SEQ ID NO: 171 - protein, 15 aa, synthetic construct
  CARBOHYD 10: note = N is linked to the N-linked glycan structure Hex(5)HexNAc(4)Fuc(0)NeuAc(2)
VTACHSSQPNATLYK

SEQ ID NO: 172 - protein, 13 aa, synthetic construct
  CARBOHYD 12: note = N is linked to the N-linked glycan structure Hex(6)HexNAc(5)Fuc(0)NeuAc(2)
CGLVPVLAENYNK

SEQ ID NO: 173 - protein, 8 aa, synthetic construct
  CARBOHYD 1: note = N is linked to the N-linked glycan structure Hex(5)HexNAc(4)Fuc(0)NeuAc(1)
NGSLFAFR

SEQ ID NO: 174 - protein, 8 aa, synthetic construct
  CARBOHYD 1: note = N is linked to the N-linked glycan structure Hex(5)HexNAc(4)Fuc(0)NeuAc(2)
NGSLFAFR

SEQ ID NO: 175 - protein, 27 aa, synthetic construct
  CARBOHYD 1: note = N is linked to the N-linked glycan structure Hex(5)HexNAc(4)Fuc(1)NeuAc(2)
NISDGFDGIPDNVDAALALPAHSYSGR

SEQ ID NO: 176 - protein, 21 aa, synthetic construct
  CARBOHYD 12: note = N is linked to the N-linked glycan structure Hex(6)HexNAc(5)Fuc(0)NeuAc(2)
VYIHPFHLVIHNESTCEQLAK

SEQ ID NO: 177 - protein, 13 aa, synthetic construct
  CARBOHYD 8: note = N is linked to the N-linked glycan structure Hex(5)HexNAc(4)Fuc(0)NeuAc(1)
VYKPSAGNNSLYR

SEQ ID NO: 178 - protein, 12 aa, synthetic construct
  CARBOHYD 6: note = N is linked to the N-linked glycan structure Hex(6)HexNAc(5)Fuc(0)NeuAc(1)
GVPQPNITWLKR

SEQ ID NO: 179 - protein, 9 aa, synthetic construct
  CARBOHYD 5: note = N is linked to the N-linked glycan structure Hex(3)HexNAc(4)Fuc(1)NeuAc(0)
EEQYNSTYR

SEQ ID NO: 180 - protein, 9 aa, synthetic construct
  CARBOHYD 5: note = N is linked to the N-linked glycan structure Hex(4)HexNAc(3)Fuc(0)NeuAc(0)
EEQYNSTYR

SEQ ID NO: 181 - protein, 9 aa, synthetic construct
  CARBOHYD 5: note = N is linked to the N-linked glycan structure Hex(5)HexNAc(4)Fuc(0)NeuAc(0)
EEQYNSTYR

SEQ ID NO: 182 - protein, 13 aa, synthetic construct
  CARBOHYD 9: note = N is linked to the N-l-inked glycan structure Hex(5)HexNAc(4)Fuc(1)NeuAc(0)
TKPREEQYNSTYR

SEQ ID NO: 183 - protein, 13 aa, synthetic construct
  CARBOHYD 9: note = N is linked to the N-linked glycan structure Hex(5)HexNAc(4)Fuc(1)NeuAc(1)
TKPREEQYNSTYR

SEQ ID NO: 184 - protein, 14 aa, synthetic construct
  CARBOHYD 4: note = N is linked to the N-linked glycan structure Hex(5)HexNAc(4)Fuc(1)NeuAc(1)
HYTNSSQDVTVPCR

SEQ ID NO: 185 - protein, 14 aa, synthetic construct
  CARBOHYD 4: note = N is linked to the N-linked glycan structure Hex(5)HexNAc(4)Fuc(1)NeuAc(2)
HYTNSSQDVTVPCR

SEQ ID NO: 186 - protein, 21 aa, synthetic construct
  CARBOHYD 9: note = N is linked to the N-linked glycan structure Hex(6)HexNAc(2)Fuc(0)NeuAc(0)
STGKPTLYNVSLVMSDTAGTC

SEQ ID NO: 187 - protein, 22 aa, synthetic construct
  CARBOHYD 8: note = N is linked to the N-linked glycan structure Hex(6)HexNAc(5)Fuc(0)NeuAc(2)
HGIQYFNNNTQHSSLFMLNEVK

SEQ ID NO: 188 - protein, 23 aa, synthetic construct
  CARBOHYD 9: note = N is linked to the N-linked glycan structure Hex(6)HexNAc(5)Fuc(0)NeuAc(2)
ITYSIVQTNCSKENFLFLTPDCK

SEQ ID NO: 189 - protein, 7 aa, synthetic construct
ECDMHYK

(19) United States
(12) Patent Application Publication
(10) Pub. No.: US 2026/0260705 A1
(43) Pub. Date: Sep. 3, 2026

(54) BIOMARKERS FOR DIAGNOSING PREECLAMPSIA

(71) Applicant: VENN BIOSCIENCES CORPORATION, South San Francisco, CA (US)

(72) Inventors: Hector Han-Li HUANG, South San Francisco, CA (US); Prasanna RAMACHANDRAN, South San Francisco, CA (US); Chi-Hung LIN, Belmont, CA (US)

(21) Appl. No.: 18/730,948

(22) PCT Filed: Jan. 31, 2023
(86) PCT No.: PCT/US2023/061692

§ 371 Date: Jul. 22, 2024

Related U.S. Application Data
(60) Provisional application No. 63/305,224, filed on Jan. 31, 2022.
(60) Provisional application No. 63/305,242, filed on Jan. 31, 2022.
(60) Provisional application No. 63/326,163, filed on Mar. 31, 2022.

(51) Int. Cl. G16B 40/00; A61K 31/166; A61K 31/195; A61K 45/06; G01N 33/68; G16H 50/30

(52) U.S. Cl. CPC G16B 40/00; A61K 31/166; A61K 31/195; A61K 45/06; G01N 33/6848; G01N 33/689; G16H 50/30; G01N 2800/368

(57) ABSTRACT

Provided herein are methods of diagnosing preeclampsia based upon the presence, absence, or amount of biomarkers, such as glycopeptides. Also provided herein are methods of treating preeclampsia based upon the presence, absence, or amount of such biomarkers and compositions comprising one or more glycopeptide.

### CROSS-REFERENCE TO RELATED APPLICATIONS

This application claims the priority benefit of U.S. Provisional Patent Application Ser. No. 63/305,224, filed Jan. 31, 2022; 63/305,242, filed Jan. 31, 2022; and 63/326,163 filed Mar. 31, 2022, which are hereby all incorporated by reference herein in their entirety.

### FIELD OF THE INVENTION

The present disclosure generally relates to methods and systems for diagnosing and/or treating preeclampsia and/or determining gestational age. More particularly, the present disclosure relates to analyzing quantification data for a set of peptide structures detected in a biological sample obtained from a subject for use in a diagnostic assessment of the subject's disease state (e.g., healthy, preeclampsia, severe preeclampsia) relating to a disease progression and/or treating the subject; analyzing quantification data for a set of peptide structures detected in a biological sample obtained from a subject for use in assessment of the gestational age of a fetus (e.g., how many weeks gestation); and/or identifying peptide structures in a biological sample obtained from a subject that are suitable for use in a diagnostic assessment of the subject's disease state (e.g., healthy, preeclampsia) relating to a disease progression and/or treating the subject.

### BACKGROUND OF THE INVENTION

Preeclampsia is a pregnancy-specific, multisystem disorder that is characterized by the development of hypertension and proteinuria. The incidence of preeclampsia is about 24 cases per 1000 deliveries in the United States. Complications arising from the hypertension attendant to preeclampsia are one of the leading causes of pregnancy-related deaths. Among the risks associated with preeclampsia are placental abruption, acute renal failure, cerebrovascular and cardiovascular complications, disseminated intravascular coagulation, and maternal death. See, generally, Wagner, L. K., “Diagnosis and Management of Preeclampsia”, American Family Physician, 70: 2317-2324, 2004.

Among the criteria for diagnosis of preeclampsia is the onset of elevated blood pressure and proteinuria after 20 weeks of gestation. Specifically, these criteria include a blood pressure of 140 mm Hg or higher systolic or 90 mm Hg diastolic after 20 weeks of gestation in a woman with previously normal blood pressure. Increased proteinuria corresponds to 0.3 grams or more of protein in a 24 hour urine collection; this generally corresponds with 1+ or greater on a urine dipstick test. More severe preeclampsia presents with more substantial blood pressure elevations and higher degrees of proteinuria. Thus, severe preeclampsia may be indicated by 160 mm Hg or higher systolic or 110 mm Hg or higher diastolic on two occasions at least six hours apart in a woman on bed rest. In severe cases, proteinuria may be elevated to 5 grams or more of protein in a 24 hour urine collection or 3+ or greater on urine dipstick testing of two random samples collected at least four hours apart. Other features of severe preeclampsia include: oliguria (less than 500 mL of urine in 24 hours), cerebral or visual disturbances, pulmonary edema or cyanosis, epigastric or right upper quadrant pain, impaired liver function, thrombocytopenia, and intrauterine growth restriction. See, generally, Wagner, L. K., “Diagnosis and Management of Preeclampsia”, American Family Physician, 70: 2317-2324, 2004.

Although diagnostic criteria for preeclampsia exist, the diagnosis of preeclampsia may be complicated by other conditions associated with pregnancy. Thus, a physician must determine how a patient's particular set of symptoms fits into the overall spectrum of hypertensive disorders of pregnancy in order to devise an effective course of treatment. In addition, there is currently no way to predict which 5-7 percent of women will develop preeclampsia, before the onset of symptoms. Reliable prediction would allow physicians to tailor an individual woman's care in order to delay or prevent the onset of preeclampsia or to reduce the consequences of the disease, including reducing the risk of developing severe preeclampsia or eclampsia.

Given the severe and even life-threatening consequences of preeclampsia, prediction of a woman's risk of developing the disease, as well as, early and unambiguous diagnosis and effective treatment strategies are imperative.

In addition to predicting preeclampsia, a reliable estimation of fetal gestational age (GA) is essential as it allows appropriate scheduling of a woman's antenatal care, informs obstetric management decisions and facilitates the correct interpretation of fetal growth assessment. Abnormal fetal growth patterns such as growth restriction or macrosomia may be missed or diagnosed incorrectly if gestational age is unknown or incorrect. Reliable gestational age estimation is also important at a population level to calculate rates of preterm delivery and small-for-gestational-age neonates at delivery.

Traditionally, GA is estimated using the first day of the last menstrual period (LMP), which assumes that ovulation occurs on day 14 of the menstrual cycle. Irregular menses, unknown or uncertain dates, oral contraceptive use or recent pregnancy or breastfeeding, issues that occur in a large proportion of women, may all influence the accuracy of this method. In such cases, early (<14 weeks' gestation) ultrasound measurement of fetal crown-rump length (CRL) is recommended. First-trimester GA assessment is more accurate than is dating in late pregnancy because, with advancing gestation, fetal ultrasound measurements have a larger absolute error and growth disturbances become more noticeable, resulting in potential underestimation of GA for an abnormally small fetus and overestimation for a macrosomic fetus. However, not all women are able to have an early ultrasound.

Clinical examination is also sometimes used to determine gestational age. In particular symphysial-pubis fundal height (SFH) and Ballard Score (BS). SFH is determined by measuring from the mother's pubic bone (symphysis pubis) to the top of the womb. The measurement is then applied to the gestation by a simple rule of thumb and compared with normal growth. Ballard score can only be used postnatally and is based on the neonate's physical and neuromuscular maturity up to 4 days after birth. The neuromuscular components are more consistent over time because the physical components mature quickly after birth. However, the neuromuscular components can be affected by illness and drugs (e.g., magnesium sulfate given during labor).

Therefore, there is a significant need for additional methods to accurately determine gestational age of a fetus.

### BRIEF SUMMARY OF THE INVENTION

In some embodiments, provided herein is a method of classifying a biological sample obtained from a subject with respect to a plurality of states associated with preeclampsia, the method comprising receiving peptide structure data corresponding to a set of glycoproteins in the biological sample; inputting quantification data identified from the peptide structure data for a set of peptide structures into a machine-learning model trained to identify a disease indicator based on the quantification data, wherein the set of peptide structures comprises at least one peptide structure identified from a plurality of peptide structures in Table 3; identifying, by the machine-learning model, the disease indicator; and classifying the biological sample with respect to a plurality of states associated with preeclampsia based upon the identified disease indicator.

Also provided herein is a method of detecting the presence of one of a plurality of states associated with preeclampsia in a subject, the method comprising receiving peptide structure data corresponding to a set of glycoproteins in a biological sample obtained from a subject, wherein the peptide structure data comprises at least one peptide structure from Table 3; inputting quantification data identified from the peptide structure data for a set of peptide structures into a machine-learning model trained to identify a disease indicator based on the quantification data; and detecting the presence of a corresponding state of the plurality of states associated with preeclampsia in response to a determination that the identified disease indicator falls within a selected range associated with the corresponding state.

In some embodiments, the plurality of states comprises at least one of a predisposition for preeclampsia, preeclampsia, severe preeclampsia, or a healthy state. In some embodiments, the machine-learning model comprises a logistic regression model. In some embodiments, the machine-learning model was trained by: generating a log error cost function based on a plurality of disease indicators; and minimizing the log error cost function based on the plurality of disease indicators and the quantification data.

In some embodiments, the method further comprises administering an effective amount of an antihypertensive, a corticosteroid, a beta blocker, or an anticonvulsant to treat preeclampsia based upon the disease indicator. In some embodiments, the antihypertensive comprises methyldopa, and the administering the effective amount comprises 0.5-3 gm/day in 2 divided doses. In some embodiments, the beta blocker comprises labetalol and the administering the effective amount comprises a 20 mg dose intravenously.

Also provided herein is a method of determining a risk for developing preeclampsia in a subject comprising: receiving peptide structure data corresponding to a set of glycoproteins in the biological sample obtained from a subject, wherein the peptide structure data comprises at least one peptide structure from Table 3; inputting quantification data for the at least one peptide structure into a machine-learning model trained to generate a risk score indicative of a risk for developing preeclampsia based on the quantification data; outputting, by the machine-learning model, the quantification data using the machine learning model to generate a risk score, thereby determining the risk for developing preeclampsia.

In a some embodiments, provided herein is a method of treating preeclampsia in a subject comprising receiving peptide structure data corresponding to a set of glycoproteins in the biological sample obtained from a subject, wherein the peptide structure data comprises at least one peptide structure from Table 3; inputting quantification data for the at least one peptide structure into a machine-learning model trained to generate a risk score indicative of a risk for developing preeclampsia based on the quantification data; outputting, by the machine-learning model, the quantification data using the machine learning model to generate a risk score; and administering an effective amount of an antihypertensive, a corticosteroid, a beta blocker, or an anticonvulsant to treat preeclampsia based upon the risk score. In some embodiments, the antihypertensive comprises methyldopa, and the administering the effective amount comprises 0.5-3 gm/day in 2 divided doses. In some embodiments, the beta blocker comprises labetalol and the administering the effective amount comprises a 20 mg dose intravenously.

Also provided herein is a method of determining a risk for developing preeclampsia in a subject comprising: receiving peptide structure data corresponding to a set of glycoproteins in a biological sample; inputting quantification data identified from the peptide structure data for a set of peptide structures into a machine-learning model trained to identify a disease indicator based on the quantification data, wherein the set of peptide structure data comprises at least one peptide structure identified from a plurality of peptide structures in Table 3; identifying, by the machine-learning model, the disease indicator; and determining a risk for preeclampsia based upon the identified disease indicator.

In some embodiments, provided herein is a method of treating preeclampsia in a subject comprising: receiving peptide structure data corresponding to a set of glycoproteins in the biological sample; inputting quantification data identified from the peptide structure data for a set of peptide structures into a machine-learning model trained to identify a disease indicator based on the quantification data, wherein the peptide structure data comprises at least one peptide structure identified from a plurality of peptide structures in Table 3; identifying, by the machine-learning model, the disease indicator; determining a risk for preeclampsia based upon the identified disease indicator; and administering an effective amount of an antihypertensive, a corticosteroid, a beta blocker, or an anticonvulsant to treat preeclampsia based upon the risk score. In some embodiments, the antihypertensive comprises methyldopa, and the administering the effective amount comprises 0.5-3 gm/day in 2 divided doses. In some embodiments, the beta blocker comprises labetalol and the administering the effective amount comprises a 20 mg dose intravenously.

Also provided herein is a method of treating preeclampsia in an individual comprising detecting the presence or amount of at least one peptide structure, wherein the at least one peptide structure comprises at least one peptide structure from Table 3, and administering an effective amount of an antihypertensive, a corticosteroid, a beta blocker, or an anticonvulsant to treat preeclampsia based upon the presence or amount of the peptide structure.

In some embodiments, provided herein is a method of treating preeclampsia in an individual comprising detecting a presence or amount of at least one peptide structure to determine a risk of preeclampsia, wherein the at least one peptide structure comprises at least one peptide structure from Table 3, and administering an effective amount of an antihypertensive, a corticosteroid, a beta blocker, or an anticonvulsant to treat preeclampsia based upon the determined risk of preeclampsia.

In some embodiments, provided herein is a method of diagnosing an individual with preeclampsia or risk of pre-term birth comprising detecting a presence or amount of at least one peptide structure, wherein the at least one peptide structure comprises at least one peptide structure from Table 3, and diagnosing the individual with preeclampsia or risk of pre-term birth based upon the presence or amount of the at least one peptide structure.

In some embodiments, provide herein is a method of determining a risk for developing preeclampsia or risk of pre-term birth comprising detecting a presence or amount of at least one peptide structure and determining the risk for developing preeclampsia or risk of pre-term birth based upon the presence or amount of the at least one peptide structure, wherein the at least one peptide structure comprises at least one peptide structure from Table 3.

Also provided herein is a method of diagnosing an individual with preeclampsia comprising detecting the presence or amount of at least one peptide structure structures from Table 3; inputting a quantification of the detected at least one peptide structure into a machine-learning model trained to generate a class label; determining if the class label is above or below a threshold for a classification; identifying a diagnostic classification for the individual based on whether the class label is above or below a threshold for the classification; and diagnosing the individual as having preeclampsia based on the diagnostic classification.

In some embodiments, the presence or amount of the at least one peptide structure is detected using mass spectrometry or ELISA. In some embodiments, the presence or amount of the at least one peptide structure is detected using MRM mass spectrometry. In some embodiments, the amount of at least one peptide structure is none, or below a detection limit. In some embodiments, the preeclampsia is severe preeclampsia. In some embodiments, the biological sample is maternal serum or maternal plasma. In some embodiments, the one or more peptide structure comprises a glycopeptide of a pregnancy-specific protein.

In some embodiments, the at least one peptide structure comprises three or more peptide structures identified in Table 3. In some embodiments, the at least one peptide structure comprises the sequence set forth in SEQ ID NOs:5-12.

In some embodiments, the method further comprises assessing one or more risk factors or clinical indicators of preeclampsia. In some embodiments, the clinical indicator of preeclampsia is selected from the group consisting of protein in the urine and high blood pressure. In some embodiments, the risk factor for preeclampsia is selected from the group consisting of history of preeclampsia, chronic hypertension, obesity, and multiple pregnancy.

In some embodiments, the individual is determined have a healthy state, wherein a healthy state comprises the absence of preeclampsia and/or a low risk for preeclampsia.

In some embodiments, the method further comprises diagnosing a placental development problem.

In some embodiments, the method further comprises generating a report that includes a diagnosis based on the corresponding state detected for the subject.

Also provided herein is a method of training a model to diagnose a subject with one of a plurality of states associated with preeclampsia, the method comprising: receiving quantification data for a panel of peptide structures for a plurality of subjects diagnosed with the plurality of states associated with preeclampsia; and training a machine-learning model to determine a state of the plurality of states a biological sample from the subject based on the quantification data. In some embodiments, the quantification data comprises at least one of an abundance, a relative abundance, a normalized abundance, a relative quantity, an adjusted quantity, a normalized quantity, a relative concentration, an adjusted concentration, or a normalized concentration. In some embodiments, the machine-learning model is trained using random forest or logical progression training methods. In some embodiments, the method further comprises pooling samples from multiple individuals stratified by gestational age. In some embodiments, training the machine-learning model to determine the state of the plurality of states comprises training the machine-learning model to generate a class label for the state of the plurality of states. In some embodiments, the machine-learning model comprises a logistic regression model.

In some embodiments, the machine-learning model was further trained by: generating a log error cost function based on the plurality of states associated with preeclampsia; and minimizing the log error cost function based on the plurality of states associated with preeclampsia and the determined state of the plurality of states. In some embodiments, the machine-learning model was further trained by: generating a cost function based on the plurality of states associated with preeclampsia; and minimizing the cost function based on the plurality of states associated with preeclampsia and the determined state of the plurality of states. In some embodiments, the cost function comprises a rectified linear unit (ReLU) cost function.

In some embodiments, at least one of the peptide structures comprises a glycopeptide.

Also provided herein is a composition comprising one or more peptide structures from Table 3.

Provided herein is a composition comprising one or more peptides comprising the sequence set forth in SEQ ID NOs:5-12.

In some embodiments, provided herein is a method of classifying a biological sample obtained from a subject with respect to a plurality of states associated with fetal gestational age, the method comprising receiving peptide structure data corresponding to a set of glycoproteins in the biological sample; inputting quantification data identified from the peptide structure data for a set of peptide structures into one or more machine-learning models trained to identify a fetal gestational age indicator based on the quantification data, wherein the set of the peptide structures comprises at least one peptide structure identified from a plurality of peptide structures in Table 10; identifying, by the one or more machine-learning models, the fetal gestational age indicator; and classifying the biological sample with respect to a plurality of states associated with fetal gestational age based upon the identified fetal gestational age indicator.

Also provided herein is a method of detecting the presence of one of a plurality of states associated with fetal gestational age, the method comprising receiving peptide structure data corresponding to a set of glycoproteins in a biological sample obtained from a subject, wherein the peptide structure data comprises at least one peptide structure identified from a plurality of peptide structures in Table 10; inputting quantification data identified from the peptide structure data for a set of peptide structures into one or more machine-learning models trained to identify a fetal gestational age indicator based on the quantification data; and detecting the presence of a corresponding state of the plurality of states associated with fetal gestational age in response to a determination that the identified fetal gestational age indicator falls within a selected range associated with the corresponding state.

In some embodiments, the plurality of states comprises a number of weeks of gestation of a fetus. In some embodiments, the one or more machine-learning models comprises an ensemble learning model. In some embodiments, the ensemble learning model comprises a plurality of decision trees, and wherein a succeeding decision tree of the plurality of decision trees is trained to correct an error of a preceding decision tree of the plurality of decision trees to identify the fetal gestational age indicator. In some embodiments, the one or more machine-learning models comprises one or more of a gradient boosting model, an adaptive boosting (AdaBoost) model, an eXtreme gradient boosting (XGBoost) model, a light gradient boosted machine (LightGBM) model, or a categorical boosting (CatBoost) model.

In some embodiments, provided herein is a method of determining fetal gestational age comprising receiving peptide structure data corresponding to a set of glycoproteins in the biological sample obtained from a subject, wherein the peptide structure data comprises at least one peptide structure from Table 10; inputting quantification data for the at least one peptide structure into one or more machine-learning models trained to generate a fetal gestational age score based on the quantification data; analyzing the quantification data using the one or more machine-learning models to generate a fetal gestational age score, thereby determining a fetal gestational age.

Also provided herein is a method of determining a fetal gestational age comprising detecting at least one peptide structure from Table 10; inputting a quantification of the at least one detected peptide structure into one or more trained machine-learning models to generate an output probability; determining if the output probability is above or below a threshold for a classification; identifying a fetal gestational age classification based on whether the output probability is above or below a threshold for a classification; and determining a fetal gestational age based upon the fetal gestational age classification.

In some embodiments, provided herein is a method of determining a gestational age of a fetus comprising detecting the presence or amount at least one peptide structure from Table 10, and determining the gestational age of the fetus based upon the presence or amount of the at least one peptide structure from Table 10.

In some embodiments, detecting the at least one peptide structure is performed using mass spectrometry or ELISA. In some embodiments, detecting the at least one peptide structure is performed using MRM mass spectrometry.

In some embodiments, the gestational age is over 20 weeks. In some embodiments, the gestational age is over 24 weeks. In some embodiments, the biological sample is maternal serum or plasma. In some embodiments, the biological sample is collected in the second or third trimester of pregnancy.

In some embodiments, the at least one peptide structure comprises a glycopeptide. In some embodiments, the glycoprotein is a pregnancy-specific protein.

In some embodiments, at least one peptide structure comprises at least three peptide structures identified in Table 10. In some embodiments, the at least one peptide structure comprises a peptide consisting of the sequence set forth in SEQ ID NOs: 16-21.

In some embodiments, the method further comprises assessing one or more additional clinical indicators for gestational age. In some embodiments, the one or more additional clinical indicators is selected from the group consisting of ultra sound fetal images, and fundal height.

In some embodiments, the method further comprises generating a report that includes the gestational age of the fetus.

In some embodiments, provided herein is a method of training a model to determine a plurality of states associated with fetal gestational age, the method comprising: receiving quantification data for a panel of peptide structures for a plurality of subjects at varying gestational ages; and training one or more machine-learning models to determine a state of the plurality of states that corresponds based on the quantification data. In some embodiments, the quantification data comprises at least one of an abundance, a relative abundance, a normalized abundance, a relative quantity, an adjusted quantity, a normalized quantity, a relative concentration, an adjusted concentration, or a normalized concentration. In some embodiments, the method further comprises pooling samples from multiple individuals stratified by gestational age. In some embodiments, training the machine-learning model to determine the state of the plurality of states comprises training the machine-learning model to generate a class label for the state of the plurality of states. In some embodiments, the one or more machine-learning models comprises an ensemble learning model. In some embodiments, the ensemble learning model comprises a plurality of decision trees, and wherein a succeeding decision tree of the plurality of decision trees is trained to correct an error of a preceding decision tree of the plurality of decision trees to identify the fetal gestational age indicator. In some embodiments, the one or more machine-learning models comprise one or more of a gradient boosting model, an adaptive boosting (AdaBoost) model, an eXtreme gradient boosting (XGBoost) model, a light gradient boosted machine (LightGBM) model, or a categorical boosting (CatBoost) model.

In some embodiments, at least one of the peptide structures comprises a glycopeptide.

Also provided herein is a composition comprising at least one peptide structure from Table 10.

Provided herein is a composition comprising at least one peptide comprising the sequence set forth in SEQ ID NO:16-21.

In some aspects, the method relates to diagnosis of preeclampsia based upon certain glycopeptide biomarkers provided herein, such as those in Table 17. In some embodiments, the methods provided herein are minimally invasive or non-invasive methods for diagnosing preeclampsia that result in early detection of preeclampsia and/or identification of a risk of preeclampsia to enable early and/or prophylactic treatment for at risk individuals.

In some embodiments, the method further comprises administering an effective amount of an antihypertensive, a corticosteroid, a beta blocker, or an anticonvulsant to treat preeclampsia based upon the disease indicator. In some embodiments, the antihypertensive comprises methyldopa, and the administering the effective amount comprises 0.5-3 gm/day in 2 divided doses. In some embodiments, the beta blocker comprises labetalol and the administering the effective amount comprises a 20 mg dose intravenously.

Also provided herein is a method of treating preeclampsia in an individual comprising detecting the presence or amount of at least one peptide structure, wherein the at least one peptide structure comprises at least one peptide structure from Table 17, and administering an effective amount of an antihypertensive, a corticosteroid, a beta blocker, or an anticonvulsant to treat preeclampsia based upon the presence or amount of the peptide structure.

In some embodiments, provided herein is a method of treating preeclampsia in an individual comprising detecting a presence or amount of at least one peptide structure to determine a risk of preeclampsia, wherein the at least one peptide structure comprises at least one peptide structure from Table 17, and administering an effective amount of an antihypertensive, a corticosteroid, a beta blocker, or an anticonvulsant to treat preeclampsia based upon the determined risk of preeclampsia.

In some embodiments, provided herein is a method of diagnosing an individual with preeclampsia comprising detecting a presence or amount of at least one peptide structure, wherein the at least one peptide structure comprises at least one peptide structure from Table 17, and diagnosing the individual with preeclampsia based upon the presence or amount of the at least one peptide structure.

In some embodiments, provide herein is a method of determining a risk for developing preeclampsia comprising detecting a presence or amount of at least one peptide structure and determining the risk for developing preeclampsia based upon the presence or amount of the at least one peptide structure, wherein the at least one peptide structure comprises at least one peptide structure from Table 17.

In some embodiments, the presence or amount of the at least one peptide structure is detected using mass spectrometry or ELISA. In some embodiments, the presence or amount of the at least one peptide structure is detected using MRM mass spectrometry. In some embodiments, the amount of at least one peptide structure is none, or below a detection limit. In some embodiments, the preeclampsia is severe preeclampsia. In some embodiments, the biological sample is maternal serum or maternal plasma. In some embodiments, the one or more peptide structure comprises a glycopeptide of a pregnancy-specific protein.

In some embodiments, the at least one peptide structure comprises three or more peptide structures identified in Table 17. In some embodiments, the at least one peptide structure comprises the sequence set forth in SEQ ID NOs: 65-188.

In some embodiments, the method further comprises assessing one or more risk factors or clinical indicators of preeclampsia. In some embodiments, the clinical indicator of preeclampsia is selected from the group consisting of protein in the urine and high blood pressure. In some embodiments, the risk factor for preeclampsia is selected from the group consisting of history of preeclampsia, chronic hypertension, obesity, and multiple pregnancy.

In some embodiments, the individual is determined have a healthy state, wherein a healthy state comprises the absence of preeclampsia and/or a low risk for preeclampsia.

In some embodiments, the method further comprises diagnosing a placental development problem.

In some embodiments, the method further comprises generating a report that includes a diagnosis based on the corresponding state detected for the subject.

In some embodiments, at least one of the peptide structures comprises a glycopeptide.

Also provided herein is a composition comprising one or more peptide structures from Table 17.

Provided herein is a composition comprising one or more peptides comprising the sequence set forth in SEQ ID NOs: 65-188.

In some embodiments, the at least one peptide structure includes a glycan bound to the at least one peptide structure peptide in accordance with Tables 3 and 4.

### BRIEF DESCRIPTION OF THE DRAWINGS

FIG. 1 shows an exemplary workflow for the detection of peptide structures associated with a disease state for use in diagnosis, treatment, and/or determining gestational age in accordance with one or more embodiments.

FIG. 2A shows a schematic diagram of a preparation workflow in accordance with one or more embodiments.

FIG. 2B shows a process of data acquisition in accordance with one or more embodiments.

FIG. 3 is a block diagram of an analysis system in accordance with one or more embodiments.

FIG. 4 is a block diagram of a computer system in accordance with one or more embodiments.

FIG. 5 is a flowchart of a process for classifying a biological sample obtained from a subject with respect to a plurality of states associated with preeclampsia in accordance with one or more embodiments.

FIG. 6 is a flowchart of a process for detecting the presence of one of a plurality of states associated with preeclampsia in a subject in accordance with one or more embodiments.

FIG. 7 is a flowchart of a process for determining a risk for developing preeclampsia in a subject in accordance with one or more embodiments.

FIG. 8 is a flowchart of a process for determining a risk for developing preeclampsia in a subject in accordance with one or more embodiments.

FIG. 9 is a flowchart of a process for training a model to diagnose a subject with one of a plurality of states associated with preeclampsia in accordance with one or more embodiments.

FIG. 10 is a flowchart of a process for treating preeclampsia in a subject in accordance with one or more embodiments.

FIG. 11 is a flowchart of a process for treating preeclampsia in a subject in accordance with one or more embodiments.

FIG. 12 is a flowchart of a process for diagnosing an individual with preeclampsia in accordance with one or more embodiments.

FIG. 13 shows an experimental workflow for sample preparation and analysis.

FIG. 14 is an illustration of a plot of the principal component analysis for sample sets using the identified peptide structures.

FIG. 15 is a heat map showing relative abundances of the identified peptide structures in control, pre-term birth, and preeclampsia groups.

FIG. 16 is a box plot showing the disease indicator's ability to distinguish between control, PTB (preterm birth), and PE (preeclampsia).

FIG. 17 shows the receiver-operating-characteristic (ROC) curve for distinguishing between the PE state and the PTB state for both the training and testing sets in accordance with one or more embodiments.

FIG. 18 is a flowchart of a process for classifying a biological sample obtained from a subject with respect to a plurality of states associated with fetal gestational age in accordance with one or more embodiments.

FIG. 19 is a flowchart of a process for detecting the presence of one of a plurality of states associated with fetal gestational age in accordance with one or more embodiments.

FIG. 20 is a flowchart of a process for training a model to determine a plurality of states associated with fetal gestational age in accordance with one or more embodiments.

FIG. 21 is a flowchart of a process for determining fetal gestational age in accordance with one or more embodiments.

FIG. 22 is a flowchart of a process for determining a fetal gestational age in a subject in accordance with one or more embodiments.

FIG. 23 is a plot showing the performance of a trained model in predicting week of gestation (wog) for samples in a sample set.

FIG. 24 shows an experimental workflow for sample preparation and analysis.

### DETAILED DESCRIPTION OF THE INVENTION

Provided herein are methods useful for diagnosing preeclampsia based upon one or more biomarkers. In some embodiments, the diagnosis is based upon the presence, absence, and/or amount of one or more peptide structures comprising a sequence set forth in SEQ ID NOs: 5-12. In some embodiments, a machine learning model is used to classify the sample with respect to a state associated with preeclampsia, such as preeclampsia, severe preeclampsia, or a healthy state.

In some embodiments, the present methods are able to predict the likelihood or risk that a pregnant individual will develop preeclampsia based upon the presence, absence, and/or amount of one or more peptide structures comprising a sequence set forth in SEQ ID NOs: 5-12. These methods are particularly useful because symptoms of preeclampsia can begin rapidly and can be life threatening. Thus, evaluating the risk of preeclampsia allows for closer monitoring of those individuals at higher risk, and/or prophylactic treatment to prevent preeclampsia.

Provided herein are methods to determine a gestational age based upon the presence, absence and/or amount of one or more biomarkers. In some embodiments, the biomarker is a glycopeptide. In some embodiments, the present methods advantageously are able to determine a fetal gestational age in the second or third trimester, when other methods (such as ultrasound) may not be accurate, or in situations where an individual does not have access to an ultrasound machine. In some embodiments, the biomarker is detected using mass spectrometry (such as MRM-MS), which is able to quantify low abundance peptides in complex mixtures without having to purify the biomarker peptides, such as glycopeptides. In some embodiments, the biomarker is a peptide from Table 10. In some embodiments, the biomarker is a peptide comprising a sequence set forth in any of SEQ ID NOs:16-21.

Provided herein are methods useful for diagnosing preeclampsia based upon one or more biomarkers. In some embodiments, the diagnosis is based upon the presence, absence, and/or amount of one or more peptide structures comprising a sequence set forth in SEQ ID NOs: 65-188.

In some embodiments, the present methods are able to predict the likelihood or risk that a pregnant individual will develop preeclampsia based upon the presence, absence, and/or amount of one or more peptide structures comprising a sequence set forth in SEQ ID NOs: 65-188. These methods are particularly useful because symptoms of preeclampsia can begin rapidly and can be life threatening. Thus, evaluating the risk of preeclampsia allows for closer monitoring of those individuals at higher risk, and/or prophylactic treatment to prevent preeclampsia.

### I. Definitions

As used herein, the term “plurality” is more than 1 and may be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list is required to be included. The item may be a particular object, thing, step, operation, process, or category. In other words, “at least one of” means any combination of items or number of items may be used from the list, but not all of the items in the list may be required. For example, without limitation, “at least one of item A, item B, and item C” intends and includes any of item A; item A and item B; item B; item A, item B, and item C; item B and item C; item C; and item A and C. It is understood that “at least one of” includes instance where more than one of any listed item is present. For example, and without limitation, at least one of item A, item B, and item C include an embodiment in which two of item A is present, one of item B is present, and ten of item C is present.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance.

The term “amino acid,” as used herein, generally refers to any organic compound that includes an amino group (e.g., —NH2), a carboxyl group (—COOH), and a side chain group (R) which varies based on a specific amino acid. Thus, “amino acid” includes organic compounds of the formula NH2-CH(HXR)—COOH where R represents an amino acid side chain group. In some instance R represents the side chain of a natural amino acid. Amino acids can be linked using peptide bonds.

The term “alkylation,” as used herein, generally refers to the transfer of an alkyl group from one molecule to another. In various embodiments, alkylation is used to react with reduced cysteines to prevent the re-formation of disulfide bonds after reduction has been performed.

The term “linking site” or “glycosylation site” as used herein generally refers to the location where a sugar molecule of a glycan or glycan structure is directly bound (e.g., covalently bound) to an amino acid of a peptide, a polypeptide, or a protein. For example, the linking site may be an amino acid residue and a glycan structure may be linked via an atom of the amino acid residue. Non-limiting examples of types of glycosylation can include N-linked glycosylation, O-linked glycosylation, C-linked glycosylation, S-linked glycosylation, and glycation.

The term “biomarker,” as used herein, generally refers to any measurable substance taken as a sample from a subject whose presence, absence and/or amount is indicative of some phenomenon. Non-limiting examples of such phenomenon can include a disease state, a condition, or exposure to a compound or environmental condition. In various embodiments described herein, biomarkers may be used for diagnostic purposes (e.g., to diagnose a disease state, a health state, an asymptomatic state, a symptomatic state, etc.). The term “biomarker” may be used interchangeably with the term “marker.” Biomarkers include peptide structures such as those listed in Table 3.

The term “denaturation,” as used herein, generally refers to protein unfolding. Non-limiting examples include proteins or nucleic acids being exposed to an external compound or environmental condition such as acid, base, temperature, pressure, radiation, etc.

The term “denatured protein,” as used herein, generally refers to a protein that loses quaternary structure, tertiary structure, and secondary structure which is present in its native state.

The terms “digestion” or “enzymatic digestion” or “proteolytic digest,” as used herein, generally refer to breaking apart a polymer (e.g., cutting a polypeptide at a cut site). Proteins may be digested in preparation for mass spectrometry using trypsin digestion protocols. Proteins may be digested using other proteases in preparation for mass spectrometry if access is limited to cleavage sites.

The term “disease progression,” as used herein, refers to a progression of a disease from no disease or a less advanced form of disease to a more advanced (e.g., severe) form of the disease. A disease progression may include any number of stages of the disease.

The term “disease state” as used herein, generally refers to a condition that affects the structure or function of an organism. Disease states can include, for example, stages of a disease progression. Disease states can include any state of a disease whether symptomatic or asymptomatic. Disease states can cause minor, moderate, or severe disruptions in the structure or function of a subject. Disease state includes preeclampsia, severe preeclampsia, disposition or likelihood of preeclampsia, or normal or healthy state with respect to preeclampsia.

The terms “glycan” or “polysaccharide” as used herein, both generally refer to a carbohydrate residue of a glycoconjugate, such as the carbohydrate portion of a glycopeptide, glycoprotein, glycolipid, or proteoglycan. Glycans can include monosaccharides.

The term “glycoprotein” or “glycopolypeptide” as used herein, generally refers to a protein having at least one glycan residue bonded thereto. In some examples, a glycoprotein is a protein with at least one oligosaccharide chain covalently bonded thereto. Examples of glycoproteins, include but are not limited SEQ ID NOs: 1, 2, and 4.

The term “glycopeptide” as used herein, refers to a fragment of a glycoprotein, unless specified otherwise to the contrary. In various embodiments, glycopeptides comprise carbohydrate moieties (e.g., one or more glycans) covalently attached to a side chain (i.e. R group) of an amino acid residue. Examples of glycopeptides, include but are not limited to SEQ ID NOs: 5-8.

The term “liquid chromatography,” as used herein, generally refers to a technique used to separate a sample into parts. Liquid chromatography can be used to separate, identify, and quantify components.

The term “mass spectrometry” as used herein, generally refers to an analytical technique used to identify molecules. In various embodiments described herein, mass spectrometry can be involved in characterization and sequencing of proteins as well as to determine the presence, absence and/or abundance or peptides or proteins.

The term “m/z” or “mass-to-charge ratio” as used herein, generally refers to an output value from a mass spectrometry instrument. In various embodiments, m/z can represent a relationship between the mass of a given ion and the number of elementary charges that it carries. The “m” in m/z stands for mass and the “z” stands for charge. In some embodiments, m/z can be displayed on an x-axis of a mass spectrum.

The term “peptide,” as used herein, refers to amino acids linked by peptide bonds less than 50 amino acids in length. Peptides can include amino acid chains shorter than 10 residues, including, oligopeptides, dipeptides, tripeptides, and tetrapeptides. Peptides includes peptides comprising consisting of, or consisting essentially of the peptide structures provided in Table 3.

The terms “protein” or “polypeptide” or may be used interchangeably herein and refer to a polymer in which the monomers are amino acid residues that are joined together through amide bonds of at least 50 amino acid residues in length. Proteins may be digested in preparation for mass spectrometry using trypsin digestion protocols. Proteins may be digested using other proteases in preparation for mass spectrometry if access is limited to cleavage sites.

The term “peptide structure,” as used herein, generally refers to peptides or a portion thereof or glycopeptides or a portion thereof. In various embodiments described herein, a peptide structure can include any molecule comprising at least two amino acids in sequence. A peptide structure of a glycopeptide includes description of the peptide amino acids sequence as well as the location and identity of the associated glycan.

The term “reduction,” as used herein, generally refers to the gain of an electron by a substance. In various embodiments, reduction may be used to break disulfide bonds between two cysteines.

The term “sample” and “biological sample” as used herein, generally refers to a sample obtained from a subject of interest. The sample may include maternal serum, maternal blood, and/or amniotic fluid. The sample may include a cell sample. The sample may include a cell line or cell culture sample. The sample can include one or more cells. The sample can include one or more microbes. The sample may include a nucleic acid sample or protein sample. The sample may also include a carbohydrate sample or a lipid sample. The sample may be derived from another sample. The sample may include a tissue sample, such as a biopsy, core biopsy, needle aspirate, or fine needle aspirate. The sample may include a fluid sample, such as a blood sample, urine sample, or saliva sample. The sample may include a skin sample. The sample may include a cheek swab. The sample may include a plasma or serum sample. The sample may include a cell free sample. A cell-free sample may include extracellular polynucleotides. The sample may originate from blood, plasma, serum, urine, saliva, mucosal excretions, sputum, stool, or tears. The sample may originate from red blood cells or white blood cells. The sample may originate from feces, spinal fluid, CNS fluid, gastric fluid, amniotic fluid, cyst fluid, peritoneal fluid, marrow, bile, other body fluids, tissue obtained from a biopsy, skin, or hair.

The term “sequence,” as used herein, generally refers to a biological sequence including one-dimensional monomers that can be assembled to generate a polymer. Non-limiting examples of sequences include nucleotide sequences (e.g., ssDNA, dsDNA, and RNA), amino acid sequences (e.g., proteins, peptides, and polypeptides), and carbohydrates.

The term “subject” or “individual” are used interchangeably herein, and refer to a human. A subject can include a healthy or asymptomatic individual, an individual that has or is suspected of having a disease (e.g., preeclampsia) or a pre-disposition to the disease, and/or an individual that needs therapy or suspected of needing therapy. A subject can be a patient. In some embodiments, the subject is a female human. In some embodiments, a subject is a pregnant human. In some embodiments, a subject is an individual in the second or third trimester of gestation.

As used herein, a “model” may include one or more algorithms, one or more mathematical techniques, one or more machine learning algorithms, or a combination thereof.

As used herein, “machine learning” may be the practice of using algorithms to parse data, learn from it, and then make a determination or prediction about something in the world. Machine learning uses algorithms that can learn from data without relying on rules-based programming. A machine learning algorithm may include a parametric model, a nonparametric model, a deep learning model, a neural network, a linear discriminant analysis model, a quadratic discriminant analysis model, a support vector machine, a random forest algorithm, a nearest neighbor algorithm, a combined discriminant analysis model, a k-means clustering algorithm, a supervised model, an unsupervised model, logistic regression model, a multivariable regression model, a penalized multivariable regression model, or another type of model.

As used herein, an “artificial neural network” or “neural network” (NN) may refer to mathematical algorithms or computational models that mimic an interconnected group of artificial nodes or neurons that processes information based on a connectionistic approach to computation. Neural networks, which may also be referred to as neural nets, can employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in two ways: when it is being trained it is in training mode and when it puts what it has learned into practice it is in inference (or prediction) mode. Neural networks learn through a feedback process (e.g., backpropagation) which allows the network to adjust the weight factors (modifying its behavior) of the individual nodes in the intermediate hidden layers so that the output matches the outputs of the training data. In other words, a neural network learns by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), or another type of neural network.

As used herein, a “target glycopeptide analyte,” may refer to a peptide structure (e.g., glycosylated or aglycosylated/non-glycosylated), a fraction of a peptide structure, a sub-structure (e.g., a glycan or a glycosylation site) of a peptide structure, a product of one or more of the above listed structures and sub-structures, associated detection molecules (e.g., signal molecule, label, or tag), or an amino acid sequence that can be measured by mass spectrometry. For example, a quadrupole mass analyzer of mass spectrometer can be configured to filter a preselected m/z value that corresponds to a target glycopeptide analyte in an ionized state.

As used herein, a “peptide data set,” may be used interchangeably with “peptide structure data” and can refer to any data of or relating to a peptide presence or abundance. For example, peptide data set or peptide structure data can be based upon a mass spectrometry run, an ELISA, or western blot. A peptide data set can comprise data obtained from a sample or biological sample using mass spectrometry. A peptide dataset can comprise data relating to a NGEP external standard, data relating to an internal standard, and data relating to a target glycopeptide analyte of a sample. A peptide data set can result from analysis originating from a single run. In some embodiments, the peptide data set can include raw abundance and mass to charge ratios for one or more peptides.

As used herein, a “non-glycosylated endogenous peptide” (“NGEP”), which may also be referred to as an aglycosylated peptide, may refer to a peptide structure that does not comprise a glycan molecule. In various embodiments, an NGEP and a target glycopeptide analyte can originate from the same subject. In various embodiments, an NGEP can be labeled with an isotope in preparation for mass spectrometry analysis.

As used herein, a “transition,” may refer to or identify a peptide structure. In some embodiments, a transition can refer to the specific pair of m/z values associated with a precursor ion and a product or fragment ion.

As used herein, an “abundance value” may refer to “abundance” or a quantitative value associated with abundance.

As used herein, “abundance,” may refer to a quantitative value generated using mass spectrometry. In various embodiments, the quantitative value may relate to an amount of a particular peptide structure (e.g., biomarker) present in a biological sample. In some embodiments, the amount may be in relation to other structures present in the sample (e.g., relative abundance). In some embodiments, the quantitative value may comprise an amount of an ion produced using mass spectrometry. In some embodiments, the quantitative value may be associated with an m/z value (e.g., abundance on x-axis and m/z on y-axis). In other embodiments, the quantitative value may be expressed in atomic mass units.

As used herein, “relative abundance,” may refer to a comparison of two or more abundances. In various embodiments, the comparison may comprise comparing one peptide structure to a total number of peptide structures. In some embodiments, the comparison may comprise comparing one peptide glycoform (e.g., two identical peptides differing by one or more glycans) to a set of peptide glycoforms. In some embodiments, the comparison may comprise comparing a number of ions having a particular m/z ratio by a total number of ions detected. In various embodiments, a relative abundance can be expressed as a ratio. In other embodiments, a relative abundance can be expressed as a percentage. Relative abundance can be presented on a y-axis of a mass spectrum plot. In some embodiments, the relative abundance can include a ratio of the number of peptide spectrum matching (PSMs) for one peptide structure and the total summation number of PSMs for all of the measured peptide structures, where the term all of the measured peptide structures can be determined by a filtering criteria (e.g., Byonic search score >250).

As used herein, an “internal standard,” may refer to something that can be contained (e.g., spiked-in) in the same sample as a target glycopeptide analyte undergoing mass spectrometry analysis. Internal standards can be used for calibration purposes. Additionally, internal standards can be used in the systems and method described herein. In some aspects, an internal standard can be selected based on similarity m/z and or retention times and can be a “surrogate” if a specific standard is too costly or unavailable. Internal standards can be heavy labeled or non-heavy labeled.

“Preeclampsia” (also known as “toxemia”) as used herein refers to a disorder characterized by the new onset of hypertension and proteinuria or the new onset of hypertension and significant end-organ dysfunction with or without proteinuria in the last half of pregnancy or post-partum. Specifically, preeclampsia may be clinically indicated by a blood pressure of 140 mm Hg or higher systolic or 90 mm Hg diastolic after 20 weeks gestation in a woman with previously normal blood pressure and 0.3 grams or more of protein in a 24 hour urine collection. Preeclampsia may further be characterized as mild preeclampsia or severe preeclampsia. Severe preeclampsia may be characterized by one or more of the following: i) a systolic blood pressure of 160 mm Hg or higher or a diastolic blood pressure of 110 mm Hg or higher on two occasions six or more hours apart in a pregnant woman who is on bed rest; ii) proteinuria, with excretion of 5 g or more of protein in a 24-hour urine specimen or 3+ or greater on two random samples collected four or more hours apart; iii) oliguria, with excretion of less than 500 mL of urine in 24 hours; iv) pulmonary edema or cyanosis; v) impairment of liver function; vi) visual or cerebral disturbances; vii) pain in the epigastric area or right upper quadrant; ix) decreased platelet count; and intrauterine growth restriction.

“Hypertension” is defined as systolic blood pressure ≥140 mmHg and/or diastolic blood pressure ≥90 mmHg. Severe hypertension is defined as systolic blood pressure ≥160 mmHg and/or diastolic blood pressure ≥110 mmHg.

“Likelihood of developing preeclampsia” means the probability, based upon one or more criteria, that a pregnant subject will develop preeclampsia during pregnancy.

“Healthy” or “normal” as used herein refers to an individual who does not have preeclampsia and/or has a low risk of preeclampsia. The individual may have other diseases, disorders, and/or conditions, which may or may not relate to pregnancy. For example, an individual who does not have preeclampsia but does have gestational diabetes is considered healthy or normal as used herein.

“Gestational age” as used herein is the number of weeks of gestation of a fetus. Gestational age can be determined using clinical examination (symphysis-pubis fundal height (SFH) and Ballard Score (BS), ultrasound, and/or biomarker detection. Gestational age can also be reported based upon the trimester of pregnancy. First trimester typically starts in week 0 and lasts until week 13. Second trimester starts in week 14 and ends in week 26. Third trimester starts in week 27 and lasts until delivery. Typically, a full term pregnancy is considered to be 39 weeks to 40 weeks and 6 days, 37-39 weeks is considered early term, 36 weeks 6 days and earlier is considered to be premature, and 41 weeks and longer is considered to be late term.

“Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology. Subjects at risk of developing a disease, such as preeclampsia, may be administered a prophylactic treatment.

### II. Exemplary Workflow

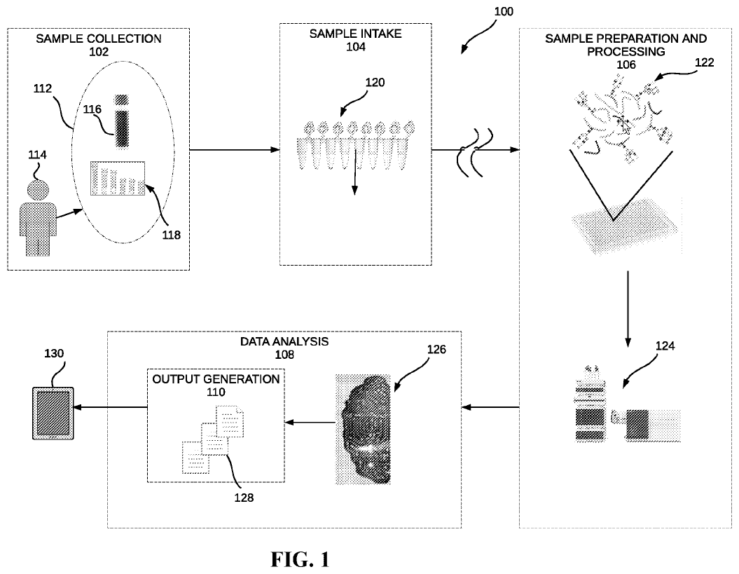

FIG. 1 is a schematic diagram of an exemplary workflow 100 for the detection of peptide structures associated with a disease state for use in diagnosis and/or treatment in accordance with one or more embodiments. Similarly, exemplary workflow 100 can be used for the detection of peptide structures associated with a gestational age for use in the determination of gestational age in accordance with one or more embodiments. Workflow 100 may include various operations including, for example, sample collection 102, sample intake 104, sample preparation and processing 106, data analysis 108, and output generation 110.

Sample collection 102 may include, for example, obtaining a biological sample 112 of one or more subjects, such as subject 114. Biological sample 112 may take the form of a specimen obtained via one or more sampling methods. Biological sample 112 may be representative of subject 114 as a whole or of a specific tissue, cell type, or other category or sub-category of interest. Biological sample 112 may be maternal serum, amniotic fluid, or maternal blood that can be collected into a vial with a septum cap. Biological sample 112 may be obtained in any of a number of different ways. In various embodiments, biological sample 112 includes whole blood sample 116 obtained via a blood draw. In other embodiments, biological sample 112 includes a set of aliquoted samples 118 that includes, for example, a serum sample, a plasma sample, a blood cell (e.g., white blood cell (WBC), red blood cell (RBC) sample, another type of sample, or a combination thereof. Biological sample 112 may include nucleotides (e.g., ssDNA, dsDNA, RNA), organelles, amino acids, peptides, proteins, carbohydrates, glycoproteins, or any combination thereof.

In various embodiments, a single run can analyze a sample (e.g., the sample including a peptide analyte), an external standard (e.g., an NGEP of a serum sample), and an internal standard. As such, abundance values (e.g., abundance or raw abundance) for the external standard, the internal standard, and target glycopeptide analyte can be determined by mass spectrometry in the same run.

In various embodiments, external standards may be analyzed prior to analyzing samples. In various embodiments, the external standards can be run independently between the samples. In some embodiments, external standards can be analyzed after every 1, 2, 3, 4, 5, 6, 7, 8, 9, 10, 11, 12, 13, 14, 15, 16, 17, 18, 19, 20, or more experiments. In various embodiments, external standard data can be used in some or all of the normalization systems and methods described herein. In additional embodiments, blank samples may be processed to prevent column fouling.

Sample intake 104 may include one or more various operations such as, for example, aliquoting, registering, processing, storing, thawing, and/or other types of operations. In one or more embodiments, when biological sample 112 includes whole blood sample 116, sample intake 104 includes aliquoting whole blood sample 116 to form a set of aliquoted samples that can then be sub-aliquoted to form set of samples 120.

Sample preparation and processing 106 may include, for example, one or more operations to form set of peptide structures 122. In various embodiments, set of peptide structures 122 may include various fragments of unfolded proteins that have undergone digestion and may be ready for analysis.

Further, sample preparation and processing 106 may include, for example, data acquisition 124 based on set of peptide structures 122. For example, data acquisition 124 may include use of, for example, but is not limited to, a liquid chromatography/mass spectrometry (LC/MS) system.

Data analysis 108 may include, for example, peptide structure analysis 126. In some embodiments, data analysis 108 also includes output generation 110. Peptide structure analysis can include determining the composition and the associated quantity for the various peptides and glycopeptides present in the sample by processing the output of a mass spectrometer. In other embodiments, output generation 110 may be considered a separate operation from data analysis 108. Output generation 110 may include, for example, generating final output 128 based on the results of peptide structure analysis 126. In various embodiments, final output 128 may be used for determining the research, diagnosis, and/or treatment of a state associated with preeclampsia.

In various embodiments, final output 128 is comprised of one or more outputs. Final output 128 may take various forms. For example, final output 128 may be a report that includes, for example, a diagnosis output, a treatment output (e.g., a treatment design output, a treatment plan output, or combination thereof), analyzed data (e.g., relativized and normalized) or combination thereof. In another embodiment, the final output 128 may include, for example, a report (e.g., clinical report) that may be provided to a clinician or a patient. In some embodiments, the report can comprise a target glycopeptide analyte concentration as a function of the NGEP concentration value and the normalized abundance value. In some embodiments, final output 128 may be an alert (e.g., a visual alert, an audible alert, etc.), a notification (e.g., a visual notification, an audible notification, an email notification, etc.), an email output, or a combination thereof. In some embodiments, final output 128 may be sent to remote system 130 for processing. Remote system 130 may include, for example, a computer system, a server, a processor, a cloud computing platform, cloud storage, a laptop, a tablet, a smartphone, some other type of mobile computing device, or a combination thereof.

In other embodiments, workflow 100 may optionally exclude one or more of the operations described herein and/or may optionally include one or more other steps or operations other than those described herein (e.g., in addition to and/or instead of those described herein). Accordingly, workflow 100 may be implemented in any of a number of different ways for use in the research, diagnosis, and/or treatment of, for example, preeclampsia or determining gestational age.

### III. Detection and Quantification of Peptide Structures

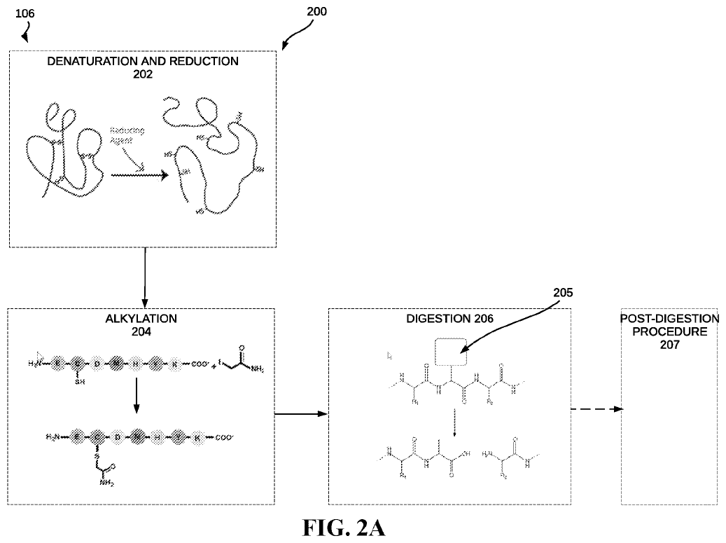

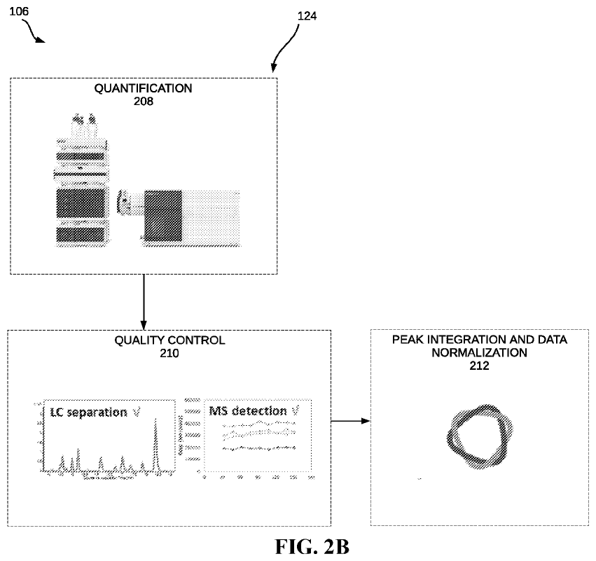

FIGS. 2A and 2B are schematic diagrams of a workflow for sample preparation and processing 106 in accordance with one or more embodiments. FIG. 2A and FIG. 2B are described with continuing reference to FIG. 1. Sample preparation and processing 106 may include, for example, preparation workflow 200 shown in FIG. 2A and data acquisition 124 shown in FIG. 2B.

### III.A. Sample Preparation and Processing

FIG. 2A is a schematic diagram of a preparation workflow 200 in accordance with one or more embodiments. Preparation workflow 200 may be used to prepare a sample, such as a sample of set of samples 120 in FIG. 1, for analysis via data acquisition 124. For example, this analysis may be performed via mass spectrometry (e.g., LC-MS). In various embodiments, preparation workflow 200 may include denaturation and reduction 202, alkylation 204, and digestion 206.

In general, polymers, such as proteins, in their native form, can fold to include secondary, tertiary, and/or other higher order structures. Such higher order structures may functionalize proteins to complete tasks (e.g., enable enzymatic activity) in a subject. Further, such higher order structures of polymers may be maintained via various interactions between side chains of amino acids within the polymers. Such interactions can include ionic bonding, hydrophobic interactions, hydrogen bonding, and disulfide linkages between cysteine residues. However, when using analytic systems and methods, including mass spectrometry, unfolding such polymers (e.g., peptide/protein molecules) may be desired to obtain sequence information. In some embodiments, unfolding a polymer may include denaturing the polymer, which may include, for example, linearizing the polymer.

In one or more embodiments, denaturation and reduction 202 can be used to disrupt higher order structures (e.g., secondary, tertiary, quaternary, etc.) of one or more proteins (e.g., polypeptides and peptides) in a sample (e.g., one of set of samples 120 in FIG. 1). Denaturation and reduction 202 includes, for example, a denaturation procedure and a reduction procedure. In some embodiments, the denaturation procedure may be performed using, for example, thermal denaturation, where heat is used as a denaturing agent (e.g. heating the sample to about 90° C. to about 100° C. for about 1 to 10 minutes). The thermal denaturation can disrupt ionic bonding, hydrophobic interactions, and/or hydrogen bonding.

In one or more embodiments, the denaturation procedure may include using one or more denaturing agents, temperature (e.g., heat), or both. These one or more denaturing agents may include, for example, but are not limited to, any number of chaotropic salts (e.g., urea, guanidine), surfactants (e.g., sodium dodecyl sulfate (SDS), beta octyl glucoside, Triton X-100), or combination thereof. In some cases, such denaturing agents may be used in combination with heat when sample preparation workflow further includes a cleanup procedure.

The resulting one or more denatured (e.g., unfolded, linearized) proteins may then undergo further processing in preparation of analysis. For example, a reduction procedure may be performed in which one or more reducing agents are applied. In various embodiments, a reducing agent can produce an alkaline pH. A reducing agent may take the form of, for example, without limitation, dithiothreitol (DTT), tris(2-carboxyethyl)phosphine (TCEP), or some other reducing agent. The reducing agent may reduce (e.g., cleave) the disulfide linkages between cysteine residues of the one or more denatured proteins to form one or more reduced proteins.

In various embodiments, the one or more reduced proteins resulting from denaturation and reduction 202 may undergo a process to prevent the reformation of disulfide linkages between, for example, the cysteine residues of the one or more reduced proteins. This process may be implemented using alkylation 204 to form one or more alkylated proteins. For example, alkylation 204 may be used to add an acetamide group to a sulfur on each cysteine residue to prevent disulfide linkages from reforming. In various embodiments, an acetamide group can be added by reacting one or more alkylating agents with a reduced protein. The one or more alkylating agents may include, for example, one or more acetamide salts. An alkylating agent may take the form of, for example, iodoacetamide (IAA), 2-chloroacetamide, some other type of acetamide salt, or some other type of alkylating agent.

In some embodiments, alkylation 204 may include a quenching procedure. The quenching procedure may be performed using one or more reducing agents (e.g., one or more of the reducing agents described above).

In various embodiments, the one or more alkylated proteins formed via alkylation 204 can then undergo digestion 206 in preparation for analysis (e.g., mass spectrometry analysis). Digestion 206 of a protein may include cleaving the protein at or around one or more cleavage sites (e.g., site 205 which may be one or more amino acid residues). For example, without limitation, an alkylated protein may be cleaved at the carboxyl side of lysine or arginine residues. This type of cleavage may break the protein into various segments, which include one or more peptide structures (e.g., glycosylated or aglycosylated).

In various embodiments, digestion 206 is performed using one or more proteolysis catalysts. For example, an enzyme can be used in digestion 206. In some embodiments, the enzyme takes the form of trypsin. In other embodiments, one or more other types of enzymes (e.g., proteases) may be used in addition to or in place of trypsin. These one or more other enzymes include, but are not limited to, LysC, LysN, AspN, GluC, and ArgC. In some embodiments, digestion 206 may be performed using tosyl phenylalanyl chloromethyl ketone (TPCK)-treated trypsin, one or more engineered forms of trypsin, one or more other formulations of trypsin, or a combination thereof. In some embodiments, digestion 206 may be performed in multiple steps, with each involving the use of one or more digestion agents. For example, a secondary digestion, tertiary digestion, etc. may be performed. In one or more embodiments, trypsin is used to digest serum samples. In one or more embodiments, trypsin/LysC cocktails are used to digest plasma samples.

In some embodiments, digestion 206 further includes a quenching procedure. The quenching procedure may be performed by acidifying the sample (e.g., to a pH <3). In some embodiments, formic acid may be used to perform this acidification.

In various embodiments, preparation workflow 200 further includes post-digestion procedure 207. Post-digestion procedure 207 may include, for example, a cleanup procedure. The cleanup procedure may include, for example, the removal of unwanted components in the sample that results from digestion 206. For example, unwanted components may include, but are not limited to, inorganic ions, surfactants, etc. In some embodiments, post-digestion procedure 207 further includes a procedure for the addition of heavy-labeled peptide internal standards. In some embodiments, post-digestion procedure 207 further includes a procedure for enrichment of glycopeptides in the digested sample. The enrichment procedure may include, for example, using a Hydrophilic Interaction Liquid Chromatography (HILIC) concentration phase.

Although preparation workflow 200 has been described with respect to a sample created or taken from biological sample 112, such as a blood-based sample 116 (e.g., a whole blood sample, a plasma sample, a serum sample, etc.), sample preparation workflow 200 may be similarly implemented for other types of samples (e.g., tears, urine, tissue, interstitial fluids, sputum, etc.) to produce set of peptides structures 122.

### III.B. Peptide Structure Identification and Quantitation

FIG. 2B is a schematic diagram of data acquisition 124 in accordance with one or more embodiments. In various embodiments, data acquisition 124 can commence following sample preparation 200 described in FIG. 2A. In various embodiments, data acquisition 124 can comprise quantification 208, quality control 210, and peak integration and normalization 212.

In various embodiments, quantification 208 of peptides and glycopeptides can incorporate use of liquid chromatography-mass spectrometry LC/MS instrumentation. For example, LC-MS/MS, or tandem MS may be used. In general, LC/MS (e.g., LC-MS/MS) can combine the physical separation capabilities of liquid chromatography (LC) with the mass analysis capabilities of mass spectrometry (MS). According to some embodiments described herein, this technique allows for the separation of digested peptides to be fed from the LC column into the MS ion source through an interface. In various embodiments, quantification 208 is targeted quantification.

In various embodiments, any LC/MS device can be incorporated into the workflow described herein. In various embodiments, an instrument or instrument system suited for identification and quantification 208 may include, for example, a Triple Quadrupole LC/MS. In various embodiments, quantification 208 is performed using multiple reaction monitoring mass spectrometry (MRM-MS). MRM is a mass spectrometry method in which a precursor ion of a particular m/z (e.g., peptide analyte) is selected in the first quadrupole (Q1) and transmitted to the second quadrupole (Q2) for fragmentation. The resulting product ions are then transmitted to the third quadrupole (Q3), which detects only product ions with selected predefined m/z values.

In various embodiments described herein, identification of a particular protein or peptide and an associated quantity can be assessed. In various embodiments described herein, identification of a particular glycopeptide and an associated quantity can be assessed. In various embodiments described herein, identification of a particular glycan and an associated quantity can be assessed. In various embodiments described herein, particular glycans can be matched to a glycosylation site on a protein or peptide and the abundance values measured. In various embodiments, a glycopeptide of any of SEQ ID Nos:5-8 and an associated quality is assessed.

In some cases, quantification 208 includes using a specific collision energy associated for the appropriate fragmentation to consistently see an abundant product ion. Glycopeptide structures may have a lower collision energy than aglycosylated peptide structures. When analyzing a sample that includes glycopeptide structures, the source voltage and gas temperature may be lowered as compared to generic proteomic analysis.

In various embodiments, quality control 210 procedures can be put in place to optimize data quality. In various embodiments, measures can be put in place allowing only errors within acceptable ranges outside of an expected value. In various embodiments, employing statistical models (e.g., using Westgard rules) can assist in quality control 210. For example, quality control 210 may include, for example, assessing the retention time and abundance of representative peptide structures (e.g., glycosylated and/or aglycosylated) and spiked-in internal standards, in either every sample, or in each quality control sample (e.g., pooled serum digest).

Peak integration and normalization 212 may be performed to process the data that has been generated and transform the data into a format for analysis. For example, peak integration and normalization 212 may include converting abundance data for various product ions that were detected for a selected peptide structure into a single quantification metric (e.g., a relative quantity, an adjusted quantity, a normalized quantity, a relative concentration, an adjusted concentration, a normalized concentration, etc.) for that peptide structure. In some embodiments, peak integration and normalization 212 may be performed using one or more of the techniques described in U.S. Patent Publication No. 2020/0372973A1 and/or US Patent Publication No. 2020/0240996A1, the disclosures of which are incorporated by reference herein in their entireties.

In some embodiments, the presence, absence, and/or amount of at least one peptide structures is determined by a method other than mass spectrometry, for example by ELISA or immunoblotting (such as western blot). In some embodiments, the presence, absence/and or amount of a peptide structure set forth in Tables 3, 10, or 17 is determined by a method other than mass spectrometry, for example by ELISA or immunoblotting (such as western blot). In some embodiments, the presence, absence/and or amount of a peptide structure comprising a sequence set forth in SEQ ID NOs:5-12, 16-21, or 65-188 is determined by a method other than mass spectrometry, for example by ELISA or immunoblotting (such as western blot).

It is worthwhile to note that Tables 3, 10 and 17 includes the term Peptide Structure (PS) Name that refers to a reference name for a peptide or glycopeptide. The Peptide Structure (PS) Name of Tables 3, 10 and 17 contains a prefix that represents an acronym for a protein abbreviation that corresponds to the Protein Abbreviation of Tables 2, 9, and 16 (respectively). The term Peptide Sequence lists the order of amino acids in a series of single letter abbreviations. The term Linking Site Pos. in Protein Sequence is a number that refers to the position of an amino acid in which a glycan is attached. For the Linking Site Pos. in Protein Sequence, the amino acid position of the peptide sequence is defined by the numbered order of amino acids based on the Uniprot ID of the corresponding protein for the peptide sequence. The term Linking Site Pos. in Peptide Sequence is a number that refers to the position of an amino acid in which a glycan is attached. For the Linking Site Pos. in peptide Sequence, the amino acid position of the peptide sequence is defined by the numbered order of amino acids (from left to right) for the peptide sequence. The term Glycan Structure GL No. is a number that corresponds to a symbol structure and a composition of the glycans as indicated in Tables 4, 11, and 18.

Referring to Tables 4 and 11, Glycan Structure GL NO's 1102 and 1111 correspond to O-linked glycans where a rightmost N-acetylgalactosamine (GalNAc) of the glycan structure is attached to a linking site position in the peptide sequence in accordance with Tables 3 and 10. Referring to Tables 4, 11, and 18, all Glycan Structure GL NO's, other than 1102 and 1111, correspond to N-linked glycans where the term Symbol Structure illustrates a geometric linking structure of the carbohydrates where the bottommost carbohydrate (e.g., GlcNAc) is bound to the amino acid. The identity of the various monosaccharides is illustrated by the Legend section located at the end of Tables 4, 11, and 18. The abbreviations of the Legend are Glc that represents glucose and is indicated by a dark circle, Gal that represents galactose and is indicated by an open circle, Man that represents mannose and is indicated by a circle with intermediate grey shading, Fuc that represents fucose and is indicated by a dark triangle, Neu5Ac that represents N-acetylneuraminic acid and is indicated by a dark diamond, GlcNAc that represents N-acetylglucosamine and is indicated by a dark square, GalNAc that represents N-acetylgalactosamine and is indicated by an open square, and ManNAc that represents N-acetylmannosamine and is indicated by a square with intermediate grey shading. The term Composition refers to the number of various classes of carbohydrates that make up the glycan. The quantity for each class of carbohydrate is depicted as a number in parenthesis to the right of an abbreviation that corresponds to the class of the carbohydrate. These abbreviations are Hex, HexNAc, Fuc, and NeuAc that respectively correspond to hexose, N-acetylhexosamine, fucose, and N-acetylneuraminic acid. It should be noted that hexose sugars include glucose, galactose, and mannose; and N-acetylhexosamine sugars includes N-acetylglucosamine, N-acetylgalactosamine, and N-acetylmannosamine.

### IV. Methods of Sample Preparation and Analysis for Obtaining Biomarkers for Preeclampsia

In some embodiments, the method of identifying one or more glycopeptide biomarkers associated with preeclampsia comprises obtaining a biological sample from a first set of one or more individuals with preeclampsia and a second control biological sample from a second set of one or more individuals who do not have preeclampsia. The biological samples may each be subsequently digested, enriched, and analyzed for quantification of at least one glycopeptide.

In some embodiments, digestion of a biological sample comprises digestion with one or more proteases. In some embodiments, one or more of the proteases are serine proteases. In some embodiments, the one or more proteases are chosen from the group comprising trypsin and endoproteinase LysC. In some embodiments, digestion of a biological sample is quenched and then halted by mixing an acid with the protease to form a proteolytic digest. In some embodiments, digestion of a biological sample is preceded by denaturing the biological sample. In some embodiments, the denaturation comprises heating the biological sample to at least 100° C. In some embodiments, the denaturation comprises heating the biological sample for at least 5 minutes. In some embodiments, denaturation further comprises the step of centrifuging the denatured biological sample. In some embodiments, the biological sample is reduced with one or more reducing agents after denaturation and prior to digestion. In some embodiments, the one or more reducing agents comprise dithiothreitol (DTT), 2-mercaptoethanol, and 2-mercaptoethylamine-HCl. In some embodiments, the biological sample is alkylated via incubation with one or more alkylating agents after reduction and prior to digestion. In some embodiments, the one or more alkylating agents comprises iodoacetamide (IAA) and iodoacetate. In some embodiments, the biological samples are incubated with one or more alkylating agents for at least 30 minutes. In some embodiments, the alkylation of the biological sample is quenched with DTT.

In some embodiments, the biological sample is enriched for at least one glycopeptide after digestion of the biological sample. In some embodiments, the enrichment comprises loading the proteolytic digest onto a use of a hydrophilic interaction liquid chromatography (HILIC) column, washing the HILIC column with a wash liquid, and eluting an enriched glycopeptide eluate from the HILIC column with an eluting liquid. In some embodiments, the HILIC sorbent material is HILICON-iSPE.

In some embodiments, the analysis of the biological sample for quantification of at least one glycopeptide comprises performing liquid chromatography mass spectrometry (LC-MS) on the biological sample. In some embodiments, a number of peptide spectral matches (PSMs) is determined for the sample based on the LC-MS data of the sample. In some embodiments, the number of PSMs for the first biological sample is used to determine a fold change of a glycopeptide of the first biological sample relative to a second control biological sample. In some embodiments, a relative abundance of a glycopeptide detected in a first biological sample is calculated by dividing the number of PSMs for the glycopeptide by the sum of number of PSMs for all glycopeptides detected in the first biological sample. In some embodiments, the fold change of the glycopeptide is calculated by dividing the relative abundance of the glycopeptide for the first biological sample by the relative abundance of the glycopeptide for the second control biological sample. In some embodiments, the glycopeptide is identified as a biomarker associated with preeclampsia if the fold change is greater than 2 or less than 0.5 in value and the sum of the number of PSMs for the first biological sample and the second control biological sample is greater than a predetermined number. In some embodiments, the predetermined number for the PSM sum is 20 or 30. In some embodiments, the predetermined number for the PSM sum is determined based on the formula 10×(number of different biological sample types being compared).

### V. Computer Implemented Systems

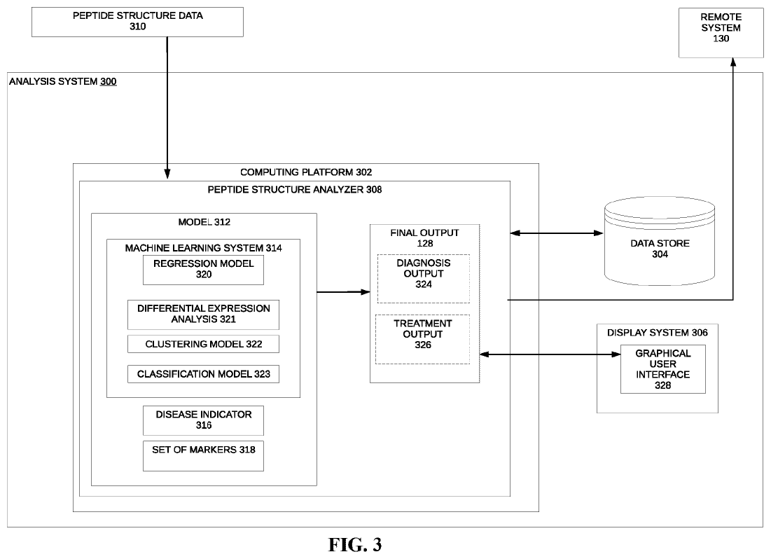

FIG. 3 is a block diagram of an analysis system 300, in accordance with the presently disclosed embodiments. For example, in accordance with the presently disclosed embodiments, the analysis system 300 may include any computing platform that may be utilized for classifying a biological sample obtained from a subject with respect to a plurality of states associated with preeclampsia; detecting the presence of one of a plurality of states associated with preeclampsia; determining a risk for developing preeclampsia in a subject; for treating preeclampsia in a subject; determining a risk for developing preeclampsia in a subject; techniques for treating preeclampsia in a subject; diagnosing an individual with preeclampsia; and training a model to diagnose a subject with one of a plurality of states associated with preeclampsia, in accordance with the presently disclosed embodiments. Analysis system 300 can be used to detect and analyze various peptide structures that have been associated with various states of preeclampsia or fetal gestational age. Analysis system 300 may be used to detect and analyze various glycopeptides that have been associated with various states of fetal gestational age. Analysis system 300 is one example of an implementation for a system that may be used to perform data analysis 108. Analysis system 300 may include computing platform 302 and data store 304.

In certain embodiments, analysis system 300 may also include display system 306. Computing platform 302 may take various forms. In certain embodiments, computing platform 302 may include a single computer (or computer system) or multiple computers in communication with each other. In other examples, computing platform 302 takes the form of a cloud computing platform. Data store 304 and display system 306 may each be in communication with computing platform 302. In some examples, data store 304, display system 306, or both may be considered part of or otherwise integrated with computing platform 302. Thus, in some examples, computing platform 302, data store 304, and display system 306 may be separate components in communication with each other, but in other examples, some combination of these components may be integrated together. Communication between these different components may be implemented using any number of wired communications links, wireless communications links, optical communications links, or a combination thereof.

In certain embodiments, analysis system 300 may include, for example, peptide structure analyzer 308, which may be implemented using hardware, software, firmware, or a combination thereof. In certain embodiments, peptide structure analyzer 308 is implemented using computing platform 302. Peptide structure analyzer 308 receives peptide structure data 310 for processing. Peptide structure data 310 may be, for example, the peptide structure data that is output from sample preparation and processing 106 in FIG. 1, FIG. 2A, and FIG. 2B. Accordingly, peptide structure data 310 may correspond to set of peptide structures 122 identified for biological sample 112 and may thereby correspond to biological sample 112. Peptide structure data 310 can be sent as input into peptide structure analyzer 308, retrieved from data store 304 or some other type of storage (e.g., cloud storage), accessed from cloud storage, or obtained in some other manner. In some cases, peptide structure data 310 may be retrieved from data store 304 in response to (e.g., directly or indirectly based on) receiving user input entered by a user via an input device. Peptide structure data 310 may include quantification data for the plurality of peptide structures. For example, peptide structure data 310 may include a set of quantification metrics for each peptide structure of a plurality of peptide structures. A quantification metric for a peptide structure may be selected as one of a relative quantity, an adjusted quantity, a normalized quantity, a relative abundance, an adjusted abundance, and a normalized abundance. In some cases, a quantification metric for a peptide structure is selected from one of a relative concentration, an adjusted concentration, and a normalized concentration. In this manner, peptide structure data 310 may provide abundance information about the plurality of peptide structures with respect to biological sample 112.

In certain embodiments, a peptide structure of set of peptide structures 312 may include a glycosylated peptide structure, or glycopeptide structure, that is defined by a peptide sequence and a glycan structure attached to a linking site of the peptide sequence. For example, the peptide structure may be a glycopeptide or a portion of a glycopeptide. In certain embodiments, a peptide structure of set of peptide structures 312 may include an aglycosylated peptide structure that is defined by a peptide sequence. For example, the peptide structure may be a tag glycopeptide or a portion of a tag glycopeptide and may be referred to as a quantification peptide. A tag peptide can be a peptide with at least one isotopically labeled amino acid. Set of peptide structures 312 may be identified as being those most predictive or relevant to the symptomatic disease state based on training of model 314. In certain embodiments, set of peptide structures 312 may include at least one, at least two, at least three, at least four, at least five, at least six, at least seven, or all eight of the peptide structures identified in Table 3 below. The number of peptide structures selected from Table 3 for inclusion in set of peptide structures 312 may be based on, for example, a desired level of accuracy. In certain embodiments, an N number of peptide structures may be selected from Table 3 for inclusion in set of peptide structures 312, in which Nis an integer from 1-8.

Peptide structure analyzer 308 may include model 314 that may be able to receive peptide structure data 310 for processing. Model 314 may be implemented in any of a number of different ways. Model 314 may be implemented using any number of models, functions, equations, algorithms, and/or other mathematical techniques. In certain embodiments, model 314 may include one or more machine learning systems 316, which may include any number of machine learning models and/or algorithms. For example, one or more machine learning systems 316 may include, without limitation, at least one of a parametric model, a non-parametric model, deep learning model, a neural network, a linear discriminant analysis model, a quadratic discriminant analysis model, a support vector machine, a random forest algorithm, a nearest neighbor algorithm (e.g., a k-Nearest Neighbors algorithm), a combined discriminant analysis model, a k-means clustering algorithm, an unsupervised model, a logistic regression model, a multivariable regression model, a penalized multivariable regression model, or another type of model. In certain embodiments, model 314 may include one or more machine learning systems 316, which may include any number of or combination of the models or algorithms described above.

For example, in certain embodiments, the one or more machine-learning systems 316 may include one or more ensemble learning models. For example, in one embodiment, the one or more ensemble learning models embodiment of the one or more machine-learning systems 316 may include a number of cascaded regression models, which may be trained such that each proceeding regression model in the number of cascaded regression models may correct an error of each proceeding regression model in the number of cascaded regression models (e.g., by reducing weight biases between the regression models). In other embodiments the one or more ensemble learning models may include a number of decision trees (e.g., dozens of decision trees, hundreds of decision trees, or thousands of decision trees), in which each succeeding decision tree of the number of decision trees is trained to correct an error and learn based on a prediction of each preceding decision tree of the number of decision trees until a final prediction is generated (e.g., by reducing weight biases between the number of decision trees). In another embodiment, the one or more ensemble learning models embodiment of the one or more machine-learning systems 316 may include one or more of a gradient boosting model, an adaptive boosting (AdaBoost) model, an eXtreme gradient boosting (XGBoost) model, a light gradient boosted machine (LightGBM) model, a categorical boosting (CatBoost) model, and so forth.

In certain embodiments, model 314 analyzes the portion (e.g., some or all of) peptide structure data 310 corresponding set of peptide structures 312 to generate disease indicator 318 that classifies biological sample 112 as evidencing a corresponding state of a plurality of states 320 associated with preeclampsia. Disease indicator 318 may take various forms. In certain embodiments, disease indicator 318 is a score that indicates a classification of the corresponding state for biological sample 112. For example, each of the states 320 may be associated with a different range of values for the score. If the score falls within a selected range associated with a particular state of the states 320, then the score indicates that biological sample 112 evidences that particular state. Thus, the score provides a classification of biological sample 112 as corresponding to that particular state.

In other embodiments, model 314 analyzes the portion (e.g., some or all of) peptide structure data 310 corresponding set of peptide structures 312 to generate gestational age indicator 318 that classifies biological sample 112 as evidencing a corresponding state of a plurality of states 320 associated with fetal gestational age. Gestational age indicator 318 may take various forms. In certain embodiments, gestational age indicator 318 is a score that indicates a classification of the corresponding state for biological sample 112. For example, each of the states 320 may be associated with a different range of values for the score. If the score falls within a selected range associated with a particular state of the states 320, then the score indicates that biological sample 112 evidences that particular state. Thus, the score provides a classification of biological sample 112 as corresponding to that particular state. In some embodiments, model 314 analyzes peptide structure data comprising one or more, two or more, three or more, four or more, five or more, or six peptides from Table 10.

In certain embodiments, disease indicator 318 may include a score that indicates a probability that a subject (e.g., subject 114 in FIG. 1) falls within one of the states 320 associated with preeclampsia. For example, disease indicator 318 may include one or more scores, each of which may indicate whether biological sample 112 evidences a corresponding state of the states 320 associated with preeclampsia. In some examples, disease indicator 318 may include a score for each of the states 320 associated with preeclampsia. A higher score (e.g., closer to the value of “1”) indicates a higher probability that biological sample 112 evidences the corresponding state, while a lower score (e.g., closer to the value of “0”) indicates a lower probability that biological sample 112 evidences the corresponding state. In certain embodiments, machine learning systems 316 may include a regression model. In one embodiment, the regression model may include, for example, one or more logistic regression models that may be trained to compute disease indicator 318. In another embodiment, the regression model may be trained to, for example, classify a biological sample obtained from a subject with respect to a plurality of states associated with preeclampsia; detect the presence of one of a plurality of states associated with preeclampsia; determine a risk for developing preeclampsia in a subject; techniques for treating preeclampsia in a subject; determine a risk for developing preeclampsia in a subject; and techniques for diagnosing an individual with preeclampsia, in accordance with the presently disclosed embodiments. The regression model may be trained to identify weight coefficients for peptide structures of set of peptide structures 312. Peptide structure analyzer 308 may generate final output 128 based on disease indicator 318 that is output by model 314. In other embodiments, final output 128 may be an output generated by model 314.

In certain embodiments, final output 128 may include disease indicator 318. In other embodiments, final output 128 may include diagnosis output 324 and/or treatment output 326. Diagnosis output 324 may include, for example, an identification of a classification of which of the states 320 evidenced by biological sample 112 based on disease indicator 318. Treatment output 326 may include, for example, at least one of an identification of a therapeutic to treat the subject, a design for the therapeutic, or a treatment plan for administering the therapeutic. In certain embodiments, the therapeutic is an antihypertensive, a corticosteroid, a beta blocker, or an anticonvulsant. Final output 128 may be sent to remote system 130 for processing in some examples. In other embodiments, final output 128 may be displayed on graphical user interface 328 in display system 306 for viewing by a human operator. The human operator may use final output 128 to diagnose and/or treat subject when final output 128 indicates the subject has preeclampsia or is at risk for preeclamisa.

In certain embodiments, final output 128 may include disease indicator 318. In other embodiments, final output 128 may include diagnosis output 324 and/or treatment output 326. Diagnosis output 324 may include, for example, an identification of a classification of which of the states 320 evidenced by biological sample 112 based on disease indicator 318. Treatment output 326 may include, for example, at least one of an identification of a therapeutic to treat the subject, a design for the therapeutic, or a treatment plan for administering the therapeutic. In certain embodiments, the therapeutic is an antihypertensive, a corticosteroid, a beta blocker, or an anticonvulsant. Final output 128 may be sent to remote system 130 for processing in some examples. In other embodiments, final output 128 may be displayed on graphical user interface 328 in display system 306 for viewing by a human operator. The human operator may use final output 128 to diagnose and/or treat subject when final output 128 indicates the subject has preeclampsia or a risk for developing preeclampsia.

In certain embodiments, gestational age indicator 318 may include a score that indicates a probability that a subject (e.g., subject 114 in FIG. 1) falls within one of the states 320 associated with fetal gestational age. For example, gestational age indicator 318 may include one or more scores, each of which may indicate whether biological sample 112 evidences a corresponding state of the states 320 associated with fetal gestational age. In some examples, gestational age indicator 318 may include a score for each of the states 320 associated with fetal gestational age. For example, in one embodiment, a lower score for each of the states 320 may correspond to a younger gestational age and a higher score for each of the states 320 may correspond to an older gestational age.

In certain embodiments, one or more machine-learning systems 316 may include one or more ensemble learning boosting models (e.g., a gradient boosting model, an adaptive boosting (AdaBoost) model, an eXtreme gradient boosting (XGBoost) model, a light gradient boosted machine (LightGBM) model, a categorical boosting (CatBoost) model). In one embodiment, the one or more ensemble learning models embodiment of the one or more machine-learning systems 316 may include a number of decision trees (e.g., dozens of decision trees, hundreds of decision trees, or thousands of decision trees), in which each succeeding decision tree of the number of decision trees is trained to correct an error and learn based on a prediction of each preceding decision tree of the number of decision trees until a gestational age indicator 318 is generated. In another embodiment, the one or more ensemble learning models may be trained to, for example, classify a biological sample obtained from a subject with respect to a plurality of states associated with fetal gestational age; detect the presence of one of a plurality of states associated with fetal gestational age; determine fetal gestational age; determine a fetal gestational age in a subject; and determine a plurality of states associated with fetal gestational age, in accordance with the presently disclosed embodiments. In one embodiment, the one or more ensemble learning models may be trained to identify weight coefficients for peptide structures of set of peptide structures 312. Peptide structure analyzer 308 may generate final output 128 based on gestational age indicator 318 that is output by model 314. In other embodiments, final output 128 may be an output generated by model 314.

In certain embodiments, final output 128 may include gestational age indicator 318. In other embodiments, final output 128 may include gestational age output 324 and/or treatment output 326. Gestational age output 324 may include, for example, an identification of a classification of which of the states 320 evidenced by biological sample 112 based on gestational age indicator 318. Treatment output 326 may include, for example, at least one of an identification of a therapeutic to treat the subject, a design for the therapeutic, or a treatment plan for administering the therapeutic. In certain embodiments, the therapeutic is an agent to promote or delay labor. Final output 128 may be sent to remote system 130 for processing in some examples. In other embodiments, final output 128 may be displayed on graphical user interface 328 in display system 306 for viewing by a human operator. The human operator may use final output 128 to determine gestational age when final output 128 indicates a gestational age (e.g., classifying a biological sample obtained from a subject with respect to a plurality of states associated with fetal gestational age; detecting the presence of one of a plurality of states associated with fetal gestational age; determining fetal gestational age; determining a fetal gestational age in a subject; training a model to determine a plurality of states associated with fetal gestational age).

In certain embodiments, final output 128 may include gestational age indicator 318. In other embodiments, final output 128 may include gestational age output 324 and/or treatment output 326. Gestational age output 324 may include, for example, an identification of a classification of which of the states 320 evidenced by biological sample 112 based on gestational age indicator 318. Treatment output 326 may include, for example, at least one of an identification of a therapeutic to treat the subject, a design for the therapeutic, or a treatment plan for administering the therapeutic. In certain embodiments, the therapeutic is an immune checkpoint inhibitor. Final output 128 may be sent to remote system 130 for processing in some examples. In other embodiments, final output 128 may be displayed on graphical user interface 328 in display system 306 for viewing by a human operator. The human operator may use final output 128 to diagnose and/or treat subject when final output 128 indicates the subject is positive a state (e.g., fetal gestational age).

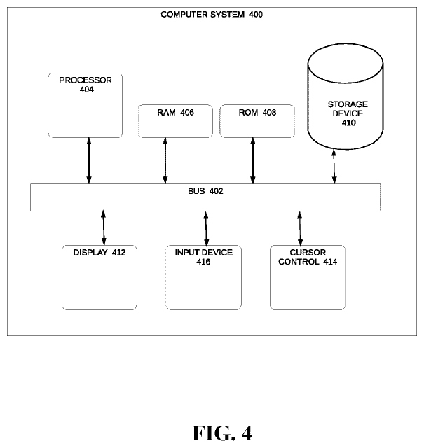

FIG. 4 illustrates a block diagram of a computer system that may be utilized for classifying a biological sample obtained from a subject with respect to a plurality of states associated with preeclampsia; detecting the presence of one of a plurality of states associated with preeclampsia; determining a risk for developing preeclampsia in a subject; for treating preeclampsia in a subject; determining a risk for developing preeclampsia in a subject; treating preeclampsia in a subject; diagnosing an individual with preeclampsia; and training a model to diagnose a subject with one of a plurality of states associated with preeclampsia, in accordance with the presently disclosed embodiments. In another embodiment, the a block diagram of FIG. 4 may be utilized for classifying a biological sample obtained from a subject with respect to a plurality of states associated with fetal gestational age; detecting the presence of one of a plurality of states associated with fetal gestational age; determining fetal gestational age; determining a fetal gestational age in a subject; and training a model to determine a plurality of states associated with fetal gestational age, in accordance with the presently disclosed embodiments. Computer system 400 may be an example of one implementation for computing platform 302 described above in FIG. 3. In certain embodiments, computer system 400 can include a bus 402 or other communication mechanism for communicating information, and a processor 404 coupled with bus 402 for processing information. In certain embodiments, computer system 400 can also include a memory, which can be a random-access memory (RAM) 406 or other dynamic storage device, coupled to bus 402 for determining instructions to be executed by processor 404. Memory also can be used for storing temporary variables or other intermediate information during execution of instructions to be executed by processor 404. In various embodiments, computer system 400 can further include a read only memory (ROM) 408 or other static storage device coupled to bus 402 for storing static information and instructions for processor 404. A storage device 410, such as a magnetic disk or optical disk, can be provided and coupled to bus 402 for storing information and instructions.

In certain embodiments, computer system 400 can be coupled via bus 402 to a display 412, such as a cathode ray tube (CRT) or liquid crystal display (LCD), for displaying information to a computer user. An input device 414, including alphanumeric and other keys, can be coupled to bus 402 for communicating information and command selections to processor 404. Another type of user input device is a cursor control 416, such as a mouse, a joystick, a trackball, a gesture input device, a gaze-based input device, or cursor direction keys for communicating direction information and command selections to processor 404 and for controlling cursor movement on display 412. This input device 414 typically has two degrees of freedom in two axes, a first axis (e.g., x) and a second axis (e.g., y), that allows the device to specify positions in a plane. However, it should be understood that input devices 414 allowing for three-dimensional (e.g., x, y, and z) cursor movement are also contemplated herein.

### VI. Methods of Diagnosing Preeclampsia or Determining Gestational Age

### VI.A.1 General Methodology Based on Table 3

In some embodiments, the methods provided herein are useful for diagnosing preeclampsia in the second or third trimester. In some embodiments the method comprises determining a risk of developing preeclampsia. In some embodiments, a diagnosis of preeclampsia is provided after 20 weeks. In some embodiments, a diagnosis of preeclampsia is provided after 27 weeks, after 30 weeks, after 33 weeks or after 36 weeks of gestation. In some embodiments, the diagnosis of preeclampsia is after delivery of the fetus.

In some embodiments, the diagnosis is based upon presence and/or amount of at least one, at least two, at least three, at least four, at least five, at least six, at least seven or eight peptide structures from Table 3. In some embodiments, the diagnosis is based upon the presence and/or amount of one or more peptides comprising the amino acid sequence of SEQ ID NO:5-12. In some embodiments, the diagnosis is based upon the presence and/or amount of two or more peptides comprising the amino acid sequence of SEQ ID NO:5-12. In some embodiments, the diagnosis is based upon the presence and/or amount of three or more peptides comprising the amino acid sequence of SEQ ID NO:5-12. In some embodiments, the diagnosis is based upon the presence and/or amount of four or more peptides comprising the amino acid sequence of SEQ ID NO:5-12. In some embodiments, the diagnosis is based upon the presence and/or amount of five or more peptides comprising the amino acid sequence of SEQ ID NO:5-12. In some embodiments, the diagnosis is based upon the presence and/or amount of six or more peptides comprising the amino acid sequence of SEQ ID NO:5-12. In some embodiments, the diagnosis is based upon the presence and/or amount of seven or more peptides comprising the amino acid sequence of SEQ ID NO:5-12. In some embodiments, the diagnosis is based upon the presence and/or amount of each of the peptides comprising the amino acid sequence of SEQ ID NO:5-12.

In some embodiments, the diagnosis is based upon the presence and/or amount of one or more peptides consisting of the amino acid sequence of SEQ ID NO:5-12. In some embodiments, the diagnosis is based upon the presence and/or amount of two or more peptides consisting of the amino acid sequence of SEQ ID NO:5-12. In some embodiments, the diagnosis is based upon the presence and/or amount of three or more peptides consisting of the amino acid sequence of SEQ ID NO:5-12. In some embodiments, the diagnosis is based upon the presence and/or amount of four or more peptides consisting of the amino acid sequence of SEQ ID NO:5-12. In some embodiments, the diagnosis is based upon the presence and/or amount of five or more peptides consisting of the amino acid sequence of SEQ ID NO:5-12. In some embodiments, the diagnosis is based upon the presence and/or amount of six or more peptides consisting of the amino acid sequence of SEQ ID NO:5-12. In some embodiments, the diagnosis is based upon the presence and/or amount of seven or more peptides consisting of the amino acid sequence of SEQ ID NO:5-12. In some embodiments, the diagnosis is based upon the presence and/or amount of each of the peptides consisting of the amino acid sequence of SEQ ID NO:5-12.

In some embodiments, the method further comprises collecting a biological sample. In some embodiments, the method comprises collecting maternal serum. In some embodiments, maternal serum is collected after 20 weeks gestation. In some embodiments, maternal serum is collected in or after 27 weeks, in or after 30 weeks, in or after 33 weeks or in or after 36 weeks of gestation.

For example, in certain embodiments, the presence or amount of the at least one peptide structure is detected using mass spectrometry, ELISA, or MRM mass spectrometry. In one embodiment, the at least one peptide structure is none, or below a detection limit. In one embodiment, the preeclampsia is severe preeclampsia. In one embodiment, the biological sample is maternal serum. In one embodiment, the one or more peptide structure includes a glycopeptide of a pregnancy-specific protein, and the at least one peptide structure comprises three or more peptide structures identified in Table 3.

In certain embodiments, the present embodiments may further include assessing one or more risk factors or clinical indicators of preeclampsia, in which a clinical indicator of preeclampsia is selected from the group consisting of protein in the urine and high blood pressure. In certain embodiments, the risk factor for preeclampsia is selected from the group consisting of history of preeclampsia, chronic hypertension, obesity, and multiple pregnancy. In certain embodiments, the individual is determined have a healthy state, in which a healthy state may include the absence of preeclampsia and/or a low risk for preeclampsia. The present embodiments may further include diagnosing a placental development problem.

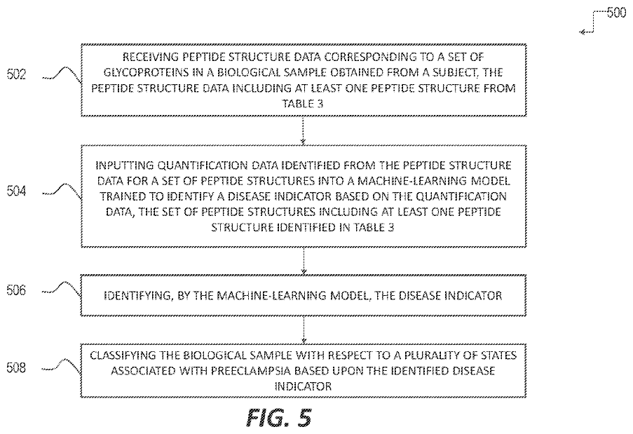

FIG. 5 illustrates a flow diagram 500 of a method for classifying a biological sample obtained from a subject with respect to a plurality of states associated with preeclampsia, in accordance with the presently disclosed embodiments. The flow diagram 500 may be performed utilizing one or more processing devices (e.g., computing platform 302 as discussed above with respect to FIG. 3) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), or any other processing device(s) that may be suitable for processing various medical profile data and making one or more decisions based thereon), software (e.g., instructions running/executing on one or more processors), firmware (e.g., microcode), or some combination thereof.

The flow diagram 500 may begin at block 502 with one or more processing devices (e.g., computing platform 302) receiving peptide structure data corresponding to a set of glycoproteins in the biological sample. The flow diagram 500 may then continue at block 504 with one or more processing devices (e.g., computing platform 302) inputting quantification data identified from the peptide structure data for a set of peptide structures into a machine-learning model trained to identify a disease indicator based on the quantification data, wherein the set of peptide structures comprises at least one peptide structure identified from a plurality of peptide structures in Table 3. The flow diagram 500 may then continue at block 506 with one or more processing devices (e.g., computing platform 302) identifying, by the machine-learning model, the disease indicator. The flow diagram 500 may then conclude at block 508 with one or more processing devices (e.g., computing platform 302) classifying the biological sample with respect to a plurality of states associated with preeclampsia based upon the identified disease indicator.

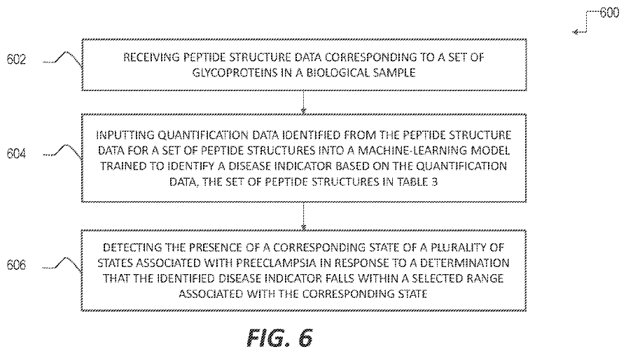

FIG. 6 illustrates a flow diagram 600 of a method for detecting the presence of one of a plurality of states associated with preeclampsia in a subject, in accordance with the presently disclosed embodiments. The flow diagram 600 may be performed utilizing one or more processing devices (e.g., computing platform 302 as discussed above with respect to FIG. 3) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), or any other processing device(s) that may be suitable for processing various medical profile data and making one or more decisions based thereon), software (e.g., instructions running/executing on one or more processors), firmware (e.g., microcode), or some combination thereof.

The flow diagram 600 may begin at block 602 with one or more processing devices (e.g., computing platform 302) receiving peptide structure data corresponding to a set of glycoproteins in a biological sample obtained from a subject, wherein the peptide structure data comprises at least one peptide structure from Table 3. The flow diagram 600 may then continue at block 604 with one or more processing devices (e.g., computing platform 302) inputting quantification data identified from the peptide structure data for a set of peptide structures into a machine-learning model trained to identify a disease indicator based on the quantification data, wherein the set of peptide structures comprises at least one peptide structure identified from a plurality of peptide structures in Table 3. The flow diagram 600 may then conclude at block 606 with one or more processing devices (e.g., computing platform 302) detecting the presence of a corresponding state of the plurality of states associated with preeclampsia in response to a determination that the identified disease indicator falls within a selected range associated with the corresponding state.

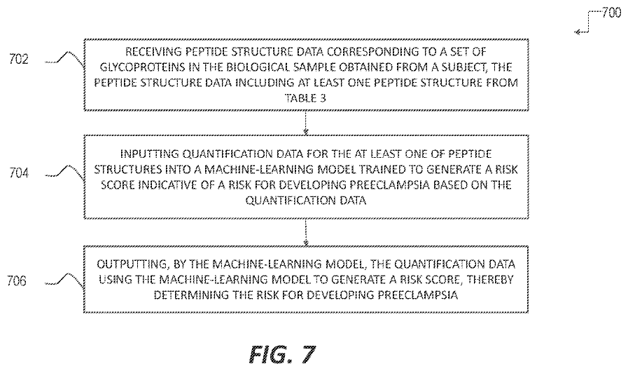

FIG. 7 illustrates a flow diagram 700 of a method for determining a risk for developing preeclampsia in a subject, in accordance with the presently disclosed embodiments. The flow diagram 700 may be performed utilizing one or more processing devices (e.g., computing platform 302 as discussed above with respect to FIG. 3) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), or any other processing device(s) that may be suitable for processing various medical profile data and making one or more decisions based thereon), software (e.g., instructions running/executing on one or more processors), firmware (e.g., microcode), or some combination thereof.

The flow diagram 700 may begin at block 702 with one or more processing devices (e.g., computing platform 302) receiving peptide structure data corresponding to a set of glycoproteins in the biological sample obtained from a subject, wherein the peptide structure data comprises at least one peptide structure from Table 3. The flow diagram 700 may then continue at block 704 with one or more processing devices (e.g., computing platform 302) inputting quantification data for at least one of the peptide structures into a machine-learning model trained to generate a risk score indicative of a risk for developing preeclampsia based on the quantification data. The flow diagram 700 may then conclude at block 706 with one or more processing devices (e.g., computing platform 302) outputting, by the machine-learning model, the quantification data using the machine learning model to generate a risk score, thereby determining the risk for developing preeclampsia.

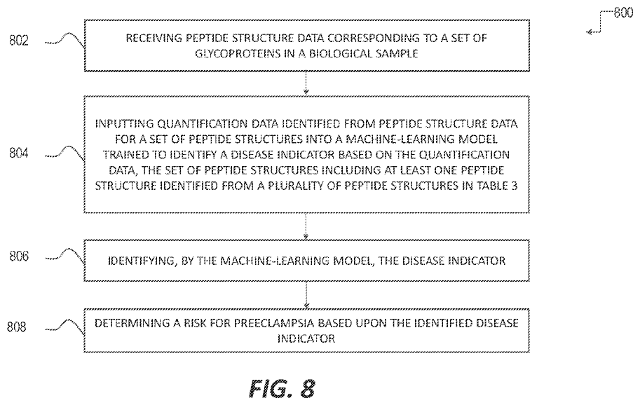

FIG. 8 illustrates a flow diagram 800 of a method for determining a risk for developing preeclampsia in a subject, in accordance with the presently disclosed embodiments. The flow diagram 800 may be performed utilizing one or more processing devices (e.g., computing platform 302 as discussed above with respect to FIG. 3) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), or any other processing device(s) that may be suitable for processing various medical profile data and making one or more decisions based thereon), software (e.g., instructions running/executing on one or more processors), firmware (e.g., microcode), or some combination thereof.

The flow diagram 800 may begin at block 802 with one or more processing devices (e.g., computing platform 302) receiving peptide structure data corresponding to a set of glycoproteins in the biological sample. The flow diagram 800 may then continue at block 804 with one or more processing devices (e.g., computing platform 302) inputting quantification data identified from the peptide structure data for a set of the peptide structures into a machine-learning model trained to identify a disease indicator based on the quantification data, wherein the set of peptide structure data comprises at least one peptide structure identified from a plurality of peptide structures in Table 3. The flow diagram 800 may then continue at block 806 with one or more processing devices (e.g., computing platform 302) identifying, by the machine-learning model, the disease indicator. The flow diagram 800 may then conclude at block 808 with one or more processing devices (e.g., computing platform 302) determining a risk for preeclampsia based upon the identified disease indicator.

In one or more examples, the plurality of states may include at least one of a predisposition for preeclampsia, preeclampsia, severe preeclampsia, or a healthy state. In one embodiment, the machine-learning model may include a logistic regression model, which was trained by generating a log error cost function based on a plurality of disease indicators and minimizing the log error cost function based on the plurality of disease indicators and the quantification data. In certain embodiments, the present embodiments may further include administering an effective amount of an antihypertensive, a corticosteroid, a beta blocker, or an anticonvulsant to treat preeclampsia based upon the presence or amount of the peptide structure.

### VI.A.2 General Methodology Based on Table 10

Provided herein are method of determining a gestational age of a fetus comprising detecting the presence or amount of at least one peptide structure from Table 10. In some embodiments, the gestational age is determined based upon presence and/or amount of at least one, at least two, at least three, at least four, at least five, or six peptide structures from Table 10. In some embodiments one or more of the peptide structures is not present in the sample. In some embodiments, gestational age is determined by the absence of one or more peptide structures, such as those in Table 10. In some embodiments, gestational age is determined by the presence and/or amount of one, two, three, four five, or six peptides comprising the sequence set forth in SEQ ID Nos:16-21. In some embodiments, the peptide structures are detected by MRM-MS. In some embodiments, the peptide structures are detected using western blot or ELISA. In some embodiments, the gestational age is determined based upon absence of at least one, at least two, at least three, at least four, at least five, or six peptide structures from Table 10.

In some embodiments, determination of gestational age is based upon the absence, presence and/or amounts of a glycopeptide comprising a sequence set forth in SEQ ID NO:16-21. In some embodiments, the glycopeptide comprising the sequence set forth in SEQ ID NO:16-21 is between 3 to 50 amino acids in length. In some embodiments, the glycopeptide comprising the sequence set forth in SEQ ID NO:16-21 is between 5 to 45, 7 to 40, 10 to 35, 3 to 15, 4 to 20, or 35 to 50 amino acids in length. In some embodiments, a peptide with a sequence set forth in SEQ ID NO:16-21 comprises a specific glycan structure linked at a specific site. For example, in some embodiments, a peptide comprising the sequence of SEQ ID NO: 16 comprises the amino acid sequence FSEFWDLDPEVRPTSAVAA with a glycan structure 1111 at position 14. In some embodiments, a peptide comprising the sequence of SEQ ID NO: 17 comprises the amino acid sequence AALAAFNAQNNGSNFQLEEISR with a glycan structure 5401 at position 11. In some embodiments, a peptide comprising the sequence of SEQ ID NO: 18 comprises the amino acid sequence AALAAFNAQNNGSNFQLEEISR with a glycan structure 6502 at position 11. In some embodiments, a peptide comprising the sequence of SEQ ID NO: 18 comprises the amino acid sequence AALAAFNAQNNGSNFQLEEISR with a glycan structure 6410 at position 11.

In some embodiments, the methods provided herein are useful for determining fetal gestational age in the second or third trimester of gestation. In some embodiments, gestational age is determined in or after 20 weeks gestation, for example, in or after 22 weeks, in or after 23 weeks, in or after 24 weeks, in or after 25 weeks, in or after 26 weeks, in or after 27 weeks, in or after 28 weeks, in or after 29 weeks, in or after 30 weeks, in or after 31 weeks, in or after 32 weeks, in or after 33 weeks, in or after 34 weeks, in or after 35 weeks, in or after 36 weeks, in or after 37 weeks, in or after 38 weeks, in or after 39 weeks or in or after 40 weeks gestation, or up to 42 weeks. In some embodiments, gestational age is determined to be between 25 to 36 weeks, such as between 25 and 30 weeks, between 30 and 36 weeks, between 25 and 28 weeks, between 32 and 36 weeks, between 28 and 32 weeks.

In some embodiments, fetal gestational age is determined by detecting the presence and/or amount of one or more pregnancy specific proteins. In some embodiments, fetal gestational age is determined by detecting the presence and/or amount of a peptide of one or more pregnancy specific proteins. In some embodiments, fetal gestational age is determined by detecting the presence and/or amount of a glycopeptide peptide of one or more pregnancy specific proteins.

In some embodiments, the methods provided herein further comprises assessing one or more additional clinical indicators of fetal gestational age. In some embodiments, last menstrual period (LMP), ultrasound fetal images, and/or fundal height (i.e. SFH) is also used to assess fetal gestational age. SFH is determined by measuring from the mother's pubic bone (symphysis pubis) to the top of the womb. The measurement is then applied to the gestation by a simple rule of thumb and compared with normal growth. In some embodiments, the ultrasound fetal images are from the first trimester of gestation. In some embodiments, the ultrasound fetal images are from the second or third trimester of gestation. In some embodiments, fetal gestational age is determined by detection and quantification of one or more peptide structures comprising SEQ ID NO:16-21 in combination with one or more additional clinical indicators of fetal gestational age.

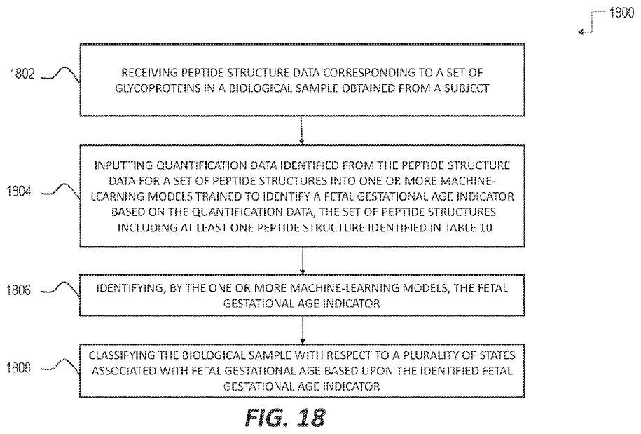

FIG. 18 illustrates a flow diagram 1800 of a method for classifying a biological sample obtained from a subject with respect to a plurality of states associated with fetal gestational age, in accordance with the presently disclosed embodiments. The flow diagram 1800 may be performed utilizing one or more processing devices (e.g., computing platform 302 as discussed above with respect to FIG. 3) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), or any other processing device(s) that may be suitable for processing various medical profile data and making one or more decisions based thereon), software (e.g., instructions running/executing on one or more processors), firmware (e.g., microcode), or some combination thereof.

The flow diagram 1800 may begin at block 1802 with one or more processing devices (e.g., computing platform 302) receiving peptide structure data corresponding to a set of glycoproteins in the biological sample. The flow diagram 1800 may then continue at block 1804 with one or more processing devices (e.g., computing platform 302) inputting quantification data identified from the peptide structure data for a set of the peptide structures into one or more machine-learning models trained to identify a fetal gestational age indicator based on the quantification data, wherein the set of peptide structures comprises at least one peptide structure identified from a plurality of peptide structures in Table 10. The flow diagram 1800 may then continue at block 1806 with one or more processing devices (e.g., computing platform 302) identifying, by the one or more machine-learning models, the fetal gestational age indicator. The flow diagram 1800 may then conclude at block 1808 with one or more processing devices (e.g., computing platform 302) classifying the biological sample with respect to a plurality of states associated with fetal gestational age based upon the identified fetal gestational age indicator.

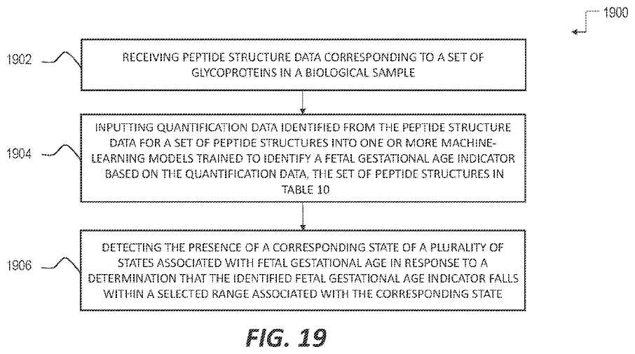

FIG. 19 illustrates a flow diagram 1900 of a method for detecting the presence of one of a plurality of states associated with fetal gestational age, in accordance with the presently disclosed embodiments. The flow diagram 1900 may be performed utilizing one or more processing devices (e.g., computing platform 302 as discussed above with respect to FIG. 3) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), or any other processing device(s) that may be suitable for processing various medical profile data and making one or more decisions based thereon), software (e.g., instructions running/executing on one or more processors), firmware (e.g., microcode), or some combination thereof.

The flow diagram 1900 may begin at block 1902 with one or more processing devices (e.g., computing platform 302) receiving peptide structure data corresponding to a set of glycoproteins in a biological sample obtained from a subject. The flow diagram 1900 may then continue at block 1904 with one or more processing devices (e.g., computing platform 302) inputting quantification data identified from the peptide structure data for a set of peptide structures into one or more machine-learning models trained to identify a gestational age indicator based on the quantification data, wherein the set of peptide structures comprises at least one peptide structure identified from a plurality of peptide structures in Table 10. The flow diagram 1900 may then conclude at block 1906 with one or more processing devices (e.g., computing platform 302) detecting the presence of a corresponding state of the plurality of states associated with fetal gestational age in response to a determination that the identified fetal gestational age indicator falls within a selected range associated with the corresponding state.

For example, in certain embodiments, the plurality of states may include a number of weeks of gestation of a fetus. In certain embodiments, the plurality of states is a number of weeks of gestation of a fetus that is more than 20 weeks or more than 24 weeks. In certain embodiments, the one or more machine-learning models may include an ensemble learning model. For example, the ensemble learning model may include a plurality of decision trees, in which a succeeding decision tree of the plurality of decision trees is trained to correct an error of a preceding decision tree of the plurality of decision trees to identify the fetal gestational age indicator. In certain embodiments, the one or more machine-learning models may include one or more of a gradient boosting model, an adaptive boosting (AdaBoost) model, an eXtreme gradient boosting (XGBoost) model, a light gradient boosted machine (LightGBM) model, or a categorical boosting (CatBoost) model.

### VI.A.3 General Methodology Based on Table 17

In some embodiments, the methods provided herein are useful for diagnosing preeclampsia in the second or third trimester. In some embodiments the method comprises determining a risk of developing preeclampsia. In some embodiments, a diagnosis of preeclampsia is provided after 20 weeks. In some embodiments, a diagnosis of preeclampsia is provided after 27 weeks, after 30 weeks, after 33 weeks or after 36 weeks of gestation. In some embodiments, the diagnosis of preeclampsia is after delivery of the fetus. In some embodiments, a high risk of preeclampsia is a greater than 50%, or greater than 60%, or greater than 70%, or greater than 80%, or greater than 90%, or greater than 95% likelihood of developing preeclampsia within the next six months from the point in time when the biological sample was collected from a subject.

In some embodiments, the diagnosis is based upon presence and/or amount of at least one, at least two, at least three, at least four, at least five, at least six, at least seven, at least eight, at least nine, at least ten, at least fifteen, at least twenty, at least twenty-five, at least thirty, at least thirty-five, at least forty, at least forty-five, at least fifty, at least fifty-five, at least sixty, at least sixty-five, at least seventy, at least seventy-five, at least eighty, at least eighty-five, at least ninety, at least ninety-five, at least one hundred, at least one hundred five, at least one hundred ten, at least one hundred fifteen, at least one hundred twenty, or one hundred twenty-four peptide structures from Table 17. In some embodiments, the presence and/or amount of the peptide is determined using mass spectrometry. In some embodiments, the diagnosis is based upon the presence and/or amount of one or more peptides comprising the amino acid sequence of SEQ ID NOs: 65-188. In some embodiments, the diagnosis is based upon the presence and/or amount of two or more peptides comprising the amino acid sequence of SEQ ID NOs: 65-188. In some embodiments, the diagnosis is based upon the presence and/or amount of three or more peptides comprising the amino acid sequence of SEQ ID NOs: 65-188. In some embodiments, the diagnosis is based upon the presence and/or amount of four or more peptides comprising the amino acid sequence of SEQ ID NOs: 65-188. In some embodiments, the diagnosis is based upon the presence and/or amount of five or more peptides comprising the amino acid sequence of SEQ ID NOs: 65-188. In some embodiments, the diagnosis is based upon the presence and/or amount of six or more peptides comprising the amino acid sequence of SEQ ID NOs: 65-188. In some embodiments, the diagnosis is based upon the presence and/or amount of seven or more peptides comprising the amino acid sequence of SEQ ID NOs: 65-188. In some embodiments, the diagnosis is based upon the presence and/or amount of eight or more peptides comprising the amino acid sequence of SEQ ID NOs: 65-188. In some embodiments, the diagnosis is based upon the presence and/or amount of nine or more peptides comprising the amino acid sequence of SEQ ID NOs: 65-188. In some embodiments, the diagnosis is based upon the presence and/or amount of ten or more peptides comprising the amino acid sequence of SEQ ID NOs: 65-188. In some embodiments, the diagnosis is based upon the presence and/or amount of fifteen or more peptides comprising the amino acid sequence of SEQ ID NOs: 65-188. In some embodiments, the diagnosis is based upon the presence and/or amount of twenty or more peptides comprising the amino acid sequence of SEQ ID NOs: 65-188. In some embodiments, the diagnosis is based upon the presence and/or amount of twenty-five or more peptides comprising the amino acid sequence of SEQ ID NOs: 65-188. In some embodiments, the diagnosis is based upon the presence and/or amount of thirty or more peptides comprising the amino acid sequence of SEQ ID NOs: 65-188. In some embodiments, the diagnosis is based upon the presence and/or amount of thirty-five or more peptides comprising the amino acid sequence of SEQ ID NOs: 65-188. In some embodiments, the diagnosis is based upon the presence and/or amount of forty or more peptides comprising the amino acid sequence of SEQ ID NOs: 65-188. In some embodiments, the diagnosis is based upon the presence and/or amount of forty-five or more peptides comprising the amino acid sequence of SEQ ID NOs: 65-188. In some embodiments, the diagnosis is based upon the presence and/or amount of fifty or more peptides comprising the amino acid sequence of SEQ ID NOs: 65-188. In some embodiments, the diagnosis is based upon the presence and/or amount of fifty-five or more peptides comprising the amino acid sequence of SEQ ID NOs: 65-188. In some embodiments, the diagnosis is based upon the presence and/or amount of sixty or more peptides comprising the amino acid sequence of SEQ ID NOs: 65-188. In some embodiments, the diagnosis is based upon the presence and/or amount of sixty-five or more peptides comprising the amino acid sequence of SEQ ID NOs: 65-188. In some embodiments, the diagnosis is based upon the presence and/or amount of seventy or more peptides comprising the amino acid sequence of SEQ ID NOs: 65-188. In some embodiments, the diagnosis is based upon the presence and/or amount of seventy-five or more peptides comprising the amino acid sequence of SEQ ID NOs: 65-188. In some embodiments, the diagnosis is based upon the presence and/or amount of eighty or more peptides comprising the amino acid sequence of SEQ ID NOs: 65-188. In some embodiments, the diagnosis is based upon the presence and/or amount of eighty-five or more peptides comprising the amino acid sequence of SEQ ID NOs: 65-188. In some embodiments, the diagnosis is based upon the presence and/or amount of ninety or more peptides comprising the amino acid sequence of SEQ ID NOs: 65-188. In some embodiments, the diagnosis is based upon the presence and/or amount of ninety-five or more peptides comprising the amino acid sequence of SEQ ID NOs: 65-188. In some embodiments, the diagnosis is based upon the presence and/or amount of one hundred or more peptides comprising the amino acid sequence of SEQ ID NOs: 65-188. In some embodiments, the diagnosis is based upon the presence and/or amount of one hundred five or more peptides comprising the amino acid sequence of SEQ ID NOs: 65-188. In some embodiments, the diagnosis is based upon the presence and/or amount of one hundred ten or more peptides comprising the amino acid sequence of SEQ ID NOs: 65-188. In some embodiments, the diagnosis is based upon the presence and/or amount of one hundred fifteen or more peptides comprising the amino acid sequence of SEQ ID NOs: 65-188. In some embodiments, the diagnosis is based upon the presence and/or amount of one hundred twenty or more peptides comprising the amino acid sequence of SEQ ID NOs: 65-188. In some embodiments, the diagnosis is based upon the presence and/or amount of each of the peptides comprising the amino acid sequence of SEQ ID NOs: 65-188. In some embodiments, the presence and/or amount of the peptide is determined using mass spectrometry.

In some embodiments, the diagnosis is based upon the presence and/or amount of one or more peptides comprising the amino acid sequence of SEQ ID NOs: 65-74. In some embodiments, the diagnosis is based upon the presence and/or amount of one or more peptides comprising the amino acid sequence of SEQ ID NOs: 65-84. In some embodiments, the diagnosis is based upon the presence and/or amount of one or more peptides comprising the amino acid sequence of SEQ ID NOs: 65-94. In some embodiments, the diagnosis is based upon the presence and/or amount of one or more peptides comprising the amino acid sequence of SEQ ID NOs: 65-104. In some embodiments, the diagnosis is based upon the presence and/or amount of one or more peptides comprising the amino acid sequence of SEQ ID NOs: 65-114. In some embodiments, the diagnosis is based upon the presence and/or amount of one or more peptides comprising the amino acid sequence of SEQ ID NOs: 65-124. In some embodiments, the diagnosis is based upon the presence and/or amount of one or more peptides comprising the amino acid sequence of SEQ ID NOs: 65-134. In some embodiments, the diagnosis is based upon the presence and/or amount of one or more peptides comprising the amino acid sequence of SEQ ID NOs: 65-144. In some embodiments, the diagnosis is based upon the presence and/or amount of one or more peptides comprising the amino acid sequence of SEQ ID NOs: 65-154. In some embodiments, the diagnosis is based upon the presence and/or amount of one or more peptides comprising the amino acid sequence of SEQ ID NOs: 65-164. In some embodiments, the diagnosis is based upon the presence and/or amount of one or more peptides comprising the amino acid sequence of SEQ ID NOs: 65-174. In some embodiments, the diagnosis is based upon the presence and/or amount of one or more peptides comprising the amino acid sequence of SEQ ID NOs: 65-184. In some embodiments, the diagnosis is based upon the presence and/or amount of one or more peptides comprising the amino acid sequence of SEQ ID NOs: 65-188. In some embodiments, the diagnosis is based upon the presence and/or amount of one or more peptides comprising the amino acid sequence of SEQ ID NOs: 74-188. In some embodiments, the diagnosis is based upon the presence and/or amount of one or more peptides comprising the amino acid sequence of SEQ ID NOs: 84-104. In some embodiments, the diagnosis is based upon the presence and/or amount of one or more peptides comprising the amino acid sequence of SEQ ID NOs: 84-124. In some embodiments, the diagnosis is based upon the presence and/or amount of one or more peptides comprising the amino acid sequence of SEQ ID NOs: 84-144. In some embodiments, the diagnosis is based upon the presence and/or amount of one or more peptides comprising the amino acid sequence of SEQ ID NOs: 84-164. In some embodiments, the diagnosis is based upon the presence and/or amount of one or more peptides comprising the amino acid sequence of SEQ ID NOs: 84-188. In some embodiments, the diagnosis is based upon the presence and/or amount of one or more peptides comprising the amino acid sequence of SEQ ID NOs: 94-188. In some embodiments, the diagnosis is based upon the presence and/or amount of one or more peptides comprising the amino acid sequence of SEQ ID NOs: 104-124. In some embodiments, the diagnosis is based upon the presence and/or amount of one or more peptides comprising the amino acid sequence of SEQ ID NOs: 104-144. In some embodiments, the diagnosis is based upon the presence and/or amount of one or more peptides comprising the amino acid sequence of SEQ ID NOs: 104-164. In some embodiments, the diagnosis is based upon the presence and/or amount of one or more peptides comprising the amino acid sequence of SEQ ID NOs: 104-188. In some embodiments, the diagnosis is based upon the presence and/or amount of one or more peptides comprising the amino acid sequence of SEQ ID NOs: 114-188. In some embodiments, the diagnosis is based upon the presence and/or amount of one or more peptides comprising the amino acid sequence of SEQ ID NOs: 124-144. In some embodiments, the diagnosis is based upon the presence and/or amount of one or more peptides comprising the amino acid sequence of SEQ ID NOs: 124-164. In some embodiments, the diagnosis is based upon the presence and/or amount of one or more peptides comprising the amino acid sequence of SEQ ID NOs: 124-188. In some embodiments, the diagnosis is based upon the presence and/or amount of one or more peptides comprising the amino acid sequence of SEQ ID NOs: 134-188. In some embodiments, the diagnosis is based upon the presence and/or amount of one or more peptides comprising the amino acid sequence of SEQ ID NOs: 144-164. In some embodiments, the diagnosis is based upon the presence and/or amount of one or more peptides comprising the amino acid sequence of SEQ ID NOs: 144-188. In some embodiments, the diagnosis is based upon the presence and/or amount of one or more peptides comprising the amino acid sequence of SEQ ID NOs: 154-188. In some embodiments, the diagnosis is based upon the presence and/or amount of one or more peptides comprising the amino acid sequence of SEQ ID NOs: 164-188. In some embodiments, the diagnosis is based upon the presence and/or amount of one or more peptides comprising the amino acid sequence of SEQ ID NOs: 174-188. In some embodiments, the diagnosis is based upon the presence and/or amount of one or more peptides comprising the amino acid sequence of SEQ ID NOs: 184-188. In some embodiments, the presence and/or amount of the peptide is determined using mass spectrometry.

In some embodiments, the diagnosis is based upon the presence and/or amount of one or more peptides comprising the amino acid sequence of SEQ ID NOs: 65-123. In some embodiments, the diagnosis is based upon the presence and/or amount of two or more peptides comprising the amino acid sequence of SEQ ID NOs: 65-123. In some embodiments, the diagnosis is based upon the presence and/or amount of three or more peptides comprising the amino acid sequence of SEQ ID NOs: 65-123. In some embodiments, the diagnosis is based upon the presence and/or amount of four or more peptides comprising the amino acid sequence of SEQ ID NOs: 65-123. In some embodiments, the diagnosis is based upon the presence and/or amount of five or more peptides comprising the amino acid sequence of SEQ ID NOs: 65-123. In some embodiments, the diagnosis is based upon the presence and/or amount of ten or more peptides comprising the amino acid sequence of SEQ ID NOs: 65-123. In some embodiments, the diagnosis is based upon the presence and/or amount of fifteen or more peptides comprising the amino acid sequence of SEQ ID NOs: 65-123. In some embodiments, the diagnosis is based upon the presence and/or amount of twenty or more peptides comprising the amino acid sequence of SEQ ID NOs: 65-123. In some embodiments, the diagnosis is based upon the presence and/or amount of twenty-five or more peptides comprising the amino acid sequence of SEQ ID NOs: 65-123. In some embodiments, the diagnosis is based upon the presence and/or amount of thirty or more peptides comprising the amino acid sequence of SEQ ID NOs: 65-123. In some embodiments, the diagnosis is based upon the presence and/or amount of thirty-five or more peptides comprising the amino acid sequence of SEQ ID NOs: 65-123. In some embodiments, the diagnosis is based upon the presence and/or amount of forty or more peptides comprising the amino acid sequence of SEQ ID NOs: 65-123. In some embodiments, the diagnosis is based upon the presence and/or amount of forty-five or more peptides comprising the amino acid sequence of SEQ ID NOs: 65-123. In some embodiments, the diagnosis is based upon the presence and/or amount of fifty or more peptides comprising the amino acid sequence of SEQ ID NOs: 65-123. In some embodiments, the diagnosis is based upon the presence and/or amount of fifty-five or more peptides comprising the amino acid sequence of SEQ ID NOs: 65-123. In some embodiments, the diagnosis is based upon the presence and/or amount of each of the peptides comprising the amino acid sequence of SEQ ID NOs: 65-123. In some embodiments, the presence and/or amount of the peptide is determined using mass spectrometry.

In some embodiments, the diagnosis is based upon the presence and/or amount of one or more peptides comprising the amino acid sequence of SEQ ID NOs: 65-69. In some embodiments, the diagnosis is based upon the presence and/or amount of one or more peptides comprising the amino acid sequence of SEQ ID NOs: 65-74. In some embodiments, the diagnosis is based upon the presence and/or amount of one or more peptides comprising the amino acid sequence of SEQ ID NOs: 65-79. In some embodiments, the diagnosis is based upon the presence and/or amount of one or more peptides comprising the amino acid sequence of SEQ ID NOs: 65-84. In some embodiments, the diagnosis is based upon the presence and/or amount of one or more peptides comprising the amino acid sequence of SEQ ID NOs: 65-89. In some embodiments, the diagnosis is based upon the presence and/or amount of one or more peptides comprising the amino acid sequence of SEQ ID NOs: 65-94. In some embodiments, the diagnosis is based upon the presence and/or amount of one or more peptides comprising the amino acid sequence of SEQ ID NOs: 65-99. In some embodiments, the diagnosis is based upon the presence and/or amount of one or more peptides comprising the amino acid sequence of SEQ ID NOs: 65-104. In some embodiments, the diagnosis is based upon the presence and/or amount of one or more peptides comprising the amino acid sequence of SEQ ID NOs: 65-109. In some embodiments, the diagnosis is based upon the presence and/or amount of one or more peptides comprising the amino acid sequence of SEQ ID NOs: 65-114. In some embodiments, the diagnosis is based upon the presence and/or amount of one or more peptides comprising the amino acid sequence of SEQ ID NOs: 65-119. In some embodiments, the diagnosis is based upon the presence and/or amount of one or more peptides comprising the amino acid sequence of SEQ ID NOs: 65-123. In some embodiments, the diagnosis is based upon the presence and/or amount of one or more peptides comprising the amino acid sequence of SEQ ID NOs: 69-123. In some embodiments, the diagnosis is based upon the presence and/or amount of one or more peptides comprising the amino acid sequence of SEQ ID NOs: 74-84. In some embodiments, the diagnosis is based upon the presence and/or amount of one or more peptides comprising the amino acid sequence of SEQ ID NOs: 74-94. In some embodiments, the diagnosis is based upon the presence and/or amount of one or more peptides comprising the amino acid sequence of SEQ ID NOs: 74-104. In some embodiments, the diagnosis is based upon the presence and/or amount of one or more peptides comprising the amino acid sequence of SEQ ID NOs: 74-114. In some embodiments, the diagnosis is based upon the presence and/or amount of one or more peptides comprising the amino acid sequence of SEQ ID NOs: 74-123. In some embodiments, the diagnosis is based upon the presence and/or amount of one or more peptides comprising the amino acid sequence of SEQ ID NOs: 84-94. In some embodiments, the diagnosis is based upon the presence and/or amount of one or more peptides comprising the amino acid sequence of SEQ ID NOs: 84-104. In some embodiments, the diagnosis is based upon the presence and/or amount of one or more peptides comprising the amino acid sequence of SEQ ID NOs: 84-114. In some embodiments, the diagnosis is based upon the presence and/or amount of one or more peptides comprising the amino acid sequence of SEQ ID NOs: 84-123. In some embodiments, the diagnosis is based upon the presence and/or amount of one or more peptides comprising the amino acid sequence of SEQ ID NOs: 89-123. In some embodiments, the diagnosis is based upon the presence and/or amount of one or more peptides comprising the amino acid sequence of SEQ ID NOs: 94-104. In some embodiments, the diagnosis is based upon the presence and/or amount of one or more peptides comprising the amino acid sequence of SEQ ID NOs: 94-114. In some embodiments, the diagnosis is based upon the presence and/or amount of one or more peptides comprising the amino acid sequence of SEQ ID NOs: 94-123. In some embodiments, the diagnosis is based upon the presence and/or amount of one or more peptides comprising the amino acid sequence of SEQ ID NOs: 99-123. In some embodiments, the diagnosis is based upon the presence and/or amount of one or more peptides comprising the amino acid sequence of SEQ ID NOs: 104-114. In some embodiments, the diagnosis is based upon the presence and/or amount of one or more peptides comprising the amino acid sequence of SEQ ID NOs: 104-123. In some embodiments, the diagnosis is based upon the presence and/or amount of one or more peptides comprising the amino acid sequence of SEQ ID NOs: 109-123. In some embodiments, the diagnosis is based upon the presence and/or amount of one or more peptides comprising the amino acid sequence of SEQ ID NOs: 114-123. In some embodiments, the diagnosis is based upon the presence and/or amount of one or more peptides comprising the amino acid sequence of SEQ ID NOs: 119-123. In some embodiments, the presence and/or amount of the peptide is determined using mass spectrometry.

In some embodiments, the diagnosis is based upon the presence and/or amount of one or more peptides comprising the amino acid sequence of SEQ ID NOs: 124-188. In some embodiments, the presence and/or amount of the peptide is determined using mass spectrometry.

In some embodiments, the diagnosis is based upon the presence and/or amount of one or more pregnancy-specific proteins. In some embodiments, the diagnosis is based upon the presence and/or amount of one or more glycosylated pregnancy-specific proteins. In some embodiments, the diagnosis is based upon the presence and/or amount of one or more of the group consisting of pregnancy-specific beta-1-glycoprotein 1 (PSG1), putative pregnancy-specific beta-1-glycoprotein 7 (PSG7), and pregnancy zone protein (PZP). In some embodiments, the diagnosis is based upon the presence and/or amount of one or more of SEQ ID NOs: 42-44. In some embodiments, the diagnosis is based upon the presence and/or amount of one or more glycopeptides originating from one or more glycosylated pregnancy-specific proteins. In some embodiments, the diagnosis is based upon the presence and/or amount of one or more glycopeptides originating from one or more of PSG1, PSG7, or PZP. In some embodiments, the diagnosis is based upon the presence and/or amount of one or more of SEQ ID NOs: 110-118.

In some embodiments, the diagnosis is based upon the presence and/or amount of one or more peptides consisting of the amino acid sequence of SEQ ID NOs: 65-188. In some embodiments, the diagnosis is based upon the presence and/or amount of two or more peptides consisting of the amino acid sequence of SEQ ID NOs: 65-188. In some embodiments, the diagnosis is based upon the presence and/or amount of three or more peptides consisting of the amino acid sequence of SEQ ID NOs: 65-188. In some embodiments, the diagnosis is based upon the presence and/or amount of four or more peptides consisting of the amino acid sequence of SEQ ID NOs: 65-188. In some embodiments, the diagnosis is based upon the presence and/or amount of five or more peptides consisting of the amino acid sequence of SEQ ID NOs: 65-188. In some embodiments, the diagnosis is based upon the presence and/or amount of six or more peptides consisting of the amino acid sequence of SEQ ID NOs: 65-188. In some embodiments, the diagnosis is based upon the presence and/or amount of seven or more peptides consisting of the amino acid sequence of SEQ ID NOs: 65-188. In some embodiments, the diagnosis is based upon the presence and/or amount of eight or more peptides consisting of the amino acid sequence of SEQ ID NOs: 65-188. In some embodiments, the diagnosis is based upon the presence and/or amount of nine or more peptides consisting of the amino acid sequence of SEQ ID NOs: 65-188. In some embodiments, the diagnosis is based upon the presence and/or amount of ten or more peptides consisting of the amino acid sequence of SEQ ID NOs: 65-188. In some embodiments, the diagnosis is based upon the presence and/or amount of fifteen or more peptides consisting of the amino acid sequence of SEQ ID NOs: 65-188. In some embodiments, the diagnosis is based upon the presence and/or amount of twenty or more peptides consisting of the amino acid sequence of SEQ ID NOs: 65-188. In some embodiments, the diagnosis is based upon the presence and/or amount of twenty-five or more peptides consisting of the amino acid sequence of SEQ ID NOs: 65-188. In some embodiments, the diagnosis is based upon the presence and/or amount of thirty or more peptides consisting of the amino acid sequence of SEQ ID NOs: 65-188. In some embodiments, the diagnosis is based upon the presence and/or amount of thirty-five or more peptides consisting of the amino acid sequence of SEQ ID NOs: 65-188. In some embodiments, the diagnosis is based upon the presence and/or amount of forty or more peptides consisting of the amino acid sequence of SEQ ID NOs: 65-188. In some embodiments, the diagnosis is based upon the presence and/or amount of forty-five or more peptides consisting of the amino acid sequence of SEQ ID NOs: 65-188. In some embodiments, the diagnosis is based upon the presence and/or amount of fifty or more peptides consisting of the amino acid sequence of SEQ ID NOs: 65-188. In some embodiments, the diagnosis is based upon the presence and/or amount of fifty-five or more peptides consisting of the amino acid sequence of SEQ ID NOs: 65-188. In some embodiments, the diagnosis is based upon the presence and/or amount of sixty or more peptides consisting of the amino acid sequence of SEQ ID NOs: 65-188. In some embodiments, the diagnosis is based upon the presence and/or amount of sixty-five or more peptides consisting of the amino acid sequence of SEQ ID NOs: 65-188. In some embodiments, the diagnosis is based upon the presence and/or amount of seventy or more peptides consisting of the amino acid sequence of SEQ ID NOs: 65-188. In some embodiments, the diagnosis is based upon the presence and/or amount of seventy-five or more peptides consisting of the amino acid sequence of SEQ ID NOs: 65-188. In some embodiments, the diagnosis is based upon the presence and/or amount of eighty or more peptides consisting of the amino acid sequence of SEQ ID NOs: 65-188. In some embodiments, the diagnosis is based upon the presence and/or amount of eighty-five or more peptides consisting of the amino acid sequence of SEQ ID NOs: 65-188. In some embodiments, the diagnosis is based upon the presence and/or amount of ninety or more peptides consisting of the amino acid sequence of SEQ ID NOs: 65-188. In some embodiments, the diagnosis is based upon the presence and/or amount of ninety-five or more peptides consisting of the amino acid sequence of SEQ ID NOs: 65-188. In some embodiments, the diagnosis is based upon the presence and/or amount of one hundred or more peptides consisting of the amino acid sequence of SEQ ID NOs: 65-188. In some embodiments, the diagnosis is based upon the presence and/or amount of one hundred five or more peptides consisting of the amino acid sequence of SEQ ID NOs: 65-188. In some embodiments, the diagnosis is based upon the presence and/or amount of one hundred ten or more peptides consisting of the amino acid sequence of SEQ ID NOs: 65-188. In some embodiments, the diagnosis is based upon the presence and/or amount of one hundred fifteen or more peptides consisting of the amino acid sequence of SEQ ID NOs: 65-188. In some embodiments, the diagnosis is based upon the presence and/or amount of one hundred twenty or more peptides consisting of the amino acid sequence of SEQ ID NOs: 65-188. In some embodiments, the diagnosis is based upon the presence and/or amount of each of the peptides consisting of the amino acid sequence of SEQ ID NOs: 65-188. In some embodiments, the presence and/or amount of the peptide is determined using mass spectrometry.

In some embodiments, the diagnosis is based upon the presence and/or amount of one or more peptides consisting of the amino acid sequence of SEQ ID NOs: 65-74. In some embodiments, the diagnosis is based upon the presence and/or amount of one or more peptides consisting of the amino acid sequence of SEQ ID NOs: 65-84. In some embodiments, the diagnosis is based upon the presence and/or amount of one or more peptides consisting of the amino acid sequence of SEQ ID NOs: 65-94. In some embodiments, the diagnosis is based upon the presence and/or amount of one or more peptides consisting of the amino acid sequence of SEQ ID NOs: 65-104. In some embodiments, the diagnosis is based upon the presence and/or amount of one or more peptides consisting of the amino acid sequence of SEQ ID NOs: 65-114. In some embodiments, the diagnosis is based upon the presence and/or amount of one or more peptides consisting of the amino acid sequence of SEQ ID NOs: 65-124. In some embodiments, the diagnosis is based upon the presence and/or amount of one or more peptides consisting of the amino acid sequence of SEQ ID NOs: 65-134. In some embodiments, the diagnosis is based upon the presence and/or amount of one or more peptides consisting of the amino acid sequence of SEQ ID NOs: 65-144. In some embodiments, the diagnosis is based upon the presence and/or amount of one or more peptides consisting of the amino acid sequence of SEQ ID NOs: 65-154. In some embodiments, the diagnosis is based upon the presence and/or amount of one or more peptides consisting of the amino acid sequence of SEQ ID NOs: 65-164. In some embodiments, the diagnosis is based upon the presence and/or amount of one or more peptides consisting of the amino acid sequence of SEQ ID NOs: 65-174. In some embodiments, the diagnosis is based upon the presence and/or amount of one or more peptides consisting of the amino acid sequence of SEQ ID NOs: 65-184. In some embodiments, the diagnosis is based upon the presence and/or amount of one or more peptides consisting of the amino acid sequence of SEQ ID NOs: 65-188. In some embodiments, the diagnosis is based upon the presence and/or amount of one or more peptides consisting of the amino acid sequence of SEQ ID NOs: 74-188. In some embodiments, the diagnosis is based upon the presence and/or amount of one or more peptides consisting of the amino acid sequence of SEQ ID NOs: 84-104. In some embodiments, the diagnosis is based upon the presence and/or amount of one or more peptides consisting of the amino acid sequence of SEQ ID NOs: 84-124. In some embodiments, the diagnosis is based upon the presence and/or amount of one or more peptides consisting of the amino acid sequence of SEQ ID NOs: 84-144. In some embodiments, the diagnosis is based upon the presence and/or amount of one or more peptides consisting of the amino acid sequence of SEQ ID NOs: 84-164. In some embodiments, the diagnosis is based upon the presence and/or amount of one or more peptides consisting of the amino acid sequence of SEQ ID NOs: 84-188. In some embodiments, the diagnosis is based upon the presence and/or amount of one or more peptides consisting of the amino acid sequence of SEQ ID NOs: 94-188. In some embodiments, the diagnosis is based upon the presence and/or amount of one or more peptides consisting of the amino acid sequence of SEQ ID NOs: 104-124. In some embodiments, the diagnosis is based upon the presence and/or amount of one or more peptides consisting of the amino acid sequence of SEQ ID NOs: 104-144. In some embodiments, the diagnosis is based upon the presence and/or amount of one or more peptides consisting of the amino acid sequence of SEQ ID NOs: 104-164. In some embodiments, the diagnosis is based upon the presence and/or amount of one or more peptides consisting of the amino acid sequence of SEQ ID NOs: 104-188. In some embodiments, the diagnosis is based upon the presence and/or amount of one or more peptides consisting of the amino acid sequence of SEQ ID NOs: 114-188. In some embodiments, the diagnosis is based upon the presence and/or amount of one or more peptides consisting of the amino acid sequence of SEQ ID NOs: 124-144. In some embodiments, the diagnosis is based upon the presence and/or amount of one or more peptides consisting of the amino acid sequence of SEQ ID NOs: 124-164. In some embodiments, the diagnosis is based upon the presence and/or amount of one or more peptides consisting of the amino acid sequence of SEQ ID NOs: 124-188. In some embodiments, the diagnosis is based upon the presence and/or amount of one or more peptides consisting of the amino acid sequence of SEQ ID NOs: 134-188. In some embodiments, the diagnosis is based upon the presence and/or amount of one or more peptides consisting of the amino acid sequence of SEQ ID NOs: 144-164. In some embodiments, the diagnosis is based upon the presence and/or amount of one or more peptides consisting of the amino acid sequence of SEQ ID NOs: 144-188. In some embodiments, the diagnosis is based upon the presence and/or amount of one or more peptides consisting of the amino acid sequence of SEQ ID NOs: 154-188. In some embodiments, the diagnosis is based upon the presence and/or amount of one or more peptides consisting of the amino acid sequence of SEQ ID NOs: 164-188. In some embodiments, the diagnosis is based upon the presence and/or amount of one or more peptides consisting of the amino acid sequence of SEQ ID NOs: 174-188. In some embodiments, the diagnosis is based upon the presence and/or amount of one or more peptides consisting of the amino acid sequence of SEQ ID NOs: 184-188. In some embodiments, the presence and/or amount of the peptide is determined using mass spectrometry.

In some embodiments, the diagnosis is based upon the presence and/or amount of one or more peptides consisting of the amino acid sequence of SEQ ID NOs: 65-123. In some embodiments, the diagnosis is based upon the presence and/or amount of two or more peptides consisting of the amino acid sequence of SEQ ID NOs: 65-123. In some embodiments, the diagnosis is based upon the presence and/or amount of three or more peptides consisting of the amino acid sequence of SEQ ID NOs: 65-123. In some embodiments, the diagnosis is based upon the presence and/or amount of four or more peptides consisting of the amino acid sequence of SEQ ID NOs: 65-123. In some embodiments, the diagnosis is based upon the presence and/or amount of five or more peptides consisting of the amino acid sequence of SEQ ID NOs: 65-123. In some embodiments, the diagnosis is based upon the presence and/or amount of ten or more peptides consisting of the amino acid sequence of SEQ ID NOs: 65-123. In some embodiments, the diagnosis is based upon the presence and/or amount of fifteen or more peptides consisting of the amino acid sequence of SEQ ID NOs: 65-123. In some embodiments, the diagnosis is based upon the presence and/or amount of twenty or more peptides consisting of the amino acid sequence of SEQ ID NOs: 65-123. In some embodiments, the diagnosis is based upon the presence and/or amount of twenty-five or more peptides consisting of the amino acid sequence of SEQ ID NOs: 65-123. In some embodiments, the diagnosis is based upon the presence and/or amount of thirty or more peptides consisting of the amino acid sequence of SEQ ID NOs: 65-123. In some embodiments, the diagnosis is based upon the presence and/or amount of thirty-five or more peptides consisting of the amino acid sequence of SEQ ID NOs: 65-123. In some embodiments, the diagnosis is based upon the presence and/or amount of forty or more peptides consisting of the amino acid sequence of SEQ ID NOs: 65-123. In some embodiments, the diagnosis is based upon the presence and/or amount of forty-five or more peptides consisting of the amino acid sequence of SEQ ID NOs: 65-123. In some embodiments, the diagnosis is based upon the presence and/or amount of fifty or more peptides consisting of the amino acid sequence of SEQ ID NOs: 65-123. In some embodiments, the diagnosis is based upon the presence and/or amount of fifty-five or more peptides consisting of the amino acid sequence of SEQ ID NOs: 65-123. In some embodiments, the diagnosis is based upon the presence and/or amount of each of the peptides consisting of the amino acid sequence of SEQ ID NOs: 65-123. In some embodiments, the presence and/or amount of the peptide is determined using mass spectrometry.

In some embodiments, the diagnosis is based upon the presence and/or amount of one or more peptides consisting of the amino acid sequence of SEQ ID NOs: 65-69. In some embodiments, the diagnosis is based upon the presence and/or amount of one or more peptides consisting of the amino acid sequence of SEQ ID NOs: 65-74. In some embodiments, the diagnosis is based upon the presence and/or amount of one or more peptides consisting of the amino acid sequence of SEQ ID NOs: 65-79. In some embodiments, the diagnosis is based upon the presence and/or amount of one or more peptides consisting of the amino acid sequence of SEQ ID NOs: 65-84. In some embodiments, the diagnosis is based upon the presence and/or amount of one or more peptides consisting of the amino acid sequence of SEQ ID NOs: 65-89. In some embodiments, the diagnosis is based upon the presence and/or amount of one or more peptides consisting of the amino acid sequence of SEQ ID NOs: 65-94. In some embodiments, the diagnosis is based upon the presence and/or amount of one or more peptides consisting of the amino acid sequence of SEQ ID NOs: 65-99. In some embodiments, the diagnosis is based upon the presence and/or amount of one or more peptides consisting of the amino acid sequence of SEQ ID NOs: 65-104. In some embodiments, the diagnosis is based upon the presence and/or amount of one or more peptides consisting of the amino acid sequence of SEQ ID NOs: 65-109. In some embodiments, the diagnosis is based upon the presence and/or amount of one or more peptides consisting of the amino acid sequence of SEQ ID NOs: 65-114. In some embodiments, the diagnosis is based upon the presence and/or amount of one or more peptides consisting of the amino acid sequence of SEQ ID NOs: 65-119. In some embodiments, the diagnosis is based upon the presence and/or amount of one or more peptides consisting of the amino acid sequence of SEQ ID NOs: 65-123. In some embodiments, the diagnosis is based upon the presence and/or amount of one or more peptides consisting of the amino acid sequence of SEQ ID NOs: 69-123. In some embodiments, the diagnosis is based upon the presence and/or amount of one or more peptides consisting of the amino acid sequence of SEQ ID NOs: 74-84. In some embodiments, the diagnosis is based upon the presence and/or amount of one or more peptides consisting of the amino acid sequence of SEQ ID NOs: 74-94. In some embodiments, the diagnosis is based upon the presence and/or amount of one or more peptides consisting of the amino acid sequence of SEQ ID NOs: 74-104. In some embodiments, the diagnosis is based upon the presence and/or amount of one or more peptides consisting of the amino acid sequence of SEQ ID NOs: 74-114. In some embodiments, the diagnosis is based upon the presence and/or amount of one or more peptides consisting of the amino acid sequence of SEQ ID NOs: 74-123. In some embodiments, the diagnosis is based upon the presence and/or amount of one or more peptides consisting of the amino acid sequence of SEQ ID NOs: 84-94. In some embodiments, the diagnosis is based upon the presence and/or amount of one or more peptides consisting of the amino acid sequence of SEQ ID NOs: 84-104. In some embodiments, the diagnosis is based upon the presence and/or amount of one or more peptides consisting of the amino acid sequence of SEQ ID NOs: 84-114. In some embodiments, the diagnosis is based upon the presence and/or amount of one or more peptides consisting of the amino acid sequence of SEQ ID NOs: 84-123. In some embodiments, the diagnosis is based upon the presence and/or amount of one or more peptides consisting of the amino acid sequence of SEQ ID NOs: 89-123. In some embodiments, the diagnosis is based upon the presence and/or amount of one or more peptides consisting of the amino acid sequence of SEQ ID NOs: 94-104. In some embodiments, the diagnosis is based upon the presence and/or amount of one or more peptides consisting of the amino acid sequence of SEQ ID NOs: 94-114. In some embodiments, the diagnosis is based upon the presence and/or amount of one or more peptides consisting of the amino acid sequence of SEQ ID NOs: 94-123. In some embodiments, the diagnosis is based upon the presence and/or amount of one or more peptides consisting of the amino acid sequence of SEQ ID NOs: 99-123. In some embodiments, the diagnosis is based upon the presence and/or amount of one or more peptides consisting of the amino acid sequence of SEQ ID NOs: 104-114. In some embodiments, the diagnosis is based upon the presence and/or amount of one or more peptides consisting of the amino acid sequence of SEQ ID NOs: 104-123. In some embodiments, the diagnosis is based upon the presence and/or amount of one or more peptides consisting of the amino acid sequence of SEQ ID NOs: 109-123. In some embodiments, the diagnosis is based upon the presence and/or amount of one or more peptides consisting of the amino acid sequence of SEQ ID NOs: 114-123. In some embodiments, the diagnosis is based upon the presence and/or amount of one or more peptides consisting of the amino acid sequence of SEQ ID NOs: 119-123. In some embodiments, the presence and/or amount of the peptide is determined using mass spectrometry.

In some embodiments, the diagnosis is based upon the presence and/or amount of one or more peptides consisting of the amino acid sequence of SEQ ID NOs: 124-188. In some embodiments, the presence and/or amount of the peptide is determined using mass spectrometry.

In some embodiments, the method further comprises collecting a biological sample. In some embodiments, the method comprises collecting maternal serum. In some embodiments, maternal serum is collected after 20 weeks gestation. In some embodiments, maternal serum is collected in or after 27 weeks, in or after 30 weeks, in or after 33 weeks or in or after 36 weeks of gestation.

For example, in certain embodiments, the presence or amount of the at least one peptide structure is detected using mass spectrometry, ELISA, or MRM mass spectrometry. In one embodiment, the at least one peptide structure is none, or below a detection limit. In one embodiment, the preeclampsia is severe preeclampsia. In one embodiment, the biological sample is maternal serum. In one embodiment, the one or more peptide structure includes a glycopeptide of a pregnancy-specific protein, and the at least one peptide structure comprises three or more peptide structures identified in Table 17.

In certain embodiments, the present embodiments may further include assessing one or more risk factors or clinical indicators of preeclampsia, in which a clinical indicator of preeclampsia is selected from the group consisting of protein in the urine and high blood pressure. In certain embodiments, the risk factor for preeclampsia is selected from the group consisting of history of preeclampsia, chronic hypertension, obesity, and multiple pregnancy. In certain embodiments, the individual is determined have a healthy state, in which a healthy state may include the absence of preeclampsia and/or a low risk for preeclampsia. The present embodiments may further include diagnosing a placental development problem.

### VI.B Training a Model

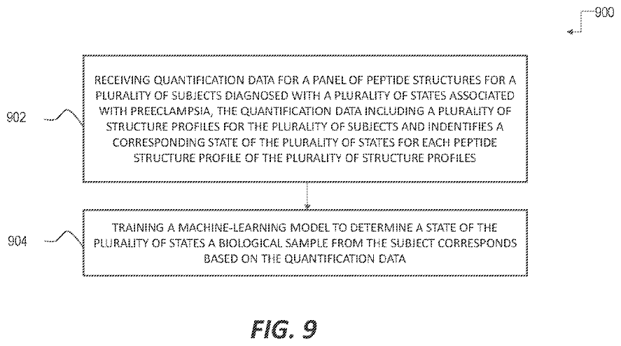

FIG. 9 illustrates a flow diagram 900 of a method for training a model to diagnose a subject with one of a plurality of states associated with preeclampsia, in accordance with the presently disclosed embodiments. The flow diagram 900 may be performed utilizing one or more processing devices (e.g., computing platform 302 as discussed above with respect to FIG. 3) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), or any other processing device(s) that may be suitable for processing various medical profile data and making one or more decisions based thereon), software (e.g., instructions running/executing on one or more processors), firmware (e.g., microcode), or some combination thereof.

The flow diagram 900 may begin at block 902 with one or more processing devices (e.g., computing platform 302) receiving quantification data for a panel of peptide structures for a plurality of subjects diagnosed with the plurality of states associated with the preeclampsia, wherein the quantification data comprises a plurality of peptide structure profiles for the plurality of subjects and identifies a corresponding state of the plurality of states for each peptide structure profile of the plurality of peptide structure profiles. The flow diagram 900 may then conclude at block 904 with one or more processing devices (e.g., computing platform 302) training a machine-learning model to determine a state of the plurality of states a biological sample from the subject corresponds based on the quantification data.

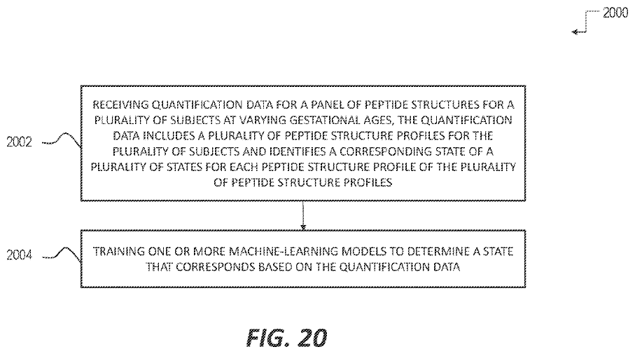

FIG. 20 illustrates a flow diagram 2000 of a method for training a model to determine a plurality of states associated with fetal gestational age, in accordance with the presently disclosed embodiments. The flow diagram 2000 may be performed utilizing one or more processing devices (e.g., computing platform 302 as discussed above with respect to FIG. 3) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), or any other processing device(s) that may be suitable for processing various medical profile data and making one or more decisions based thereon), software (e.g., instructions running/executing on one or more processors), firmware (e.g., microcode), or some combination thereof.

The flow diagram 2000 may begin at block 2002 with one or more processing devices (e.g., computing platform 302) receiving quantification data for a panel of peptide structures for a plurality of subjects at varying gestational ages, wherein the quantification data comprises a plurality of peptide structure profiles for the plurality of subjects and identifies a corresponding state of the plurality of states for each peptide structure profile of the plurality of peptide structure profiles. The flow diagram 2000 may then conclude at block 2004 with one or more processing devices (e.g., computing platform 302) training one or more machine-learning models to determine a state of the plurality of states that corresponds based on the quantification data.

For example, in certain embodiments, the quantification data for a peptide structure of the set of peptide structures may include an abundance, a relative abundance, a normalized abundance, a relative quantity, an adjusted quantity, a normalized quantity, a relative concentration, an adjusted concentration, or a normalized concentration. In certain embodiments, the machine-learning model is trained using random forest or logical progression training methods. In certain embodiments, training the machine-learning model to determine the state of the plurality of states may include training the machine-learning model to generate a class label for the state of the plurality of states. For example, in one embodiment, the machine-learning model may include a logistic regression model, which may be further trained by generating a log error cost function based on the plurality of states associated with preeclampsia and minimizing the log error cost function based on the plurality of states associated with preeclampsia and the determined state of the plurality of states.

### VI.C Diagnosis and/or Treatment

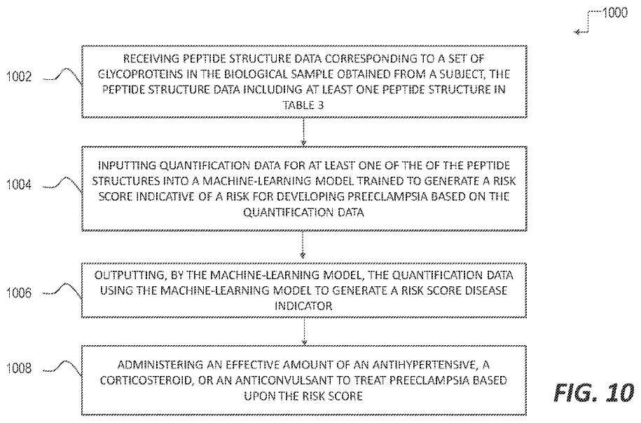

FIG. 10 illustrates a flow diagram 1000 of a method for treating preeclampsia in a subject, in accordance with the presently disclosed embodiments. The flow diagram 1000 may be performed utilizing one or more processing devices (e.g., computing platform 302 as discussed above with respect to FIG. 3) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), or any other processing device(s) that may be suitable for processing various medical profile data and making one or more decisions based thereon), software (e.g., instructions running/executing on one or more processors), firmware (e.g., microcode), or some combination thereof.

The flow diagram 1000 may begin at block 1002 with one or more processing devices (e.g., computing platform 302) receiving peptide structure data corresponding to a set of glycoproteins in the biological sample obtained from a subject, wherein the peptide structure data comprises at least one peptide structure from Table 3. The flow diagram 1000 may then continue at block 1004 with one or more processing devices (e.g., computing platform 302) inputting quantification data for at least one of the peptide structures into a machine-learning model trained to generate a risk score indicative of a risk for developing preeclampsia based on the quantification datal. The flow diagram 1000D may then continue at block 1006 with one or more processing devices (e.g., computing platform 302) outputting, by the machine-learning model, the quantification data using the machine learning model to generate a risk score. The flow diagram 1000 may then conclude at block 1008 with one or more processing devices (e.g., computing platform 302) administering an effective amount of an antihypertensive.

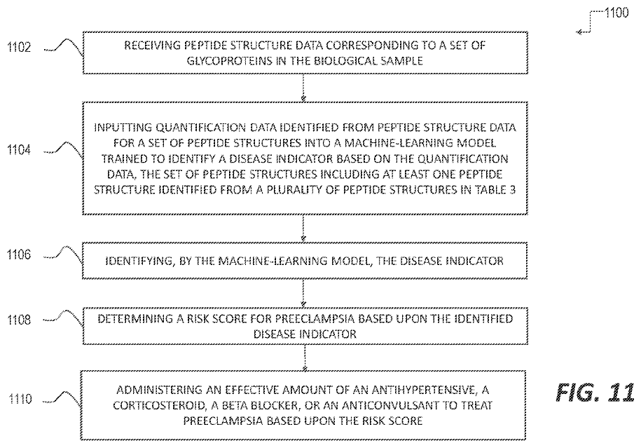

FIG. 11 illustrates a flow diagram 1100 of a method for treating preeclampsia in a subject, in accordance with the presently disclosed embodiments. The flow diagram 1100 may be performed utilizing one or more processing devices (e.g., computing platform 302 as discussed above with respect to FIG. 3) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), or any other processing device(s) that may be suitable for processing various medical profile data and making one or more decisions based thereon), software (e.g., instructions running/executing on one or more processors), firmware (e.g., microcode), or some combination thereof.

The flow diagram 1100 may begin at block 1102 with one or more processing devices (e.g., computing platform 302) receiving peptide structure data corresponding to a set of glycoproteins in the biological sample. The flow diagram 1100 may then continue at block 1104 with one or more processing devices (e.g., computing platform 302) inputting quantification data identified from the peptide structure data for a set of the peptide structures into a machine-learning model trained to identify a disease indicator based on the quantification data, wherein the set of peptide structure data comprises at least one peptide structure identified from a plurality of peptide structures in Table 3. The flow diagram 1100 may then continue at block 1106 with one or more processing devices (e.g., computing platform 302) identifying, by the machine-learning model, the disease indicator. The flow diagram 1100 may then continue at block 1108 with one or more processing devices (e.g., computing platform 302) determining a risk score for preeclampsia based upon the identified disease indicator. The flow diagram 1100 may then conclude at block 1110 with one or more processing devices (e.g., computing platform 302) administering an effective amount of an antihypertensive, a corticosteroid, a beta blocker, or an anticonvulsant to treat preeclampsia based upon the risk score.

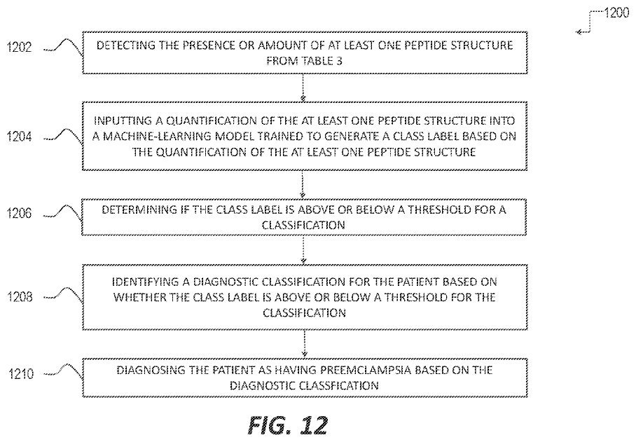

FIG. 12 illustrates a flow diagram 1200 of a method for diagnosing an individual with preeclampsia, in accordance with the presently disclosed embodiments. The flow diagram 1200 may be performed utilizing one or more processing devices (e.g., computing platform 302 as discussed above with respect to FIG. 3) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), or any other processing device(s) that may be suitable for processing various medical profile data and making one or more decisions based thereon), software (e.g., instructions running/executing on one or more processors), firmware (e.g., microcode), or some combination thereof.

The flow diagram 1200 may begin at block 1202 with one or more processing devices (e.g., computing platform 302) detecting the presence or amount of at least one peptide structure structures from Table 3. The flow diagram 1200 may then continue at block 1204 with one or more processing devices (e.g., computing platform 302) inputting a quantification of the detected at least one peptide structure into a machine-learning model trained to generate a class label 1. The flow diagram 1200 may then continue at block 1206 with one or more processing devices (e.g., computing platform 302) determining if the class label is above or below a threshold for a classification. The flow diagram 1200 may then continue at block 1208 with one or more processing devices (e.g., computing platform 302) identifying a diagnostic classification for a patient based on whether the class label is above or below a threshold for the classification. The flow diagram 1200 may then conclude at block 1210 with one or more processing devices (e.g., computing platform 302) diagnosing the patient as having preeclampsia based on the diagnostic classification.

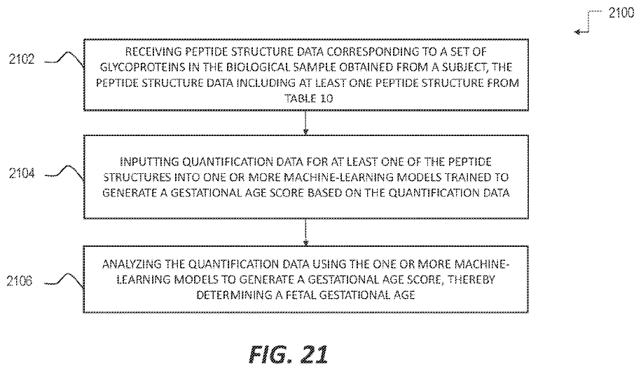

FIG. 21 illustrates a flow diagram 2100 of a method for determining fetal gestational age, in accordance with the presently disclosed embodiments. The flow diagram 2100 may be performed utilizing one or more processing devices (e.g., computing platform 302 as discussed above with respect to FIG. 3) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), or any other processing device(s) that may be suitable for processing various medical profile data and making one or more decisions based thereon), software (e.g., instructions running/executing on one or more processors), firmware (e.g., microcode), or some combination thereof.

The flow diagram 2100 may begin at block 2102 with one or more processing devices (e.g., computing platform 302) receiving peptide structure data corresponding to a set of glycoproteins in the biological sample obtained from a subject, wherein the peptide structure data comprises at least one peptide structure from Table 10. The flow diagram 2100 may then continue at block 2104 with one or more processing devices (e.g., computing platform 302) inputting quantification data identified from the peptide structure data for the at least one peptide structure into one or more machine-learning models trained to generate a gestational age score. The flow diagram 2100 may then conclude at block 2106 with one or more processing devices (e.g., computing platform 302) analyzing the quantification data using the one or more machine-learning model to generate a gestational age score, thereby determining a fetal gestational age.

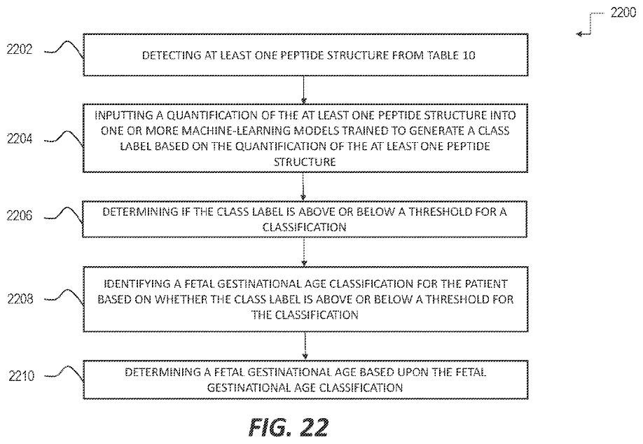

FIG. 22 illustrates a flow diagram 2200 of a method for determining a fetal gestational age in a subject, in accordance with the presently disclosed embodiments. The flow diagram 2200 may be performed utilizing one or more processing devices (e.g., computing platform 302 as discussed above with respect to FIG. 3) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), or any other processing device(s) that may be suitable for processing various medical profile data and making one or more decisions based thereon), software (e.g., instructions running/executing on one or more processors), firmware (e.g., microcode), or some combination thereof.

The flow diagram 2200 may begin at block 902 with one or more processing devices (e.g., computing platform 302) detecting at least one peptide structure from Table 10. The flow diagram 2200 may then continue at block 904 with one or more processing devices (e.g., computing platform 302) inputting a quantification of the detected peptide structure into one or more trained machine-learning models to generate a class label. The flow diagram 2200 may then continue at block 906 with one or more processing devices (e.g., computing platform 302) determining if the class label is above or below a threshold for a classification. The flow diagram 2200 may then continue at block 908 with one or more processing devices (e.g., computing platform 302) identifying a fetal gestational age classification for the patient based on whether the class label is above or below a threshold for a classification. The flow diagram 2200 may then conclude at block 910 with one or more processing devices (e.g., computing platform 302) determining a fetal gestational age based upon the fetal gestational age classification.

For example, in certain embodiments, determining a gestational age of a fetus may include detecting the presence or amount a peptide structure from Table 10, and further determining the gestational age of the fetus based upon the presence or amount of the at least one peptide structure from Table 10. In one embodiment, detecting the peptide structure may be performed using mass spectrometry, ELISA, or MRM mass spectrometry. In one embodiment, the gestational age may be over 20 weeks. In another embodiment, the gestational age may be over 24 weeks. In one embodiment, the biological sample may include maternal serum, which may be collected in the second or third trimester of pregnancy. In some embodiments, the peptide structure may include a glycopeptide, including a pregnancy-specific protein. In other embodiments, the peptide structure may include at least three peptide structures identified in Table 10.

In certain embodiments, the one or more machine-learning models may include an ensemble learning model. For example, the ensemble learning model may include a plurality of decision trees, in which a succeeding decision tree of the plurality of decision trees is trained to correct an error of a preceding decision tree of the plurality of decision trees to identify the fetal gestational age indicator. In certain embodiments, the one or more machine-learning models may include one or more of a gradient boosting model, an adaptive boosting (AdaBoost) model, an eXtreme gradient boosting (XGBoost) model, a light gradient boosted machine (LightGBM) model, or a categorical boosting (CatBoost) model.

### VII Methods of Treatment

In some embodiments, provided herein are methods of treating preeclampsia based upon the presence, amount, and/or relative amount of one or more biomarkers provided herein. In some embodiments, the method comprises classifying a biological sample with respect to a plurality of states associated with preeclampsia based upon one or more peptides structure provided herein and administering a treatment for preeclampsia based upon the classification. In some embodiments, the method comprises inputting quantification data identified from peptide structure data for a set of peptides to identify a disease indicator, detecting the presence of a corresponding state associated with preeclampsia in response that the disease indicator falls within a selected range, and diagnosing preeclampsia. In some embodiments, the method further comprises administering an effective amount of a therapy for preeclampsia. In some embodiments, the method further comprises selecting a particular therapy based upon the disease indicator.

In some embodiments, provided herein is a method of treating preeclampsia in a subject comprising inputting quantification data for at least one peptide structure into a machine learning model to generate a risk score, and administering an effective amount of a treatment for preeclampsia based upon the risk score. In some embodiments, a specific treatment is selected based upon a risk score. In some embodiments, a risk score corresponding to a higher risk of developing preeclampsia results in selection of a therapy for treating preeclampsia. In some embodiments, a risk score corresponding to a lower risk of developing preeclampsia results in selection of no therapy for treating preeclampsia.

In some embodiments, provided herein is a method of treating preeclampsia comprising detecting the presence (or absence) or amount of at least one peptide structure from Table 3 and administering an effective amount of a preeclampsia therapy to the individual. In some embodiments, the method further comprises selecting a therapy based upon the presence, and/or amount of the at least peptide structure from Table 3. In some embodiments, the diagnosis and/or treatment is based upon the presence and/or amount of at least two, at least three, at least four, at least five, at least six, at least seven or eight peptide structures from Table 3.

In some embodiments, the diagnosis and/or treatment is based upon the presence and/or amount of at least one, at least two, at least three, at least four, at least five, at least six, at least seven, at least eight, at least nine, at least ten, at least fifteen, at least twenty, at least twenty-five, at least thirty, at least thirty-five, at least forty, at least forty-five, at least fifty, at least fifty-five, at least sixty, at least sixty-five, at least seventy, at least seventy-five, at least eighty, at least eighty-five, at least ninety, at least ninety-five, at least one hundred, at least one hundred five, at least one hundred ten, at least one hundred fifteen, at least one hundred twenty, or one hundred twenty-four peptide structures from Table 17. In some embodiments, the presence and/or amount of the peptide structure is determined using mass spectrometry.

In some embodiments, the therapy for preeclampsia is selected from the group consisting of an antihypertensive, a corticosteroid, a beta blocker, or an anticonvulsant. In some embodiments, the therapy comprises magnesium sulfate. In some embodiments, the treatment for preeclampsia comprises medicine to control blood pressure, prevent seizures or other complications, and/or steroids to speed the development of the fetus's lungs. In some embodiments, treatment for preeclampsia is to deliver the fetus. In some embodiments, the fetus is delivered if preeclampsia is diagnosed after 34 weeks gestation. In some embodiments, the fetus is delivered if preeclampsia is diagnosed after 37 weeks gestation.

In some embodiments, the diagnosis results in further monitoring of the patient for progression of preeclampsia. In some embodiments, monitoring comprises detecting platelet counts, liver enzyme levels, kidney function, and urinary protein levels. In some embodiments, the individual is admitted to the hospital for monitoring.

In some embodiments, the diagnosis results in further monitoring of the fetus, for example ultrasound, heart rate monitoring, assessment of fetal growth, and amniotic fluid assessment.

In some embodiments the diagnosis results in administration of one or more therapies to treat preeclampsia prophylactically. In some embodiments, an antihypertensive, a corticosteroid, a beta blocker, or an anticonvulsant is administered prophylactically based upon the determination that an individual is at risk for preeclampsia. In some embodiments, the therapy comprises magnesium sulfate. In some embodiments, the treatment for preeclampsia comprises medicine to control blood pressure, prevent seizures or other complications, and/or steroids to speed the development of the fetus's lungs

In some embodiments, the method further comprises assessing one or more risk factors associated with preeclampsia or clinical indicators of preeclampsia to provide a diagnosis. In some embodiments, the risk factor for preeclampsia is any of history of preeclampsia, chronic hypertension, obesity, and multiple pregnancy

In some embodiments, protein in the urine, platelet count, liver function, kidney problems, fluid in the lungs, headaches, visual disturbances, high blood pressure, blood test, urine analysis, fetal ultrasound, nonstress test, or biophysical profile is assessed. A nonstress test is a procedure that checks how the fetus reacts when it moves. A biophysical profile uses an ultrasound to measure fetal breathing, muscle tone, movement and the volume of amniotic fluid in the uterus. The images of the fetus created during the ultrasound exam allows estimated fetal weight and the amount of fluid in the uterus (amniotic fluid). In some embodiments, the level of creatine in the urine is assessed. In some embodiments, the level of other proteins in the urine relative to creatine is assessed.

In some embodiments, the risk factors for preeclampsia comprise history of hypertensive disease during a previous pregnancy or a maternal disease including chronic kidney disease, autoimmune diseases, diabetes, or chronic hypertension. Women are at moderate risk if they are nulliparous, 240 years of age, have a body mass index (BMI) ≥35 kg/m, a family history of preeclampsia, a multifetal pregnancy, or a pregnancy interval of more than 10 years. In some embodiments, the individual has 1, 2, 3, 4, 5, 6, or more risk factors for preeclampsia.

Also provided herein is a method of preventing and/or reducing the risk of preeclampsia in an individual determined to have a risk of developing preeclampsia. In some embodiments, the method comprises administering one or more therapies to treat preeclampsia prophylactically to the individual. In some embodiments, the method results in a delayed progression of preeclampsia. In some embodiments, the method results in decreased severity of preeclampsia.

In some embodiments, provided herein is a method of determining a gestational age of a fetus and administering a therapy based upon the determined gestational age. For example, in some embodiments, steroids may be administered to develop a fetus' lungs if a determination is made that the fetus is of a certain gestational age. In some embodiments, a therapy to induce or stop labor may be administered based upon the determined fetal gestational age.

### VIII. Compositions and Kits

In some embodiments, provided herein is a composition comprising one or more peptide structures from Table 3. In some embodiments, provided herein is a composition comprising two peptide structures from Table 3. In some embodiments, provided herein is a composition comprising three peptide structures from Table 3. In some embodiments, provided herein is a composition comprising four peptide structures from Table 3. In some embodiments, provided herein is a composition comprising five peptide structures from Table 3. In some embodiments, provided herein is a composition comprising six peptide structures from Table 3. In some embodiments, provided herein is a composition comprising seven peptide structures from Table 3. In some embodiments, provided herein is a composition comprising eight peptide structures from Table 3. In some embodiments, the composition is from a biological sample. In some embodiments, the composition comprises one or more purified peptide structures. In some embodiments, the composition comprises enzymatically digested peptide fragments, such as those in Table 3. In some embodiments, the composition comprises one, two, three, four, five, six, seven, or eight peptides comprising a sequence set forth in SEQ ID NOs:5-12.

In some embodiments, provided herein is a composition comprising at least one peptide comprising a sequence set forth in SEQ ID NOs:5-12. In some embodiments, provided herein is a composition comprising at least two peptides comprising a sequence set forth in SEQ ID NOs:5-12. In some embodiments, provided herein is a composition comprising at least three peptides comprising a sequence set forth in SEQ ID NOs:5-12. In some embodiments, provided herein is a composition comprising at least four peptides comprising a sequence set forth in SEQ ID NOs:5-12. In some embodiments, provided herein is a composition comprising at least five peptides comprising a sequence set forth in SEQ ID NOs:5-12. In some embodiments, provided herein is a composition comprising at least six peptides comprising a sequence set forth in SEQ ID NOs:5-12. In some embodiments, provided herein is a composition comprising at least seven peptides comprising a sequence set forth in SEQ ID NOs:5-12. In some embodiments, provided herein is a composition comprising eight peptides comprising sequences set forth in SEQ ID NOs:5-12.

In some embodiments, provided herein are peptides set forth in Table 3. In some embodiments, provided herein are peptides comprising a sequence set forth in SEQ ID NOs:5-12.

In some embodiments, a kit is provided, the kit comprising at least one agent for quantifying at least one peptide structure identified in Table 3 to carry out part or all of any one or more of the methods disclosed herein.

In some embodiments, a kit is provided, the kit comprising at least one of a glycopeptide standard, a buffer, or a set of peptide sequences to carry out part or all of any one or more of the methods disclosed herein. A peptide sequence of the set of peptide sequences is identified by a corresponding one of SEQ ID NOS:5-12.

In some embodiments, provided herein is a composition comprising one or more peptide structures from Table 10. In some embodiments, provided herein is a composition comprising two peptide structures from Table 10. In some embodiments, provided herein is a composition comprising three peptide structures from Table 10. In some embodiments, provided herein is a composition comprising four peptide structures from Table 10. In some embodiments, provided herein is a composition comprising five peptide structures from Table 10. In some embodiments, provided herein is a composition comprising six peptide structures from Table 10. In some embodiments, the composition is from a biological sample. In some embodiments, the composition comprises one or more purified peptide structures. In some embodiments, the composition comprises enzymatically digested peptide fragments, such as those in Table 10. In some embodiments, the composition comprises one, two, three four, five, or six peptides comprising a sequence set forth in SEQ ID Nos:16-21.

In some embodiments, provided herein are peptides set forth in Table 10. In some embodiments, provided herein are peptides comprising a sequence set forth in SEQ ID Nos:16-21.

In some embodiments, a kit is provided, the kit comprising at least one agent for quantifying at least one peptide structure identified in Table 10 to carry out part or all of any one or more of the methods disclosed herein.

In some embodiments, a kit is provided, the kit comprising at least one of a glycopeptide standard, a buffer, or a set of peptide sequences to carry out part or all of any one or more of the methods disclosed herein. A peptide sequence of the set of peptide sequences is identified by a corresponding one of SEQ ID NOS:16-21.

In some embodiments, provided herein is a composition comprising one or more peptide structures from Table 17. In some embodiments, provided herein is a composition comprising two or more peptide structures from Table 17. In some embodiments, provided herein is a composition comprising three or more peptide structures from Table 17. In some embodiments, provided herein is a composition comprising four or more peptide structures from Table 17. In some embodiments, provided herein is a composition comprising five or more peptide structures from Table 17. In some embodiments, provided herein is a composition comprising six or more peptide structures from Table 17. In some embodiments, provided herein is a composition comprising seven or more peptide structures from Table 17. In some embodiments, provided herein is a composition comprising eight or more peptide structures from Table 17. In some embodiments, provided herein is a composition comprising nine or more peptide structures from Table 17. In some embodiments, provided herein is a composition comprising ten or more peptide structures from Table 17. In some embodiments, provided herein is a composition comprising fifteen or more peptide structures from Table 17. In some embodiments, provided herein is a composition comprising twenty or more peptide structures from Table 17. In some embodiments, provided herein is a composition comprising twenty-five or more peptide structures from Table 17. In some embodiments, provided herein is a composition comprising thirty or more peptide structures from Table 17. In some embodiments, provided herein is a composition comprising thirty-five or more peptide structures from Table 17. In some embodiments, provided herein is a composition comprising forty or more peptide structures from Table 17. In some embodiments, provided herein is a composition comprising forty-five or more peptide structures from Table 17. In some embodiments, provided herein is a composition comprising fifty or more peptide structures from Table 17. In some embodiments, provided herein is a composition comprising fifty-five or more peptide structures from Table 17. In some embodiments, provided herein is a composition comprising sixty or more peptide structures from Table 17. In some embodiments, provided herein is a composition comprising sixty-five or more peptide structures from Table 17. In some embodiments, provided herein is a composition comprising seventy or more peptide structures from Table 17. In some embodiments, provided herein is a composition comprising seventy-five or more peptide structures from Table 17. In some embodiments, provided herein is a composition comprising eighty or more peptide structures from Table 17. In some embodiments, provided herein is a composition comprising eighty-five or more peptide structures from Table 17. In some embodiments, provided herein is a composition comprising ninety or more peptide structures from Table 17. In some embodiments, provided herein is a composition comprising ninety-five or more peptide structures from Table 17. In some embodiments, provided herein is a composition comprising one hundred or more peptide structures from Table 17. In some embodiments, provided herein is a composition comprising one hundred five or more peptide structures from Table 17. In some embodiments, provided herein is a composition comprising one hundred ten or more peptide structures from Table 17. In some embodiments, provided herein is a composition comprising one hundred fifteen or more peptide structures from Table 17. In some embodiments, provided herein is a composition comprising one hundred twenty or more peptide structures from Table 17. In some embodiments, provided herein is a composition comprising one hundred twenty-four peptide structures from Table 17. In some embodiments, the composition is from a biological sample. In some embodiments, the composition comprises one or more purified peptide structures. In some embodiments, the composition comprises enzymatically digested peptide fragments, such as those in Table 17. In some embodiments, the composition comprises one, two, three, four, five, six, seven, eight, nine, ten, fifteen, twenty, twenty-five, thirty, thirty-five, forty, forty-five, fifty, fifty-five, sixty, sixty-five, seventy, seventy-five, eighty, eighty-five, ninety, ninety-five, one hundred, one hundred five, one hundred ten, one hundred fifteen, one hundred twenty, or one hundred twenty-four peptides comprising a sequence set forth in SEQ ID NOs: 65-188.

In some embodiments, the composition comprises one, two, three, four, or five peptides comprising a sequence set forth in SEQ ID NOs: 65-74. In some embodiments, the composition comprises one, two, three, four, or five peptides comprising a sequence set forth in SEQ ID NOs: 65-84. In some embodiments, the composition comprises one, two, three, four, or five peptides comprising a sequence set forth in SEQ ID NOs: 65-94. In some embodiments, the composition comprises one, two, three, four, or five peptides comprising a sequence set forth in SEQ ID NOs: 65-104. In some embodiments, the composition comprises one, two, three, four, or five peptides comprising a sequence set forth in SEQ ID NOs: 65-114. In some embodiments, the composition comprises one, two, three, four, or five peptides comprising a sequence set forth in SEQ ID NOs: 65-124. In some embodiments, the composition comprises one, two, three, four, or five peptides comprising a sequence set forth in SEQ ID NOs: 65-134. In some embodiments, the composition comprises one, two, three, four, or five peptides comprising a sequence set forth in SEQ ID NOs: 65-144. In some embodiments, the composition comprises one, two, three, four, or five peptides comprising a sequence set forth in SEQ ID NOs: 65-154. In some embodiments, the composition comprises one, two, three, four, or five peptides comprising a sequence set forth in SEQ ID NOs: 65-164. In some embodiments, the composition comprises one, two, three, four, or five peptides comprising a sequence set forth in SEQ ID NOs: 65-174. In some embodiments, the composition comprises one, two, three, four, or five peptides comprising a sequence set forth in SEQ ID NOs: 65-184. In some embodiments, the composition comprises one, two, three, four, or five peptides comprising a sequence set forth in SEQ ID NOs: 65-188. In some embodiments, the composition comprises one, two, three, four, or five peptides comprising a sequence set forth in SEQ ID NOs: 74-188. In some embodiments, the composition comprises one, two, three, four, or five peptides comprising a sequence set forth in SEQ ID NOs: 84-104. In some embodiments, the composition comprises one, two, three, four, or five peptides comprising a sequence set forth in SEQ ID NOs: 84-124. In some embodiments, the composition comprises one, two, three, four, or five peptides comprising a sequence set forth in SEQ ID NOs: 84-144. In some embodiments, the composition comprises one, two, three, four, or five peptides comprising a sequence set forth in SEQ ID NOs: 84-164. In some embodiments, the composition comprises one, two, three, four, or five peptides comprising a sequence set forth in SEQ ID NOs: 84-188. In some embodiments, the composition comprises one, two, three, four, or five peptides comprising a sequence set forth in SEQ ID NOs: 94-188. In some embodiments, the composition comprises one, two, three, four, or five peptides comprising a sequence set forth in SEQ ID NOs: 104-124. In some embodiments, the composition comprises one, two, three, four, or five peptides comprising a sequence set forth in SEQ ID NOs: 104-144. In some embodiments, the composition comprises one, two, three, four, or five peptides comprising a sequence set forth in SEQ ID NOs: 104-164. In some embodiments, the composition comprises one, two, three, four, or five peptides comprising a sequence set forth in SEQ ID NOs: 104-188. In some embodiments, the composition comprises one, two, three, four, or five peptides comprising a sequence set forth in SEQ ID NOs: 114-188. In some embodiments, the composition comprises one, two, three, four, or five peptides comprising a sequence set forth in SEQ ID NOs: 124-144. In some embodiments, the composition comprises one, two, three, four, or five peptides comprising a sequence set forth in SEQ ID NOs: 124-164. In some embodiments, the composition comprises one, two, three, four, or five peptides comprising a sequence set forth in SEQ ID NOs: 124-188. In some embodiments, the composition comprises one, two, three, four, or five peptides comprising a sequence set forth in SEQ ID NOs: 134-188. In some embodiments, the composition comprises one, two, three, four, or five peptides comprising a sequence set forth in SEQ ID NOs: 144-164. In some embodiments, the composition comprises one, two, three, four, or five peptides comprising a sequence set forth in SEQ ID NOs: 144-188. In some embodiments, the composition comprises one, two, three, four, or five peptides comprising a sequence set forth in SEQ ID NOs: 154-188. In some embodiments, the composition comprises one, two, three, four, or five peptides comprising a sequence set forth in SEQ ID NOs: 164-188. In some embodiments, the composition comprises one, two, three, four, or five peptides comprising a sequence set forth in SEQ ID NOs: 174-188. In some embodiments, the composition comprises one, two, three, four, or five peptides comprising a sequence set forth in SEQ ID NOs: 184-188.

In some embodiments, the composition comprises one, two, three, four, five, ten, fifteen, twenty, twenty-five, thirty, thirty-five, forty, forty-five, fifty, fifty-five, or fifty-nine peptides comprising a sequence set forth in SEQ ID NOs: 65-123.

In some embodiments, the composition comprises one, two, three, four, or five peptides comprising a sequence set forth in SEQ ID NOs: 65-69. In some embodiments, the composition comprises one, two, three, four, or five peptides comprising a sequence set forth in SEQ ID NOs: 65-74. In some embodiments, the composition comprises one, two, three, four, or five peptides comprising a sequence set forth in SEQ ID NOs: 65-79. In some embodiments, the composition comprises one, two, three, four, or five peptides comprising a sequence set forth in SEQ ID NOs: 65-84. In some embodiments, the composition comprises one, two, three, four, or five peptides comprising a sequence set forth in SEQ ID NOs: 65-89. In some embodiments, the composition comprises one, two, three, four, or five peptides comprising a sequence set forth in SEQ ID NOs: 65-94. In some embodiments, the composition comprises one, two, three, four, or five peptides comprising a sequence set forth in SEQ ID NOs: 65-99. In some embodiments, the composition comprises one, two, three, four, or five peptides comprising a sequence set forth in SEQ ID NOs: 65-104. In some embodiments, the composition comprises one, two, three, four, or five peptides comprising a sequence set forth in SEQ ID NOs: 65-109. In some embodiments, the composition comprises one, two, three, four, or five peptides comprising a sequence set forth in SEQ ID NOs: 65-114. In some embodiments, the composition comprises one, two, three, four, or five peptides comprising a sequence set forth in SEQ ID NOs: 65-119. In some embodiments, the composition comprises one, two, three, four, or five peptides comprising a sequence set forth in SEQ ID NOs: 65-123. In some embodiments, the composition comprises one, two, three, four, or five peptides comprising a sequence set forth in SEQ ID NOs: 69-123. In some embodiments, the composition comprises one, two, three, four, or five peptides comprising a sequence set forth in SEQ ID NOs: 74-84. In some embodiments, the composition comprises one, two, three, four, or five peptides comprising a sequence set forth in SEQ ID NOs: 74-94. In some embodiments, the composition comprises one, two, three, four, or five peptides comprising a sequence set forth in SEQ ID NOs: 74-104. In some embodiments, the composition comprises one, two, three, four, or five peptides comprising a sequence set forth in SEQ ID NOs: 74-114. In some embodiments, the composition comprises one, two, three, four, or five peptides comprising a sequence set forth in SEQ ID NOs: 74-123. In some embodiments, the composition comprises one, two, three, four, or five peptides comprising a sequence set forth in SEQ ID NOs: 84-94. In some embodiments, the composition comprises one, two, three, four, or five peptides comprising a sequence set forth in SEQ ID NOs: 84-104. In some embodiments, the composition comprises one, two, three, four, or five peptides comprising a sequence set forth in SEQ ID NOs: 84-114. In some embodiments, the composition comprises one, two, three, four, or five peptides comprising a sequence set forth in SEQ ID NOs: 84-123. In some embodiments, the composition comprises one, two, three, four, or five peptides comprising a sequence set forth in SEQ ID NOs: 89-123. In some embodiments, the composition comprises one, two, three, four, or five peptides comprising a sequence set forth in SEQ ID NOs: 94-104. In some embodiments, the composition comprises one, two, three, four, or five peptides comprising a sequence set forth in SEQ ID NOs: 94-114. In some embodiments, the composition comprises one, two, three, four, or five peptides comprising a sequence set forth in SEQ ID NOs: 94-123. In some embodiments, the composition comprises one, two, three, four, or five peptides comprising a sequence set forth in SEQ ID NOs: 99-123. In some embodiments, the composition comprises one, two, three, four, or five peptides comprising a sequence set forth in SEQ ID NOs: 104-114. In some embodiments, the composition comprises one, two, three, four, or five peptides comprising a sequence set forth in SEQ ID NOs: 104-123. In some embodiments, the composition comprises one, two, three, four, or five peptides comprising a sequence set forth in SEQ ID NOs: 109-123. In some embodiments, the composition comprises one, two, three, four, or five peptides comprising a sequence set forth in SEQ ID NOs: 114-123. In some embodiments, the composition comprises one, two, three, four, or five peptides comprising a sequence set forth in SEQ ID NOs: 119-23.

In some embodiments, the composition comprises one, two, three, four, or five peptides comprising a sequence set forth in SEQ ID NOs: 124-188.

In some embodiments, provided herein is a composition comprising at least one peptide comprising a sequence set forth in SEQ ID NOs: 65-188. In some embodiments, provided herein is a composition comprising at least two peptides comprising a sequence set forth in SEQ ID NOs: 65-188. In some embodiments, provided herein is a composition comprising at least three peptides comprising a sequence set forth in SEQ ID NOs: 65-188. In some embodiments, provided herein is a composition comprising at least four peptides comprising a sequence set forth in SEQ ID NOs: 65-188. In some embodiments, provided herein is a composition comprising at least five peptides comprising a sequence set forth in SEQ ID NOs: 65-188. In some embodiments, provided herein is a composition comprising at least six peptides comprising a sequence set forth in SEQ ID NOs: 65-188. In some embodiments, provided herein is a composition comprising at least seven peptides comprising a sequence set forth in SEQ ID NOs: 65-188. In some embodiments, provided herein is a composition comprising at least eight peptides comprising a sequence set forth in SEQ ID NOs: 65-188. In some embodiments, provided herein is a composition comprising at least nine peptides comprising a sequence set forth in SEQ ID NOs: 65-188. In some embodiments, provided herein is a composition comprising at least ten peptides comprising a sequence set forth in SEQ ID NOs: 65-188. In some embodiments, provided herein is a composition comprising at least fifteen peptides comprising a sequence set forth in SEQ ID NOs: 65-188. In some embodiments, provided herein is a composition comprising at least twenty peptides comprising a sequence set forth in SEQ ID NOs: 65-188. In some embodiments, provided herein is a composition comprising at least twenty-five peptides comprising a sequence set forth in SEQ ID NOs: 65-188. In some embodiments, provided herein is a composition comprising at least thirty peptides comprising a sequence set forth in SEQ ID NOs: 65-188. In some embodiments, provided herein is a composition comprising at least thirty-five peptides comprising a sequence set forth in SEQ ID NOs: 65-188. In some embodiments, provided herein is a composition comprising at least forty peptides comprising a sequence set forth in SEQ ID NOs: 65-188. In some embodiments, provided herein is a composition comprising at least forty-five peptides comprising a sequence set forth in SEQ ID NOs: 65-188. In some embodiments, provided herein is a composition comprising at least fifty peptides comprising a sequence set forth in SEQ ID NOs: 65-188. In some embodiments, provided herein is a composition comprising at least fifty-five peptides comprising a sequence set forth in SEQ ID NOs: 65-188. In some embodiments, provided herein is a composition comprising at least sixty peptides comprising a sequence set forth in SEQ ID NOs: 65-188. In some embodiments, provided herein is a composition comprising at least sixty-five peptides comprising a sequence set forth in SEQ ID NOs: 65-188. In some embodiments, provided herein is a composition comprising at least seventy peptides comprising a sequence set forth in SEQ ID NOs: 65-188. In some embodiments, provided herein is a composition comprising at least seventy-five peptides comprising a sequence set forth in SEQ ID NOs: 65-188. In some embodiments, provided herein is a composition comprising at least eighty peptides comprising a sequence set forth in SEQ ID NOs: 65-188. In some embodiments, provided herein is a composition comprising at least eighty-five peptides comprising a sequence set forth in SEQ ID NOs: 65-188. In some embodiments, provided herein is a composition comprising at least ninety peptides comprising a sequence set forth in SEQ ID NOs: 65-188. In some embodiments, provided herein is a composition comprising at least ninety-five peptides comprising a sequence set forth in SEQ ID NOs: 65-188. In some embodiments, provided herein is a composition comprising at least one hundred peptides comprising a sequence set forth in SEQ ID NOs: 65-188. In some embodiments, provided herein is a composition comprising at least one hundred five peptides comprising a sequence set forth in SEQ ID NOs: 65-188. In some embodiments, provided herein is a composition comprising at least one hundred ten peptides comprising a sequence set forth in SEQ ID NOs: 65-188. In some embodiments, provided herein is a composition comprising at least one hundred fifteen peptides comprising a sequence set forth in SEQ ID NOs: 65-188. In some embodiments, provided herein is a composition comprising at least one hundred twenty peptides comprising a sequence set forth in SEQ ID NOs: 65-188. In some embodiments, provided herein is a composition comprising one hundred twenty-four peptides comprising a sequence set forth in SEQ ID NOs: 65-188.

In some embodiments, provided herein is a composition comprising at least one peptide comprising a sequence set forth in SEQ ID NOs: 65-74. In some embodiments, provided herein is a composition comprising at least one peptide comprising a sequence set forth in SEQ ID NOs: 65-84. In some embodiments, provided herein is a composition comprising at least one peptide comprising a sequence set forth in SEQ ID NOs: 65-94. In some embodiments, provided herein is a composition comprising at least one peptide comprising a sequence set forth in SEQ ID NOs: 65-104. In some embodiments, provided herein is a composition comprising at least one peptide comprising a sequence set forth in SEQ ID NOs: 65-114. In some embodiments, provided herein is a composition comprising at least one peptide comprising a sequence set forth in SEQ ID NOs: 65-124. In some embodiments, provided herein is a composition comprising at least one peptide comprising a sequence set forth in SEQ ID NOs: 65-134. In some embodiments, provided herein is a composition comprising at least one peptide comprising a sequence set forth in SEQ ID NOs: 65-144. In some embodiments, provided herein is a composition comprising at least one peptide comprising a sequence set forth in SEQ ID NOs: 65-154. In some embodiments, provided herein is a composition comprising at least one peptide comprising a sequence set forth in SEQ ID NOs: 65-164. In some embodiments, provided herein is a composition comprising at least one peptide comprising a sequence set forth in SEQ ID NOs: 65-174. In some embodiments, provided herein is a composition comprising at least one peptide comprising a sequence set forth in SEQ ID NOs: 65-184. In some embodiments, provided herein is a composition comprising at least one peptide comprising a sequence set forth in SEQ ID NOs: 65-188. In some embodiments, provided herein is a composition comprising at least one peptide comprising a sequence set forth in SEQ ID NOs: 74-188. In some embodiments, provided herein is a composition comprising at least one peptide comprising a sequence set forth in SEQ ID NOs: 84-104. In some embodiments, provided herein is a composition comprising at least one peptide comprising a sequence set forth in SEQ ID NOs: 84-124. In some embodiments, provided herein is a composition comprising at least one peptide comprising a sequence set forth in SEQ ID NOs: 84-144. In some embodiments, provided herein is a composition comprising at least one peptide comprising a sequence set forth in SEQ ID NOs: 94-164. In some embodiments, provided herein is a composition comprising at least one peptide comprising a sequence set forth in SEQ ID NOs: 84-188. In some embodiments, provided herein is a composition comprising at least one peptide comprising a sequence set forth in SEQ ID NOs: 94-188. In some embodiments, provided herein is a composition comprising at least one peptide comprising a sequence set forth in SEQ ID NOs: 104-124. In some embodiments, provided herein is a composition comprising at least one peptide comprising a sequence set forth in SEQ ID NOs: 104-144. In some embodiments, provided herein is a composition comprising at least one peptide comprising a sequence set forth in SEQ ID NOs: 104-164. In some embodiments, provided herein is a composition comprising at least one peptide comprising a sequence set forth in SEQ ID NOs: 104-188. In some embodiments, provided herein is a composition comprising at least one peptide comprising a sequence set forth in SEQ ID NOs: 114-188. In some embodiments, provided herein is a composition comprising at least one peptide comprising a sequence set forth in SEQ ID NOs: 124-144. In some embodiments, provided herein is a composition comprising at least one peptide comprising a sequence set forth in SEQ ID NOs: 124-164. In some embodiments, provided herein is a composition comprising at least one peptide comprising a sequence set forth in SEQ ID NOs: 124-188. In some embodiments, provided herein is a composition comprising at least one peptide comprising a sequence set forth in SEQ ID NOs: 134-188. In some embodiments, provided herein is a composition comprising at least one peptide comprising a sequence set forth in SEQ ID NOs: 144-164. In some embodiments, provided herein is a composition comprising at least one peptide comprising a sequence set forth in SEQ ID NOs: 144-188. In some embodiments, provided herein is a composition comprising at least one peptide comprising a sequence set forth in SEQ ID NOs: 154-188. In some embodiments, provided herein is a composition comprising at least one peptide comprising a sequence set forth in SEQ ID NOs: 164-188. In some embodiments, provided herein is a composition comprising at least one peptide comprising a sequence set forth in SEQ ID NOs: 174-188. In some embodiments, provided herein is a composition comprising at least one peptide comprising a sequence set forth in SEQ ID NOs: 184-188.

In some embodiments, provided herein is a composition comprising at least one peptide comprising a sequence set forth in SEQ ID NOs: 65-123. In some embodiments, provided herein is a composition comprising at least two peptides comprising a sequence set forth in SEQ ID NOs: 65-123. In some embodiments, provided herein is a composition comprising at least three peptides comprising a sequence set forth in SEQ ID NOs: 65-123. In some embodiments, provided herein is a composition comprising at least four peptides comprising a sequence set forth in SEQ ID NOs: 65-123. In some embodiments, provided herein is a composition comprising at least five peptides comprising a sequence set forth in SEQ ID NOs: 65-123. In some embodiments, provided herein is a composition comprising at least ten peptides comprising a sequence set forth in SEQ ID NOs: 65-123. In some embodiments, provided herein is a composition comprising at least fifteen peptides comprising a sequence set forth in SEQ ID NOs: 65-123. In some embodiments, provided herein is a composition comprising at least twenty peptides comprising a sequence set forth in SEQ ID NOs: 65-123. In some embodiments, provided herein is a composition comprising at least twenty-five peptides comprising a sequence set forth in SEQ ID NOs: 65-123. In some embodiments, provided herein is a composition comprising at least thirty peptides comprising a sequence set forth in SEQ ID NOs: 65-123. In some embodiments, provided herein is a composition comprising at least thirty-five peptides comprising a sequence set forth in SEQ ID NOs: 65-123. In some embodiments, provided herein is a composition comprising at least forty peptides comprising a sequence set forth in SEQ ID NOs: 65-123. In some embodiments, provided herein is a composition comprising at least forty-five peptides comprising a sequence set forth in SEQ ID NOs: 65-123. In some embodiments, provided herein is a composition comprising at least fifty peptides comprising a sequence set forth in SEQ ID NOs: 65-123. In some embodiments, provided herein is a composition comprising at least fifty-five peptides comprising a sequence set forth in SEQ ID NOs: 65-123. In some embodiments, provided herein is a composition comprising fifty-nine peptides comprising a sequence set forth in SEQ ID NOs: 65-123.

In some embodiments, provided herein is a composition comprising at least one peptide comprising a sequence set forth in SEQ ID NOs: 65-69. In some embodiments, provided herein is a composition comprising at least one peptide comprising a sequence set forth in SEQ ID NOs: 65-74. In some embodiments, provided herein is a composition comprising at least one peptide comprising a sequence set forth in SEQ ID NOs: 65-79. In some embodiments, provided herein is a composition comprising at least one peptide comprising a sequence set forth in SEQ ID NOs: 65-84. In some embodiments, provided herein is a composition comprising at least one peptide comprising a sequence set forth in SEQ ID NOs: 65-89. In some embodiments, provided herein is a composition comprising at least one peptide comprising a sequence set forth in SEQ ID NOs: 65-94. In some embodiments, provided herein is a composition comprising at least one peptide comprising a sequence set forth in SEQ ID NOs: 65-99. In some embodiments, provided herein is a composition comprising at least one peptide comprising a sequence set forth in SEQ ID NOs: 65-104. In some embodiments, provided herein is a composition comprising at least one peptide comprising a sequence set forth in SEQ ID NOs: 65-109. In some embodiments, provided herein is a composition comprising at least one peptide comprising a sequence set forth in SEQ ID NOs: 65-114. In some embodiments, provided herein is a composition comprising at least one peptide comprising a sequence set forth in SEQ ID NOs: 65-119. In some embodiments, provided herein is a composition comprising at least one peptide comprising a sequence set forth in SEQ ID NOs: 65-123. In some embodiments, provided herein is a composition comprising at least one peptide comprising a sequence set forth in SEQ ID NOs: 69-123. In some embodiments, provided herein is a composition comprising at least one peptide comprising a sequence set forth in SEQ ID NOs: 74-84. In some embodiments, provided herein is a composition comprising at least one peptide comprising a sequence set forth in SEQ ID NOs: 74-94. In some embodiments, provided herein is a composition comprising at least one peptide comprising a sequence set forth in SEQ ID NOs: 74-104. In some embodiments, provided herein is a composition comprising at least one peptide comprising a sequence set forth in SEQ ID NOs: 74-114. In some embodiments, provided herein is a composition comprising at least one peptide comprising a sequence set forth in SEQ ID NOs: 74-123. In some embodiments, provided herein is a composition comprising at least one peptide comprising a sequence set forth in SEQ ID NOs: 84-94. In some embodiments, provided herein is a composition comprising at least one peptide comprising a sequence set forth in SEQ ID NOs: 84-104. In some embodiments, provided herein is a composition comprising at least one peptide comprising a sequence set forth in SEQ ID NOs: 84-114. In some embodiments, provided herein is a composition comprising at least one peptide comprising a sequence set forth in SEQ ID NOs: 84-123. In some embodiments, provided herein is a composition comprising at least one peptide comprising a sequence set forth in SEQ ID NOs: 89-123. In some embodiments, provided herein is a composition comprising at least one peptide comprising a sequence set forth in SEQ ID NOs: 94-104. In some embodiments, provided herein is a composition comprising at least one peptide comprising a sequence set forth in SEQ ID NOs: 94-114. In some embodiments, provided herein is a composition comprising at least one peptide comprising a sequence set forth in SEQ ID NOs: 94-123. In some embodiments, provided herein is a composition comprising at least one peptide comprising a sequence set forth in SEQ ID NOs: 99-123. In some embodiments, provided herein is a composition comprising at least one peptide comprising a sequence set forth in SEQ ID NOs: 104-114. In some embodiments, provided herein is a composition comprising at least one peptide comprising a sequence set forth in SEQ ID NOs: 104-123. In some embodiments, provided herein is a composition comprising at least one peptide comprising a sequence set forth in SEQ ID NOs: 109-123. In some embodiments, provided herein is a composition comprising at least one peptide comprising a sequence set forth in SEQ ID NOs: 114-123. In some embodiments, provided herein is a composition comprising at least one peptide comprising a sequence set forth in SEQ ID NOs: 119-123.

In some embodiments, provided herein is a composition comprising at least one peptide comprising a sequence set forth in SEQ ID NOs: 124-188.

In some embodiments, provided herein are peptides set forth in Table 17. In some embodiments, provided herein are peptides comprising a sequence set forth in SEQ ID NOs: 65-188.

In some embodiments, a kit is provided, the kit comprising at least one agent for quantifying at least one peptide structure identified in Table 17 to carry out part or all of any one or more of the methods disclosed herein.

In some embodiments, a kit is provided, the kit comprising at least one of a glycopeptide standard, a buffer, or a set of peptide sequences to carry out part or all of any one or more of the methods disclosed herein. A peptide sequence of the set of peptide sequences is identified by a corresponding one of SEQ ID NOs: 65-188.

### IX. Examples

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1. Sample Preparation and MS Data Production for Training a Diagnostic Model

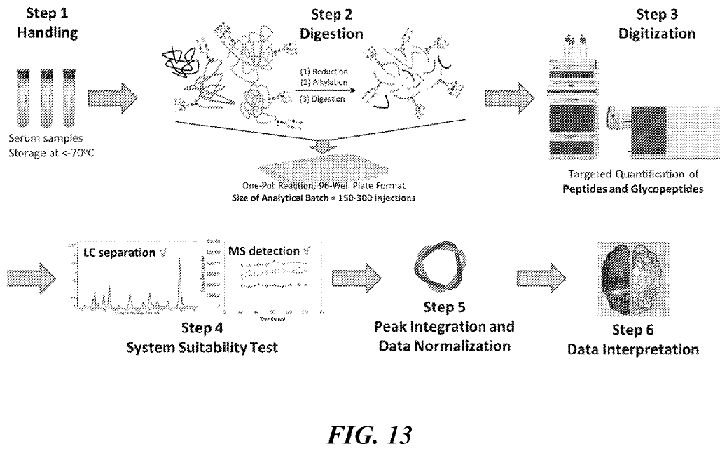

A schematic for the overall workflow for sample preparation and analysis is given in FIG. 13. A summary of the sample population used for the experiments, including ranges of the patient ages and week of gestation for sample collections, is given in Table 1. The sample set consisted of plasma samples from 6 pregnancy control patients (EDTA plasma), 12 patients who had undergone pre-term birth (PTB; double-spun EDTA plasma), and 14 patients with severe pre-eclampsia (sPE; double-spun EDTA plasma). Clinical diagnosis of patients with sPE was based on measuring elevated blood pressure of 160 mm Hg or higher systolic or 110 mm Hg or higher diastolic blood pressures on two occasions at least six hours apart for a patient on bed rest. Clinical diagnosis of patients with sPE could further be based on elevated proteinuria content of 5 grams or more of protein in a 24 hour urine collection. Clinical diagnosis of patients with PTB was based on the patients experiencing preterm pregnancies between 24 to 36 weeks of gestation.

TABLE 1
Summary of age and week of gestation for the patients

| | Number of | Age | Week of |
|---|---|---|---|
| | samples | range | gestation |
| Pregnancy control | 6 | 21-37 | 23-36 |
| Pre-term birth | 12 | 21-36 | 27-36 |
| Severe pre- | 14 | 18-41 | 26-37 |
| eclampsia | | | |

Pooled human serum for assay normalization and calibration purposes, dithiothreitol (DTT), and iodoacetamide (IAA) were purchased from Millipore Sigma (St. Louis, MO). Sequencing grade trypsin was purchased from Promega (Madison, WI). Acetonitrile (LC-MS grade) was purchased from Honeywell (Muskegon, MI). All other reagents used were procured from Millipore Sigma, VWR, and Fisher Scientific.

Prior to analysis, plasma samples were reduced with DTT in a water bath at 60° C. for 50 min, then alkylated with IAA followed by digestion with trypsin in a water bath at 37° C. for 18 hours. To quench the digestion, formic acid was added to each sample after incubation to a final concentration of 1% (v/v). A pool of 18 stable isotope-labeled synthetic peptides matching the sequence of 18 endogenous peptide targets were included at a known concentration for the purpose of determining absolute endogenous protein concentrations in the samples.

Digested plasma samples were injected into an Agilent 6495B triple quadrupole mass spectrometer equipped with an Agilent 1290 Infinity ultra-high-pressure (UHP)-LC system and an Agilent ZORBAX Eclipse Plus C18 column (2.1 mm×150 mm i.d., 1.8 μm particle size). Separation of the peptides and glycopeptides was performed using a 49-min binary gradient. The aqueous mobile phase A was 3% acetonitrile, 0.1% formic acid in water (v/v), and the organic mobile phase B was 90% acetonitrile, 0.1% formic acid in water (v/v). The flow rate was set at 0.5 mL/min. Electrospray ionization (ESI) was used as the ionization source and was operated in positive ion mode. The triple quadrupole MS was operated in dynamic multiple reaction monitoring (dMRM) mode. Samples were injected in a randomized fashion with regard to underlying phenotype, and reference pooled serum digests were injected interspersed with study samples, at every 10^{th} sample position throughout the run. The python library Scikit-learn (https://scikit-learn.org/stable/) was used for statistical analyses and for building machine learning models.

Concentration of peptides and glycopeptides were calculated by the following formula.

$$peptide concentration = ( raw abundance of the peptide / raw abundance of corresponding ISTD ) * spike - in concentration * dilution factor$$

An example ISTD could be an GWVTDGFSSLK* where the terminal lysine is a heavy stable isotope labeled lysine for the peptide with SEQ ID NO: 12—APOC3—GWVTDGFSSLK as listed in Table 3.

$$glycopeptide site - occupancy = raw abundance of glycopeptide / ( sum of raw abundance of all glycopeptides from the same glycoprotein )$$

$$approximate glycopeptide concentration = glycopeptide site - occupancy * peptide concentration of the quantification peptide from the same glycoprotein$$

### Example 2. Analysis of Peptide Structure Data

A MRM analysis was performed on serum samples from pregnancy control, PTB, and sPE patients. Concentrations of glycopeptides and peptides were calculated as described in Example 1. Concentrations of 4 glycopeptides and 4 peptides were found to be significantly different between the control and sPE populations and between the PTB and sPE populations. The proteins and glycoproteins associated with these peptides and glycopeptides, respectively, are summarized in Table 2. The amino acid sequences and other characteristics of the significantly different peptides and glycopeptides are provided in Table 3 and the structures of the glycans for the glycopeptides are provided in Table 4. LC-MRM-MS parameters for the peptide structures are summarized in Table 5.

TABLE 2
Glycoproteins associated with pregnancy control, PTB, and sPE

| SEQ | | | | |
|---|---|---|---|---|
| ID | Protein | | Uniprot | |
| NO | Abbreviation | Protein Name | ID | Protein Sequence |
| SEQ. | AGP1 | Alpha-1-acid | P02763 | MALSWVLTVLSLLPLLEAQIPLCANLV |
| ID | | glycoprotein 1 | | PVPITNATLDQITGKWFYIASAFRNEEY |
| NO: 1 | | | | NKSVQEIQATFFYFTPNKTEDTIFLREY |
| | | | | QTRQDQCIYNTTYLNVQRENGTISRYV |
| | | | | GGQEHFAHLLILRDTKTYMLAFDVND |
| | | | | EKNWGLSVYADKPETTKEQLGEFYEA |
| | | | | LDCLRIPKSDVVYTDWKKDKCEPLEK |
| | | | | QHEKERKQEEGES |
| SEQ | APOC3 | Apolipoprotein | P02656 | MQPRVLLVVALLALLASARASEAEDA |
| ID | | C-III | | SLLSFMQGYMKHATKTAKDALSSVQE |
| NO: 2 | | | | SQVAQQARGWVTDGFSSLKDYWSTV |
| | | | | KDKFSEFWDLDPEVRPTSAVAA |
| SEQ | B2M | Beta-2- | P61769 | MSRSVALAVLALLSLSGLEAIQRTPKIQ |
| ID | | microglobulin | | VYSRHPAENGKSNFLNCYVSGFHPSDI |
| NO: 3 | | | | EVDLLKNGERIEKVEHSDLSFSKDWSF |
| | | | | YLLYYTEFTPTEKDEYACRVNHVTLSQ |
| | | | | PKIVKWDRDM |
| SEQ | IGG1 | Immunoglobulin | P01857 | ASTKGPSVFPLAPSSKSTSGGTAALGC |
| ID | | heavy constant | | LVKDYFPEPVTVSWNSGALTSGVHTFP |
| NO: 4 | | gamma 1 | | AVLQSSGLYSLSSVVTVPSSSLGTQTYI |
| | | | | CNVNHKPSNTKVDKKVEPKSCDKTHT |
| | | | | CPPCPAPELLGGPSVFLFPPKPKDTLMI |
| | | | | SRTPEVTCVVVDVSHEDPEVKFNWYV |
| | | | | DGVEVHNAKTKPREEQYNSTYRVVSV |
| | | | | LTVLHQDWLNGKEYKCKVSNKALPAP |
| | | | | IEKTISKAKGQPREPQVYTLPPSRDELT |
| | | | | KNQVSLTCLVKGFYPSDIAVEWESNG |
| | | | | QPENNYKTTPPVLDSDGSFFLYSKLTV |
| | | | | DKSRWQQGNVFSCSVMHEALHNHYT |
| | | | | QKSLSLSPGK |

TABLE 3
Details of glycopeptides displaying statistically significant different abundances
in pregnancy control, PTB, and sPE sample sets

| | | | Linking | Linking | |
|---|---|---|---|---|---|
| | | | Site Pos. in | Site Pos. in | |
| SEQ ID | Peptide Structure (PS) | | Protein | Peptide | Glycan |
| NO | NAME | Peptide Sequence | Sequence | Sequence | Structure |
| SEQ ID | AGP1 (33) - 6501 | MALSWVLTVLS | 33 | 33 | 6501 |
| NO: 5 | | LLPLLEAQIPLCA | | | |
| | | NLVPVPITNATL | | | |
| | | DQITGK | | | |
| SEQ ID | AGP1 (33) - 6502 | MALSWVLTVLS | 33 | 33 | 6502 |
| NO: 6 | | LLPLLEAQIPLCA | | | |
| | | NLVPVPITNATL | | | |
| | | DQITGK | | | |
| SEQ ID | AGP1 (93) -7604 | QDQCIYNTTYLN | 93 | 7 | 7604 |
| NO: 7 | | VQR | | | |
| SEQ ID | APOC3 (74) - 1102 | FSEFWDLDPEVR | 74 | 14 | 1102 |
| NO: 8 | | PTSAVAA | | | |
| SEQ ID | B2M - VNHVTLSQPK | VNHVTLSQPK | N/A | N/A | N/A |
| NO: 9 | | | | | |
| SEQ ID | APOC3 - DYWSTVK | DYWSTVK | N/A | N/A | N/A |
| NO: 10 | | | | | |
| SEQ ID | IGG1 - DTLMISR | DTLMISR | N/A | N/A | N/A |
| NO: 11 | | | | | |
| SEQ ID | APOC3 - GWVTDGFSSLK | GWVTDGFSSLK | N/A | N/A | N/A |
| NO: 12 | | | | | |

In some embodiments, the methionine of SEQ ID NO:11 (DTLMISR) is an oxidized methionine.

TABLE 4
Glycan structure GL NO, structure, and composition

| Glycan Structure GL | | |
|---|---|---|
| NO. | Structure | Composition |
| 1102 | 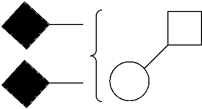 | Hex(1)HexNAc(1)Fuc(0)NeuAc(2) |
| 6501 | 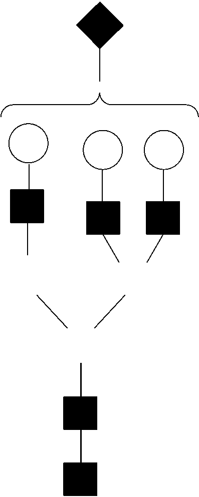 | Hex(6)HexNAc(5)Fuc(0)NeuAc(1) |
| 6502 | 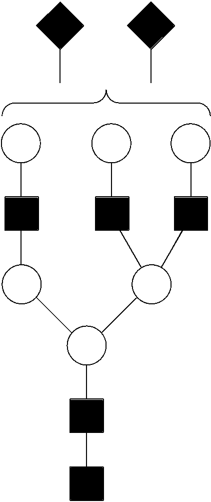 | Hex(6)HexNAc(5)Fuc(0)NeuAc(2) |
| 7604 | 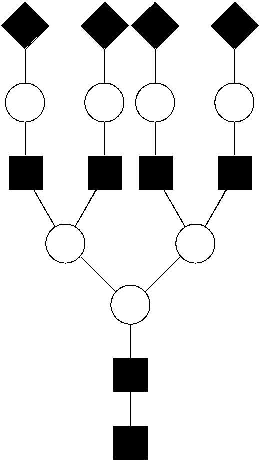 | Hex(7)HexNAc(6)Fuc(0)NeuAc(4) |

Legend for Table 4
Glc

[symbol]

Gal
◯
Man

[symbol]

Fuc

[symbol]

NeuSAc

[symbol]

GlcNAc

[symbol]

GalNAc
□
ManNAc

[symbol]

TABLE 5
LC-MRM-MS parameters for peptide structures associated
with pregnancy control, PTB, and sPE

| | | | 1^{st} | 1^{st} | 1^{st} | 1^{st} |
|---|---|---|---|---|---|---|
| SEQ ID | RT | Collision | Precursor | Precursor | product | product |
| NO | (min) | Energy (V) | m/z | charge | m/z | charge |
| SEQ ID | 28.58 | 30 | 1215 | 2 | 366.1 | 1 |
| NO: 5 | | | | | | |
| SEQ ID | 29.23 | 32 | 1287.7 | 2 | 366.1 | 1 |
| NO: 6 | | | | | | |
| SEQ ID | 23.8 | 27 | 1087.7 | 5 | 366.1 | 1 |
| NO: 7 | | | | | | |
| SEQ ID | 38.1 | 20 | 1028.8 | 3 | 274.1 | 1 |
| NO: 8 | | | | | | |
| SEQ ID | 9.5 | 25 | 561.8 | 2 | 244.2 | 1 |
| NO: 9 | | | | | | |
| SEQ ID | 17.1 | 10 | 449.7 | 2 | 620.3 | 1 |
| NO: 10 | | | | | | |
| SEQ ID | 11.9 | 8 | 426.2 | 2 | 522.3 | 1 |
| NO: 11 | | | | | | |
| SEQ ID | 29.2 | 17 | 598.8 | 2 | 244.1 | 1 |
| NO: 12 | | | | | | |

Table 5 shows various parameters associated with the identification of the peptide and glycopeptides using LC and MRM-MS. The retention time (RT) represents the amount of time in minutes for the peptide elute from the chromatography column. The collision energy represents the energy applied to the peptide for creating fragments (i.e., product ions) such as, for example, in the 2^{nd} quadrupole of the triple quadrupole MS. The first precursor m/z represents a ratio value associated with an ionized form having a first precursor charge for the peptide or glycopeptide. The first precursor ion is associated with a first product ion having a m/z ratio that was formed from a collision.

To demonstrate the statistical significance in the peptide structure concentration difference between the control and PTB populations, between the control and sPE populations, and between the PTB and sPE populations, the fold changes, p-values, and false discovery rates (FDR) are provided in Table 6. Fold-changes for individual peptides and glycopeptides, were calculated on normalized abundances of control vs. PTB samples, control vs. sPE samples, as well as on PTB vs. sPE samples, after adjusting for age and week of gestation. False discovery rate was calculated using the Benjamini-Hochberg method.

TABLE 6
Differential expression analysis for pregnancy control, PTB, and sPE sample sets

| | Control/ | Control/ | Control/ | Control/ | Control/ | Control/ | sPE/PTB | | |
|---|---|---|---|---|---|---|---|---|---|
| SEQ ID | PTB fold | PTB | PTB | sPE fold | sPE | sPE | fold | sPE/PTB | sPE/PTB |
| NO | change | p-value | FDR | change | p-value | FDR | change | p-value | FDR |
| SEQ ID | 2.3918 | 0.0069 | 0.0272 | 3.8442 | 0.0001 | 0.0238 | 1.6288 | 0.0372 | 0.9038 |
| NO: 5 | | | | | | | | | |
| SEQ ID | 5.0053 | 0.0004 | 0.0038 | 8.1080 | 1.03E−05 | 0.0022 | 1.6774 | 0.0356 | 0.9038 |
| NO: 6 | | | | | | | | | |
| SEQ ID | 4.1174 | 0.0018 | 0.0105 | 5.9525 | 1.13E−05 | 0.0025 | 1.5041 | 0.0414 | 0.9038 |
| NO: 7 | | | | | | | | | |
| SEQ ID | 3.9614 | 8.43E−05 | 0.0015 | 6.3324 | 1.16E−05 | 0.0025 | 1.5976 | 0.0031 | 0.3352 |
| NO: 8 | | | | | | | | | |
| SEQ ID | 1.4881 | 0.035 | 0.1072 | 1.9853 | 6.65E−05 | 0.0112 | 1.4195 | 0.0086 | 0.4834 |
| NO: 9 | | | | | | | | | |
| SEQ ID | 4.7738 | 0.0001 | 0.0016 | 7.0524 | 3.77E−06 | 0.0006 | 1.4598 | 0.015 | 0.6326 |
| NO: 10 | | | | | | | | | |
| SEQ ID | 0.4888 | 0.0038 | 0.0176 | 0.4135 | 0.0027 | 0.4578 | 0.7709 | 0.0236 | 0.7761 |
| NO: 11 | | | | | | | | | |
| SEQ ID | 4.0691 | 0.0001 | 0.0016 | 5.7899 | 1.95E−06 | 0.0003 | 1.4117 | 0.032 | 0.7761 |
| NO: 12 | | | | | | | | | |

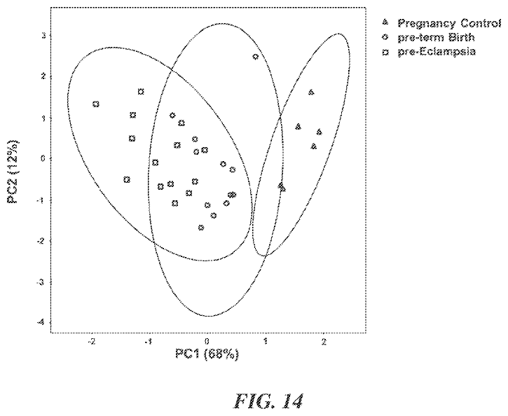

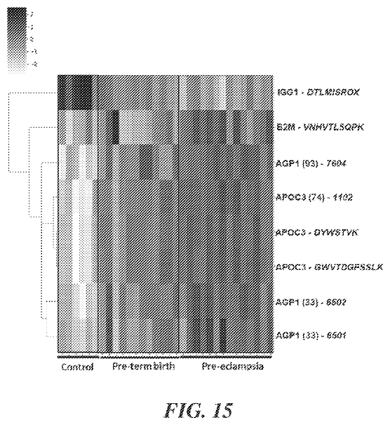

Principal component analysis (PCA) was performed to assess the segregation between the three phenotypes across first and second principal components. Prior to performing the PCA, concentrations were scaled so that the distribution had a mean value of 0 and a standard deviation of 1. The results of the PCA are provided in FIG. 14. As can be seen in FIG. 14, the sPE samples segregate quite distinctly from pregnancy control samples and fairly well from PTB samples in the first principle component. A heat map of the scaled concentrations of the 8 markers for each sample population is depicted in FIG. 15 and the separation between the three phenotypes is evident.

### Example 3. Training and Validation of a Diagnostic Model

The quantified concentrations of various peptide structures (e.g., SEQ. ID NO:5-12 identified in Table 3) across the entire sample set were used to train a multivariate logistic regression model to generate a disease indicator for a subject. The disease indicator was generated as a score (e.g., a probability score) in which the range in which the score falls enables diagnosis or classification as a non-sPE state or a sPE state. The same markers were used to train logistic regression models to separate PTB and sPE. Coefficients for the multivariate logistic models are provided in Table 7.

TABLE 7
Coefficients for each marker used in the
multivariate logistic regression models

| | Coefficients | Coefficients | Coefficients |
|---|---|---|---|
| SEQ ID NO | (Control vs. Rest) | (PTB vs. Rest) | (sPE vs. Rest) |
| SEQ ID NO: 5 | 0 | −4.2962 | 4.5694 |
| SEQ ID NO: 6 | −2.3862 | −0.4396 | 15.3936 |
| SEQ ID NO: 7 | −5.4512 | −2.8752 | 1.2544 |
| SEQ ID NO: 8 | 1.6369 | −2.4566 | −8.2925 |
| SEQ ID NO: 9 | 0 | 5.1958 | 23.8227 |
| SEQ ID NO: 10 | −3.3873 | −0.4262 | 8.1170 |
| SEQ ID NO: 11 | 0 | 0.4169 | −17.9772 |
| SEQ ID NO: 12 | −1.5554 | 0.0719 | 10.2767 |

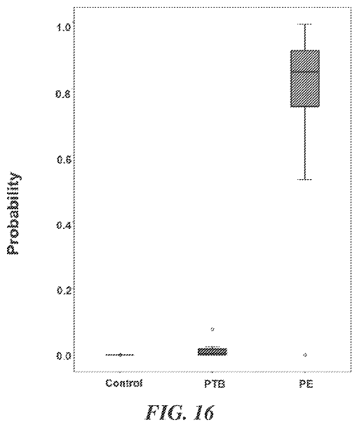

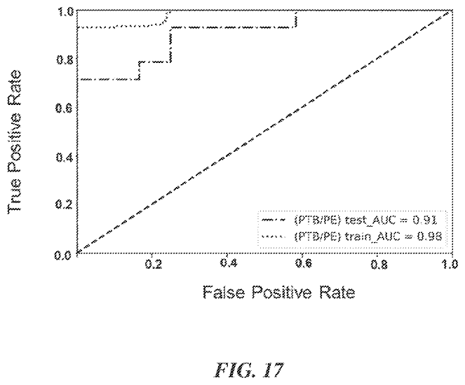

FIG. 16 is a diagram illustrating validation of the disease indicator's ability to distinguish between the sPE state and the PTB and control states in accordance with one or more embodiments. As depicted, a disease indicator of about 0.5 to about 1.00 was generally accurate in classifying as a sPE state. FIG. 17 is a diagram of the receiver-operating characteristic (ROC) curve for distinguishing between the sPE state and the PTB state for both the training and testing sets in accordance with one or more embodiments. Leave one out cross validation (LOOCV) was performed on normalized concentrations of the samples from both sPE and PTB patients. A logistic regression model with LASSO regularization was iteratively trained on all samples except for one sample that was left out in that iteration. The trained model was then used to predict on the sample that was left out. As shown in FIG. 17, the area under the ROC curve (AUROC) for the training set was found to be 0.98, while the AUROC for the testing set was found to be 0.91. the relative contribution of each biomarker in this model can be correlated to the magnitude (e.g., absolute value) of each logistic regression coefficient for SEQ ID NOS:5-12, with greater magnitudes corresponding to a greater contribution to the model's predictions.

This result demonstrates that the identified peptide structures in Table 3 and a trained model using the peptide structures can be used to diagnose preeclampsia.

### Example 4. Sample Preparation and MS Data Production for Training a Predictive Model

A schematic for the overall workflow for sample preparation and analysis is given in FIG. 13. In order to assemble data to train a predictive model of gestational age, a sample set consisting of plasma samples (double-spun EDTA plasma) was collected, originating from 26 pregnant patients aged 18-41. The week of gestation at the time of collection of each sample for the entire sample population is given in Table 8. The sample population consisted of patients that experienced pre-term birth (PTB) and patients that experienced severe pre-eclampsia (sPE). The condition of each sample source is also noted in Table 8.

TABLE 8
Summary of week of gestation and condition for the sample set

| Week of | |
|---|---|
| gestation | Condition |
| 25.6 | PTB |
| 26.5 | sPE |
| 27 | PTB |
| 27.5 | PTB |
| 28.2 | PTB |
| 29 | sPE |
| 29.1 | sPE |
| 30.1 | sPE |
| 31 | sPE |
| 32 | PTB |
| 32 | PTB |
| 32 | sPE |
| 32.3 | PTB |
| 32.5 | sPE |
| 33 | PTB |
| 33 | sPE |
| 33 | sPE |
| 33.1 | sPE |
| 33.1 | sPE |
| 34.1 | sPE |
| 35.2 | PTB |
| 35.6 | PTB |
| 35.6 | sPE |
| 36 | PTB |
| 36 | PTB |
| 36.6 | sPE |

Prior to analysis, plasma samples were prepared and digested in a manner similar to Example 1.

### Example 5. Analysis of Peptide Structure Data

A MRM analysis was performed on plasma samples from the sample set from Example 4. Concentrations of glycopeptides and peptides were calculated as described in Example 4. Concentrations of 4 glycopeptides and 2 peptides were found to have a Pearson coefficient of correlation between week of gestation and abundance that was greater than 0.5. Furthermore, the concentrations of these peptide structures were found to be significantly associated with week of gestation after adjusting for age, signified by a p-value less than 0.05. The proteins and glycoproteins associated with these peptides and glycopeptides are summarized in Table 9. The amino acid sequences and other characteristics of the significantly different peptides and glycopeptides are provided in Table 10 and the structures of the glycans for the glycopeptides are provided in Table 11. LC-MRM-MS parameters for the peptide structures are summarized in Table 12.

TABLE 9
Glycoproteins associated with gestational age

| SEQ | | | | |
|---|---|---|---|---|
| ID | Protein | | Uniprot | |
| NO | Abbreviation | Protein Name | ID | Protein Sequence |
| SEQ | APOC3 | Apolipoprotein | P02656 | MQPRVLLVVALLALLASARASEAEDA |
| ID | | C-III | | SLLSFMQGYMKHATKTAKDALSSVQE |
| NO: | | | | SQVAQQARGWVTDGFSSLKDYWSTV |
| 13 | | | | KDKFSEFWDLDPEVRPTSAVAA |
| SEQ | FETUA | Alpha-2-HS- | P02765 | MKSLVLLLCLAQLWGCHSAPHGPGLI |
| ID | | glycoprotein | | YRQPNCDDPETEEAALVAIDYINQNLP |
| NO: | | | | WGYKHTLNQIDEVKVWPQQPSGELFE |
| 14 | | | | IEIDTLETTCHVLDPTPVARCSVRQLKE |
| | | | | HAVEGDCDFQLLKLDGKFSVVYAKCD |
| | | | | SSPDSAEDVRKVCQDCPLLAPLNDTRV |
| | | | | VHAAKAALAAFNAQNNGSNFQLEEIS |
| | | | | RAQLVPLPPSTYVEFTVSGTDCVAKEA |
| | | | | TEAAKCNLLAEKQYGFCKATLSEKLG |
| | | | | GAEVAVTCMVFQTQPVSSQPQPEGAN |
| | | | | EAVPTPVVDPDAPPSPPLGAPGLPPAGS |
| | | | | PPDSHVLLAAPPGHQLHRAHYDLRHT |
| | | | | FMGVVSLGSPSGEVSHPRKTRTVVQPS |
| | | | | VGAAAGPVVPPCPGRIRHFKV |
| SEQ | SHBG | Sex hormone- | P04278 | MESRGPLATSRLLLLLLLLLLRHTRQG |
| ID | | binding globulin | | WALRPVLPTQSAHDPPAVHLSNGPGQ |
| NO: | | | | EPIAVMTFDLTKITKTSSSFEVRTWDPE |
| 15 | | | | GVIFYGDTNPKDDWFMLGLRDGRPEI |
| | | | | QLHNHWAQLTVGAGPRLDDGRWHQV |
| | | | | EVKMEGDSVLLEVDGEEVLRLRQVSG |
| | | | | PLTSKRHPIMRIALGGLLFPASNLRLPL |
| | | | | VPALDGCLRRDSWLDKQAEISASAPTS |
| | | | | LRSCDVESNPGIFLPPGTQAEFNLRDIP |
| | | | | QPHAEPWAFSLDLGLKQAAGSGHLLA |
| | | | | LGTPENPSWLSLHLQDQKVVLSSGSGP |
| | | | | GLDLPLVLGLPLQLKLSMSRVVLSQGS |
| | | | | KMKALALPPLGLAPLLNLWAKPQGRL |
| | | | | FLGALPGEDSSTSFCLNGLWAQGQRL |
| | | | | DVDQALNRSHEIWTHSCPQSPGNGTD |
| | | | | ASH |

TABLE 10
Details of glycopeptides and peptides displaying significant associated with
week of gestation

| | | | Linking | Linking | |
|---|---|---|---|---|---|
| | | | Site Pos. in | Site Pos. in | |
| SEQ ID | Peptide Structure (PS) | | Protein | Peptide | Glycan |
| NO | NAME | Peptide Sequence | Sequence | Sequence | Structure |
| SEQ ID | APOC3 (74) - 1111 | FSEFWDLDPEVR | 74 | 14 | 1111 |
| NO: 16 | | PTSAVAA | | | |
| SEQ ID | FETUA (156) - 5401 | AALAAFNAQNN | 156 | 11 | 5401 |
| NO: 17 | | GSNFQLEEISR | | | |
| SEQ ID | FETUA (156) - 6502 | AALAAFNAQNN | 156 | 11 | 6502 |
| NO: 18 | | GSNFQLEEISR | | | |
| SEQ ID | FETUA (156) - 6410 | AALAAFNAQNN | 156 | 11 | 6410 |
| NO: 19 | | GSNFQLEEISR | | | |
| SEQ ID | SHBG - | IALGGLLFPASN | N/A | N/A | N/A |
| NO: 20 | IALGGLLFPASNLR | LR | | | |
| SEQ ID | SHBG - DDWFMLGLR | DDWFMLGLR | N/A | N/A | N/A |
| NO: 21 | | | | | |

TABLE 11
Glycan structure GL NO, structure, and composition

| Glycan Structure GL | | |
|---|---|---|
| NO. | Structure | Composition |
| 1111 | 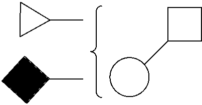 | Hex(1)HexNAc(1)Fuc(1)NeuAc(1) |
| 5401 | 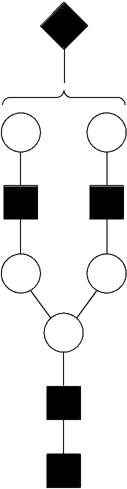 | Hex(5)HexNAc(4)Fuc(0)NeuAc(1) |
| 6502 | 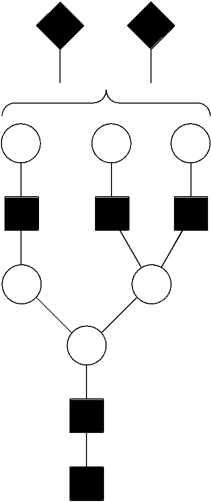 | Hex(6)HexNAc(5)Fuc(0)NeuAc(2) |
| 6410 | 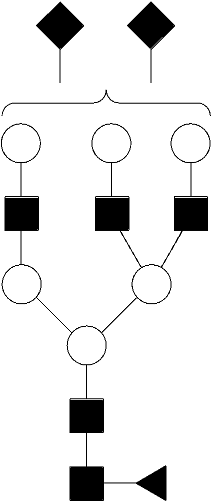 | Hex(6)HexNAc(4)Fuc(1)NeuAc(0) |

Legend for Table 11
Glc

[symbol]

Gal
◯
Man

[symbol]

Fuc

[symbol]

Neu5Ac

[symbol]

GlcNAc

[symbol]

GalNAc
□
ManNAc

[symbol]

TABLE 12
LC-MRM-MS parameters for peptide structures
associated with gestational age

| | | | | | 1^{st} | 1^{st} |
|---|---|---|---|---|---|---|
| SEQ ID | RT | Collision | Precursor | Precursor | product | product |
| NO | (min) | Energy (V) | m/z | charge | m/z | charge |
| SEQ ID | 37.1 | 24 | 980.4 | 2 | 274.1 | 1 |
| NO: 16 | | | | | | |
| SEQ ID | 26.3 | 31 | 1229.2 | 2 | 366.1 | 1 |
| NO: 17 | | | | | | |
| SEQ ID | 26.9 | 27 | 1086.2 | 2 | 366.1 | 1 |
| NO: 18 | | | | | | |
| SEQ ID | 21.35 | 25 | 1234.9 | 2 | 366.1 | 1 |
| NO: 19 | | | | | | |
| SEQ ID | 40 | 30 | 721.4 | 2 | 657.4 | 1 |
| NO: 20 | | | | | | |
| SEQ ID | 39.5 | 15 | 576.8 | 2 | 589.3 | 1 |
| NO: 21 | | | | | | |

Table 12 shows various parameters associated with the identification of the peptide and glycopeptides using LC and MRM-MS. The retention time (RT) represents the amount of time in minutes for the peptide elute from the chromatography column. The collision energy represents the energy applied to the peptide for creating fragments (i.e., product ions) such as, for example, in the 2^{nd} quadrupole of the triple quadrupole MS. The first precursor m/z represents a ratio value associated with an ionized form having a first precursor charge for the peptide or glycopeptide. The first precursor ion is associated with a first product ion having a m/z ratio that was formed from a collision.

### Example 6. Training and Validation of a Predictive Model

The quantified concentrations of various peptide structures (e.g., SEQ ID NO:16-21 identified in Table 10) across the entire sample set were used to train an ensemble learning model (e.g., gradient boosting model, an adaptive boosting (AdaBoost) model, an eXtreme gradient boosting (XGBoost) model, a light gradient boosted machine (LightGBM) model, a categorical boosting (CatBoost) model, and so forth) to generate a gestational age indicator for a subject. The ensemble learning model included a number of decision trees (e.g., dozens of decision trees, hundreds of decision trees, or thousands of decision trees), in which each succeeding decision tree of the number of decision trees is trained to correct an error and learn based on a prediction of each preceding decision tree of the number of decision trees until a final prediction is generated. The gestational age indicator was generated as a final score (e.g., a final probability score) in which the range in which the final score falls enables the designation of the patient with a specific week of gestation. A Pearson coefficient of correlation between week of gestation and marker abundance as well as the feature rank in the regression model for each of SEQ ID NO:16-21 are provided in Table 13.

TABLE 13
Pearson correlation coefficient and model feature rank
for peptide structures associated with gestational age

| | Pearson's correlation | Feature rank in |
|---|---|---|
| | between abundance | gradient boost |
| Marker | and week of gestation | regression model |
| SEQ ID NO: 16 | 0.63 | 2 |
| SEQ ID NO: 17 | 0.6 | 6 |
| SEQ ID NO: 18 | 0.58 | 3 |
| SEQ ID NO: 19 | 0.53 | 4 |
| SEQ ID NO: 20 | 0.62 | 1 |
| SEQ ID NO: 21 | 0.54 | 5 |

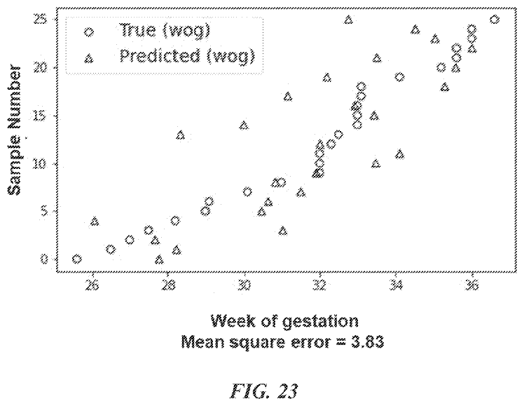

FIG. 23 is a diagram illustrating the gestational age indicator's performance when predicting week of gestation for the entire sample set in accordance with one or more embodiments. The predicted week of gestation (wog) and actual week of gestation for each sample in the sample set is provided in Table 14. The mean squared error between the predicted week of gestation and the true week of gestation for a patient was 3.83 weeks.

This result demonstrates that the identified peptide structures in Table 10 and a trained model using the peptide structures can be used to predict week of gestation.

TABLE 14
Summary of true and predicted week of gestation
from the trained regression model

| True Week | Predicted Week |
|---|---|
| of Gestation | of Gestation |
| 25.6 | 27.8 |
| 26.5 | 28.2 |
| 27 | 27.7 |
| 27.5 | 31 |
| 28.2 | 26.1 |
| 29 | 30.5 |
| 29.1 | 30.7 |
| 30.1 | 31.5 |
| 31 | 30.8 |
| 32 | 33.5 |
| 32 | 34.1 |
| 32 | 31.9 |
| 32.3 | 32 |
| 32.5 | 28.3 |
| 33 | 30 |
| 33 | 33.4 |
| 33 | 32.9 |
| 33.1 | 31.2 |
| 33.1 | 35.3 |
| 34.1 | 32.2 |
| 35.2 | 35.6 |
| 35.6 | 36 |
| 35.6 | 33.5 |
| 36 | 35 |
| 36 | 34.5 |
| 36.6 | 32.8 |

### Example 7. Digestion of Samples Prior to Enrichment and Analysis

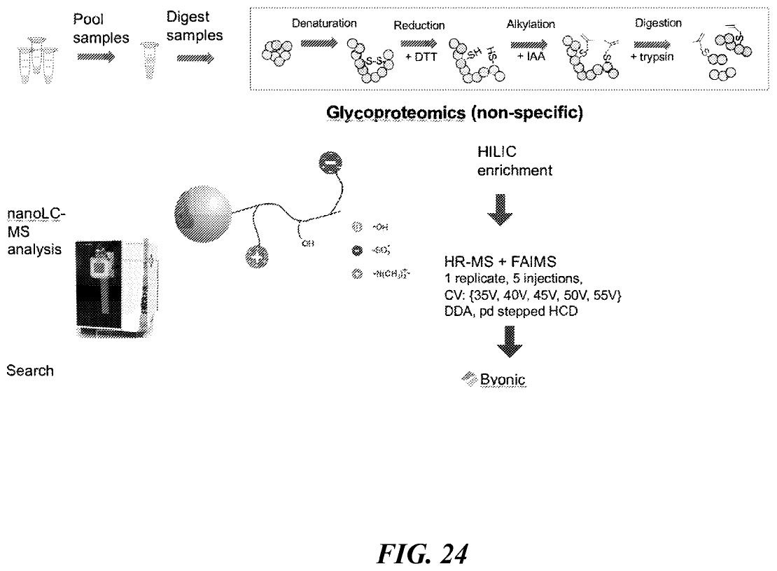

A schematic for the overall workflow for sample preparation and analysis is given in FIG. 24 for identifying new glycoproteins and glycoforms that are suitable for use as biomarkers for diagnosing preeclampsia. Biological samples were enriched for glycopeptides by pooling the plasma of 3 female subjects with a similar condition. Samples were stratified by gestational age to minimize the effect of gestational age in the comparison. A summary of the sample population used for the experiments, including the week of gestation for sample collections, is given in Table 15. The sample set consisted of 3 pooled plasma samples that were 3 pregnancy control subjects (EDTA plasma), 3 subjects with early stage severe pre-eclampsia (early sPE; double-spun EDTA plasma, 26.5 to 29.1 weeks of gestation), and 3 subjects with late stage severe pre-eclampsia (late sPE; double-spun EDTA plasma, 34 to 36.5 weeks of gestation). Clinical diagnosis of patients with sPE was based on measuring elevated blood pressure of 160 mm Hg or higher systolic or 110 mm Hg or higher diastolic blood pressures on two occasions at least six hours apart for a patient on bed rest. Clinical diagnosis of patients with sPE could further be based on elevated proteinuria content of 5 grams or more of protein in a 24 hour urine collection.

TABLE 15
Summary of the sample population of the biological samples tested

| | Number of samples | |
|---|---|---|
| | pooled together | Week of gestation |
| Pregnancy control | 3 | 27, 32, and 34 |
| (Control) | | |
| Early Stage Severe | 3 | 26.5, 29, and 29.1 |
| pre-eclampsia | | |
| (Early sPE) | | |
| Late Stage Severe | 3 | 34, 35.6 and 36.5 |
| pre-eclampsia | | |
| (Late sPE) | | |

Pooled human serum for assay normalization and calibration purposes, dithiothreitol (DTT), and iodoacetamide (IAA) were purchased from Millipore Sigma (St. Louis, MO). Sequencing grade trypsin was purchased from Promega (Madison, WI). Acetonitrile (LC-MS grade) was purchased from Honeywell (Muskegon, MI). All other reagents used were procured from Millipore Sigma, VWR, and Fisher Scientific.

In the first step described for the method herein, ammonium bicarbonate (50 mM) and dithiothreitol (DTT) (50 mM) solutions were freshly prepared. The ammonium bicarbonate solution was used to make the DTT solution. Immediately prior to transfer, each biological sample and control was gently vortexed for 10 seconds. Using a single channel pipette, 5 μL of biological sample or control (e.g., plasma or serum) was transferred into a deep-well digestion plate, wherein the plate is compatible with thermal cycling. To this, the 35 μL of 50 mM ammonium bicarbonate solution was added. The plates were then sealed with a foil heat seal using a plate sealer. To ensure all samples were mixed thoroughly, the plates were vortexed at 1400 RPM for 1 minute on a microplate mixer, followed by centrifugation at 370×g for 1 minute.

The sample plate containing the sample was incubated in a thermal cycler for 5 minutes, wherein the thermal cycler was set to 100° C. with a lid temperature of 105° C. All heated plates were allowed to cool to room temperature before removing from the respective heat source and spinning at 370×g for 1 minute. After the spin, the plate seals were removed.

After protein denaturation, all samples were reduced by adding 20 μL of the 50 mM DTT solution into each sample and control well. The plates were then sealed with a foil heat seal using a plate sealer. To ensure all samples were mixed thoroughly, the plates were vortexed at 1400 RPM for 1 minute on a microplate mixer, followed by centrifugation at 370×g for 1 minute. Plates were then incubated in a 60° C. water bath for 50 minutes. Plates were then removed from the water bath and centrifuged at 4,800×g for 1 minute before removing the plate seals.

Prior to the completion of this reduction incubation, a fresh 90 mM iodoacetamide (IAA) solution was prepared, and the container with the IAA solution was covered in foil. When ready, samples were alkylated by adding 20 μL of the 90 mM IAA solution into each sample and control well. The plates were then sealed with a foil heat seal using a plate sealer. To ensure all samples were mixed thoroughly, the plates were vortexed at 1400 RPM for 1 minute on a microplate mixer, followed by centrifugation at 370×g for 1 minute. Plates were then incubated in the dark at room temperature for 30 minutes. After the incubation, plate seals were removed and 10 μL of the 50 mM DTT solution was added to quench any remaining IAA in solution. The plates were then sealed with a foil heat seal using a plate sealer and vortexed at 1400 RPM for 1 minute on a microplate mixer. Plates were centrifuged at 370×g for 1 minute and the plate seals were removed.

Prior to the completion of this alkylation incubation, fresh protease solutions were prepared that was a combination of trypsin/LysC. For example, for the trypsin/LysC solution, trypsin/LysC powder was dissolved in the 50 mM ammonium bicarbonate solution for a final concentration of 0.333 μg/μL trypsin/LysC solution. To the quenched biological samples and controls, 60 μL of the 0.333 μg/μL trypsin/LysC solution was added to each well. The plates were then sealed with a foil heat seal using a plate sealer. To ensure all samples were mixed thoroughly, the plates were vortexed at 1400 RPM for 1 minute on a microplate mixer, followed by centrifugation at 370×g for 1 minute. Plates were then incubated in a 37° C. water bath for 18 hours. Plates were then removed from the water bath and centrifuged at 4,800×g for 1 minute before removing the plate seals.

20 μL of freshly prepared 9% formic acid solution was added to each well containing the proteolytic digested samples to stop the enzyme reaction and form the tryptically digested samples. The plates were then sealed with a foil heat seal using a plate sealer. To ensure all samples were mixed thoroughly, the plates were vortexed at 1400 RPM for 1 minute on a microplate mixer, followed by centrifugation at 370×g for 1 minute.

### Example 8. Enrichment of Digested Samples

Tryptically digested samples from Example 7 were enriched for glycopeptides using a hydrophilic interaction liquid chromatography (HILIC) concentration phase. The HILIC sorbent material used in this example was the HILICON iSPE which was a mixed-mode HILIC phase of charge modulated hydroxyethyl amide silicas which are covalently bonded with neutral, positively charged quaternary ammonium groups [—N(CH₃)₃⁺], and negatively charged [—SO₄⁻]hydrophilic functional groups. This enrichment process increased the proportion of glycopeptides with respect to the peptides in the sample (e.g., >85% glycopeptides vs peptides) by increasing the interactions between the glycans and the sorbent material. These interactions were dominated by H-bonding between the glycan hydroxyl groups or sialic acid carboxylic acid group, and the sorbent functional groups.

250 μL of serum digest was diluted with 1 mL of 1% trifluoracetic acid (TFA) in acetonitrile (ACN) and then mixed with vortexing. An iSPE®-HILIC SPE Cartridge (96-well, 50 mg, 50 μm particle format) was conditioned by adding a 1 mL portion of water to each well and then a mild vacuum of up to 10 mm Hg was applied. The liquid flowed through the HILIC sorbent and then was collected in a waste collection basin. Next, a 2 mL aliquot of 1% TFA and 80% ACN solution in water was added to each well along with the mild vacuum of up to 10 mm Hg to equilibrate the HILIC sorbent. Each of the diluted serum digest samples was loaded onto a well containing the equilibrated HILIC sorbent and allowed for the liquid to flow through. To each well, 3 mL of 1% TFA & 80% ACN in water was added while applying a mild vacuum to wash the HILIC sorbent. The waste collection basin was replaced with a 96-well collection plate with >1 mL well capacity. To each well, 1 mL of 0.1% TFA in water was added and the enriched sample was collected into the 96-well collection plate. The liquid for each well of the collection plate was removed through evaporation with a SpeedVac evaporating device to form a dried sample. 50 μL of 0.1% Formic acid & 3% ACN in water was added to each of the dried samples to reconstitute the sample so that they can be injected into a LC-MS system.

### Example 9. Mass Spectrometry of the Enriched Samples

The HILIC enriched samples were analysed with LC-MS. More specifically, samples were delivered using the UltiMate 3000 LC System (Thermo Scientific) with a Acclaim™ PepMap™ 100 C18 HPLC Columns (0.075 mm×150 mm) (Thermo Scientific) coupled to a FAIMS Pro device (Thermo Scientific) and Orbitrap Exploris 480 mass spectrometer (Thermo Scientific). High field asymmetric waveform ion mobility spectrometry (FAIMS) is an atmospheric pressure ion mobility technique that separates gas-phase ions by their behavior in strong and weak electric fields. Samples were separated and delivered into the FAIMS-MS system at a flow rate of 0.4 μL/min with a gradient from 99% buffer A (water containing 0.1% formic acid) and 1% buffer B (ACN containing 0.1% formic acid) to 66% buffer A and 34% buffer B in 68 minutes. Each sample was acquired using a product dependent data dependent acquisition (pd-DDA) method with FAIMS operated at five different compensation voltage (CV) values of −35V, −40V, −45V, −50V, −55 V. MS parameters were as follows: spray voltage of 2.2 kV; ion transfer capillary temperature of 300° C.; MS1 resolution (FWHM) at m/z 200 set to 120,000; custom MS1 automatic gain control set to 300%; MS maximum injection time mode set to auto; MS/MS resolution (FWHM) at m/z 200 set to 60,000; custom MS/MS automatic gain control at 300%; MS/MS maximum injection time mode set to auto; isolation width of 1.6.

The raw files were searched using a Byonic glycopeptide search engine. The search results were filtered by a confidence cutoff score of 250 that identified 2208 unique glycopeptides. The results were merged and sorted by each glycopeptide and numbers of its associated peptide spectrum matching (PSM) for each sample cohort. A PSM is the MS/MS spectrum that supports the identification of the glycopeptide. The PSM Count of a given glycopeptide was used as an indicator of the abundance of the given glycopeptide. The abundance of a given glycopeptide in a cohort was further normalized by the number of total PSM in the associated cohort (e.g., the total number of PSM found for the 2208 unique glycopeptides in either the control, early sPE, or late sPE cohort) to yield a relative abundance for better comparison among different samples. For each of the identified glycopeptides, the total number of PSM from each of the 3 cohorts were summed together (e.g., see values in “Combined PSM Count” column of Table 19) and filtered to a first subgroup that have a Combined PSM Count equal or greater than 30 to indicate that a significant number of that particular glycopeptide was measured in the experiment. The first subgroup resulted in 431 glycopeptides. The first subgroup was then further filtered by determining the fold change (e.g., see values in “Fold Change” column of Table 19), defined as the ratio of relative abundances, and then retaining the glycopeptides that had a fold change greater than 2 or less than 0.5 for at least one of the Early sPE/control or Late sPE/Control. The fold change was calculated for each glycopeptide of the first subgroup by dividing the relative abundance for early sPE by the relative abundance for healthy control or by dividing the relative abundance for late sPE by the relative abundance for healthy control. The retained glycopeptides from the first subgroup after filtering based on the fold change yielded 124 glycopeptides to form the second subgroup that is associated with the identification of preeclampsia in view of the control group as shown in Table 19. Table 16 summarizes details concerning the glycoproteins studied. Table 17 presents specifics for the 124 glycopeptides identified in the filtering procedure. Table 18 defines the structure and composition of glycans and Table 19 summarizes the PSM count, relative abundance, and fold change for the 124 glycopeptides. The 124 identified glycopeptides represent either up-regulation or down-regulation in early sPE or late sPE when compared to healthy control.

TABLE 16
Glycoproteins associated with pregnancy control and sPE

| SEQ | Protein | | | |
|---|---|---|---|---|
| ID NO | Abbreviation | Protein Name | Uniprot ID | Protein Sequence |
| 22 | A1AG1 | Alpha-1-acid | P02763 | MALSWVLTVLSLLPLLEAQIPLCANLVPVP |
| | | glycoprotein 1 | | ITNATLDRITGKWFYIASAFRNEEYNKSVQ |
| | | | | EIQATFFYFTPNKTEDTIFLREYQTRQDQCI |
| | | | | YNTTYLNVQRENGTISRYVGGQEHFAHLLI |
| | | | | LRDTKTYMLAFDVNDEKNWGLSVYADKP |
| | | | | ETTKEQLGEFYEALDCLRIPKSDVVYTDW |
| | | | | KKDKCEPLEKQHEKERKQEEGES |
| 23 | A1AT | Alpha-1-antitrypsin | P01009 | MPSSVSWGILLLAGLCCLVPVSLAEDPQG |
| | | | | DAAQKTDTSHHDQDHPTFNKITPNLAEFA |
| | | | | FSLYRQLAHQSNSTNIFFSPVSIATAFAMLS |
| | | | | LGTKADTHDEILEGLNFNLTEIPEAQIHEGF |
| | | | | QELLRTLNQPDSQLQLTTGNGLFLSEGLKL |
| | | | | VDKFLEDVKKLYHSEAFTVNFGDTEEAKK |
| | | | | QINDYVEKGTQGKIVDLVKELDRDTVFAL |
| | | | | VNYIFFKGKWERPFEVKDTEEEDFHVDQV |
| | | | | TTVKVPMMKRLGMFNIQHCKKLSSWVLL |
| | | | | MKYLGNATAIFFLPDEGKLQHLENELTHDI |
| | | | | ITKFLENEDRRSASLHLPKLSITGTYDLKSV |
| | | | | LGQLGITKVFSNGADLSGVTEEAPLKLSKA |
| | | | | VHKAVLTIDEKGTEAAGAMFLEAIPMSIPP |
| | | | | EVKFNKPFVFLMIEQNTKSPLFMGKVVNP |
| | | | | TQK |
| 24 | A2MG | Alpha-2- | P01023 | MGKNKLLHPSLVLLLLVLLPTDASVSGKP |
| | | macroglobulin | | QYMVLVPSLLHTETTEKGCVLLSYLNETV |
| | | | | VSASLESVRGNRSLFTDLEAENDVLHCV |
| | | | | AFAVPKSSSNEEVMFLTVQVKGPTQEFKK |
| | | | | RTTVMVKNEDSLVFVQTDKSIYKPGQTVK |
| | | | | FRVVSMDENFHPLNELIPLVYIQDPKGNRI |
| | | | | AQWQSFQLEGGLKQFSFPLSSEPFQGSYKV |
| | | | | VVQKKSGGRTEHPFTVEEFVLPKFEVQVT |
| | | | | VPKIITILEEEMNVSVCGLYTYGKPVPGHV |
| | | | | TVSICRKYSDASDCHGEDSQAFCEKFSGQL |
| | | | | NSHGCFYQQVKTKVFQLKRKEYEMKLHT |
| | | | | EAQIQEEGTVVELTGRQSSEITRTITKLSFV |
| | | | | KVDSHFRQGIPFFGQVRLVDGKGVPIPNKV |
| | | | | IFIRGNEANYYSNATTDEHGLVQFSINTTN |
| | | | | VMGTSLTVRVNYKDRSPCYGYQWVSEEH |
| | | | | EEAHHTAYLVFSPSKSFVHLEPMSHELPCG |
| | | | | HTQTVQAHYILNGGTLLGLKKLSFYYLIM |
| | | | | AKGGIVRTGTHGLLVKQEDMKGHFSISIPV |
| | | | | KSDIAPVARLLIYAVLPTGDVIGDSAKYDV |
| | | | | ENCLANKVDLSFSPSQSLPASHAHLRVTAA |
| | | | | PQSVCALRAVDQSVLLMKPDAELSASSVY |
| | | | | NLLPEKDLTGFPGPLNDQDNEDCINRHNV |
| | | | | YINGITYTPVSSTNEKDMYSFLEDMGLKAF |
| | | | | TNSKIRKPKMCPQLQQYEMHGPEGLRVGF |
| | | | | YESDVMGRGHARLVHVEEPHTETVRKYFP |
| | | | | ETWIWDLVVVNSAGVAEVGVTVPDTITE |
| | | | | WKAGAFCLSEDAGLGISSTASLRAFQPFFV |
| | | | | ELTMPYSVIRGEAFTLKATVLNYLPKCIRV |
| | | | | SVQLEASPAFLAVPVEKEQAPHCICANGRQ |
| | | | | TVSWAVTPKSLGNVNFTVSAEALESQELC |
| | | | | GTEVPSVPEHGRKDTVIKPLLVEPEGLEKE |
| | | | | TTFNSLLCPSGGEVSEELSLKLPPNVVEESA |
| | | | | RASVSVLGDILGSAMQNTQNLLQMPYGCG |
| | | | | EQNMVLFAPNIYVLDYLNETQQLTPEIKSK |
| | | | | AIGYLNTGYQRQLNYKHYDGSYSTFGERY |
| | | | | GRNQGNTWLTAFVLKTFAQARAYIFIDEA |
| | | | | HITQALIWLSQRQKDNGCFRSSGSLLNNAI |
| | | | | KGGVEDEVTLSAYITIALLEIPLTVTHPVVR |
| | | | | NALFCLESAWKTAQEGDHGSHVYTKALL |
| | | | | AYAFALAGNQDKRKEVLKSLNEEAVKKD |
| | | | | NSVHWERPQKPKAPVGHFYEPQAPSAEVE |
| | | | | MTSYVLLAYLTAQPAPTSEDLTSATNIVK |
| | | | | WITKQQNAQGGFSSTQDTVVALHALSKYG |
| | | | | AATFTRTGKAAQVTIQSSGTFSSKFQVDNN |
| | | | | NRLLLQQVSLPELPGEYSMKVTGEGCVYL |
| | | | | QTSLKYNILPEKEEFPFALGVQTLPQTCDEP |
| | | | | KAHTSFQISLSVSYTGSRSASNMAIVDVKM |
| | | | | VSGFIPLKPTVKMLERSNHVSRTEVSSNHV |
| | | | | LIYLDKVSNQTLSLFFTVLQDVPVRDLKPA |
| | | | | IVKVYDYYETDEFAIAEYNAPCSKDLGNA |
| 25 | AACT | Alpha-1- | P01011 | MERMLPLLALGLLAAGFCPAVLCHPNSPL |
| | | antichymotrypsin | | DEENLTQENQDRGTHVDLGLASANVDFAF |
| | | | | SLYKQLVLKAPDKNVIFSPLSISTALAFLSL |
| | | | | GAHNTTLTEILKGLKFNLTETSEAEIHQSFQ |
| | | | | HLLRTLNQSSDELQLSMGNAMFVKEQLSL |
| | | | | LDRFTEDAKRLYGSEAFATDFQDSAAAKK |
| | | | | LINDYVKNGTRGKITDLIKDLDSQTMMVL |
| | | | | VNYIFFKAKWEMPFDPQDTHQSRFYLSKK |
| | | | | KWVMVPMMSLHHLTIPYFRDEELSCTVVE |
| | | | | LKYTGNASALFILPDQDKMEEVEAMLLPE |
| | | | | TLKRWRDSLEFREIGELYLPKFSISRDYNL |
| | | | | NDILLQLGIEEAFTSKADLSGITGARNLAVS |
| | | | | QVVHKAVLDVFEEGTEASAATAVKITLLS |
| | | | | ALVETRTIVRFNRPFLMIIVPTDTQNIFFMS |
| | | | | KVTNPKQA |
| 26 | AMBP | Protein AMBP | P02760 | MRSLGALLLLLSACLAVSAGPVPTPPDNIQ |
| | | | | VQENFNISRIYGKWYNLAIGSTCPWLKKIM |
| | | | | DRMTVSTLVLGEGATEAEISMTSTRWRKG |
| | | | | VCEETSGAYEKTDTDGKFLYHKSKWNITM |
| | | | | ESYVVHTNYDEYAIFLTKKFSRHHGPTITA |
| | | | | KLYGRAPQLRETLLQDFRVVAQGVGIPED |
| | | | | SIFTMADRGECVPGEQEPEPILIPRVRRAVL |
| | | | | PQEEEGSGGGQLVTEVTKKEDSCQLGYSA |
| | | | | GPCMGMTSRYFYNGTSMACETFQYGGCM |
| | | | | GNGNNFVTEKECLQTCRTVAACNLPIVRG |
| | | | | PCRAFIQLWAFDAVKGKCVLFPYGGCQGN |
| | | | | GNKFYSEKECREYCGVPGDGDEELLRFSN |
| 27 | ANGT | Angiotensinogen | P01019 | MAPAGVSLRATILCLLAWAGLAAGDRVYI |
| | | | | HPFHLVIHNESTCEQLAKANAGKPKDPTFI |
| | | | | PAPIQAKTSPVDEKALQDQLVLVAAKLDT |
| | | | | EDKLRAAMVGMLANFLGFRIYGMHSELW |
| | | | | GVVHGATVLSPTAVFGTLASLYLGALDHT |
| | | | | ADRLQAILGVPWKDKNCTSRLDAHKVLSA |
| | | | | LQAVQGLLVAQGRADSQAQLLLSTVVGV |
| | | | | FTAPGLHLKQPFVQGLALYTPVVLPRSLDF |
| | | | | TELDVAAEKIDRFMQAVTGWKTGCSLMG |
| | | | | ASVDSTLAFNTYVHFQGKMKGFSLLAEPQ |
| | | | | EFWVDNSTSVSVPMLSGMGTFQHWSDIQD |
| | | | | NFSVTQVPFTESACLLLIQPHYASDLDKVE |
| | | | | GLTFQQNSLNWMKKLSPRTIHLTMPQLVL |
| | | | | QGSYDLQDLLAQAELPAILHTELNLQKLSN |
| | | | | DRIRVGEVLNSIFFELEADEREPTESTQQLN |
| | | | | KPEVLEVTLNRPFLFAVYDQSATALHFLGR |
| | | | | VANPLSTA |
| 28 | ANT3 | Antithrombin-III | P01008 | MYSNVIGTVTSGKRKVYLLSLLLIGFWDC |
| | | | | VTCHGSPVDICTAKPRDIPMNPMCIYRSPE |
| | | | | KKATEDEGSEQKIPEATNRRVWELSKANS |
| | | | | RFATTFYQHLADSKNDNDNIFLSPLSISTAF |
| | | | | AMTKLGACNDTLQQLMEVFKFDTISEKTS |
| | | | | DQIHFFFAKLNCRLYRKANKSSKLVSANR |
| | | | | LFGDKSLTFNETYQDISELVYGAKLQPLDF |
| | | | | KENAEQSRAAINKWVSNKTEGRITDVIPSE |
| | | | | AINELTVLVLVNTIYFKGLWKSKFSPENTR |
| | | | | KELFYKADGESCSASMMYQEGKFRYRRV |
| | | | | AEGTQVLELPFKGDDITMVLILPKPEKSLA |
| | | | | KVEKELTPEVLQEWLDELEEMMLVVHMP |
| | | | | RFRIEDGFSLKEQLQDMGLVDLFSPEKSKL |
| | | | | PGIVAEGRDDLYVSDAFHKAFLEVNEEGS |
| | | | | EAAASTAVVIAGRSLNPNRVTFKANRPFLV |
| | | | | FIREVPLNTIIFMGRVANPCVK |
| 29 | AP1M2 | AP-1 complex | Q9Y6Q5 | MSASAVFILDVKGKPLISRNYKGDVAMSKI |
| | | subunit mu-2 | | EHFMPLLVQREEEGALAPLLSHGQVHFLW |
| | | | | IKHSNLYLVATTSKNANASLVYSFLYKTIE |
| | | | | VFCEYFKELEEESIRDNFVIVYELLDELMD |
| | | | | FGFPQTTDSKILQEYITQQSNKLETGKSRVP |
| | | | | PTVTNAVSWRSEGIKYKKNEVFIDVIESVN |
| | | | | LLVNANGSVLLSEIVGTIKLKVFLSGMPEL |
| | | | | RLGLNDRVLFELTGRSKNKSVELEDVKFH |
| | | | | QCVRLSRFDNDRTISFIPPDGDFELMSYRLS |
| | | | | TQVKPLIWIESVIEKFSHSRVEIMVKAKGQ |
| | | | | FKKQSVANGVEISVPVPSDADSPRFKTSVG |
| | | | | SAKYVPERNVVIWSIKSFPGGKEYLMRAH |
| | | | | FGLPSVEKEEVEGRPPIGVKFEIPYFTVSGI |
| | | | | QVRYMKIIEKSGYQALPWVRYITQSGDYQ |
| | | | | LRTS |
| 30 | APOB | Apolipoprotein B- | P04114 | MDPPRPALLALLALPALLLLLLAGARAEEE |
| | | 100 | | MLENVSLVCPKDATRFKHLRKYTYNYEAE |
| | | | | SSSGVPGTADSRSATRINCKVELEVPQLCS |
| | | | | FILKTSQCTLKEVYGFNPEGKALLKKTKNS |
| | | | | EEFAAAMSRYELKLAIPEGKQVFLYPEKDE |
| | | | | PTYILNIKRGIISALLVPPETEEAKQVLFLDT |
| | | | | VYGNCSTHFTVKTRKGNVATEISTERDLG |
| | | | | QCDRFKPIRTGISPLALIKGMTRPLSTLISSS |
| | | | | QSCQYTLDAKRKHVAEAICKEQHLFLPFS |
| | | | | YKNKYGMVAQVTQTLKLEDTPKINSRFFG |
| | | | | EGTKKMGLAFESTKSTSPPKQAEAVLKTL |
| | | | | QELKKLTISEQNIQRANLFNKLVTELRGLS |
| | | | | DEAVTSLLPQLIEVSSPITLQALVQCGQPQC |
| | | | | STHILQWLKRVHANPLLIDVVTYLVALIPE |
| | | | | PSAQQLREIFNMARDQRSRATLYALSHAV |
| | | | | NNYHKTNPTGTQELLDIANYLMEQIQDDC |
| | | | | TGDEDYTYLILRVIGNMGQTMEQLTPELK |
| | | | | SSILKCVQSTKPSLMIQKAAIQALRKMEPK |
| | | | | DKDQEVLLQTFLDDASPGDKRLAAYLML |
| | | | | MRSPSQADINKIVQILPWEQNEQVKNFVAS |
| | | | | HIANILNSEELDIQDLKKLVKEALKESQLPT |
| | | | | VMDFRKFSRNYQLYKSVSLPSLDPASAKIE |
| | | | | GNLIFDPNNYLPKESMLKTTLTAFGFASAD |
| | | | | LIEIGLEGKGFEPTLEALFGKQGFFPDSVNK |
| | | | | ALYWVNGQVPDGVSKVLVDHFGYTKDD |
| | | | | KHEQDMVNGIMLSVEKLIKDLKSKEVPEA |
| | | | | RAYLRILGEELGFASLHDLQLLGKLLLMG |
| | | | | ARTLQGIPQMIGEVIRKGSKNDFFLHYIFM |
| | | | | ENAFELPTGAGLQLQISSSGVIAPGAKAGV |
| | | | | KLEVANMQAELVAKPSVSVEFVTNMGIIIP |
| | | | | DFARSGVQMNTNFFHESGLEAHVALKAG |
| | | | | KLKFIIPSPKRPVKLLSGGNTLHLVSTTKTE |
| | | | | VIPPLIENRQSWSVCKQVFPGLNYCTSGAY |
| | | | | SNASSTDSASYYPLTGDTRLELELRPTGEIE |
| | | | | QYSVSATYELQREDRALVDTLKFVTQAEG |
| | | | | AKQTEATMTFKYNRQSMTLSSEVQIPDFD |
| | | | | VDLGTILRVNDESTEGKTSYRLTLDIQNKK |
| | | | | ITEVALMGHLSCDTKEERKIKGVISIPRLQA |
| | | | | EARSEILAHWSPAKLLLQMDSSATAYGST |
| | | | | VSKRVAWHYDEEKIEFEWNTGTNVDTKK |
| | | | | MTSNFPVDLSDYPKSLHMYANRLLDHRVP |
| | | | | QTDMTFRHVGSKLIVAMSSWLQKASGSLP |
| | | | | YTQTLQDHLNSLKEFNLQNMGLPDFHIPE |
| | | | | NLFLKSDGRVKYTLNKNSLKIEIPLPFGGK |
| | | | | SSRDLKMLETVRTPALHFKSVGFHLPSREF |
| | | | | QVPTFTIPKLYQLQVPLLGVLDLSTNVYSN |
| | | | | LYNWSASYSGGNTSTDHFSLRARYHMKA |
| | | | | DSVVDLLSYNVQGSGETTYDHKNTFTLSY |
| | | | | DGSLRHKFLDSNIKFSHVEKLGNNPVSKGL |
| | | | | LIFDASSSWGPQMSASVHLDSKKKQHLFV |
| | | | | KEVKIDGQFRVSSFYAKGTYGLSCQRDPN |
| | | | | TGRLNGESNLRFNSSYLQGTNQITGRYEDG |
| | | | | TLSLTSTSDLQSGIIKNTASLKYENYELTLK |
| | | | | SDTNGKYKNFATSNKMDMTFSKQNALLR |
| | | | | SEYQADYESLRFFSLLSGSLNSHGLELNAD |
| | | | | ILGTDKINSGAHKATLRIGQDGISTSATTNL |
| | | | | KCSLLVLENELNAELGLSGASMKLTTNGR |
| | | | | FREHNAKFSLDGKAALTELSLGSAYQAMI |
| | | | | LGVDSKNIFNFKVSQEGLKLSNDMMGSYA |
| | | | | EMKFDHTNSLNIAGLSLDFSSKLDNIYSSD |
| | | | | KFYKQTVNLQLQPYSLVTTLNSDLKYNAL |
| | | | | DLTNNGKLRLEPLKLHVAGNLKGAYQNN |
| | | | | EIKHIYAISSAALSASYKADTVAKVQGVEF |
| | | | | SHRLNTDIAGLASAIDMSTNYNSDSLHFSN |
| | | | | VFRSVMAPFTMTIDAHTNGNGKLALWGE |
| | | | | HTGQLYSKFLLKAEPLAFTFSHDYKGSTSH |
| | | | | HLVSRKSISAALEHKVSALLTPAEQTGTW |
| | | | | KLKTQFNNNEYSQDLDAYNTKDKIGVELT |
| | | | | GRTLADLTLLDSPIKVPLLLSEPINIIDALEM |
| | | | | RDAVEKPQEFTIVAFVKYDKNQDVHSINLP |
| | | | | FFETLQEYFERNRQTIIVVLENVQRNLKHIN |
| | | | | IDQFVRKYRAALGKLPQQANDYLNSFNWE |
| | | | | RQVSHAKEKLTALTKKYRITENDIQIALDD |
| | | | | AKINFNEKLSQLQTYMIQFDQYIKDSYDLH |
| | | | | DLKIAIANIIDEIIEKLKSLDEHYHIRVNLVK |
| | | | | TIHDLHLFIENIDENKSGSSTASWIQNVDTK |
| | | | | YQIRIQIQEKLQQLKRHIQNIDIQHLAGKLK |
| | | | | QHIEAIDVRVLLDQLGTTISFERINDILEHV |
| | | | | KHFVINLIGDFEVAEKINAFRAKVHELIERY |
| | | | | EVDQQIQVLMDKLVELAHQYKLKETIQKL |
| | | | | SNVLQQVKIKDYFEKLVGFIDDAVKKLNE |
| | | | | LSFKTFIEDVNKFLDMLIKKLKSFDYHQFV |
| | | | | DETNDKIREVTQRLNGEIQALELPQKAEAL |
| | | | | KLFLEETKATVAVYLESLQDTKITLIINWL |
| | | | | QEALSSASLAHMKAKFRETLEDTRDRMYQ |
| | | | | MDIQQELQRYLSLVGQVYSTLVTYISDWW |
| | | | | TLAAKNLTDFAEQYSIQDWAKRMKALVE |
| | | | | QGFTVPEIKTILGTMPAFEVSLQALQKATF |
| | | | | QTPDFIVPLTDLRIPSVQINFKDLKNIKIPSR |
| | | | | FSTPEFTILNTFHIPSFTIDFVEMKVKIIRTID |
| | | | | QMLNSELQWPVPDIYLRDLKVEDIPLARIT |
| | | | | LPDFRLPEIAIPEFIIPTLNLNDFQVPDLHIPE |
| | | | | FQLPHISHTIEVPTFGKLYSILKIQSPLFTLD |
| | | | | ANADIGNGTTSANEAGIAASITAKGESKLE |
| | | | | VLNFDFQANAQLSNPKINPLALKESVKFSS |
| | | | | KYLRTEHGSEMLFFGNAIEGKSNTVASLHT |
| | | | | EKNTLELSNGVIVKINNQLTLDSNTKYFHK |
| | | | | LNIPKLDFSSQADLRNEIKTLLKAGHIAWT |
| | | | | SSGKGSWKWACPRFSDEGTHESQISFTIEG |
| | | | | PLTSFGLSNKINSKHLRVNQNLVYESGSLN |
| | | | | FSKLEIQSQVDSQHVGHSVLTAKGMALFG |
| | | | | EGKAEFTGRHDAHLNGKVIGTLKNSLFFS |
| | | | | AQPFEITASTNNEGNLKVRFPLRLTGKIDFL |
| | | | | NNYALFLSPSAQQASWQVSARFNQYKYN |
| | | | | QNFSAGNNENIMEAHVGINGEANLDFLNIP |
| | | | | LTIPEMRLPYTIITTPPLKDFSLWEKTGLKE |
| | | | | FLKTTKQSFDLSVKAQYKKNKHRHSITNPL |
| | | | | AVLCEFISQSIKSFDRHFEKNRNNALDFVT |
| | | | | KSYNETKIKFDKYKAEKSHDELPRTFQIPG |
| | | | | YTVPVVNVEVSPFTIEMSAFGYVFPKAVS |
| | | | | MPSFSILGSDVRVPSYTLILPSLELPVLHVP |
| | | | | RNLKLSLPDFKELCTISHIFIPAMGNITYDFS |
| | | | | FKSSVITLNTNAELFNQSDIVAHLLSSSSSVI |
| | | | | DALQYKLEGTTRLTRKRGLKLATALSLSN |
| | | | | KFVEGSHNSTVSLTTKNMEVSVATTTKAQ |
| | | | | IPILRMNFKQELNGNTKSKPTVSSSMEFKY |
| | | | | DFNSSMLYSTAKGAVDHKLSLESLTSYFSI |
| | | | | ESSTKGDVKGSVLSREYSGTIASEANTYLN |
| | | | | SKSTRSSVKLQGTSKIDDIWNLEVKENFAG |
| | | | | EATLQRIYSLWEHSTKNHLQLEGLFFTNGE |
| | | | | HTSKATLELSPWQMSALVQVHASQPSSFH |
| | | | | DFPDLGQEVALNANTKNQKIRWKNEVRIH |
| | | | | SGSFQSQVELSNDQEKAHLDIAGSLEGHLR |
| | | | | FLKNIILPVYDKSLWDFLKLDVTTSIGRRQ |
| | | | | HLRVSTAFVYTKNPNGYSFSIPVKVLADKF |
| | | | | IIPGLKLNDLNSVLVMPTFHVPFTDLQVPS |
| | | | | CKLDFREIQIYKKLRTSSFALNLPTLPEVKF |
| | | | | PEVDVLTKYSQPEDSLIPFFEITVPESQLTVS |
| | | | | QFTLPKSVSDGIAALDLNAVANKIADFELP |
| | | | | TIIVPEQTIEIPSIKFSVPAGIVIPSFQALTARF |
| | | | | EVDSPVYNATWSASLKNKADYVETVLDST |
| | | | | CSSTVQFLEYELNVLGTHKIEDGTLASKTK |
| | | | | GTFAHRDFSAEYEEDGKYEGLQEWEGKA |
| | | | | HLNIKSPAFTDLHLRYQKDKKGISTSAASP |
| | | | | AVGTVGMDMDEDDDFSKWNFYYSPQSSP |
| | | | | DKKLTIFKTELRVRESDEETQIKVNWEEEA |
| | | | | ASGLLTSLKDNVPKATGVLYDYVNKYHW |
| | | | | EHTGLTLREVSSKLRRNLQNNAEWVYQG |
| | | | | AIRQIDDIDVRFQKAASGTTGTYQEWKDK |
| | | | | AQNLYQELLTQEGQASFQGLKDNVFDGLV |
| | | | | RVTQEFHMKVKHLIDSLIDFLNFPRFQFPG |
| | | | | KPGIYTREELCTMFIREVGTVLSQVYSKVH |
| | | | | NGSEILFSYFQDLVITLPFELRKHKLIDVIS |
| | | | | MYRELLKDLSKEAQEVFKAIQSLKTTEVL |
| | | | | RNLQDLLQFIFQLIEDNIKQLKEMKFTYLIN |
| | | | | YIQDEINTIFSDYIPYVFKLLKENLCLNLHK |
| | | | | FNEFIQNELQEASQELQQIHQYIMALREEY |
| | | | | FDPSIVGWTVKYYELEEKIVSLIKNLLVAL |
| | | | | KDFHSEYIVSASNFTSQLSSQVEQFLHRNIQ |
| | | | | EYLSILTDPDGKGKEKIAELSATAQEIIKSQ |
| | | | | AIATKKIISDYHQQFRYKLQDFSDQLSDYY |
| | | | | EKFIAESKRLIDLSIQNYHTFLIYITELLKKL |
| | | | | QSTTVMNPYMKLAPGELTIIL |
| 31 | CERU | Ceruloplasmin | P00450 | MKILILGIFLFLCSTPAWAKEKHYYIGIIETT |
| | | | | WDYASDHGEKKLISVDTEHSNIYLQNGPD |
| | | | | RIGRLYKKALYLQYTDETFRTTIEKPVWLG |
| | | | | FLGPIIKAETGDKVYVHLKNLASRPYTFHS |
| | | | | HGITYYKEHEGAIYPDNTTDFQRADDKVY |
| | | | | PGEQYTYMLLATEEQSPGEGDGNCVTRIY |
| | | | | HSHIDAPKDIASGLIGPLIICKKDSLDKEKE |
| | | | | KHIDREFVVMFSVVDENFSWYLEDNIKTY |
| | | | | CSEPEKVDKDNEDFQESNRMYSVNGYTFG |
| | | | | SLPGLSMCAEDRVKWYLFGMGNEVDVHA |
| | | | | AFFHGQALTNKNYRIDTINLFPATLFDAYM |
| | | | | VAQNPGEWMLSCQNLNHLKAGLQAFFQV |
| | | | | QECNKSSSKDNIRGKHVRHYYIAAEEIIWN |
| | | | | YAPSGIDIFTKENLTAPGSDSAVFFEQGTTR |
| | | | | IGGSYKKLVYREYTDASFTNRKERGPEEEH |
| | | | | LGILGPVIWAEVGDTIRVTFHNKGAYPLSIE |
| | | | | PIGVRFNKNNEGTYYSPNYNPQSRSVPPSA |
| | | | | SHVAPTETFTYEWTVPKEVGPTNADPVCL |
| | | | | AKMYYSAVDPTKDIFTGLIGPMKICKKGSL |
| | | | | HANGRQKDVDKEFYLFPTVFDENESLLLE |
| | | | | DNIRMFTTAPDQVDKEDEDFQESNKMHS |
| | | | | MNGFMYGNQPGLTMCKGDSVVWYLFSA |
| | | | | GNEADVHGIYFSGNTYLWRGERRDTANLF |
| | | | | PQTSLTLHMWPDTEGTFNVECLTTDHYTG |
| | | | | GMKQKYTVNQCRRQSEDSTFYLGERTYYI |
| | | | | AAVEVEWDYSPQREWEKELHHLQEQNVS |
| | | | | NAFLDKGEFYIGSKYKKVVYRQYTDSTFR |
| | | | | VPVERKAEEEHLGILGPQLHADVGDKVKII |
| | | | | FKNMATRPYSIHAHGVQTESSTVTPTLPGE |
| | | | | TLTYVWKIPERSGAGTEDSACIPWAYYST |
| | | | | VDQVKDLYSGLIGPLIVCRRPYLKVFNPRR |
| | | | | KLEFALLFLVFDENESWYLDDNIKTYSDHP |
| | | | | EKVNKDDEEFIESNKMHAINGRMFGNLQG |
| | | | | LTMHVGDEVNWYLMGMGNEIDLHTVHF |
| | | | | HGHSFQYKHRGVYSSDVFDIFPGTYQTLE |
| | | | | MFPRTPGIWLLHCHVTDHIHAGMETTYTV |
| | | | | LQNEDTKSG |
| 32 | CFAB | Complement factor | P00751 | MGSNLSPQLCLMPFILGLLSGGVTTTPWSL |
| | | B | | ARPQGSCSLEGVEIKGGSFRLLQEGQALEY |
| | | | | VCPSGFYPYPVQTRTCRSTGSWSTLKTQD |
| | | | | QKTVRKAECRAIHCPRPHDFENGEYWPRS |
| | | | | PYYNVSDEISFHCYDGYTLRGSANRTCQV |
| | | | | NGRWSGQTAICDNGAGYCSNPGIPIGTRK |
| | | | | VGSQYRLEDSVTYHCSRGLTLRGSQRRTC |
| | | | | QEGGSWSGTEPSCQDSFMYDTPQEVAEAF |
| | | | | LSSLTETIEGVDAEDGHGPGEQQKRKIVLD |
| | | | | PSGSMNIYLVLDGSDSIGASNFTGAKKCLV |
| | | | | NLIEKVASYGVKPRYGLVTYATYPKIWVK |
| | | | | VSEADSSNADWVTKQLNEINYEDHKLKSG |
| | | | | TNTKKALQAVYSMMSWPDDVPPEGWNRT |
| | | | | RHVIILMTDGLHNMGGDPITVIDEIRDLLYI |
| | | | | GKDRKNPREDYLDVYVFGVGPLVNQVNI |
| | | | | NALASKKDNEQHVFKVKDMENLEDVFYQ |
| | | | | MIDESQSLSLCGMVWEHRKGTDYHKQPW |
| | | | | QAKISVIRPSKGHESCMGAVVSEYFVLTAA |
| | | | | HCFTVDDKEHSIKVSVGGEKRDLEIEVVLF |
| | | | | HPNYNINGKKEAGIPEFYDYDVALIKLKNK |
| | | | | LKYGQTIRPICLPCTEGTTRALRLPPTTTCQ |
| | | | | QQKEELLPAQDIKALFVSEEEKKLTRKEVY |
| | | | | IKNGDKKGSCERDAQYAPGYDKVKDISEV |
| | | | | VTPRFLCTGGVSPYADPNTCRGDSGGPLIV |
| | | | | HKRSRFIQVGVISWGVVDVCKNQKRQKQ |
| | | | | VPAHARDFHINLFQVLPWLKEKLQDEDLG |
| | | | | FL |
| 33 | CO4A | Complement C4-A | POCOL4 | MRLLWGLIWASSFFTLSLQKPRLLLFSPSV |
| | | | | VHLGVPLSVGVQLQDVPRGQVVKGSVFLR |
| | | | | NPSRNNVPCSPKVDFTLSSERDFALLSLQV |
| | | | | PLKDAKSCGLHQLLRGPEVQLVAHSPWLK |
| | | | | DSLSRTTNIQGINLLFSSRRGHLFLQTDQPI |
| | | | | YNPGQRVRYRVFALDQKMRPSTDTITVMV |
| | | | | ENSHGLRVRKKEVYMPSSIFQDDFVIPDISE |
| | | | | PGTWKISARFSDGLESNSSTQFEVKKYVLP |
| | | | | NFEVKITPGKPYILTVPGHLDEMQLDIQAR |
| | | | | YIYGKPVQGVAYVRFGLLDEDGKKTFFRG |
| | | | | LESQTKLVNGQSHISLSKAEFQDALEKLN |
| | | | | MGITDLQGLRLYVAAAIIESPGGEMEEAEL |
| | | | | TSWYFVSSPFSLDLSKTKRHLVPGAPFLLQ |
| | | | | ALVREMSGSPASGIPVKVSATVSSPGSVPE |
| | | | | VQDIQQNTDGSGQVSIPIIIPQTISELQLSVS |
| | | | | AGSPHPAIARLTVAAPPSGGPGFLSIERPDS |
| | | | | RPPRVGDTLNLNLRAVGSGATFSHYYYMI |
| | | | | LSRGQIVFMNREPKRTLTSVSVFVDHHLAP |
| | | | | SFYFVAFYYHGDHPVANSLRVDVQAGAC |
| | | | | EGKLELSVDGAKQYRNGESVKLHLETDSL |
| | | | | ALVALGALDTALYAAGSKSHKPLNMGKV |
| | | | | FEAMNSYDLGCGPGGGDSALQVFQAAGL |
| | | | | AFSDGDQWTLSRKRLSCPKEKTTRKKRNV |
| | | | | NFQKAINEKLGQYASPTAKRCCQDGVTRL |
| | | | | PMMRSCEQRAARVQQPDCREPFLSCCQFA |
| | | | | ESLRKKSRDKGQAGLQRALEILQEEDLIDE |
| | | | | DDIPVRSFFPENWLWRVETVDRFQILTLWL |
| | | | | PDSLTTWEIHGLSLSKTKGLCVATPVQLRV |
| | | | | FREFHLHLRLPMSVRRFEQLELRPVLYNYL |
| | | | | DKNLTVSVHVSPVEGLCLAGGGGLAQQV |
| | | | | LVPAGSARPVAFSVVPTAAAAVSLKVVAR |
| | | | | GSFEFPVGDAVSKVLQIEKEGAIHREELVY |
| | | | | ELNPLDHRGRTLEIPGNSDPNMIPDGDENS |
| | | | | YVRVTASDPLDTLGSEGALSPGGVASLLRL |
| | | | | PRGCGEQTMIYLAPTLAASRYLDKTEQWS |
| | | | | TLPPETKDHAVDLIQKGYMRIQQFRKADG |
| | | | | SYAAWLSRDSSTWLTAFVLKVLSLAQEQV |
| | | | | GGSPEKLQETSNWLLSQQQADGSFQDPCP |
| | | | | VLDRSMQGGLVGNDETVALTAFVTIALHH |
| | | | | GLAVFQDEGAEPLKQRVEASISKANSFLGE |
| | | | | KASAGLLGAHAAAITAYALTLTKAPVDLL |
| | | | | GVAHNNLMAMAQETGDNLYWGSVTGSQ |
| | | | | SNAVSPTPAPRNPSDPMPQAPALWIETTAY |
| | | | | ALLHLLLHEGKAEMADQASAWLTRQGSF |
| | | | | QGGFRSTQDTVIALDALSAYWIASHTTEER |
| | | | | GLNVTLSSTGRNGFKSHALQLNNRQIRGLE |
| | | | | EELQFSLGSKINVKVGGNSKGTLKVLRTY |
| | | | | NVLDMKNTTCQDLQIEVTVKGHVEYTME |
| | | | | ANEDYEDYEYDELPAKDDPDAPLQPVTPL |
| | | | | QLFEGRRNRRRREAPKVVEEQESRVHYTV |
| | | | | CIWRNGKVGLSGMAIADVTLLSGFHALRA |
| | | | | DLEKLTSLSDRYVSHFETEGPHVLLYFDSV |
| | | | | PTSRECVGFEAVQEVPVGLVQPASATLYD |
| | | | | YYNPERRCSVFYGAPSKSRLLATLCSAEVC |
| | | | | QCAEGKCPRQRRALERGLQDEDGYRMKF |
| | | | | ACYYPRVEYGFQVKVLREDSRAAFRLFET |
| | | | | KITQVLHFTKDVKAAANQMRNFLVRASCR |
| | | | | LRLEPGKEYLIMGLDGATYDLEGHPQYLL |
| | | | | DSNSWIEEMPSERLCRSTRQRAACAQLND |
| | | | | FLQEYGTQGCQV |
| 34 | FIBB | Fibrinogen beta | P02675 | MKRMVSWSFHKLKTMKHLLLLLLCVFLV |
| | | chain | | KSQGVNDNEEGFFSARGHRPLDKKREEAP |
| | | | | SLRPAPPPISGGGYRARPAKAAATQKKVER |
| | | | | KAPDAGGCLHADPDLGVLCPTGCQLQEAL |
| | | | | LQQERPIRNSVDELNNNVEAVSQTSSSSFQ |
| | | | | YMYLLKDLWQKRQKQVKDNENVVNEYS |
| | | | | SELEKHQLYIDETVNSNIPTNLRVLRSILEN |
| | | | | LRSKIQKLESDVSAQMEYCRTPCTVSCNIP |
| | | | | VVSGKECEEIIRKGGETSEMYLIQPDSSVKP |
| | | | | YRVYCDMNTENGGWTVIQNRQDGSVDFG |
| | | | | RKWDPYKQGFGNVATNTDGKNYCGLPGE |
| | | | | YWLGNDKISQLTRMGPTELLIEMEDWKGD |
| | | | | KVKAHYGGFTVQNEANKYQISVNKYRGT |
| | | | | AGNALMDGASQLMGENRTMTIHNGMFFS |
| | | | | TYDRDNDGWLTSDPRKQCSKEDGGGWW |
| | | | | YNRCHAANPNGRYYWGGQYTWDMAKH |
| | | | | GTDDGVVWMNWKGSWYSMRKMSMKIRP |
| | | | | FFPQQ |
| 35 | FINC | Fibronectin | P02751 | MLRGPGPGLLLLAVQCLGTAVPSTGASKS |
| | | | | KRQAQQMVQPQSPVAVSQSKPGCYDNGK |
| | | | | HYQINQQWERTYLGNALVCTCYGGSRGF |
| | | | | NCESKPEAEETCFDKYTGNTYRVGDTYER |
| | | | | PKDSMIWDCTCIGAGRGRISCTIANRCHEG |
| | | | | GQSYKIGDTWRRPHETGGYMLECVCLGN |
| | | | | GKGEWTCKPIAEKCFDHAAGTSYVVGET |
| | | | | WEKPYQGWMMVDCTCLGEGSGRITCTSR |
| | | | | NRCNDQDTRTSYRIGDTWSKKDNRGNLL |
| | | | | QCICTGNGRGEWKCERHTSVQTTSSGSGPF |
| | | | | TDVRAAVYQPQPHPQPPPYGHCVTDSGVV |
| | | | | YSVGMQWLKTQGNKQMLCTCLGNGVSC |
| | | | | QETAVTQTYGGNSNGEPCVLPFTYNGRTF |
| | | | | YSCTTEGRQDGHLWCSTTSNYEQDQKYSF |
| | | | | CTDHTVLVQTRGGNSNGALCHFPFLYNNH |
| | | | | NYTDCTSEGRRDNMKWCGTTQNYDADQ |
| | | | | KFGFCPMAAHEEICTTNEGVMYRIGDQWD |
| | | | | KQHDMGHMMRCTCVGNGRGEWTCIAYS |
| | | | | QLRDQCIVDDITYNVNDTFHKRHEEGHML |
| | | | | NCTCFGQGRGRWKCDPVDQCQDSETGTF |
| | | | | YQIGDSWEKYVHGVRYQCYCYGRGIGEW |
| | | | | HCQPLQTYPSSSGPVEVFITETPSQPNSHPI |
| | | | | QWNAPQPSHISKYILRWRPKNSVGRWKEA |
| | | | | TIPGHLNSYTIKGLKPGVVYEGQLISIQQYG |
| | | | | HQEVTRFDFTTTSTSTPVTSNTVTGETTPFS |
| | | | | PLVATSESVTEITASSFVVSWVSASDTVSG |
| | | | | FRVEYELSEEGDEPQYLDLPSTATSVNIPDL |
| | | | | LPGRKYIVNVYQISEDGEQSLILSTSQTTAP |
| | | | | DAPPDTTVDQVDDTSIVVRWSRPQAPITGY |
| | | | | RIVYSPSVEGSSTELNLPETANSVTLSDLQP |
| | | | | GVQYNITIYAVEENQESTPVVIQQETTGTP |
| | | | | RSDTVPSPRDLQFVEVTDVKVTIMWTPPES |
| | | | | AVTGYRVDVIPVNLPGEHGQRLPISRNTFA |
| | | | | EVTGLSPGVTYYFKVFAVSHGRESKPLTA |
| | | | | QQTTKLDAPTNLQFVNETDSTVLVRWTPP |
| | | | | RAQITGYRLTVGLTRRGQPRQYNVGPSVS |
| | | | | KYPLRNLQPASEYTVSLVAIKGNQESPKAT |
| | | | | GVFTTLQPGSSIPPYNTEVTETTIVITWTPA |
| | | | | PRIGFKLGVRPSQGGEAPREVTSDSGSIVVS |
| | | | | GLTPGVEYVYTIQVLRDGQERDAPIVNKV |
| | | | | VTPLSPPTNLHLEANPDTGVLTVSWERSTT |
| | | | | PDITGYRITTTPTNGQQGNSLEEVVHADQS |
| | | | | SCTFDNLSPGLEYNVSVYTVKDDKESVPIS |
| | | | | DTIIPEVPQLTDLSFVDITDSSIGLRWTPLNS |
| | | | | STIIGYRITVVAAGEGIPIFEDFVDSSVGYYT |
| | | | | VTGLEPGIDYDISVITLINGGESAPTTLTQQ |
| | | | | TAVPPPTDLRFTNIGPDTMRVTWAPPPSID |
| | | | | LTNFLVRYSPVKNEEDVAELSISPSDNAVV |
| | | | | LTNLLPGTEYVVSVSSVYEQHESTPLRGRQ |
| | | | | KTGLDSPTGIDFSDITANSFTVHWIAPRATI |
| | | | | TGYRIRHHPEHFSGRPREDRVPHSRNSITLT |
| | | | | NLTPGTEYVVSIVALNGREESPLLIGQQST |
| | | | | VSDVPRDLEVVAATPTSLLISWDAPAVTV |
| | | | | RYYRITYGETGGNSPVQEFTVPGSKSTATIS |
| | | | | GLKPGVDYTITVYAVTGRGDSPASSKPISIN |
| | | | | YRTEIDKPSQMQVTDVQDNSISVKWLPSSS |
| | | | | PVTGYRVTTTPKNGPGPTKTKTAGPDQTE |
| | | | | MTIEGLQPTVEYVVSVYAQNPSGESQPLV |
| | | | | QTAVTNIDRPKGLAFTDVDVDSIKIAWESP |
| | | | | QGQVSRYRVTYSSPEDGIHELFPAPDGEED |
| | | | | TAELQGLRPGSEYTVSVVALHDDMESQPLI |
| | | | | GTQSTAIPAPTDLKFTQVTPTSLSAQWTPP |
| | | | | NVQLTGYRVRVTPKEKTGPMKEINLAPDS |
| | | | | SSVVVSGLMVATKYEVSVYALKDTLTSRP |
| | | | | AQGVVTTLENVSPPRRARVTDATETTITIS |
| | | | | WRTKTETITGFQVDAVPANGQTPIQRTIKP |
| | | | | DVRSYTITGLQPGTDYKIYLYTLNDNARSS |
| | | | | PVVIDASTAIDAPSNLRFLATTPNSLLVSW |
| | | | | QPPRARITGYIIKYEKPGSPPREVVPRPRPG |
| | | | | VTEATITGLEPGTEYTIYVIALKNNQKSEPL |
| | | | | IGRKKTDELPQLVTLPHPNLHGPEILDVPST |
| | | | | VQKTPFVTHPGYDTGNGIQLPGTSGQQPSV |
| | | | | GQQMIFEEHGFRRTTPPTTATPIRHRPRPYP |
| | | | | PNVGEEIQIGHIPREDVDYHLYPHGPGLNP |
| | | | | NASTGQEALSQTTISWAPFQDTSEYIISCHP |
| | | | | VGTDEEPLQFRVPGTSTSATLTGLTRGATY |
| | | | | NVIVEALKDQQRHKVREEVVTVGNSVNE |
| | | | | GLNQPTDDSCFDPYTVSHYAVGDEWERM |
| | | | | SESGFKLLCQCLGFGSGHFRCDSSRWCHD |
| | | | | NGVNYKIGEKWDRQGENGQMMSCTCLG |
| | | | | NGKGEFKCDPHEATCYDDGKTYHVGEQW |
| | | | | QKEYLGAICSCTCFGGQRGWRCDNCRRPG |
| | | | | GEPSPEGTTGQSYNQYSQRYHQRTNTNVN |
| | | | | CPIECFMPLDVQADREDSRE |
| 36 | HEMO | Hemopexin | P02790 | MARVLGAPVALGLWSLCWSLAIATPLPPT |
| | | | | SAHGNVAEGETKPDPDVTERCSDGWSFDA |
| | | | | TTLDDNGTMLFFKGEFVWKSHKWDRELIS |
| | | | | ERWKNFPSPVDAAFRQGHNSVFLIKGDKV |
| | | | | WVYPPEKKEKGYPKLLQDEFPGIPSPLDAA |
| | | | | VECHRGECQAEGVLFFQGDREWFWDLAT |
| | | | | GTMKERSWPAVGNCSSALRWLGRYYCFQ |
| | | | | GNQFLRFDPVRGEVPPRYPRDVRDYFMPC |
| | | | | PGRGHGHRNGTGHGNSTHHGPEYMRCSP |
| | | | | HLVLSALTSDNHGATYAFSGTHYWRLDTS |
| | | | | RDGWHSWPIAHQWPQGPSAVDAAFSWEE |
| | | | | KLYLVQGTQVYVFLTKGGYTLVSGYPKRL |
| | | | | EKEVGTPHGIILDSVDAAFICPGSSRLHIMA |
| | | | | GRRLWWLDLKSGAQATWTELPWPHEKV |
| | | | | DGALCMEKSLGPNSCSANGPGLYLIHGPN |
| | | | | LYCYSDVEKLNAAKALPQPQNVTSLLGCT |
| | | | | H |
| 37 | IC1 | Plasma protease C1 | P05155 | MASRLTLLTLLLLLLAGDRASSNPNATSSS |
| | | inhibitor | | SQDPESLQDRGEGKVATTVISKMLFVEPIL |
| | | | | EVSSLPTTNSTTNSATKITANTTDEPTTQPT |
| | | | | TEPTTQPTIQPTQPTTQLPTDSPTQPTTGSFC |
| | | | | PGPVTLCSDLESHSTEAVLGDALVDFSLKL |
| | | | | YHAFSAMKKVETNMAFSPFSIASLLTQVLL |
| | | | | GAGENTKTNLESILSYPKDFTCVHQALKGF |
| | | | | TTKGVTSVSQIFHSPDLAIRDTFVNASRTLY |
| | | | | SSSPRVLSNNSDANLELINTWVAKNTNNKI |
| | | | | SRLLDSLPSDTRLVLLNAIYLSAKWKTTFD |
| | | | | PKKTRMEPFHFKNSVIKVPMMNSKKYPVA |
| | | | | HFIDQTLKAKVGQLQLSHNLSLVILVPQNL |
| | | | | KHRLEDMEQALSPSVFKAIMEKLEMSKFQ |
| | | | | PTLLTLPRIKVTTSQDMLSIMEKLEFFDFSY |
| | | | | DLNLCGLTEDPDLQVSAMQHQTVLELTET |
| | | | | GVEAAAASAISVARTLLVFEVQQPFLFVL |
| | | | | WDQQHKFPVFMGRVYDPRA |
| 38 | IGHM | Immunoglobulin | P01871 | GSASAPTLFPLVSCENSPSDTSSVAVGCLA |
| | | heavy constant mu | | QDFLPDSITFSWKYKNNSDISSTRGFPSVLR |
| | | | | GGKYAATSQVLLPSKDVMQGTDEHVVCK |
| | | | | VQHPNGNKEKNVPLPVIAELPPKVSVFVPP |
| | | | | RDGFFGNPRKSKLICQATGFSPRQIQVSWL |
| | | | | REGKQVGSGVTTDQVQAEAKESGPTTYK |
| | | | | VTSTLTIKESDWLGQSMFTCRVDHRGLTF |
| | | | | QQNASSMCVPDQDTAIRVFAIPPSFASIFLT |
| | | | | KSTKLTCLVTDLTTYDSVTISWTRQNGEA |
| | | | | VKTHTNISESHPNATFSAVGEASICEDDWN |
| | | | | SGERFTCTVTHTDLPSPLKQTISRPKGVAL |
| | | | | HRPDVYLLPPAREQLNLRESATITCLVTGF |
| | | | | SPADVFVQWMQRGQPLSPEKYVTSAPMPE |
| | | | | PQAPGRYFAHSILTVSEEEWNTGETYTCVV |
| | | | | AHEALPNRVTERTVDKSTGKPTLYNVSLV |
| | | | | MSDTAGTCY |
| 39 | ITIH1 | Inter-alpha-trypsin | P19827 | MDGAMGPRGLLLCMYLVSLLILQAMPAL |
| | | inhibitor heavy | | GSATGRSKSSEKRQAVDTAVDGVFIRSLK |
| | | chain H1 | | VNCKVTSRFAHYVVTSQVVNTANEAREV |
| | | | | AFDLEIPKTAFISDFAVTADGNAFIGDIKDK |
| | | | | VTAWKQYRKAAISGENAGLVRASGRTME |
| | | | | QFTIHLTVNPQSKVTFQLTYEEVLKRNHM |
| | | | | QYEIVIKVKPKQLVHHFEIDVDIFEPQGISK |
| | | | | LDAQASFLPKELAAQTIKKSFSGKKGHVLF |
| | | | | RPTVSQQQSCPTCSTSLLNGHFKVTYDVSR |
| | | | | DKICDLLVANNHFAHFFAPQNLTNMNKNV |
| | | | | VFVIDISGSMRGQKVKQTKEALLKILGDM |
| | | | | QPGDYFDLVLFGTRVQSWKGSLVQASEAN |
| | | | | LQAAQDFVRGFSLDEATNLNGGLLRGIEIL |
| | | | | NQVQESLPELSNHASILIMLTDGDPTEGVT |
| | | | | DRSQILKNVRNAIRGRFPLYNLGFGHNVDF |
| | | | | NFLEVMSMENNGRAQRIYEDHDATQQLQ |
| | | | | GFYSQVAKPLLVDVDLQYPQDAVLALTQ |
| | | | | NHHKQYYEGSEIVVAGRIADNKQSSFKAD |
| | | | | VQAHGEGQEFSITCLVDEEEMKKLLRERG |
| | | | | HMLENHVERLWAYLTIQELLAKRMKVDR |
| | | | | EERANLSSQALQMSLDYGFVTPLTSMSIRG |
| | | | | MADQDGLKPTIDKPSEDSPPLEMLGPRRTF |
| | | | | VLSALQPSPTHSSSNTQRLPDRVTGVDTDP |
| | | | | HFIIHVPQKEDTLCFNINEEPGVILSLVQDP |
| | | | | NTGFSVNGQLIGNKARSPGQHDGTYFGRL |
| | | | | GIANPATDFQLEVTPQNITLNPGFGGPVFS |
| | | | | WRDQAVLRQDGVVVTINKKRNLVVSVDD |
| | | | | GGTFEVVLHRVWKGSSVHQDFLGFYVLDS |
| | | | | HRMSARTHGLLGQFFHPIGFEVSDIHPGSD |
| | | | | PTKPDATMVVRNRRLTVTRGLQKDYSKDP |
| | | | | WHGAEVSCWFIHNNGAGLIDGAYTDYIVP |
| | | | | DIF |
| 40 | MYCT | Proton myo- | Q96QE2 | MSRKASENVEYTLRSLSSLMGERRRKQPE |
| | | inositol | | PDAASAAGECSLLAAAESSTSLQSAGAGG |
| | | cotransporter | | GGVGDLERAARRQFQQDETPAFVYVVAV |
| | | | | FSALGGFLFGYDTGVVSGAMLLLKRQLSL |
| | | | | DALWQELLVSSTVGAAAVSALAGGALNG |
| | | | | VFGRRAAILLASALFTAGSAVLAAANNKE |
| | | | | TLLAGRLVVGLGIGIASMTVPVYIAEVSPP |
| | | | | NLRGRLVTINTLFITGGQFFASVVDGAFSY |
| | | | | LQKDGWRYMLGLAAVPAVIQFFGFLFLPE |
| | | | | SPRWLIQKGQTQKARRILSQMRGNQTIDEE |
| | | | | YDSIKNNIEEEEKEVGSAGPVICRMLSYPPT |
| | | | | RRALIVGCGLQMFQQLSGINTIMYYSATIL |
| | | | | QMSGVEDDRLAIWLASVTAFTNFIFTLVG |
| | | | | VWLVEKVGRRKLTFGSLAGTTVALIILALG |
| | | | | FVLSAQVSPRITFKPIAPSGQNATCTRYSYC |
| | | | | NECMLDPDCGFCYKMNKSTVIDSSCVPVN |
| | | | | KASTNEAAWGRCENETKFKTEDIFWAYNF |
| | | | | CPTPYSWTALLGLILYLVFFAPGMGPMPW |
| | | | | TVNSEIYPLWARSTGNACSSGINWIFNVLV |
| | | | | SLTFLHTAEYLTYYGAFFLYAGFAAVGLLF |
| | | | | IYGCLPETKGKKLEEIESLFDNRLCTCGTSD |
| | | | | SDEGRYIEYIRVKGSNYHLSDNDASDVE |
| 41 | PHLD | Phosphatidylinositol- | P80108 | MSAFRLWPGLLIMLGSLCHRGSPCGLSTH |
| | | glycan-specific | | VEIGHRALEFLQLHNGRVNYRELLLEHQD |
| | | phospholipase D | | AYQAGIVFPDCFYPSICKGGKFHDVSESTH |
| | | | | WTPFLNASVHYIRENYPLPWEKDTEKLVA |
| | | | | FLFGITSHMAADVSWHSLGLEQGFLRTMG |
| | | | | AIDFHGSYSEAHSAGDFGGDVLSQFEFNFN |
| | | | | YLARRWYVPVKDLLGIYEKLYGRKVITEN |
| | | | | VIVDCSHIQFLEMYGEMLAVSKLYPTYSTK |
| | | | | SPFLVEQFQEYFLGGLDDMAFWSTNIYHL |
| | | | | TSFMLENGTSDCNLPENPLFIACGGQQNHT |
| | | | | QGSKMQKNDFHRNLTTSLTESVDRNINYT |
| | | | | ERGVFFSVNSWTPDSMSFIYKALERNIRTM |
| | | | | FIGGSQLSQKHVSSPLASYFLSFPYARLGW |
| | | | | AMTSADLNQDGHGDLVVGAPGYSRPGHI |
| | | | | HIGRVYLIYGNDLGLPPVDLDLDKEAHRIL |
| | | | | EGFQPSGRFGSALAVLDFNVDGVPDLAVG |
| | | | | APSVGSEQLTYKGAVYVYFGSKQGGMSSS |
| | | | | PNITISCQDIYCNLGWTLLAADVNGDSEPD |
| | | | | LVIGSPFAPGGGKQKGIVAAFYSGPSLSDK |
| | | | | EKLNVEAANWTVRGEEDFSWFGYSLHGV |
| | | | | TVDNRTLLLVGSPTWKNASRLGHLLHIRD |
| | | | | EKKSLGRVYGYFPPNGQSWFTISGDKAMG |
| | | | | KLGTSLSSGHVLMNGTLKQVLLVGAPTYD |
| | | | | DVSKVAFLTVTLHQGGATRMYALTSDAQ |
| | | | | PLLLSTFSGDRRFSRFGGVLHLSDLDDDGL |
| | | | | DEIIMAAPLRIADVTSGLIGGEDGRVYVYN |
| | | | | GKETTLGDMTGKCKSWITPCPEEKAQYVL |
| | | | | ISPEASSRFGSSLITVRSKAKNQVVIAAGRS |
| | | | | SLGARLSGALHVYSLGSD |
| 42 | PSG1 | Pregnancy-specific | P11464 | MGTLSAPPCTQRIKWKGLLLTASLLNFWN |
| | | beta-1-glycoprotein | | LPTTAQVTIEAEPTKVSEGKDVLLLVHNLP |
| | | 1 | | QNLTGYIWYKGQMRDLYHYITSYVVDGEI |
| | | | | IIYGPAYSGRETAYSNASLLIQNVTREDAG |
| | | | | SYTLHIIKGDDGTRGVTGRFTFTLHLETPKP |
| | | | | SISSSNLNPRETMEAVSLTCDPETPDASYL |
| | | | | WWMNGQSLPMTHSLKLSETNRTLFLLGV |
| | | | | TKYTAGPYECEIRNPVSASRSDPVTLNLLP |
| | | | | KLPKPYITINNLNPRENKDVLNFTCEPKSE |
| | | | | NYTYIWWLNGQSLPVSPRVKRPIENRILILP |
| | | | | SVTRNETGPYQCEIRDRYGGIRSDPVTLNV |
| | | | | LYGPDLPRIYPSFTYYRSGEVLYLSCSADS |
| | | | | NPPAQYSWTINEKFQLPGQKLFIRHITTKHS |
| | | | | GLYVCSVRNSATGKESSKSMTVEVSDWTV |
| | | | | P |
| 43 | PSG7 | Putative | Q13046 | MGPLSAPPCTQHITWKGLLLTASLLNFWN |
| | | Pregnancy-specific | | PPTTAQVTIEAQPPKVSEGKDVLLLVHNLP |
| | | beta-1-glycoprotein | | QNLTGYIWYKGQIRDLYHYVTSYIVDGQII |
| | | 7 | | KYGPAYSGRETVYSNASLLIQNVTQEDTG |
| | | | | SYTLHIIKRGDGTGGVTGRFTFTLYLETPKP |
| | | | | SISSSNFNPREATEAVILTCDPETPDASYLW |
| | | | | WMNGQSLPMTHSLQLSETNRTLYLFGVTN |
| | | | | YTAGPYECEIRNPVSASRSDPVTLNLLPKL |
| | | | | PKPYITINNLNPRENKDVSTFTCEPKSENYT |
| | | | | YIWWLNGQSLPVSPRVKRRIENRILILPSVT |
| | | | | RNETGPYQCEIRDRYGGIRSDPVTLNVLYG |
| | | | | PDLPRIYPSFTYYHSGQNLYLSCFADSNPP |
| | | | | AQYSWTINGKFQLSGQKLSIPQITTKHSGL |
| | | | | YACSVRNSATGKESSKSVTVRVSDWTLP |
| 44 | PZP | Pregnancy zone | P20742 | MRKDRLLHLCLVLLLILLSASDSNSTEPQY |
| | | protein | | MVLVPSLLHTEAPKKGCVLLSHLNETVTV |
| | | | | SASLESGRENRSLFTDLVAEKDLFHCVSFT |
| | | | | LPRISASSEVAFLSIQIKGPTQDFRKRNTVL |
| | | | | VLNTQSLVFVQTDKPMYKPGQTVRFRVVS |
| | | | | VDENFRPRNELIPLIYLENPRRNRIAQWQSL |
| | | | | KLEAGINQLSFPLSSEPIQGSYRVVVQTESG |
| | | | | GRIQHPFTVEEFVLPKFEVKVQVPKIISIMD |
| | | | | EKVNITVCGEYTYGKPVPGLATVSLCRKLS |
| | | | | RVLNCDKQEVCEEFSQQLNSNGCITQQVH |
| | | | | TKMLQITNTGFEMKLRVEARIREEGTDLEV |
| | | | | TANRISEITNIVSKLKFVKVDSHFRQGIPFF |
| | | | | AQVLLVDGKGVPIPNKLFFISVNDANYYSN |
| | | | | ATTNEQGLAQFSINTTSISVNKLFVRVFTV |
| | | | | HPNLCFHYSWVAEDHQGAQHTANRVFSL |
| | | | | SGSYIHLEPVAGTLPCGHTETITAHYTLNR |
| | | | | QAMGELSELSFHYLIMAKGVIVRSGTHTLP |
| | | | | VESGDMKGSFALSFPVESDVAPIARMFIFAI |
| | | | | LPDGEVVGDSEKFEIENCLANKVDLSFSPA |
| | | | | QSPPASHAHLQVAAAPQSLCALRAVDQSV |
| | | | | LLMKPEAELSVSSVYNLLTVKDLTNFPDN |
| | | | | VDQQEEEQGHCPRPFFIHNGAIYVPLSSNE |
| | | | | ADIYSFLKGMGLKVFTNSKIRKPKSCSVIPS |
| | | | | VSAGAVGQGYYGAGLGVVERPYVPQLGT |
| | | | | YNVIPLNNEQSSGPVPETVRSYFPETWIWE |
| | | | | LVAVNSSGVAEVGVTVPDTITEWKAGAFC |
| | | | | LSEDAGLGISSTASLRAFQPFFVELTMPYSV |
| | | | | IRGEVFTLKATVLNYLPKCIRVSVQLKASP |
| | | | | AFLASQNTKGEESYCICGNERQTLSWTVTP |
| | | | | KTLGNVNFSVSAEAMQSLELCGNEVVEVP |
| | | | | EIKRKDTVIKTLLVEAEGIEQEKTFSSMTCA |
| | | | | SGANVSEQLSLKLPSNVVKESARASFSVLG |
| | | | | DILGSAMQNIQNLLQMPYGCGEQNMVLFA |
| | | | | PNIYVLNYLNETQQLTQEIKAKAVGYLITG |
| | | | | YQRQLNYKHQDGSYSTFGERYGRNQGNT |
| | | | | WLTAFVLKTFAQARSYIFIDEAHITQSLTW |
| | | | | LSQMQKDNGCFRSSGSLLNNAIKGGVEDE |
| | | | | ATLSAYVTIALLEIPLPVTNPIVRNALFCLE |
| | | | | SAWNVAKEGTHGSHVYTKALLAYAFSLL |
| | | | | GKQNQNREILNSLDKEAVKEDNLVHWERP |
| | | | | QRPKAPVGHLYQTQAPSAEVEMTSYVLLA |
| | | | | YLTAQPAPTSGDLTSATNIVKWIMKQQNA |
| | | | | QGGFSSTQDTVVALHALSRYGAATFTRTE |
| | | | | KTAQVTVQDSQTFSTNFQVDNNNLLLLQQ |
| | | | | ISLPELPGEYVITVTGERCVYLQTSMKYNIL |
| | | | | PEKEDSPFALKVQTVPQTCDGHKAHTSFQI |
| | | | | SLTISYTGNRPASNMVIVDVKMVSGFIPLK |
| | | | | PTVKMLERSSSVSRTEVSNNHVLIYVEQVT |
| | | | | NQTLSFSFMVLQDIPVGDLKPAIVKVYDY |
| | | | | YETDESVVAEYIAPCSTDTEHGNV |
| 45 | SHBG | Sex hormone- | P04278 | MESRGPLATSRLLLLLLLLLLRHTRQGWA |
| | | binding globulin | | LRPVLPTQSAHDPPAVHLSNGPGQEPIAVM |
| | | | | TFDLTKITKTSSSFEVRTWDPEGVIFYGDT |
| | | | | NPKDDWFMLGLRDGRPEIQLHNHWAQLT |
| | | | | VGAGPRLDDGRWHQVEVKMEGDSVLLEV |
| | | | | DGEEVLRLRQVSGPLTSKRHPIMRIALGGL |
| | | | | LFPASNLRLPLVPALDGCLRRDSWLDKQA |
| | | | | EISASAPTSLRSCDVESNPGIFLPPGTQAEF |
| | | | | NLRDIPQPHAEPWAFSLDLGLKQAAGSGH |
| | | | | LLALGTPENPSWLSLHLQDQKVVLSSGSGP |
| | | | | GLDLPLVLGLPLQLKLSMSRVVLSQGSKM |
| | | | | KALALPPLGLAPLLNLWAKPQGRLFLGAL |
| | | | | PGEDSSTSFCLNGLWAQGQRLDVDQALNR |
| | | | | SHEIWTHSCPQSPGNGTDASH |
| 46 | TRFE | Serotransferrin | P02787 | MRLAVGALLVCAVLGLCLAVPDKTVRWC |
| | | | | AVSEHEATKCQSFRDHMKSVIPSDGPSVA |
| | | | | CVKKASYLDCIRAIAANEADAVTLDAGLV |
| | | | | YDAYLAPNNLKPVVAEFYGSKEDPQTFYY |
| | | | | AVAVVKKDSGFQMNQLRGKKSCHTGLGR |
| | | | | SAGWNIPIGLLYCDLPEPRKPLEKAVANFF |
| | | | | SGSCAPCADGTDFPQLCQLCPGCGCSTLN |
| | | | | QYFGYSGAFKCLKDGAGDVAFVKHSTIFE |
| | | | | NLANKADRDQYELLCLDNTRKPVDEYKD |
| | | | | CHLAQVPSHTVVARSMGGKEDLIWELLNQ |
| | | | | AQEHFGKDKSKEFQLFSSPHGKDLLFKDS |
| | | | | AHGFLKVPPRMDAKMYLGYEYVTAIRNL |
| | | | | REGTCPEAPTDECKPVKWCALSHHERLKC |
| | | | | DEWSVNSVGKIECVSAETTEDCIAKIMNGE |
| | | | | ADAMSLDGGFVYIAGKCGLVPVLAENYN |
| | | | | KSDNCEDTPEAGYFAIAVVKKSASDLTWD |
| | | | | NLKGKKSCHTAVGRTAGWNIPMGLLYNKI |
| | | | | NHCRFDEFFSEGCAPGSKKDSSLCKLCMG |
| | | | | SGLNLCEPNNKEGYYGYTGAFRCLVEKGD |
| | | | | VAFVKHQTVPQNTGGKNPDPWAKNLNEK |
| | | | | DYELLCLDGTRKPVEEYANCHLARAPNHA |
| | | | | VVTRKDKEACVHKILRQQQHLFGSNVTDC |
| | | | | SGNFCLFRSETKDLLFRDDTVCLAKLHDR |
| | | | | NTYEKYLGEEYVKAVGNLRKCSTSSLLEA |
| | | | | CTFRRP |
| 47 | VTNC | Vitronectin | P04004 | MAPLRPLLILALLAWVALADQESCKGRCT |
| | | | | EGFNVDKKCQCDELCSYYQSCCTDYTAEC |
| | | | | KPQVTRGDVFTMPEDEYTVYDDGEEKNN |
| | | | | ATVHEQVGGPSLTSDLQAQSKGNPEQTPV |
| | | | | LKPEEEAPAPEVGASKPEGIDSRPETLHPGR |
| | | | | PQPPAEEELCSGKPFDAFTDLKNGSLFAFR |
| | | | | GQYCYELDEKAVRPGYPKLIRDVWGIEGPI |
| | | | | DAAFTRINCQGKTYLFKGSQYWRFEDGVL |
| | | | | DPDYPRNISDGFDGIPDNVDAALALPAHSY |
| | | | | SGRERVYFFKGKQYWEYQFQHQPSQEECE |
| | | | | GSSLSAVFEHFAMMQRDSWEDIFELLFWG |
| | | | | RTSAGTRQPQFISRDWHGVPGQVDAAMA |
| | | | | GRIYISGMAPRPSLAKKQRFRHRNRKGYRS |
| | | | | QRGHSRGRNQNSRRPSRATWLSLFSSEESN |
| | | | | LGANNYDDYRMDWLVPATCEPIQSVFFFS |
| | | | | GDKYYRVNLRTRRVDTVDPPYPRSIAQYW |
| | | | | LGCPAPGHL |
| 48 | ZA2G | Zinc-alpha-2- | P25311 | MVRMVPVLLSLLLLLGPAVPQENQDGRYS |
| | | glycoprotein | | LTYIYTGLSKHVEDVPAFQALGSLNDLQFF |
| | | | | RYNSKDRKSQPMGLWRQVEGMEDWKQD |
| | | | | SQLQKAREDIFMETLKDIVEYYNDSNGSH |
| | | | | VLQGRFGCEIENNRSSGAFWKYYYDGKD |
| | | | | YIEFNKEIPAWVPFDPAAQITKQKWEAEPV |
| | | | | YVQRAKAYLEEECPATLRKYLKYSKNILD |
| | | | | RQDPPSVVVTSHQAPGEKKKLKCLAYDFY |
| | | | | PGKIDVHWTRAGEVQEPELRGDVLHNGN |
| | | | | GTYQSWVVVAVPPQDTAPYSCHVQHSSL |
| | | | | AQPLVVPWEAS |
| 49 | AFAM | Afamin | P43652 | MKLLKLTGFIFFLFFLTESLTLPTQPRDIENF |
| | | | | NSTQKFIEDNIEYITIIAFAQYVQEATFEEM |
| | | | | EKLVKDMVEYKDRCMADKTLPECSKLPN |
| | | | | NVLQEKICAMEGLPQKHNFSHCCSKVDAQ |
| | | | | RRLCFFYNKKSDVGFLPPFPTLDPEEKCQA |
| | | | | YESNRESLLNHFLYEVARRNPFVFAPTLLT |
| | | | | VAVHFEEVAKSCCEEQNKVNCLQTRAIPV |
| | | | | TQYLKAFSSYQKHVCGALLKFGTKVVHFI |
| | | | | YIAILSQKFPKIEFKELISLVEDVSSNYDGC |
| | | | | CEGDVVQCIRDTSKVMNHICSKQDSISSKI |
| | | | | KECCEKKIPERGQCIINSNKDDRPKDLSLRE |
| | | | | GKFTDSENVCQERDADPDTFFAKFTFEYSR |
| | | | | RHPDLSIPELLRIVQIYKDLLRNCCNTENPP |
| | | | | GCYRYAEDKFNETTEKSLKMVQQECKHF |
| | | | | QNLGKDGLKYHYLIRLTKIAPQLSTEELVS |
| | | | | LGEKMVTAFTTCCTLSEEFACVDNLADLV |
| | | | | FGELCGVNENRTINPAVDHCCKTNFAFRRP |
| | | | | CFESLKADKTYVPPPFSQDLFTFHADMCQS |
| | | | | QNEELQRKTDRFLVNLVKLKHELTDEELQ |
| | | | | SLFTNFANVVDKCCKAESPEVCFNEESPKI |
| | | | | GN |
| 50 | CFAI | Complement factor | P05156 | MKLLHVFLLFLCFHLRFCKVTYTSQEDLV |
| | | I | | EKKCLAKKYTHLSCDKVFCQPWQRCIEGT |
| | | | | CVCKLPYQCPKNGTAVCATNRRSFPTYCQ |
| | | | | QKSLECLHPGTKFLNNGTCTAEGKFSVSLK |
| | | | | HGNTDSEGIVEVKLVDQDKTMFICKSSWS |
| | | | | MREANVACLDLGFQQGADTQRRFKLSDLS |
| | | | | INSTECLHVHCRGLETSLAECTFTKRRTMG |
| | | | | YQDFADVVCYTQKADSPMDDFFQCVNGK |
| | | | | YISQMKACDGINDCGDQSDELCCKACQGK |
| | | | | GFHCKSGVCIPSQYQCNGEVDCITGEDEVG |
| | | | | CAGFASVTQEETEILTADMDAERRRIKSLL |
| | | | | PKLSCGVKNRMHIRRKRIVGGKRAQLGDL |
| | | | | PWQVAIKDASGITCGGIYIGGCWILTAAHC |
| | | | | LRASKTHRYQIWTTVVDWIHPDLKRIVIEY |
| | | | | VDRIIFHENYNAGTYQNDIALIEMKKDGN |
| | | | | KKDCELPRSIPACVPWSPYLFQPNDTCIVS |
| | | | | GWGREKDNERVFSLQWGEVKLISNCSKFY |
| | | | | GNRFYEKEMECAGTYDGSIDACKGDSGGP |
| | | | | LVCMDANNVTYVWGVVSWGENCGKPEF |
| | | | | PGVYTKVANYFDWISYHVGRPFISQYNV |
| 51 | CO3 | Complement C3 | P01024 | MGPTSGPSLLLLLLTHLPLALGSPMYSIITP |
| | | | | NILRLESEETMVLEAHDAQGDVPVTVTVH |
| | | | | DFPGKKLVLSSEKTVLTPATNHMGNVTFTI |
| | | | | PANREFKSEKGRNKFVTVQATFGTQVVEK |
| | | | | VVLVSLQSGYLFIQTDKTIYTPGSTVLYRIF |
| | | | | TVNHKLLPVGRTVMVNIENPEGIPVKQDSL |
| | | | | SSQNQLGVLPLSWDIPELVNMGQWKIRAY |
| | | | | YENSPQQVFSTEFEVKEYVLPSFEVIVEPTE |
| | | | | KFYYIYNEKGLEVTITARFLYGKKVEGTAF |
| | | | | VIFGIQDGEQRISLPESLKRIPIEDGSGEVVL |
| | | | | SRKVLLDGVQNPRAEDLVGKSLYVSATVI |
| | | | | LHSGSDMVQAERSGIPIVTSPYQIHFTKTPK |
| | | | | YFKPGMPFDLMVFVTNPDGSPAYRVPVAV |
| | | | | QGEDTVQSLTQGDGVAKLSINTHPSQKPLS |
| | | | | ITVRTKKQELSEAEQATRTMQALPYSTVG |
| | | | | NSNNYLHLSVLRTELRPGETLNVNFLLRM |
| | | | | DRAHEAKIRYYTYLIMNKGRLLKAGRQVR |
| | | | | EPGQDLVVLPLSITTDFIPSFRLVAYYTLIG |
| | | | | ASGQREVVADSVWVDVKDSCVGSLVVKS |
| | | | | GQSEDRQPVPGQQMTLKIEGDHGARVVLV |
| | | | | AVDKGVFVLNKKNKLTQSKIWDVVEKAD |
| | | | | IGCTPGSGKDYAGVFSDAGLTFTSSSGQQT |
| | | | | AQRAELQCPQPAARRRRSVQLTEKRMDK |
| | | | | VGKYPKELRKCCEDGMRENPMRFSCQRR |
| | | | | TRFISLGEACKKVELDCCNYITELRRQHAR |
| | | | | ASHLGLARSNLDEDIIAEENIVSRSEFPESW |
| | | | | LWNVEDLKEPPKNGISTKLMNIFLKDSITT |
| | | | | WEILAVSMSDKKGICVADPFEVTVMQDFFI |
| | | | | DLRLPYSVVRNEQVEIRAVLYNYRQNQEL |
| | | | | KVRVELLHNPAFCSLATTKRRHQQTVTIPP |
| | | | | KSSLSVPYVIVPLKTGLQEVEVKAAVYHH |
| | | | | FISDGVRKSLKVVPEGIRMNKTVAVRTLDP |
| | | | | ERLGREGVQKEDIPPADLSDQVPDTESETR |
| | | | | ILLQGTPVAQMTEDAVDAERLKHLIVTPSG |
| | | | | CGEQNMIGMTPTVIAVHYLDETEQWEKFG |
| | | | | LEKRQGALELIKKGYTQQLAFRQPSSAFAA |
| | | | | FVKRAPSTWLTAYVVKVFSLAVNLIAIDSQ |
| | | | | VLCGAVKWLILEKQKPDGVFQEDAPVIHQ |
| | | | | EMIGGLRNNNEKDMALTAFVLISLQEAKDI |
| | | | | CEEQVNSLPGSITKAGDFLEANYMNLQRS |
| | | | | YTVAIAGYALAQMGRLKGPLLNKFLTTAK |
| | | | | DKNRWEDPGKQLYNVEATSYALLALLQL |
| | | | | KDFDFVPPVVRWLNEQRYYGGGYGSTQA |
| | | | | TFMVFQALAQYQKDAPDHQELNLDVSLQ |
| | | | | LPSRSSKITHRIHWESASLLRSEETKENEGF |
| | | | | TVTAEGKGQGTLSVVTMYHAKAKDQLTC |
| | | | | NKFDLKVTIKPAPETEKRPQDAKNTMILEI |
| | | | | CTRYRGDQDATMSILDISMMTGFAPDTDD |
| | | | | LKQLANGVDRYISKYELDKAFSDRNTLIIY |
| | | | | LDKVSHSEDDCLAFKVHQYFNVELIQPGA |
| | | | | VKVYAYYNLEESCTRFYHPEKEDGKLNKL |
| | | | | CRDELCRCAEENCFIQKSDDKVTLEERLDK |
| | | | | ACEPGVDYVYKTRLVKVQLSNDFDEYIMA |
| | | | | IEQTIKSGSDEVQVGQQRTFISPIKCREALK |
| | | | | LEEKKHYLMWGLSSDFWGEKPNLSYIIGK |
| | | | | DTWVEHWPEEDECQDEENQKQCQDLGAF |
| | | | | TESMVVFGCPN |
| 52 | FETUA | Alpha-2-HS- | P02765 | MKSLVLLLCLAQLWGCHSAPHGPGLIYRQ |
| | | glycoprotein | | PNCDDPETEEAALVAIDYINQNLPWGYKH |
| | | | | TLNQIDEVKVWPQQPSGELFEIEIDTLETTC |
| | | | | HVLDPTPVARCSVRQLKEHAVEGDCDFQL |
| | | | | LKLDGKFSVVYAKCDSSPDSAEDVRKVCQ |
| | | | | DCPLLAPLNDTRVVHAAKAALAAFNAQN |
| | | | | NGSNFQLEEISRAQLVPLPPSTYVEFTVSGT |
| | | | | DCVAKEATEAAKCNLLAEKQYGFCKATLS |
| | | | | EKLGGAEVAVTCMVFQTQPVSSQPQPEGA |
| | | | | NEAVPTPVVDPDAPPSPPLGAPGLPPAGSP |
| | | | | PDSHVLLAAPPGHQLHRAHYDLRHTFMG |
| | | | | VVSLGSPSGEVSHPRKTRTVVQPSVGAAA |
| | | | | GPVVPPCPGRIRHFKV |
| 53 | HEP2 | Heparin cofactor 2 | P05546 | MKHSLNALLIFLIITSAWGGSKGPLDQLEK |
| | | | | GGETAQSADPQWEQLNNKNLSMPLLPADF |
| | | | | HKENTVINDWIPEGEEDDDYLDLEKIFSED |
| | | | | DDYIDIVDSLSVSPTDSDVSAGNILQLFHG |
| | | | | KSRIQRLNILNAKFAFNLYRVLKDQVNTFD |
| | | | | NIFIAPVGISTAMGMISLGLKGETHEQVHSI |
| | | | | LHFKDFVNASSKYEITTIHNLFRKLTHRLFR |
| | | | | RNFGYTLRSVNDLYIQKQFPILLDFKTKVR |
| | | | | EYYFAEAQIADFSDPAFISKTNNHIMKLTK |
| | | | | GLIKDALENIDPATQMMILNCIYFKGSWVN |
| | | | | KFPVEMTHNHNFRLNEREVVKVSMMQTK |
| | | | | GNFLAANDQELDCDILQLEYVGGISMLIVV |
| | | | | PHKMSGMKTLEAQLTPRVVERWQKSMTN |
| | | | | RTREVLLPKFKLEKNYNLVESLKLMGIRM |
| | | | | LFDKNGNMAGISDQRIAIDLFKHQGTITVN |
| | | | | EEGTQATTVTTVGFMPLSTQVRFTVDRPFL |
| | | | | FLIYEHRTSCLLFMGRVANPSRS |
| 54 | HPT | Haptoglobin | P00738 | MSALGAVIALLLWGQLFAVDSGNDVTDIA |
| | | | | DDGCPKPPEIAHGYVEHSVRYQCKNYYKL |
| | | | | RTEGDGVYTLNDKKQWINKAVGDKLPEC |
| | | | | EADDGCPKPPEIAHGYVEHSVRYQCKNYY |
| | | | | KLRTEGDGVYTLNNEKQWINKAVGDKLP |
| | | | | ECEAVCGKPKNPANPVQRILGGHLDAKGS |
| | | | | FPWQAKMVSHHNLTTGATLINEQWLLTTA |
| | | | | KNLFLNHSENATAKDIAPTLTLYVGKKQL |
| | | | | VEIEKVVLHPNYSQVDIGLIKLKQKVSVNE |
| | | | | RVMPICLPSKDYAEVGRVGYVSGWGRNA |
| | | | | NFKFTDHLKYVMLPVADQDQCIRHYEGST |
| | | | | VPEKKTPKSPVGVQPILNEHTFCAGMSKY |
| | | | | QEDTCYGDAGSAFAVHDLEEDTWYATGIL |
| | | | | SFDKSCAVAEYGVYVKVTSIQDWVQKTIA |
| | | | | EN |
| 55 | IGHA1 | Immunoglobulin | P01876 | ASPTSPKVFPLSLCSTQPDGNVVIACLVQG |
| | | heavy constant | | FFPQEPLSVTWSESGQGVTARNFPPSQDAS |
| | | alpha 1 | | GDLYTTSSQLTLPATQCLAGKSVTCHVKH |
| | | | | YTNPSQDVTVPCPVPSTPPTPSPSTPPTPSPS |
| | | | | CCHPRLSLHRPALEDLLLGSEANLTCTLTG |
| | | | | LRDASGVTFTWTPSSGKSAVQGPPERDLC |
| | | | | GCYSVSSVLPGCAEPWNHGKTFTCTAAYP |
| | | | | ESKTPLTATLSKSGNTFRPEVHLLPPPSEEL |
| | | | | ALNELVTLTCLARGFSPKDVLVRWLQGSQ |
| | | | | ELPREKYLTWASRQEPSQGTTTFAVTSILR |
| | | | | VAAEDWKKGDTFSCMVGHEALPLAFTQK |
| | | | | TIDRLAGKPTHVNVSVVMAEVDGTCY |
| 56 | IGHG2 | Immunoglobulin | P01859 | ASTKGPSVFPLAPCSRSTSESTAALGCLVK |
| | | heavy constant | | DYFPEPVTVSWNSGALTSGVHTFPAVLQSS |
| | | gamma 2 | | GLYSLSSVVTVPSSNFGTQTYTCNVDHKPS |
| | | | | NTKVDKTVERKCCVECPPCPAPPVAGPSV |
| | | | | FLFPPKPKDTLMISRTPEVTCVVVDVSHED |
| | | | | PEVQFNWYVDGVEVHNAKTKPREEQFNS |
| | | | | TFRVVSVLTVVHQDWLNGKEYKCKVSNK |
| | | | | GLPAPIEKTISKTKGQPREPQVYTLPPSREE |
| | | | | MTKNQVSLTCLVKGFYPSDISVEWESNGQ |
| | | | | PENNYKTTPPMLDSDGSFFLYSKLTVDKSR |
| | | | | WQQGNVFSCSVMHEALHNHYTQKSLSLSP |
| | | | | GK |
| 57 | IGJ | Immunoglobulin J | P01591 | MKNHLLFWGVLAVFIKAVHVKAQEDERI |
| | | chain | | VLVDNKCKCARITSRIIRSSEDPNEDIVERN |
| | | | | IRIIVPLNNRENISDPTSPLRTRFVYHLSDLC |
| | | | | KKCDPTEVELDNQIVTATQSNICDEDSATE |
| | | | | TCYTYDRNKCYTAVVPLVYGGETKMVET |
| | | | | ALTPDACYPD |
| 58 | KLKB1 | Plasma kallikrein | P03952 | MILFKQATYFISLFATVSCGCLTQLYENAF |
| | | | | FRGGDVASMYTPNAQYCQMRCTFHPRCL |
| | | | | LFSFLPASSINDMEKRFGCFLKDSVTGTLP |
| | | | | KVHRTGAVSGHSLKQCGHQISACHRDIYK |
| | | | | GVDMRGVNFNVSKVSSVEECQKRCTNNIR |
| | | | | CQFFSYATQTFHKAEYRNNCLLKYSPGGT |
| | | | | PTAIKVLSNVESGFSLKPCALSEIGCHMNIF |
| | | | | QHLAFSDVDVARVLTPDAFVCRTICTYHP |
| | | | | NCLFFTFYTNVWKIESQRNVCLLKTSESGT |
| | | | | PSSSTPQENTISGYSLLTCKRTLPEPCHSKIY |
| | | | | PGVDFGGEELNVTFVKGVNVCQETCTKMI |
| | | | | RCQFFTYSLLPEDCKEEKCKCFLRLSMDGS |
| | | | | PTRIAYGTQGSSGYSLRLCNTGDNSVCTTK |
| | | | | TSTRIVGGTNSSWGEWPWQVSLQVKLTAQ |
| | | | | RHLCGGSLIGHQWVLTAAHCFDGLPLQDV |
| | | | | WRIYSGILNLSDITKDTPFSQIKEIIIHQNYK |
| | | | | VSEGNHDIALIKLQAPLNYTEFQKPICLPSK |
| | | | | GDTSTIYTNCWVTGWGFSKEKGEIQNILQ |
| | | | | KVNIPLVTNEECQKRYQDYKITQRMVCAG |
| | | | | YKEGGKDACKGDSGGPLVCKHNGMWRL |
| | | | | VGITSWGEGCARREQPGVYTKVAEYMDW |
| | | | | ILEKTQSSDGKAQMQSPA |
| 59 | THBG | Thyroxine-binding | P05543 | MSPFLYLVLLVLGLHATIHCASPEGKVTAC |
| | | globulin | | HSSQPNATLYKMSSINADFAFNLYRRFTVE |
| | | | | TPDKNIFFSPVSISAALVMLSFGACCSTQTE |
| | | | | IVETLGFNLTDTPMVEIQHGFQHLICSLNFP |
| | | | | KKELELQIGNALFIGKHLKPLAKFLNDVKT |
| | | | | LYETEVFSTDFSNISAAKQEINSHVEMQTK |
| | | | | GKVVGLIQDLKPNTIMVLVNYIHFKAQWA |
| | | | | NPFDPSKTEDSSSFLIDKTTTVQVPMMHQ |
| | | | | MEQYYHLVDMELNCTVLQMDYSKNALA |
| | | | | LFVLPKEGQMESVEAAMSSKTLKKWNRL |
| | | | | LQKGWVDLFVPKFSISATYDLGATLLKMG |
| | | | | IQHAYSENADFSGLTEDNGLKLSNAAHKA |
| | | | | VLHIGEKGTEAAAVPEVELSDQPENTFLHP |
| | | | | IIQIDRSFMLLILERSTRSILFLGKVVNPTEA |
| 60 | APOH | Beta-2- | P02749 | MISPVLILFSSFLCHVAIAGRTCPKPDDLPFS |
| | | glycoprotein 1 | | TVVPLKTFYEPGEEITYSCKPGYVSRGGMR |
| | | | | KFICPLTGLWPINTLKCTPRVCPFAGILENG |
| | | | | AVRYTTFEYPNTISFSCNTGFYLNGADSAK |
| | | | | CTEEGKWSPELPVCAPIICPPPSIPTFATLRV |
| | | | | YKPSAGNNSLYRDTAVFECLPQHAMFGND |
| | | | | TITCTTHGNWTKLPECREVKCPFPSRPDNG |
| | | | | FVNYPAKPTLYYKDKATFGCHDGYSLDGP |
| | | | | EEIECTKLGNWSAMPSCKASCKVPVKKAT |
| | | | | VVYQGERVKIQEKFKNGMLHGDKVSFFC |
| | | | | KNKEKKCSYTEDAQCIDGTIEVPKCFKEHS |
| | | | | SLAFWKTDASDVKPC |
| 61 | ATL3 | ADAMTS-like | P82987 | MASWTSPWWVLIGMVFMHSPLPQTTAEK |
| | | protein 3 | | SPGAYFLPEFALSPQGSFLEDTTGEQFLTYR |
| | | | | YDDQTSRNTRSDEDKDGNWDAWGDWSD |
| | | | | CSRTCGGGASYSLRRCLTGRNCEGQNIRY |
| | | | | KTCSNHDCPPDAEDFRAQQCSAYNDVQY |
| | | | | QGHYYEWLPRYNDPAAPCALKCHAQGQN |
| | | | | LVVELAPKVLDGTRCNTDSLDMCISGICQA |
| | | | | VGCDRQLGSNAKEDNCGVCAGDGSTCRL |
| | | | | VRGQSKSHVSPEKREENVIAVPLGSRSVRI |
| | | | | TVKGPAHLFIESKTLQGSKGEHSFNSPGVF |
| | | | | LVENTTVEFQRGSERQTFKIPGPLMADFIF |
| | | | | KTRYTAAKDSVVQFFFYQPISHQWRQTDF |
| | | | | FPCTVTCGGGYQLNSAECVDIRLKRVVPD |
| | | | | HYCHYYPENVKPKPKLKECSMDPCPSSDG |
| | | | | FKEIMPYDHFQPLPRWEHNPWTACSVSCG |
| | | | | GGIQRRSFVCVEESMHGEILQVEEWKCMY |
| | | | | APKPKVMQTCNLFDCPKWIAMEWSQCTV |
| | | | | TCGRGLRYRVVLCINHRGEHVGGCNPQLK |
| | | | | LHIKEECVIPIPCYKPKEKSPVEAKLPWLKQ |
| | | | | AQELEETRIATEEPTFIPEPWSACSTTCGPG |
| | | | | VQVREVKCRVLLTFTQTETELPEEECEGPK |
| | | | | LPTERPCLLEACDESPASRELDIPLPEDSET |
| | | | | TYDWEYAGFTPCTATCVGGHQEAIAVCLH |
| | | | | IQTQQTVNDSLCDMVHRPPAMSQACNTEP |
| | | | | CPPRWHVGSWGPCSATCGVGIQTRDVYCL |
| | | | | HPGETPAPPEECRDEKPHALQACNQFDCPP |
| | | | | GWHIEEWQQCSRTCGGGTQNRRVTCRQL |
| | | | | LTDGSFLNLSDELCQGPKASSHKSCARTDC |
| | | | | PPHLAVGDWSKCSVSCGVGIQRRKQVCQR |
| | | | | LAAKGRRIPLSEMMCRDLPGLPLVRSCQM |
| | | | | PECSKIKSEMKTKLGEQGPQILSVQRVYIQ |
| | | | | TREEKRINLTIGSRAYLLPNTSVIIKCPVRRF |
| | | | | QKSLIQWEKDGRCLQNSKRLGITKSGSLKI |
| | | | | HGLAAPDIGVYRCIAGSAQETVVLKLIGTD |
| | | | | NRLIARPALREPMREYPGMDHSEANSLGV |
| | | | | TWHKMRQMWNNKNDLYLDDDHISNQPF |
| | | | | LRALLGHCSNSAGSTNSWELKNKQFEAAV |
| | | | | KQGAYSMDTAQFDELIRNMSQLMETGEVS |
| | | | | DDLASQLIYQLVAELAKAQPTHMQWRGIQ |
| | | | | EETPPAAQLRGETGSVSQSSHAKNSGKLTF |
| | | | | KPKGPVLMRQSQPPSISFNKTINSRIGNTVY |
| | | | | ITKRTEVINILCDLITPSEATYTWTKDGTLL |
| | | | | QPSVKIILDGTGKIQIQNPTRKEQGIYECSV |
| | | | | ANHLGSDVESSSVLYAEAPVILSVERNITK |
| | | | | PEHNHLSVVVGGIVEAALGANVTIRCPVK |
| | | | | GVPQPNITWLKRGGSLSGNVSLLFNGSLLL |
| | | | | QNVSLENEGTYVCIATNALGKAVATSVLH |
| | | | | LLERRWPESRIVFLQGHKKYILQATNTRTN |
| | | | | SNDPTGEPPPQEPFWEPGNWSHCSATCGH |
| | | | | LGARIQRPQCVMANGQEVSEALCDHLQKP |
| | | | | LAGFEPCNIRDCPARWFTSVWSQCSVSCG |
| | | | | EGYHSRQVTCKRTKANGTVQVVSPRACAP |
| | | | | KDRPLGRKPCFGHPCVQWEPGNRCPGRC |
| | | | | MGRAVRMQQRHTACQHNSSDSNCDDRKR |
| | | | | PTLRRNCTSGACDVCWHTGPWKPCTAAC |
| | | | | GRGFQSRKVDCIHTRSCKPVAKRHCVQKK |
| | | | | KPISWRHCLGPSCDRDCTDTTHYCMFVKH |
| | | | | LNLCSLDRYKQRCCQSCQEG |
| 62 | IGG1 | Immunoglobulin | PODOX5 | QVQLVQSGGGVVQPGRSLRLSCAASGFTF |
| | | gamma-1 heavy | | SRYTIHWVRQAPGKGLEWVAVMSYNGNN |
| | | chain | | KHYADSVNGRFTISRNDSKNTLYLNMNSL |
| | | | | RPEDTAVYYCARIRDTAMFFAHWGQGTL |
| | | | | VTVSSASTKGPSVFPLAPSSKSTSGGTAAL |
| | | | | GCLVKDYFPEPVTVSWNSGALTSGVHTFP |
| | | | | AVLQSSGLYSLSSVVTVPSSSLGTQTYICN |
| | | | | VNHKPSNTKVDKKVEPKSCDKTHTCPPCP |
| | | | | APELLGGPSVFLFPPKPKDTLMISRTPEVTC |
| | | | | VVVDVSHEDPEVKFNWYVDGVEVHNAKT |
| | | | | KPREEQYNSTYRVVSVLTVLHQDWLNGK |
| | | | | EYKCKVSNKALPAPIEKTISKAKGQPREPQ |
| | | | | VYTLPPSRDELTKNQVSLTCLVKGFYPSDI |
| | | | | AVEWESNGQPENNYKTTPPVLDSDGSFFL |
| | | | | YSKLTVDKSRWQQGNVFSCSVMHEALHN |
| | | | | HYTQKSLSLSPGK |
| 63 | IGHA2 | Immunoglobulin | P01877 | ASPTSPKVFPLSLDSTPQDGNVVVACLVQG |
| | | heavy constant | | FFPQEPLSVTWSESGQNVTARNFPPSQDAS |
| | | alpha 2 | | GDLYTTSSQLTLPATQCPDGKSVTCHVKH |
| | | | | YTNSSQDVTVPCRVPPPPPCCHPRLSLHRP |
| | | | | ALEDLLLGSEANLTCTLTGLRDASGATFT |
| | | | | WTPSSGKSAVQGPPERDLCGCYSVSSVLP |
| | | | | GCAQPWNHGETFTCTAAHPELKTPLTANI |
| | | | | TKSGNTFRPEVHLLPPPSEELALNELVTLTC |
| | | | | LARGFSPKDVLVRWLQGSQELPREKYLTW |
| | | | | ASRQEPSQGTTTYAVTSILRVAAEDWKKG |
| | | | | ETFSCMVGHEALPLAFTQKTIDRMAGKPT |
| | | | | HINVSVVMAEADGTCY |
| 64 | KNG1 | Kininogen-1 | P01042 | MKLITILFLCSRLLLSLTQESQSEEIDCNDK |
| | | | | DLFKAVDAALKKYNSQNQSNNQFVLYRIT |
| | | | | EATKTVGSDTFYSFKYEIKEGDCPVQSGKT |
| | | | | WQDCEYKDAAKAATGECTATVGKRSSTK |
| | | | | FSVATQTCQITPAEGPVVTAQYDCLGCVH |
| | | | | PISTQSPDLEPILRHGIQYFNNNTQHSSLFM |
| | | | | LNEVKRAQRQVVAGLNFRITYSIVQTNCS |
| | | | | KENFLFLTPDCKSLWNGDTGECTDNAYIDI |
| | | | | QLRIASFSQNCDIYPGKDFVQPPTKICVGCP |
| | | | | RDIPTNSPELEETLTHTITKLNAENNATFYF |
| | | | | KIDNVKKARVQVVAGKKYFIDFVARETTC |
| | | | | SKESNEELTESCETKKLGQSLDCNAEVYV |
| | | | | VPWEKKIYPTVNCQPLGMISLMKRPPGFSP |
| | | | | FRSSRIGEIKEETTVSPPHTSMAPAQDEERD |
| | | | | SGKEQGHTRRHDWGHEKQRKHNLGHGH |
| | | | | KHERDQGHGHQRGHGLGHGHEQQHGLG |
| | | | | HGHKFKLDDDLEHQGGHVLDHGHKHKH |
| | | | | GHGHGKHKNKGKKNGKHNGWKTEHLAS |
| | | | | SSEDSTTPSAQTQEKTEGPTPIPSLAKPGVT |
| | | | | VTFSDFQDSDLIATMMPPISPAPIQSDDDWI |
| | | | | PDIQIDPNGLSFNPISDFPDTTSPKCPGRPW |
| | | | | KSVSEINPTTQMKESYYFDLTDGLS |

TABLE 17
Details of glycopeptides with different abundances in pregnancy control and
sPE sample sets

| | | | Linking Site | Linking Site | |
|---|---|---|---|---|---|
| | | | Pos. in | Pos. in | Glycan |
| SEQ ID | Peptide Structure | | Protein | Peptide | Structure |
| NO | (PS) NAME | Peptide Sequence | Sequence | Sequence | GL No |
| 65 | A1AG1 (103)-5402 | ENGTISR | 103 | 2 | 5402 |
| 66 | A1AG1 (56)-5412 | NEEYNK | 56 | 5 | 5412 |
| 67 | A1AG1 (72)-5402 | SVQEIQATFFYFTP | 72 | 15 | 5402 |
| | | NK | | | |
| 68 | A1AG1 (72)-6501 | SVQEIQATFFYFTP | 72 | 15 | 6501 |
| | | NK | | | |
| 69 | A1AG1 (72)-6502 | SVQEIQATFFYFTP | 72 | 15 | 6502 |
| | | NK | | | |
| 70 | A1AT (271)-4300 | YLGNATAIFFLPDE | 271 | 4 | 4300 |
| | | GK | | | |
| 71 | A1AT (271)-4301 | YLGNATAIFFLPDE | 271 | 4 | 4301 |
| | | GK | | | |
| 72 | A1AT (271)-6502 | YLGNATAIFFLPDE | 271 | 4 | 6502 |
| | | GK | | | |
| 73 | A1AT (70)-4301 | QLAHQSNSTNIFFS | 70 | 7 | 4301 |
| | | PVSIATAFAMLSL | | | |
| | | GTK | | | |
| 74 | A1AT (70)-6502 | QLAHQSNSTNIFFS | 70 | 7 | 6502 |
| | | PVSIATAFAMLSL | | | |
| | | GTK | | | |
| 75 | A2MG (1424)-5400 | VSNQTLSLFFTVL | 1424 | 3 | 5400 |
| | | QDVPVR | | | |
| 76 | A2MG (869)-4401 | SLGNVNFTVSAEA | 869 | 6 | 4401 |
| | | LESQELCGTEVPS | | | |
| | | VPEHGRK | | | |
| 77 | A2MG (869)-5400 | SLGNVNFTVSAEA | 869 | 6 | 5400 |
| | | LESQELCGTEVPS | | | |
| | | VPEHGRK | | | |
| 78 | AACT (106)-6502 | FNLTETSEAEIHQS | 106 | 2 | 6502 |
| | | FQHLLR | | | |
| 79 | AACT (93)-5402 | APDKNVIFSPLSIS | 93 | 26 | 5402 |
| | | TALAFLSLGAHNT | | | |
| | | TLTEILK | | | |
| 80 | AMBP (250)-5402 | YFYNGTSMACETF | 250 | 4 | 5402 |
| | | QYGGCMGNGNNF | | | |
| | | VTEKECLQTCR | | | |
| 81 | ANGT (161)-5402 | DKNCTSR | 161 | 3 | 5402 |
| 82 | ANT3 (128)-5401 | LGACNDTLQQLM | 128 | 5 | 5401 |
| | | EVFK | | | |
| 83 | AP1M2 (76)-5401 | NANASLVYSFLYK | 76 | 3 | 5401 |
| | | TIEVFCEYFK | | | |
| 84 | APOB (3224)-5401 | SYNETK | 3224 | 3 | 5401 |
| 85 | CERU (762)-5401 | ELHHLQEQNVSNA | 762 | 9 | 5401 |
| | | FLDKGEFYIGSK | | | |
| 86 | CFAB (378)-5402 | KALQAVYSMMSW | 378 | 22 | 5402 |
| | | PDDVPPEGWNR | | | |
| 87 | CO4A (1391)-5402 | TYNVLDMKNTTC | 1391 | 9 | 5402 |
| | | QDLQIEVTVK | | | |
| 88 | CO4A (226)-5200 | FSDGLESNSSTQFE | 226 | 8 | 5200 |
| | | VK | | | |
| 89 | CO4A (226)-9200 | FSDGLESNSSTQFE | 226 | 8 | 9200 |
| | | VKK | | | |
| 90 | FIBB (394)-2200 | GTAGNALMDGAS | 394 | 18 | 2200 |
| | | QLMGENR | | | |
| 91 | FIBB (394)-3200 | GTAGNALMDGAS | 394 | 18 | 3200 |
| | | QLMGENR | | | |
| 92 | FIBB (394)-4301 | GTAGNALMDGAS | 394 | 18 | 4301 |
| | | QLMGENR | | | |
| 93 | FIBB (395)-4300 | YRGTAGNALMDG | 395 | 21 | 4300 |
| | | ASQLMGENR | | | |
| 94 | FIBB (395)-5401 | YRGTAGNALMDG | 395 | 21 | 5401 |
| | | ASQLMGENR | | | |
| 95 | FINC (430)-5401 | GGNSNGALCHFPF | 430 | 19 | 5401 |
| | | LYNNHNYTDCTSE | | | |
| | | GRR | | | |
| 96 | FINC (542)-5401 | HEEGHMLNCTCF | 542 | 8 | 5401 |
| | | GQGR | | | |
| 97 | FINC (542)-5401 | RHEEGHMLNCTCF | 542 | 9 | 5401 |
| | | GQGR | | | |
| 98 | HEMO (240)-5402 | GHGHRNGTGHGN | 240 | 6 | 5402 |
| | | STHHGPEYMR | | | |
| 99 | IC1 (238)-5401 | DTFVNASR | 238 | 5 | 5401 |
| 100 | IGHM (209)-5402 | GLTFQQNASSMCV | 209 | 7 | 5402 |
| | | PDQDTAIR | | | |
| 101 | IGHM (209)-5410 | GLTFQQNASSMCV | 209 | 7 | 5410 |
| | | PDQDTAIR | | | |
| 102 | IGHM (209)-6401 | GLTFQQNASSMCV | 209 | 7 | 6401 |
| | | PDQDTAIR | | | |
| 103 | IGHM (209)-6501 | GLTFQQNASSMCV | 209 | 7 | 6501 |
| | | PDQDTAIR | | | |
| 104 | IGHM (209)-6502 | GLTFQQNASSMCV | 209 | 7 | 6502 |
| | | PDQDTAIR | | | |
| 105 | ITIH1 (588)-5401 | ANLSSQALQMSLD | 588 | 2 | 5401 |
| | | YGFVTPLTSMSIR | | | |
| 106 | ITIH1 (588)-5412 | ANLSSQALQMSLD | 588 | 2 | 5412 |
| | | YGFVTPLTSMSIR | | | |
| 107 | MYCT (458)-4301 | MNKSTVIDSSCVP | 458 | 2 | 4301 |
| | | VNK | | | |
| 108 | MYCT (458)-5401 | MNKSTVIDSSCVP | 458 | 2 | 5401 |
| | | VNK | | | |
| 109 | PHLD (604)-6502 | NASR | 604 | 1 | 6502 |
| 110 | PSG1 (259)-5411 | ENKDVLNFTCEPK | 259 | 7 | 5411 |
| 111 | PSG1 (268)-5411 | SENYTYIWWLNG | 268 | 3 | 5411 |
| | | QSLPVSPR | | | |
| 112 | PSG1 (303)-5411 | ILILPSVTRNETGP | 303 | 10 | 5411 |
| | | YQCEIR | | | |
| 113 | PSG1 (303)-5422 | ILILPSVTRNETGP | 303 | 10 | 5422 |
| | | YQCEIR | | | |
| 114 | PSG7 (268)-5412 | SENYTYIWWLNG | 268 | 3 | 5412 |
| | | QSLPVSPR | | | |
| 115 | PSG7 (303)-5412 | ILILPSVTRNETGP | 303 | 10 | 5412 |
| | | YQCEIR | | | |
| 116 | PSG7 (303)-5422 | ILILPSVTRNETGP | 303 | 10 | 5422 |
| | | YQCEIR | | | |
| 117 | PZP (54)-5401 | KGCVLLSHLNETV | 54 | 10 | 5401 |
| | | TVSASLESGR | | | |
| 118 | PZP (54)-5402 | KGCVLLSHLNETV | 54 | 10 | 5402 |
| | | TVSASLESGR | | | |
| 119 | SHBG (396)-5402 | SHEIWTHSCPQSP | 396 | 15 | 5402 |
| | | GNGTDAS | | | |
| 120 | TRFE (432)-4300 | CGLVPVLAENYN | 432 | 12 | 4300 |
| | | K | | | |
| 121 | VTNC (242)-5401 | NISDGFDGIPDNV | 242 | 1 | 5401 |
| | | DAALALPAHSYSG | | | |
| | | R | | | |
| 122 | ZA2G (109)-5402 | AREDIFMETLKDI | 109 | 18 | 5402 |
| | | VEYYNDSNGSHV | | | |
| | | LQGR | | | |
| 123 | ZA2G (109)-5402 | DIVEYYNDSNGSH | 109 | 7 | 5402 |
| | | VLQGR | | | |
| 124 | A1AT (271)-5401 | YLGNATAIFFLPDE | 271 | 4 | 5401 |
| | | GK | | | |
| 125 | A1AT (271)-5402 | YLGNATAIFFLPDE | 271 | 4 | 5402 |
| | | GK | | | |
| 126 | A1AT (271)-5412 | YLGNATAIFFLPDE | 271 | 4 | 5412 |
| | | GK | | | |
| 127 | A2MG (1424)-5412 | VSNQTLSLFFTVL | 1424 | 3 | 5412 |
| | | QDVPVR | | | |
| 128 | A2MG (247)-5401 | IITILEEEMNVSVC | 247 | 10 | 5401 |
| | | GLYTYGKPVPGH | | | |
| | | VTVSICR | | | |
| 129 | A2MG (247)-5402 | IITILEEEMNVSVC | 247 | 10 | 5402 |
| | | GLYTYGK | | | |
| 130 | A2MG (247)-5402 | IITILEEEMNVSVC | 247 | 10 | 5402 |
| | | GLYTYGKPVPGH | | | |
| | | VTVSICR | | | |
| 131 | A2MG (869)-5401 | SLGNVNFTVSAEA | 869 | 6 | 5401 |
| | | LESQELCGTEVPS | | | |
| | | VPEHGRK | | | |
| 132 | AACT (271)-6502 | YTGNASALFILPD | 271 | 4 | 6502 |
| | | QDKMEEVEAMLL | | | |
| | | PETLKR | | | |
| 133 | AFAM (109)-5402 | HNFSHCCSK | 109 | 2 | 5402 |
| 134 | AFAM (109)-5402 | ICAMEGLPQKHNF | 109 | 12 | 5402 |
| | | SHCCSK | | | |
| 135 | ANT3 (128)-5402 | LGACNDTLQQLM | 128 | 5 | 5402 |
| | | EVFKFDTISEK | | | |
| 136 | APOB (983)-5401 | QVFPGLNYCTSGA | 983 | 16 | 5401 |
| | | YSNASSTDSASYY | | | |
| | | PLTGDTR | | | |
| 137 | CERU (397)-5412 | ENLTAPGSDSAVF | 397 | 2 | 5412 |
| | | FEQGTTR | | | |
| 138 | CERU (762)-5402 | ELHHLQEQNVSNA | 762 | 9 | 5402 |
| | | FLDK | | | |
| 139 | CERU (762)-5412 | ELHHLQEQNVSNA | 762 | 9 | 5412 |
| | | FLDK | | | |
| 140 | CERU (762)-6502 | ELHHLQEQNVSNA | 762 | 9 | 6502 |
| | | FLDK | | | |
| 141 | CFAI (70)-5401 | NGTAVCATNR | 70 | 1 | 5401 |
| 142 | CO3 (85)-5200 | TVLTPATNHMGN | 85 | 9 | 5200 |
| | | VTFTIPANR | | | |
| 143 | FETUA (156)-6502 | VCQDCPLLAPLND | 156 | 12 | 6502 |
| | | TR | | | |
| 144 | FINC (542)-5402 | HEEGHMLNCTCF | 542 | 8 | 5402 |
| | | GQGR | | | |
| 145 | FINC (542)-5402 | RHEEGHMLNCTCF | 542 | 9 | 5402 |
| | | GQGR | | | |
| 146 | HEMO (453)-6502 | ALPQPQNVTSLLG | 453 | 7 | 6502 |
| | | CTH | | | |
| 147 | HEP2 (49)-5402 | GGETAQSADPQW | 49 | 19 | 5402 |
| | | EQLNNKNLSMPLL | | | |
| | | PADFHK | | | |
| 148 | HEP2 (49)-5412 | GGETAQSADPQW | 49 | 19 | 5412 |
| | | EQLNNKNLSMPLL | | | |
| | | PADFHK | | | |
| 149 | HPT (184)-6502 | MVSHHNLTTGAT | 184 | 6 | 6502 |
| | | LINEQWLLTTAK | | | |
| 150 | IC1 (253)-5402 | VLSNNSDANLELI | 253 | 4 | 5402 |
| | | NTWVAK | | | |
| 151 | IC1 (352)-5402 | VGQLQLSHNLSLV | 352 | 9 | 5402 |
| | | ILVPQNLK | | | |
| 152 | IGHA1 (144)-5401 | LSLHRPALEDLLL | 144 | 18 | 5401 |
| | | GSEANLTCTLTGL | | | |
| | | R | | | |
| 153 | IGHA1 (144)-5402 | LSLHRPALEDLLL | 144 | 18 | 5402 |
| | | GSEANLTCTLTGL | | | |
| | | R | | | |
| 154 | IGHA1 (144)-5501 | LSLHRPALEDLLL | 144 | 18 | 5501 |
| | | GSEANLTCTLTGL | | | |
| | | R | | | |
| 155 | IGHG2 (176)-3410 | EEQFNSTFR | 176 | 5 | 3410 |
| 156 | IGHG2 (176)-4410 | EEQFNSTFR | 176 | 5 | 4410 |
| 157 | IGHG2 (176)-4410 | TKPREEQFNSTFR | 176 | 9 | 4410 |
| 158 | IGHG2 (176)-5410 | TKPREEQFNSTFR | 176 | 9 | 5410 |
| 159 | IGHG2 (176)-5411 | TKPREEQFNSTFR | 176 | 9 | 5411 |
| 160 | IGJ (71)-5401 | ENISDPTSPLR | 71 | 2 | 5401 |
| 161 | IGHM (209)-5401 | GLTFQQNASSMCV | 209 | 7 | 5401 |
| | | PDQDTAIR | | | |
| 162 | IGHM (209)-5412 | GLTFQQNASSMCV | 209 | 7 | 5412 |
| | | PDQDTAIR | | | |
| 163 | IGHM (209)-5501 | GLTFQQNASSMCV | 209 | 7 | 5501 |
| | | PDQDTAIR | | | |
| 164 | IGHM (209)-5510 | GLTFQQNASSMCV | 209 | 7 | 5510 |
| | | PDQDTAIR | | | |
| 165 | IGHM (46)-5401 | YKNNSDISSTR | 46 | 3 | 5401 |
| 166 | IGHM (46)-5401 | YKNNSDISSTR | 46 | 3 | 5401 |
| 167 | IGHM (46)-5411 | NNSDISSTR | 46 | 1 | 5411 |
| 168 | IGHM (46)-5501 | YKNNSDISSTR | 46 | 3 | 5501 |
| 169 | IGHM (46)-6311 | YKNNSDISSTR | 46 | 3 | 6311 |
| 170 | KLKB1 (127)-5402 | GVNFNVSK | 127 | 5 | 5402 |
| 171 | THBG (36)-5402 | VTACHSSQPNATL | 36 | 10 | 5402 |
| | | YK | | | |
| 172 | TRFE (432)-6502 | CGLVPVLAENYN | 432 | 12 | 6502 |
| | | K | | | |
| 173 | VTNC (169)-5401 | NGSLFAFR | 169 | 1 | 5401 |
| 174 | VTNC (169)-5402 | NGSLFAFR | 169 | 1 | 5402 |
| 175 | VTNC (242)-5412 | NISDGFDGIPDNV | 242 | 1 | 5412 |
| | | DAALALPAHSYSG | | | |
| | | R | | | |
| 176 | ANGT (38)-6502 | VYIHPFHLVIHNES | 38 | 12 | 6502 |
| | | TCEQLAK | | | |
| 177 | APOH (162)-5401 | VYKPSAGNNSLYR | 162 | 8 | 5401 |
| 178 | ATL3 (1330)-6501 | GVPQPNITWLKR | 1330 | 6 | 6501 |
| 179 | IGG1 (299)-3410 | EEQYNSTYR | 299 | 5 | 3410 |
| 180 | IGG1 (299)-4300 | EEQYNSTYR | 299 | 5 | 4300 |
| 181 | IGG1 (299)-5400 | EEQYNSTYR | 299 | 5 | 5400 |
| 182 | IGG1 (299)-5410 | TKPREEQYNSTYR | 299 | 9 | 5410 |
| 183 | IGG1 (299)-5411 | TKPREEQYNSTYR | 299 | 9 | 5411 |
| 184 | IGHA2 (92)-5411 | HYTNSSQDVTVPC | 92 | 4 | 5411 |
| | | R | | | |
| 185 | IGHA2 (92)-5412 | HYTNSSQDVTVPC | 92 | 4 | 5412 |
| | | R | | | |
| 186 | IGHM (440)-6200 | STGKPTLYNVSLV | 440 | 9 | 6200 |
| | | MSDTAGTC | | | |
| 187 | KNG1 (169)-6502 | HGIQYFNNNTQHS | 169 | 8 | 6502 |
| | | SLFMLNEVK | | | |
| 188 | KNG1 (205)-6502 | ITYSIVQTNCSKEN | 205 | 9 | 6502 |
| | | FLFLTPDCK | | | |

TABLE 18
Glycan structure GL NO, symbol structure, and composition

| Glycan Structure GL NO. | Symbol Structure | Composition |
|---|---|---|
| 2200 | 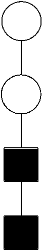 | Hex(2)HexNAc(2)Fuc(0)NeuAc(0) |
| 3200 | 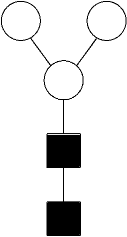 | Hex(3)HexNAc(2)Fuc(0)NeuAc(0) |
| 3410 | 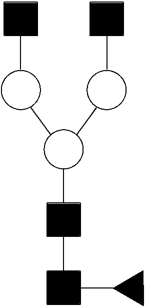 | Hex(3)HexNAc(4)Fuc(1)NeuAc(0) |
| 4300 | 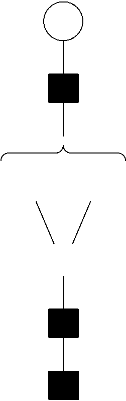 | Hex(4)HexNAc(3)Fuc(0)NeuAc(0) |
| 4301 | 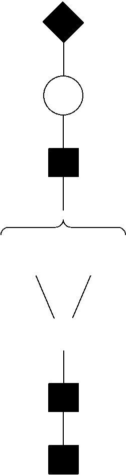 | Hex(4)HexNAc(3)Fuc(0)NeuAc(1) |
| 4401 | 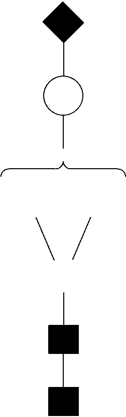 | Hex(4)HexNAc(4)Fuc(0)NeuAc(1) |
| 4410 | 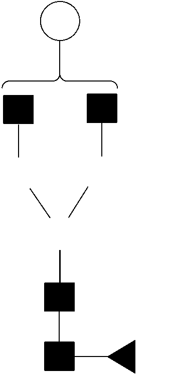 | Hex(4)HexNAc(4)Fuc(1)NeuAc(0) |
| 5200 | 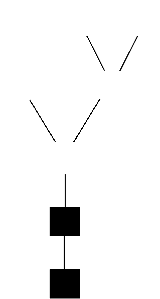 | Hex(5)HexNAc(2)Fuc(0)NeuAc(0) |
| 5400 | 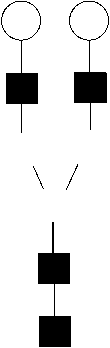 | Hex(5)HexNAc(4)Fuc(0)NeuAc(0) |
| 5401 | 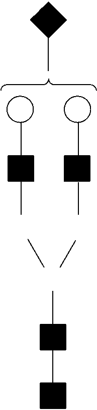 | Hex(5)HexNAc(4)Fuc(0)NeuAc(1) |
| 5402 | 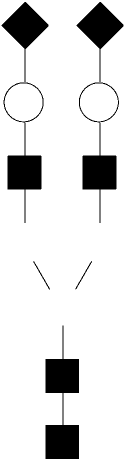 | Hex(5)HexNAc(4)Fuc(0)NeuAc(2) |
| 5410 | 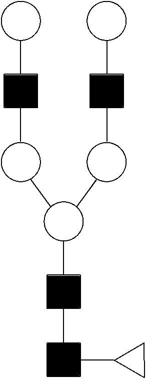 | Hex(5)HexNAc(4)Fuc(1)NeuAc(0) |
| 5411 | 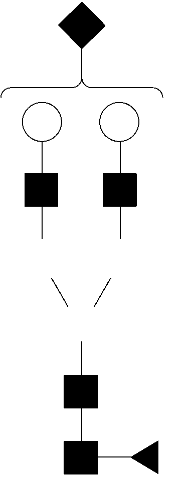 | Hex(5)HexNAc(4)Fuc(1)NeuAc(1) |
| 5412 | 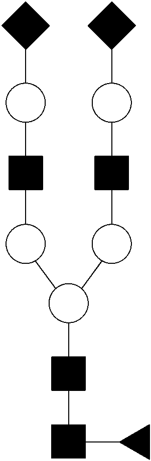 | Hex(5)HexNAc(4)Fuc(1)NeuAc(2) |
| 5422 | 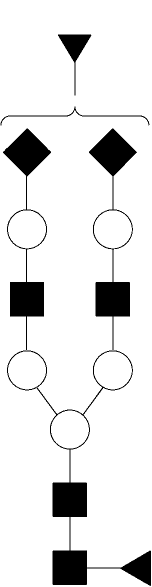 | Hex(5)HexNAc(4)Fuc(2)NeuAc(2) |
| 5501 | 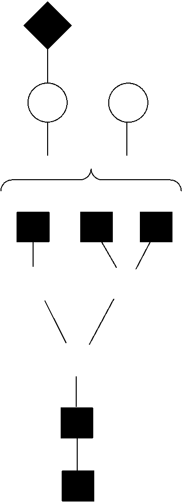 | Hex(5)HexNAc(5)Fuc(0)NeuAc(1) |
| 5510 | 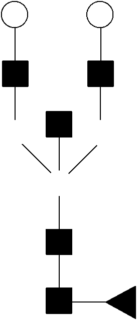 | Hex(5)HexNAc(5)Fuc(1)NeuAc(0) |
| 6200 | 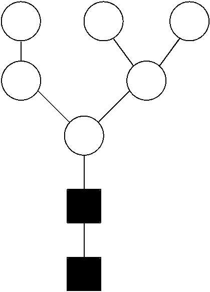 | Hex(6)HexNAc(2)Fuc(0)NeuAc(0) |
| 6311 | 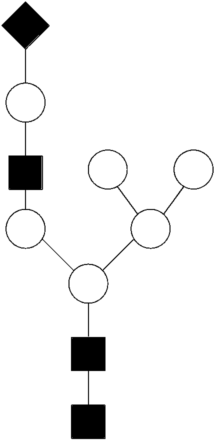 | Hex(6)HexNAc(3)Fuc(1)NeuAc(1) |
| 6401 | 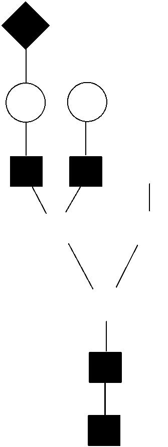 | Hex(6)HexNAc(4)Fuc(0)NeuAc(1) |
| 6501 | 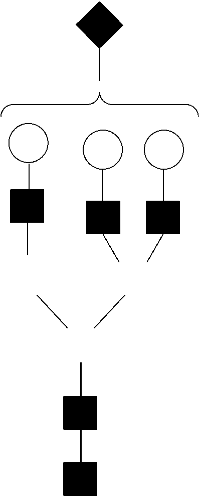 | Hex(6)HexHAc(5)Fuc(0)NeuAc(1) |
| 6502 | 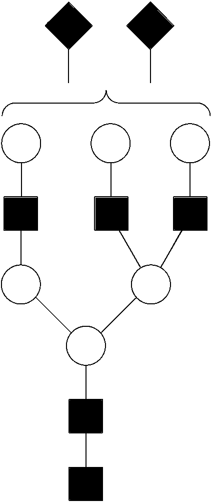 | Hex(6)HexNAc(5)Fuc(0)NeuAc(2) |
| 9200 | 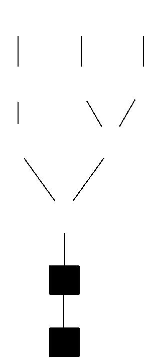 | Hex(9)HexNAc(2)Fuc(0)NeuAc(0) |

Glc

[symbol]

Gal
◯
Man

[symbol]

Fuc

[symbol]

Neu5Ac

[symbol]

GlcNAc

[symbol]

GalNAc
□
ManNAc

[symbol]

TABLE 19
Differential expression analysis for pregnancy control and sPE sample sets

| | PSM Count | | | Combined | Relative Abundance | | | Fold Change¹ | |
|---|---|---|---|---|---|---|---|---|---|
| SEQ ID | | Early | Late | PSM | | Early | Late | Early sPE/ | Late sPE/ |
| NO | Control | sPE | sPE | Count | Control | sPE | sPE | Control | Control |
| 65 | 16 | 17 | 5 | 38 | 9.800E−04 | 1.051E−03 | 3.864E−04 | 9.321E−01 | 3.675E−01 |
| 66 | 16 | 17 | 3 | 36 | 9.800E−04 | 1.051E−03 | 2.319E−04 | 9.321E−01 | 2.205E−01 |
| 67 | 27 | 30 | 6 | 63 | 1.654E−03 | 1.855E−03 | 4.637E−04 | 8.913E−01 | 2.499E−01 |
| 68 | 29 | 20 | 1 | 50 | 1.776E−03 | 1.237E−03 | 7.729E−05 | 1.436E+00 | 6.248E−02 |
| 69 | 120 | 111 | 24 | 255 | 7.350E−03 | 6.865E−03 | 1.855E−03 | 1.071E+00 | 2.702E−01 |
| 70 | 62 | 72 | 16 | 150 | 3.797E−03 | 4.453E−03 | 1.237E−03 | 8.528E−01 | 2.777E−01 |
| 71 | 15 | 22 | 5 | 42 | 9.187E−04 | 1.361E−03 | 3.864E−04 | 6.752E−01 | 2.840E−01 |
| 72 | 33 | 38 | 11 | 82 | 2.021E−03 | 2.350E−03 | 8.501E−04 | 8.600E−01 | 3.617E−01 |
| 73 | 14 | 5 | 19 | 38 | 8.575E−04 | 3.092E−04 | 1.468E−03 | 2.773E+00 | 4.749E+00 |
| 74 | 28 | 19 | 45 | 92 | 1.715E−03 | 1.175E−03 | 3.478E−03 | 1.459E+00 | 2.960E+00 |
| 75 | 20 | 12 | 3 | 35 | 1.225E−03 | 7.422E−04 | 2.319E−04 | 1.651E+00 | 3.124E−01 |
| 76 | 10 | 7 | 13 | 30 | 6.125E−04 | 4.329E−04 | 1.005E−03 | 1.415E+00 | 2.321E+00 |
| 77 | 9 | 7 | 17 | 33 | 5.512E−04 | 4.329E−04 | 1.314E−03 | 1.273E+00 | 3.035E+00 |
| 78 | 45 | 34 | 12 | 91 | 2.756E−03 | 2.103E−03 | 9.274E−04 | 1.311E+00 | 4.410E−01 |
| 79 | 9 | 9 | 15 | 33 | 5.512E−04 | 5.566E−04 | 1.159E−03 | 9.903E−01 | 2.083E+00 |
| 80 | 6 | 1 | 37 | 44 | 3.675E−04 | 6.185E−05 | 2.860E−03 | 5.942E+00 | 4.624E+01 |
| 81 | 31 | 30 | 9 | 70 | 1.899E−03 | 1.855E−03 | 6.956E−04 | 1.023E+00 | 3.749E−01 |
| 82 | 9 | 8 | 36 | 53 | 5.512E−04 | 4.948E−04 | 2.782E−03 | 1.114E+00 | 5.623E+00 |
| 83 | 11 | 3 | 20 | 34 | 6.737E−04 | 1.855E−04 | 1.546E−03 | 3.631E+00 | 8.331E+00 |
| 84 | 16 | 21 | 3 | 40 | 9.800E−04 | 1.299E−03 | 2.319E−04 | 7.545E−01 | 1.785E−01 |
| 85 | 14 | 12 | 4 | 30 | 8.575E−04 | 7.422E−04 | 3.091E−04 | 1.155E+00 | 4.165E−01 |
| 86 | 21 | 17 | 28 | 66 | 1.286E−03 | 1.051E−03 | 2.164E−03 | 1.223E+00 | 2.058E+00 |
| 87 | 6 | 1 | 23 | 30 | 3.675E−04 | 6.185E−05 | 1.778E−03 | 5.942E+00 | 2.874E+01 |
| 88 | 13 | 10 | 18 | 41 | 7.962E−04 | 6.185E−04 | 1.391E−03 | 1.287E+00 | 2.249E+00 |
| 89 | 11 | 6 | 26 | 43 | 6.737E−04 | 3.711E−04 | 2.009E−03 | 1.816E+00 | 5.415E+00 |
| 90 | 17 | 21 | 8 | 46 | 1.041E−03 | 1.299E−03 | 6.183E−04 | 8.017E−01 | 4.761E−01 |
| 91 | 62 | 61 | 23 | 146 | 3.797E−03 | 3.773E−03 | 1.778E−03 | 1.007E+00 | 4.712E−01 |
| 92 | 16 | 19 | 7 | 42 | 9.800E−04 | 1.175E−03 | 5.410E−04 | 8.340E−01 | 4.604E−01 |
| 93 | 11 | 8 | 14 | 33 | 6.737E−04 | 4.948E−04 | 1.082E−03 | 1.362E+00 | 2.187E+00 |
| 94 | 55 | 39 | 64 | 158 | 3.369E−03 | 2.412E−03 | 4.946E−03 | 1.397E+00 | 2.051E+00 |
| 95 | 13 | 6 | 11 | 30 | 7.962E−04 | 3.711E−04 | 8.501E−04 | 2.146E+00 | 2.291E+00 |
| 96 | 49 | 20 | 17 | 86 | 3.001E−03 | 1.237E−03 | 1.314E−03 | 2.426E+00 | 1.062E+00 |
| 97 | 32 | 10 | 10 | 52 | 1.960E−03 | 6.185E−04 | 7.729E−04 | 3.169E+00 | 1.250E+00 |
| 98 | 9 | 23 | 31 | 63 | 5.512E−04 | 1.422E−03 | 2.396E−03 | 3.875E−01 | 1.684E+00 |
| 99 | 14 | 13 | 3 | 30 | 8.575E−04 | 8.040E−04 | 2.319E−04 | 1.067E+00 | 2.884E−01 |
| 100 | 4 | 31 | 13 | 48 | 2.450E−04 | 1.917E−03 | 1.005E−03 | 1.278E−01 | 5.240E−01 |
| 101 | 7 | 38 | 26 | 71 | 4.287E−04 | 2.350E−03 | 2.009E−03 | 1.824E−01 | 8.550E−01 |
| 102 | 47 | 102 | 88 | 237 | 2.879E−03 | 6.308E−03 | 6.801E−03 | 4.563E−01 | 1.078E+00 |
| 103 | 8 | 30 | 25 | 63 | 4.900E−04 | 1.855E−03 | 1.932E−03 | 2.641E−01 | 1.041E+00 |
| 104 | 4 | 27 | 23 | 54 | 2.450E−04 | 1.670E−03 | 1.778E−03 | 1.467E−01 | 1.065E+00 |
| 105 | 16 | 17 | 2 | 35 | 9.800E−04 | 1.051E−03 | 1.546E−04 | 9.321E−01 | 1.470E−01 |
| 106 | 34 | 25 | 3 | 62 | 2.082E−03 | 1.546E−03 | 2.319E−04 | 1.347E+00 | 1.500E−01 |
| 107 | 12 | 15 | 4 | 31 | 7.350E−04 | 9.277E−04 | 3.091E−04 | 7.923E−01 | 3.332E−01 |
| 108 | 12 | 16 | 6 | 34 | 7.350E−04 | 9.895E−04 | 4.637E−04 | 7.427E−01 | 4.686E−01 |
| 109 | 21 | 5 | 27 | 53 | 1.286E−03 | 3.092E−04 | 2.087E−03 | 4.159E+00 | 6.748E+00 |
| 110 | 12 | 9 | 18 | 39 | 7.350E−04 | 5.566E−04 | 1.391E−03 | 1.320E+00 | 2.499E+00 |
| 111 | 12 | 28 | 14 | 54 | 7.350E−04 | 1.732E−03 | 1.082E−03 | 4.244E−01 | 6.248E−01 |
| 112 | 5 | 20 | 21 | 46 | 3.062E−04 | 1.237E−03 | 1.623E−03 | 2.476E−01 | 1.312E+00 |
| 113 | 11 | 25 | 27 | 63 | 6.737E−04 | 1.546E−03 | 2.087E−03 | 4.357E−01 | 1.350E+00 |
| 114 | 12 | 28 | 14 | 54 | 7.350E−04 | 1.732E−03 | 1.082E−03 | 4.244E−01 | 6.248E−01 |
| 115 | 9 | 20 | 21 | 50 | 5.512E−04 | 1.237E−03 | 1.623E−03 | 4.456E−01 | 1.312E+00 |
| 116 | 7 | 25 | 27 | 59 | 4.287E−04 | 1.546E−03 | 2.087E−03 | 2.773E−01 | 1.350E+00 |
| 117 | 21 | 25 | 8 | 54 | 1.286E−03 | 1.546E−03 | 6.183E−04 | 8.319E−01 | 3.999E−01 |
| 118 | 21 | 25 | 9 | 55 | 1.286E−03 | 1.546E−03 | 6.956E−04 | 8.319E−01 | 4.499E−01 |
| 119 | 7 | 14 | 14 | 35 | 4.287E−04 | 8.659E−04 | 1.082E−03 | 4.952E−01 | 1.250E+00 |
| 120 | 13 | 21 | 4 | 38 | 7.962E−04 | 1.299E−03 | 3.091E−04 | 6.131E−01 | 2.380E−01 |
| 121 | 13 | 18 | 5 | 36 | 7.962E−04 | 1.113E−03 | 3.864E−04 | 7.152E−01 | 3.471E−01 |
| 122 | 10 | 6 | 14 | 30 | 6.125E−04 | 3.711E−04 | 1.082E−03 | 1.651E+00 | 2.916E+00 |
| 123 | 16 | 24 | 5 | 45 | 9.800E−04 | 1.484E−03 | 3.864E−04 | 6.602E−01 | 2.603E−01 |
| 124 | 82 | 87 | 25 | 194 | 5.022E−03 | 5.381E−03 | 1.932E−03 | 9.334E−01 | 3.591E−01 |
| 125 | 13 | 15 | 4 | 32 | 7.962E−04 | 9.277E−04 | 3.091E−04 | 8.583E−01 | 3.332E−01 |
| 126 | 372 | 445 | 94 | 911 | 2.278E−02 | 2.752E−02 | 7.265E−03 | 8.279E−01 | 2.640E−01 |
| 127 | 45 | 34 | 12 | 91 | 2.756E−03 | 2.103E−03 | 9.274E−04 | 1.311E+00 | 4.410E−01 |
| 128 | 80 | 103 | 288 | 471 | 4.900E−03 | 6.370E−03 | 2.226E−02 | 7.692E−01 | 3.494E+00 |
| 129 | 74 | 98 | 3 | 175 | 4.532E−03 | 6.061E−03 | 2.319E−04 | 7.478E−01 | 3.825E−02 |
| 130 | 8 | 23 | 49 | 80 | 4.900E−04 | 1.422E−03 | 3.787E−03 | 3.445E−01 | 2.662E+00 |
| 131 | 19 | 18 | 43 | 80 | 1.164E−03 | 1.113E−03 | 3.323E−03 | 1.045E+00 | 2.985E+00 |
| 132 | 28 | 11 | 18 | 57 | 1.715E−03 | 6.803E−04 | 1.391E−03 | 2.521E+00 | 2.045E+00 |
| 133 | 14 | 12 | 4 | 30 | 8.575E−04 | 7.422E−04 | 3.091E−04 | 1.155E+00 | 4.165E−01 |
| 134 | 0 | 0 | 32 | 32 | 0.000E+00 | 0.000E+00 | 2.473E−03 | Undefined | Undefined |
| 135 | 21 | 16 | 3 | 40 | 1.286E−03 | 9.895E−04 | 2.319E−04 | 1.300E+00 | 2.343E−01 |
| 136 | 26 | 21 | 6 | 53 | 1.592E−03 | 1.299E−03 | 4.637E−04 | 1.226E+00 | 3.570E−01 |
| 137 | 9 | 16 | 6 | 31 | 5.512E−04 | 9.895E−04 | 4.637E−04 | 5.571E−01 | 4.686E−01 |
| 138 | 26 | 26 | 9 | 61 | 1.592E−03 | 1.608E−03 | 6.956E−04 | 9.903E−01 | 4.326E−01 |
| 139 | 18 | 27 | 6 | 51 | 1.102E−03 | 1.670E−03 | 4.637E−04 | 6.602E−01 | 2.777E−01 |
| 140 | 25 | 29 | 7 | 61 | 1.531E−03 | 1.794E−03 | 5.410E−04 | 8.537E−01 | 3.016E−01 |
| 141 | 13 | 18 | 7 | 38 | 7.962E−04 | 1.113E−03 | 5.410E−04 | 7.152E−01 | 4.860E−01 |
| 142 | 24 | 30 | 12 | 66 | 1.470E−03 | 1.855E−03 | 9.274E−04 | 7.923E−01 | 4.999E−01 |
| 143 | 15 | 13 | 23 | 51 | 9.187E−04 | 8.040E−04 | 1.778E−03 | 1.143E+00 | 2.211E+00 |
| 144 | 22 | 9 | 11 | 42 | 1.347E−03 | 5.566E−04 | 8.501E−04 | 2.421E+00 | 1.527E+00 |
| 145 | 30 | 12 | 11 | 53 | 1.837E−03 | 7.422E−04 | 8.501E−04 | 2.476E+00 | 1.145E+00 |
| 146 | 23 | 26 | 57 | 106 | 1.409E−03 | 1.608E−03 | 4.405E−03 | 8.761E−01 | 2.740E+00 |
| 147 | 5 | 9 | 19 | 33 | 3.062E−04 | 5.566E−04 | 1.468E−03 | 5.502E−01 | 2.638E+00 |
| 148 | 30 | 30 | 50 | 110 | 1.837E−03 | 1.855E−03 | 3.864E−03 | 9.903E−01 | 2.083E+00 |
| 149 | 34 | 32 | 63 | 129 | 2.082E−03 | 1.979E−03 | 4.869E−03 | 1.052E+00 | 2.460E+00 |
| 150 | 29 | 23 | 9 | 61 | 1.776E−03 | 1.422E−03 | 6.956E−04 | 1.249E+00 | 4.890E−01 |
| 151 | 19 | 24 | 8 | 51 | 1.164E−03 | 1.484E−03 | 6.183E−04 | 7.840E−01 | 4.165E−01 |
| 152 | 46 | 12 | 27 | 85 | 2.817E−03 | 7.422E−04 | 2.087E−03 | 3.796E+00 | 2.812E+00 |
| 153 | 25 | 6 | 18 | 49 | 1.531E−03 | 3.711E−04 | 1.391E−03 | 4.126E+00 | 3.749E+00 |
| 154 | 29 | 12 | 26 | 67 | 1.776E−03 | 7.422E−04 | 2.009E−03 | 2.393E+00 | 2.708E+00 |
| 155 | 55 | 48 | 16 | 119 | 3.369E−03 | 2.969E−03 | 1.237E−03 | 1.135E+00 | 4.165E−01 |
| 156 | 18 | 16 | 4 | 38 | 1.102E−03 | 9.895E−04 | 3.091E−04 | 1.114E+00 | 3.124E−01 |
| 157 | 25 | 6 | 25 | 56 | 1.531E−03 | 3.711E−04 | 1.932E−03 | 4.126E+00 | 5.207E+00 |
| 158 | 20 | 13 | 34 | 67 | 1.225E−03 | 8.040E−04 | 2.628E−03 | 1.524E+00 | 3.268E+00 |
| 159 | 22 | 16 | 37 | 75 | 1.347E−03 | 9.895E−04 | 2.860E−03 | 1.362E+00 | 2.890E+00 |
| 160 | 28 | 21 | 8 | 57 | 1.715E−03 | 1.299E−03 | 6.183E−04 | 1.320E+00 | 4.761E−01 |
| 161 | 8 | 32 | 19 | 59 | 4.900E−04 | 1.979E−03 | 1.468E−03 | 2.476E−01 | 7.420E−01 |
| 162 | 51 | 130 | 103 | 284 | 3.124E−03 | 8.040E−03 | 7.960E−03 | 3.885E−01 | 9.901E−01 |
| 163 | 9 | 37 | 32 | 78 | 5.512E−04 | 2.288E−03 | 2.473E−03 | 2.409E−01 | 1.081E+00 |
| 164 | 7 | 39 | 21 | 67 | 4.287E−04 | 2.412E−03 | 1.623E−03 | 1.778E−01 | 6.729E−01 |
| 165 | 43 | 40 | 10 | 93 | 2.634E−03 | 2.474E−03 | 7.729E−04 | 1.065E+00 | 3.124E−01 |
| 166 | 38 | 40 | 16 | 94 | 2.327E−03 | 2.474E−03 | 1.237E−03 | 9.408E−01 | 4.999E−01 |
| 167 | 23 | 28 | 3 | 54 | 1.409E−03 | 1.732E−03 | 2.319E−04 | 8.135E−01 | 1.339E−01 |
| 168 | 13 | 17 | 6 | 36 | 7.962E−04 | 1.051E−03 | 4.637E−04 | 7.573E−01 | 4.410E−01 |
| 169 | 52 | 32 | 11 | 95 | 3.185E−03 | 1.979E−03 | 8.501E−04 | 1.609E+00 | 4.296E−01 |
| 170 | 12 | 26 | 23 | 61 | 7.350E−04 | 1.608E−03 | 1.778E−03 | 4.571E−01 | 1.105E+00 |
| 171 | 15 | 10 | 20 | 45 | 9.187E−04 | 6.185E−04 | 1.546E−03 | 1.485E+00 | 2.499E+00 |
| 172 | 9 | 20 | 4 | 33 | 5.512E−04 | 1.237E−03 | 3.091E−04 | 4.456E−01 | 2.499E−01 |
| 173 | 15 | 15 | 5 | 35 | 9.187E−04 | 9.277E−04 | 3.864E−04 | 9.903E−01 | 4.165E−01 |
| 174 | 34 | 40 | 13 | 87 | 2.082E−03 | 2.474E−03 | 1.005E−03 | 8.418E−01 | 4.061E−01 |
| 175 | 24 | 24 | 7 | 55 | 1.470E−03 | 1.484E−03 | 5.410E−04 | 9.903E−01 | 3.645E−01 |
| 176 | 10 | 19 | 7 | 36 | 6.125E−04 | 1.175E−03 | 5.410E−04 | 5.212E−01 | 4.604E−01 |
| 177 | 19 | 15 | 4 | 38 | 1.164E−03 | 9.277E−04 | 3.091E−04 | 1.254E+00 | 3.332E−01 |
| 178 | 14 | 18 | 2 | 34 | 8.575E−04 | 1.113E−03 | 1.546E−04 | 7.703E−01 | 1.388E−01 |
| 179 | 14 | 15 | 2 | 31 | 8.575E−04 | 9.277E−04 | 1.546E−04 | 9.243E−01 | 1.666E−01 |
| 180 | 17 | 13 | 5 | 35 | 1.041E−03 | 8.040E−04 | 3.864E−04 | 1.295E+00 | 4.806E−01 |
| 181 | 12 | 20 | 4 | 36 | 7.350E−04 | 1.237E−03 | 3.091E−04 | 5.942E−01 | 2.499E−01 |
| 182 | 47 | 29 | 65 | 141 | 2.879E−03 | 1.794E−03 | 5.024E−03 | 1.605E+00 | 2.801E+00 |
| 183 | 78 | 41 | 89 | 208 | 4.777E−03 | 2.536E−03 | 6.878E−03 | 1.884E+00 | 2.713E+00 |
| 184 | 47 | 14 | 6 | 67 | 2.879E−03 | 8.659E−04 | 4.637E−04 | 3.325E+00 | 5.356E−01 |
| 185 | 19 | 7 | 5 | 31 | 1.164E−03 | 4.329E−04 | 3.864E−04 | 2.688E+00 | 8.926E−01 |
| 186 | 34 | 33 | 9 | 76 | 2.082E−03 | 2.041E−03 | 6.956E−04 | 1.020E+00 | 3.408E−01 |
| 187 | 5 | 17 | 13 | 35 | 3.062E−04 | 1.051E−03 | 1.005E−03 | 2.913E−01 | 9.556E−01 |
| 188 | 0 | 0 | 32 | 32 | 0.000E+00 | 0.000E+00 | 2.473E−03 | Undefined | Undefined |

¹Undefined values are those where the denominator corresponds to an undetectable amount of the biomarker.

### X. Additional Considerations

A1. A method of classifying a biological sample obtained from a subject with respect to a plurality of states associated with fetal gestational age, the method comprising:
receiving peptide structure data corresponding to a set of glycoproteins in the biological sample;
inputting quantification data identified from the peptide structure data for a set of peptide structures into one or more machine-learning models trained to identify a fetal gestational age indicator based on the quantification data, wherein the set of the peptide structures comprises at least one peptide structure identified from a plurality of peptide structures in Table 10;
identifying, by the one or more machine-learning models, the fetal gestational age indicator; and
classifying the biological sample with respect to a plurality of states associated with fetal gestational age based upon the identified fetal gestational age indicator.

A2. A method of detecting the presence of one of a plurality of states associated with fetal gestational age, the method comprising:
receiving peptide structure data corresponding to a set of glycoproteins in a biological sample obtained from a subject, wherein the peptide structure data comprises at least one peptide structure identified from a plurality of peptide structures in Table 10;
inputting quantification data identified from the peptide structure data for a set of peptide structures into one or more machine-learning models trained to identify a fetal gestational age indicator based on the quantification data; and
detecting the presence of a corresponding state of the plurality of states associated with fetal gestational age in response to a determination that the identified fetal gestational age indicator falls within a selected range associated with the corresponding state.

A3. The method of aspect A1 or A2, wherein the plurality of states comprises a number of weeks of gestation of a fetus.

A4. The method of any one of aspects A1-A3, wherein the one or more machine-learning models comprises an ensemble learning model.

A5. The method of any one of aspects A1-A4, wherein the ensemble learning model comprises a plurality of decision trees, and wherein a succeeding decision tree of the plurality of decision trees is trained to correct an error of a preceding decision tree of the plurality of decision trees to identify the fetal gestational age indicator.

A6. The method of any one of aspects A1-A5, wherein the one or more machine-learning models comprises one or more of a gradient boosting model, an adaptive boosting (AdaBoost) model, an eXtreme gradient boosting (XGBoost) model, a light gradient boosted machine (LightGBM) model, or a categorical boosting (CatBoost) model.

A7. A method of determining fetal gestational age comprising
receiving peptide structure data corresponding to a set of glycoproteins in the biological sample obtained from a subject, wherein the peptide structure data comprises at least one peptide structure from Table 10;
inputting quantification data for the at least one peptide structure into one or more machine-learning models trained to generate a fetal gestational age score based on the quantification data;
analyzing the quantification data using the one or more machine-learning models to generate a fetal gestational age score, thereby determining a fetal gestational age.

A8. A method of determining a fetal gestational age comprising
detecting at least one peptide structure from Table 10;
inputting a quantification of the at least one detected peptide structure into one or more trained machine-learning models to generate an output probability;
determining if the output probability is above or below a threshold for a classification;
identifying a fetal gestational age classification based on whether the output probability is above or below a threshold for a classification; and
determining a fetal gestational age based upon the fetal gestational age classification.

A9. A method of determining a gestational age of a fetus comprising detecting the presence or amount at least one peptide structure from Table 10, and determining the gestational age of the fetus based upon the presence or amount of the at least one peptide structure from Table 10.

A10. The method of aspect A8 or A9, wherein detecting the at least one peptide structure is performed using mass spectrometry or ELISA.

A11. The method of aspect A10, wherein detecting the at least one peptide structure is performed using MRM mass spectrometry.

A12. The method of any one of aspects A1-A11, wherein the gestational age is over 20 weeks.

A13. The method of any one of aspects A1-A12, wherein the gestational age is over 24 weeks.

A14. The method of any one of aspects A1-A13, wherein the biological sample is maternal serum or plasma.

A15. The method of aspect A14, wherein the biological sample is collected in the second or third trimester of pregnancy.

A16. The method of any one of aspects A1-A15, wherein the at least one peptide structure comprises a glycopeptide.

A17. The method of any one of aspects A1-1A6, wherein the glycoprotein is a pregnancy-specific protein.

A18. The method of any one of aspects A1-A17, wherein the at least one peptide structure comprises at least three peptide structures identified in Table 10.

A19. The method of any one of aspects A1-A18, wherein the at least one peptide structure comprises a peptide consisting of the sequence set forth in SEQ ID NOs:16-21.

A20. The method of any one of aspects A1-A19, further comprising assessing one or more additional clinical indicators for gestational age.

A21. The method of aspect A20, wherein the one or more additional clinical indicators is selected from the group consisting of ultrasound fetal images, and fundal height.

A22. The method of any one of aspects A1-A21, further comprising generating a report that includes the gestational age of the fetus.

A23. A method of training a model to determine a plurality of states associated with fetal gestational age, the method comprising:
receiving quantification data for a panel of peptide structures for a plurality of subjects at varying gestational ages; and
training one or more machine-learning models to determine a state of the plurality of states that corresponds based on the quantification data.

A24. The method of aspect A23, wherein the quantification data comprises at least one of an abundance, a relative abundance, a normalized abundance, a relative quantity, an adjusted quantity, a normalized quantity, a relative concentration, an adjusted concentration, or a normalized concentration.

A25. The method of any one of aspects A23-A24, further comprising pooling samples from multiple individuals stratified by gestational age.

A26. The method of any one of aspects A23-A25, wherein training the machine-learning model to determine the state of the plurality of states comprises training the machine-learning model to generate a class label for the state of the plurality of states.

A27. The method of any one of aspects A23-A26, wherein the one or more machine-learning models comprises an ensemble learning model.

A28. The method of any one of aspects A23-A27, wherein the ensemble learning model comprises a plurality of decision trees, and wherein a succeeding decision tree of the plurality of decision trees is trained to correct an error of a preceding decision tree of the plurality of decision trees to identify the fetal gestational age indicator.

A29. The method of any one of aspects A23-A28, wherein the one or more machine-learning models comprise one or more of a gradient boosting model, an adaptive boosting (AdaBoost) model, an eXtreme gradient boosting (XGBoost) model, a light gradient boosted machine (LightGBM) model, or a categorical boosting (CatBoost) model.

A30. The method of any one of aspects A1-A29, wherein at least one of the peptide structures comprises a glycopeptide.

A31. A composition comprising at least one peptide structure from Table 10.

A32. A composition comprising at least one peptide consisting of the sequence set forth in SEQ ID NO:16-21.

A33. With respect to A1 to A32, the at least one peptide structure includes a glycan bound to the at least one peptide structure peptide in accordance with Tables 10 and 11.
B1. A method of diagnosing an individual with preeclampsia comprising detecting the presence or amount of at least one peptide structure from Table 17 in a biological sample obtained from the individual and thereby diagnosing the individual as having preeclampsia or not having preeclampsia based upon the presence or amount of the at least one peptide structure from Table 17.

B2. A method for determining a risk of an individual for developing preeclampsia comprising:
detecting the presence or amount of at least one peptide structure from Table 17 in a biological sample obtained from the individual and thereby determining the risk of the individual for developing preeclampsia based upon the presence or amount of the at least one peptide structure from Table 17.

B3. The method of aspect B1 or aspect B2, wherein if the individual is determined to have preeclampsia or to have a high risk of preeclampsia, an effective amount of an antihypertensive, a corticosteroid, a beta blocker, or an anticonvulsant to treat preeclampsia is administered to the individual.

B4. A method of treating preeclampsia in an individual comprising detecting the presence or amount of at least one peptide structure from Table 17 in a biological sample and administering an effective amount of one or more of an antihypertensive, a corticosteroid, a beta blocker, or an anticonvulsant to treat preeclampsia based upon the determined risk of preeclampsia.

B5. The method of any one of aspects B1-B4, wherein the amount of at least one peptide structure is none, or below a detection limit.

B6. The method of any one of aspects B1-B5, wherein the preeclampsia is severe preeclampsia.

B7. The method of any one of aspects B1-B6, wherein the biological sample is maternal serum or maternal plasma.

B8. The method of any one of aspects B1-B7, wherein the one or more peptide structure comprises a glycopeptide of a pregnancy-specific protein.

B9. The method of any one of aspects B1-B8, wherein the at least one peptide structure is a glycopeptide.

B10. The method of any one of aspects B1-B9, wherein the at least one peptide structure comprises three or more, five or more, 10 or more, 20 or more, 50 or more, or 100 or more different peptide structures identified in Table 17.

B11. The method of any one of aspects B1-B10, wherein the at least one peptide structure comprises a sequence set forth in SEQ ID NOs: 65-188.

B12. The method of any one of aspects B1-B11 wherein the at least one peptide structure comprises three or more, five or more, 10 or more, 20 or more, 50 or more, or 100 or more different peptide structures comprising a sequence set forth in SEQ ID NOs: 65-188.

B13. The method of any one of aspects B1-B11, wherein the at least one peptide structure comprises one or more, two or more, three or more, four or more, five or more, 10 or more, 20 or more, or 50 or more different peptide structures comprising a sequence set forth in SEQ ID NOs: 65-123.

B14. The method of any one of aspects B1-B11, wherein the at least one peptide structure comprises at least one, at least two, at least three, at least four, at least five, at least six, at least seven, or at least eight different peptide structures comprising a sequence set forth in SEQ ID NOs: 110-118.

B15. The method of any one of aspects B1-B14, wherein the at least one peptide structure comprises a peptide fragment of a glycoprotein identified in Table 16.

B16. The method of aspect B15, wherein the at least one peptide structure comprises a glycopeptide or peptide of a glycoprotein comprising the amino acid sequence set forth in SEQ ID NOs:22-64.

B17. The method of any one of aspects B1-B16, further comprising assessing one or more risk factors or clinical indicators of the individual for preeclampsia.

B18. The method of aspect B17, wherein a clinical indicator of preeclampsia is assessed, and wherein the clinical indicator of preeclampsia is selected from the group consisting of protein in the urine and high blood pressure.

B19. The method of aspect B18, wherein a risk factor for preeclampsia is assessed, and wherein the risk factor for preeclampsia is selected from the group consisting of history of preeclampsia, chronic hypertension, obesity, and multiple pregnancy.

B20. The method of any one of aspects B1-B19, wherein the individual is determined to have a healthy state, wherein the healthy state comprises an absence of preeclampsia and/or a low risk for preeclampsia.

B21. The method of any one of aspects B1-B20, wherein the presence or amount of the at least one peptide structure is detected using western blot, mass spectrometry or ELISA.

B22. The method of aspect B21, wherein the presence or amount of the at least one peptide structure is detected using MS/MS or MRM mass spectrometry.

B23. The method of any one of aspects B1-B22, further comprising comparing the amount of the one or more peptide structure from Table 17 between the biological sample from the individual and a control sample, wherein the control sample is a sample from one or more individuals who do not have preeclampsia.

B24. The method of any one of aspects B1-2B3, wherein the individual is human.

B25. The method of any one of aspects B1-B24, wherein the individual is pregnant.

B26. The method of aspect B22, wherein the individual is in the second or third trimester.

B27. The method of any one of aspects B1-2B4, wherein the individual has recently given birth.

B28. The method of any one of aspects B1-B27, wherein the individual has one or more risk factors associated with preeclampsia.

B29. The method of any one of aspects B1-B28, wherein the at least one peptide structure comprises a peptide sequence and a glycan structure, wherein the glycan structure is attached to a linking site position in the peptide sequence in accordance with Table 17.

B30. The method of aspect B29, wherein the glycan structure of the peptide sequence comprises a glycan structure GL number in accordance with Table 17, wherein the glycan structure comprises a composition in accordance with the glycan structure GL number and Table 18. The glycan structure is associated with the GL number provided in Table 18.

B31. The method of aspect B29, wherein the glycan structure of the peptide sequence comprises a glycan structure GL number in accordance with Table 17, wherein the glycan structure comprises a symbol structure in accordance with the glycan structure GL number and Table 18.

B32. A composition comprising at least one peptide structure set forth in Table 17.

B33. The composition of aspect B32, wherein the at least one peptide structure comprises a peptide sequence and a glycan structure, wherein the glycan structure is attached to a linking site position in the peptide sequence, wherein the peptide sequence and the linking site position in the peptide sequence are in accordance with Table 17.

B34. The composition of aspect B32, wherein the glycan structure of the peptide sequence comprises a glycan structure GL number in accordance with Table 17, wherein the glycan structure comprises a composition in accordance with the glycan structure GL number and Table 18. The glycan structure is associated with the GL number provided in Table 18.

B35. The composition of aspect B33, wherein the glycan structure of the peptide sequence includes a glycan structure GL number in accordance with Table 17, wherein the glycan structure comprises a symbol structure in accordance with the glycan structure GL number and Table 18.

B36. A method of identifying one or more glycopeptide biomarker associated with preeclampsia comprising obtaining a first biological sample from a first set of one or more individuals with preeclampsia and a second control biological sample from a second set of one or more individuals who do not have preeclampsia,
digesting the first biological sample and the second control biological sample with a protease,
enriching the first biological sample and the second control biological sample for at least one glycopeptide,
performing liquid chromatography mass spectrometry (LC/MS) on the first biological sample and the second control biological sample to determine a relative abundance of the glycopeptide, and
comparing the relative abundance between the first biological sample and the second control biological sample to determine a fold change of the glycopeptide, wherein the glycopeptide is identified as a biomarker associated with preeclampsia if the fold change is greater than 2 or less than 0.5.

B37. The method of aspect B36, wherein the fold change of the glycopeptide is calculated by dividing the relative abundance of the glycopeptide for the first biological sample by the relative abundance of the glycopeptide for the second control biological sample.

B38. The method of aspect B37, wherein the glycopeptide is identified as the biomarker associated with preeclampsia if the fold change is greater than 2 or less than 0.5, and the sum of the peptide spectral matches (PSMs) of the glycopeptide for the first biological sample and the second control biological sample was greater than a predetermined number.

B39. The method of any one of aspects B36-B38, further comprising denaturing the first biological sample and the second control biological sample prior to digesting the first biological sample and the second control biological sample.

B40. The method of aspect B39, wherein denaturing the first biological sample and the second control biological sample comprises heating the first biological sample and the second control biological sample to at least 100° C.

B41. The method of aspect B39 or aspect B40, further comprising reducing the first biological sample and the second control biological sample after denaturing the first biological sample and the second control biological sample prior to digesting the first biological sample and the second control biological sample.

B42. The method of aspect B41, wherein reducing the first biological sample and the second control biological sample comprises incubating the first biological sample and the second control biological sample with a reducing agent.

B43. The method of aspect B42, wherein the reducing agent is dithiothreitol (DTT).

B44. The method of any one of aspects B41-B43, further comprising incubating the first biological sample and the second control biological sample with an alkylating agent following the reducing the first biological sample and the second control biological sample, and then, quenching a remaining portion of the alkylating agent with DTT for both the first biological sample and the second control biological sample prior to digesting the first biological sample and the second control biological sample.

B45. The method of any one of aspects B41-B44, wherein the digesting the first biological sample and the second control biological sample with the protease followed the quenching the remaining portion, and then, stopping the digesting by mixing an acid with the protease to form a proteolytic digest.

B46. The method of any one of aspects B36-B44, wherein the enriching for glycopeptides comprises loading the proteolytic digest onto a HILIC (hydrophilic interaction liquid chromatography) column, washing the HILIC column with a wash liquid, and eluting an enriched glycopeptide eluate from the HILIC column with an eluting liquid.

B47. The method of any one of aspects B36-B46, further comprising stratifying the first biological sample and the second control biological sample by gestational age, such that the first set of individuals and the second set of individuals have similar gestational ages.

B48. The method of any one of aspects B36-B47, wherein the first biological sample and the second control biological sample are each pooled from at least three individuals.

B49. The method of any one of aspects B36-B48, wherein the first set of individuals and the second set of individuals are human.

B50. The method of any one of aspects B36-B49, wherein the first set of individuals and the second set of individuals are pregnant.

B51. The method of aspect B50, wherein the first set of individuals and the second set of individuals are in the second or third trimester.

B52. The method of any one of aspects B36-B49, wherein the first set of individuals and the second set of individuals have recently given birth.

B53. The method of any one of aspects B36-B52, wherein the LC/MS comprises MS/MS.

B54. The method of any one of aspects B36-B53, wherein the glycopeptide is determined to be a biomarker of preeclampsia if the fold change is greater than 4 or less than 0.25 in the first biological sample compared to the second control biological sample.

B55. With respect to B1 to B54, the at least one peptide structure includes a glycan bound to the at least one peptide structure peptide in accordance with Tables 17 and 18.

1. A method of classifying a biological sample obtained from a subject with respect to a plurality of states associated with preeclampsia, the method comprising:
receiving peptide structure data corresponding to a set of glycoproteins in the biological sample;
inputting quantification data identified from the peptide structure data for a set of peptide structures into a machine-learning model trained to identify a disease indicator based on the quantification data, wherein the set of peptide structures comprises at least one peptide structure identified from a plurality of peptide structures in Table 3;
identifying, by the machine-learning model, the disease indicator; and
classifying the biological sample with respect to a plurality of states associated with preeclampsia based upon the identified disease indicator.

2. A method of detecting the presence of one of a plurality of states associated with preeclampsia in a subject, the method comprising:
receiving peptide structure data corresponding to a set of glycoproteins in a biological sample obtained from a subject, wherein the peptide structure data comprises at least one peptide structure from Table 3;
inputting quantification data identified from the peptide structure data for a set of peptide structures into a machine-learning model trained to identify a disease indicator based on the quantification data; and
detecting the presence of a corresponding state of the plurality of states associated with preeclampsia in response to a determination that the identified disease indicator falls within a selected range associated with the corresponding state.

3. The method of claim 1 or 2, wherein the plurality of states comprises at least one of a predisposition for preeclampsia, preeclampsia, severe preeclampsia, or a healthy state.

4. The method of any one of claims 1-3, wherein the machine-learning model comprises a logistic regression model.

5. The method of any one of claims 1-4, wherein the machine-learning model was trained by:
generating a log error cost function based on a plurality of disease indicators; and
minimizing the log error cost function based on the plurality of disease indicators and the quantification data.

6. The method of any one of claims 1-5, further comprising administering an effective amount of an antihypertensive, a corticosteroid, a beta blocker, or an anticonvulsant to treat preeclampsia based upon the disease indicator.

7. The method of claim 6, wherein the antihypertensive comprises methyldopa, and the administering the effective amount comprises 0.5-3 gm/day in 2 divided doses.

8. The method of claim 6, wherein the beta blocker comprises labetalol and the administering the effective amount comprises a 20 mg dose intravenously.

9. A method of determining a risk for developing preeclampsia in a subject comprising:
receiving peptide structure data corresponding to a set of glycoproteins in the biological sample obtained from a subject, wherein the peptide structure data comprises at least one peptide structure from Table 3;
inputting quantification data for the at least one peptide structure into a machine-learning model trained to generate a risk score indicative of a risk for developing preeclampsia based on the quantification data;
outputting, by the machine-learning model, the quantification data using the machine learning model to generate a risk score, thereby determining the risk for developing preeclampsia.

10. A method of treating preeclampsia in a subject comprising
receiving peptide structure data corresponding to a set of glycoproteins in the biological sample obtained from a subject, wherein the peptide structure data comprises at least one peptide structure from Table 3;
inputting quantification data for the at least one peptide structure into a machine-learning model trained to generate a risk score indicative of a risk for developing preeclampsia based on the quantification data;
outputting, by the machine-learning model, the quantification data using the machine learning model to generate a risk score,
administering an effective amount of an antihypertensive, a corticosteroid, a beta blocker, or an anticonvulsant to treat preeclampsia based upon the risk score.

11. The method of claim 10, wherein the antihypertensive comprises methyldopa, and the administering the effective amount comprises 0.5-3 gm/day in 2 divided doses.

12. The method of claim 10, wherein the beta blocker comprises labetalol and the administering the effective amount comprises a 20 mg dose intravenously.

13. A method of determining a risk for developing preeclampsia in a subject comprising:
receiving peptide structure data corresponding to a set of glycoproteins in a biological sample;
inputting quantification data identified from the peptide structure data for a set of peptide structures into a machine-learning model trained to identify a disease indicator based on the quantification data, wherein the set of peptide structure data comprises at least one peptide structure identified from a plurality of peptide structures in Table 3;
identifying, by the machine-learning model, the disease indicator; and
determining a risk for preeclampsia based upon the identified disease indicator.

14. A method of treating preeclampsia in a subject comprising:
receiving peptide structure data corresponding to a set of glycoproteins in the biological sample;
inputting quantification data identified from the peptide structure data for a set of peptide structures into a machine-learning model trained to identify a disease indicator based on the quantification data, wherein the peptide structure data comprises at least one peptide structure identified from a plurality of peptide structures in Table 3;
identifying, by the machine-learning model, the disease indicator;
determining a risk for preeclampsia based upon the identified disease indicator; and
administering an effective amount of an antihypertensive, a corticosteroid, a beta blocker, or an anticonvulsant to treat preeclampsia based upon the risk score.

15. The method of any one of claim 14, wherein the antihypertensive comprises methyldopa, and the administering the effective amount comprises 0.5-3 gm/day in 2 divided doses.

16. The method of any one of claim 14, wherein the beta blocker comprises labetalol and the administering the effective amount comprises a 20 mg dose intravenously.

17. A method of treating preeclampsia in an individual comprising detecting the presence or amount of at least one peptide structure, wherein the at least one peptide structure comprises at least one peptide structure from Table 3, and administering an effective amount of an antihypertensive, a corticosteroid, a beta blocker, or an anticonvulsant to treat preeclampsia based upon the presence or amount of the peptide structure.

18. A method of treating preeclampsia in an individual comprising detecting a presence or amount of at least one peptide structure to determine a risk of preeclampsia, wherein the at least one peptide structure comprises at least one peptide structure from Table 3, and administering an effective amount of an antihypertensive, a corticosteroid, a beta blocker, or an anticonvulsant to treat preeclampsia based upon the determined risk of preeclampsia.

19. A method of diagnosing an individual with preeclampsia or risk of pre-term birth comprising detecting a presence or amount of at least one peptide structure, wherein the at least one peptide structure comprises at least one peptide structure from Table 3, and diagnosing the individual with preeclampsia or risk of pre-term birth based upon the presence or amount of the at least one peptide structure.

20. A method of determining a risk for developing preeclampsia or risk of pre-term birth comprising detecting a presence or amount of at least one peptide structure and determining the risk for developing preeclampsia or risk of pre-term birth based upon the presence or amount of the at least one peptide structure, wherein the at least one peptide structure comprises at least one peptide structure from Table 3.

21. A method of diagnosing an individual with preeclampsia comprising
detecting the presence or amount of at least one peptide structure structures from Table 3;
inputting a quantification of the detected at least one peptide structure into a machine-learning model trained to generate a class label,
determining if the class label is above or below a threshold for a classification;
identifying a diagnostic classification for the individual based on whether the class label is above or below a threshold for the classification; and
diagnosing the individual as having preeclampsia based on the diagnostic classification.

22. The method of any one of claims 14-21, wherein the presence or amount of the at least one peptide structure is detected using mass spectrometry or ELISA.

23. The method of claim 22, wherein the presence or amount of the at least one peptide structure is detected using MRM mass spectrometry.

24. The method of any one of claims 14-23, wherein the amount of at least one peptide structure is none, or below a detection limit.

25. The method of any one of claims 14-24, wherein the preeclampsia is severe preeclampsia.

26. The method of any one of claims 1-19, wherein the biological sample is maternal serum or maternal plasma.

27. The method of any one of claims 1-26, wherein the one or more peptide structure comprises a glycopeptide of a pregnancy-specific protein.

28. The method of any one of claims 1-27, wherein the at least one peptide structure comprises three or more peptide structures identified in Table 3.

29. The method of any one of claims 1-28 wherein the at least one peptide structure comprises the sequence set forth in SEQ ID NOs:5-12.

30. The method of any one of claims 1-29, further comprising assessing one or more risk factors or clinical indicators of preeclampsia.

31. The method of claim 30, wherein a clinical indicator of preeclampsia is assessed, and wherein the clinical indicator of preeclampsia is selected from the group consisting of protein in the urine and high blood pressure.

32. The method of claim 30, wherein a risk factor for preeclampsia is assessed, and wherein the risk factor for preeclampsia is selected from the group consisting of history of preeclampsia, chronic hypertension, obesity, and multiple pregnancy.

33. The method of any one of claims 1-32, wherein the individual is determined have a healthy state, wherein a healthy state comprises the absence of preeclampsia and/or a low risk for preeclampsia.

34. The method of any one of claims 1-33, further comprising diagnosing a placental development problem.

35. The method of any one of claims 1-3, further comprising generating a report that includes a diagnosis based on the corresponding state detected for the subject.

36. A method of training a model to diagnose a subject with one of a plurality of states associated with preeclampsia, the method comprising:
receiving quantification data for a panel of peptide structures for a plurality of subjects diagnosed with the plurality of states associated with preeclampsia; and
training a machine-learning model to determine a state of the plurality of states a biological sample from the subject based on the quantification data.

37. The method of claim 1-10 and 36, wherein the quantification data comprises at least one of an abundance, a relative abundance, a normalized abundance, a relative quantity, an adjusted quantity, a normalized quantity, a relative concentration, an adjusted concentration, or a normalized concentration.

38. The method of claim 36 or claim 37, wherein the machine-learning model is trained using random forest or logical progression training methods.

39. The method of any one of claims 36-38, further comprising pooling samples from multiple individuals stratified by gestational age.

40. The method of any one of claims 36-39, wherein training the machine-learning model to determine the state of the plurality of states comprises training the machine-learning model to generate a class label for the state of the plurality of states.

41. The method of any one of claims 36-40, wherein the machine-learning model comprises a logistic regression model.

42. The method of claim 41, wherein the machine-learning model was further trained by:
generating a log error cost function based on the plurality of states associated with preeclampsia; and
minimizing the log error cost function based on the plurality of states associated with preeclampsia and the determined state of the plurality of states.

43. The method of claim 42, wherein the machine-learning model was further trained by:
generating a cost function based on the plurality of states associated with preeclampsia; and
minimizing the cost function based on the plurality of states associated with preeclampsia and the determined state of the plurality of states.

44. The method of claim 43, wherein the cost function comprises a rectified linear unit (ReLU) cost function.

45. The method of any one of claims 1-44, wherein at least one of the peptide structures comprises a glycopeptide.

46. A composition comprising one or more peptide structures from Table 3.

47. A composition comprising one or more peptides comprising the sequence set forth in SEQ ID NOs: 5-12.

48. A method of diagnosing an individual with preeclampsia comprising detecting the presence or amount of at least one peptide structure from Table 17 in a biological sample obtained from the individual and thereby diagnosing the individual as having preeclampsia or not having preeclampsia based upon the presence or amount of the at least one peptide structure from Table 17.

49. The method of any of one of claims 1-45 and 47, in which the at least one peptide structure includes a glycan bound to the at least one peptide structure peptide in accordance with Tables 3 and 4.
